SEQUENCE LISTING (62 sequences)

SEQ ID NO: 1 - DNA, 85915 bp, Streptomyces bikiniensis
ggggcccgccggacggggctgcccggctctcggcggtgcccggtgggccgggtgcgggctcgcccgcggcgagatgctccaggacctccgccagttcccggcaggcgcggcgtaccgagcggcgggtcgcacgctgctccgtgatgacggaggcgagaagcagggcggtcagtgcggcggaaccgttgaacgcctggagcttggccatgatctcgacgtccgagaggtggaggaaccctccccgtccggcgttcgcctcgaaggtggcgagcacggaggcgaagagcgcgcagagcatgcttccggtgagctggaagcggagcgccgcccagatcagcagggggaagacgaggaagagcatgcccaccgggctgagcacggccatgggcatgaggatcagggtcgcgagacccagcagggccgcctccttccagcgccgtacgcggaaccgtccggccggccccgcgaggacgaggaggagcggggcgacgagcagcacccccatcgtgtcgcccacccaccaggccagccagacgggccagaactcggtcgtgtccagggagctcttcgccacctgcagtccgaccccggcggtcgcgctgatcagcatggcgccgaacccgccgaggaagaccagggagagtccgtcccgcagccgtgccatgtcgagccggaagccggcccgtgtcagcagcaggaaggcgcagagcggcgcgacggtgttgctgaccacggtgaccacggtggtgggccccggcgtggtgagggaggcgatgacgaggaaggagccgagggcgatcccgggccagacgcgcgcgccgagcagcagcagggcggcgacggcgacgccggtgggaggccagatgggggtgaccaccacgccttcgacgacgaggcggcccatcaggccgagtcgtccggccgcgtagtagagcaccgccacggccagcgacatcagcgccgtcgccgcgggggaccggtactgccgaatatccaacacgtctgccatcagacaccgaccgggttgccgcgcccctgcatggccgcctgcgtgtcggggacacgccgcccggcccggaccgggccgcggggaggaccggcccgaggcgggtcccctcgctcactccccggtcgccggccggggcggaccgccgtcgtggccgacgacgaggacggccgcgtcgtcctcgtgccccaccgtggccgccagtcgcatgacggcggtggcgagcgcgtcgacgtccagcccggcgacggccgtgatcccggcgagtctcgtcacccggtcgagaccctcgtcgatgtggagcgagggcccctccacgaccccgtcggtcaggaggacgaagacgccgtcggcggtgagccggtggcgcgtggccgggtagtcggcgccgcgcagcacgccgagaggaggcccgccctcgctgtcgtcgatgccggagcggccgtcggcggtggcccagatgtgggggatgtggccggcccgggcgcattccagggtgccggcggcggggtcgagccgcaggaaggtgcaggtggcgaagaggtcggcgccgagggagacgagcaggtcgttggtgcggccgagcagctctcccgggtccgcggtgacggaggccagcgcgcgcagtgccacgcggacctgtcccatgaaggcggcggcctcgatgttgtgtccctggacgtcgccgatggacatgccgatccgcccgccaggcagggggaaggcgtcgtaccagtcgccgccgacgttgagcccgtggttggcgggctcgtaccggacggccagccgggcacccggaagactgggcaggtccgagggcagcatgccgcgctgcagggccacggcgagctcgacacgggaccgctgcgtctcggccagctcccgcgccctggcggtcagcgacccgagccggaccagaaggtcctcgctgctgtcggccgggcgtctgcggaacatcgatcactccgacgtcacgacaatcctcgcatcactccgtcccgtctccagcacgcgggaccacaggggaccaccccggtacgaacaggtccttcccactgtgcccggagggggcggggtccgcatctcatcggcgggagagcgcggtggatcccagggggcccgctcaggtcaccgaaaacgagcaaacgttcgataatgtggtcgcgccggtctgtgcggccgttcagcgttcgacggtcactgcggcggccgcgatgccgtcgcgcaccagccaccggccggtgaacccgcccagctgccgtccgtcgaccgtcggcccgggcacgagcagccgggccctgaaacggccgcccggctggaagtcgacggccgcctccccgaagtcgagccggctccgggtgagcgggaaccacgccttgtagacggcctccttggcgcagaacagcagcctgtcccacgggatgccgggccgggccagtgccagagcggccagcggcccgcgctcatccgcggtcgtcaccgcctcgaagacgccgtgcggcagcggaagggcgggctcggcgtcgatgcccagagacgccacgtccgcctcggacgccaccgtcgcggcgcggtagccggcgcagtgggtcatgctgccgacgacaccgggcggccatcccggcgcaccccgctcaccgggcacgagggccaccggcccccaccccagaccggccagcgcgcgtcgcgcgcagaagcgcacggaggtgaactccctgcgtcgcttgtcgaccgcgcgcccgatcgccttctcctcgtcggcgaacagccgcgcctcggggtaccggtcggcatccaggacgtcgccgaagacgtccaccgacaccgcggcgggcggcagcagtgcgcagatcagcccggcccaccgcggcaccgacacggcggcgtcggcctcggcacggtcgtgcacgcacgccgacgccgcggcgtcggcgagggggcccgtgcggctgccgtccccggaccgccgcgccgaggacgccgccggcgggaagccgtccctcacacgggcaccgtggcgtcgtcgtgcgtgcgtccgagcgcgttcagccgggccagctggccatcggagagagcgatgcccgacgccgcgacgttctccctcagatgcgcgggcgagctggtgccggggatgggaatgaccgccggcgagcggtgcagcagccacgcgagcgccacctgaccggccgacacctccagctcggtggcgatgtcggccaccgggctccccccggcggcgagcgcaccgcgggcgatcggcagccaggcgatgaacgcgatctcgtgcttctcgcagtactcgaccacctcgtcgttgcgccggtcggtcaggttgtacacgttctgcacgctggccacggtgatgtgctcacgggcggcctccacttcccggacggtgaccttggacagcccgatgtgccggatcttcccttcgtcctgcagttccccgagcgcaccgaactgctcggccgccggcaccttcggatcgatccggtgcagctggaagaggtcgatgcggtcgagtcgcagcctgcgcaggctcagctcggcctgctggcggaggtactccggtcggccgcacggcacccactggtcgggacggggccggcactgtccggccttcgtcgcgatcacgaggccgtcggggtacgggcgcagtgcttcggccagcagctcctcgttgcttcccagcccgtaggaatcggccgtgtcgatgaaggacacaccgaggtccacggccagccgggccgtgctgatcgcagcctcccggtcctccggcgggccccagtaccccggaccggtcagccggagggcgccgaaacccaaccggtgtaccgagaggtcgcctcccagagggaaggtggtcctgccgggctgtgccatgcgttcctcctggacgacgtccgtgcactcgggtggggcgcgtggtgacctcggtgggggcgggccgatgccgaccgtccgaacacggtggatcccccggttcagggagccgtcgtggagggcacccgctcgtcggcccgtcgggtcacctcgtgtccccgctccagagtgagccgcacgatgtcgcagacccgacggatgtcctcgcgagagacggtggggccggtgggcagggcgaggaccttccgcgcgagccgttcggtgtggggcagggagaccggccgctccgagcggtagggctccatctcgtggcatccgggggagaagtaccgctggcacatgatgttctccgccctcagcacctcgtccagcaggtcccggtggaccccggtcaccgcggcgtcgacctccacgaccagatagtggtagttgttgcgctcggcacggtcgaactccatgaccttcagcccggcgagcccggcgagttccgaccggtagtcgtcgtggttggcctcgttccggcgaaccgtctcctcgaaggcgtcgagcgaggtgagtcccatcgcggcggcggcttcggtcatcttcccgttggtgccggtctccgtggacacccgcccctgggtgaatccgaagttgtgcatcgcgcggacccgctgggccagcctctcgtcatcggtcaccaccgcgccgccctcgaaggcgttgaccaccttggtggcgtggaagctgaacacctcggcttgtccgaagccgccgatcggctgcccttccgacgtgcagccgagtccgtgcgcggcatcgtagaagagcctgagaccgtgatcggccgcgaccttcgccagccggtcgaccgcgcagggccgaccccacagatggaccggcacgaccgcactcgtacggggggtgatcgccgcctcgatcagatccgggtccaggcagttggtggccgggtcgatgtcgacgaagaccggggtcagacccagccagcggaacgcctgggcggtcgccgggaaggtcagcgacggcatgatgacctcgcccgacagatcggcggcgcgggccagcaactggagggcgacggtcgcgttgcaggtcgacacgcagtagcgcacgccggcgagttccgccacgcgctgctcgaactcccgggccagcggcccgccgttggtcagccactggtggtccagcgcccagttcacacggtccaggaaccgcgctcggtcgcccacgttgggccgccccacgtgaagtggttgcaggaacgccgacgggccgccgaacacggcgagatcaccgagattgcgtttcatggtcatacctccccggtgcacgggacggtgcacccggcagcagccggcacaggacgtggacgtaccggagggacaggggccgcagcgcacgacggcgtctccgtcgtgcgtacgcgaaaggggcgcacggaacggatcagtgctcgcggcgccagtagacgcccgtcacgtccaccgtctcgatcggctcgtcgatgccgtgttcgctccggtagtcgtggacggcctgcttgcacgccgggatcaggtagtcgtccacgatcacgaacccgcccacggacagcttggggtagaggttgaccagcgcgtccctcgtcgactcgtacaggtcgccgtccacccgcagcaccgcgagccggtcgatcggcgcggtcggcagcgtgtcggcgaacatgccgggcaggaacctgacctgctcgtccagcaggccgtagcgggcgaagttctccctcacctgctcctcggaacagctcagtacccagttcaggtggtggaactccatcgcccggtcgagcgggtggctgtcctcggacgtcaccgggacaccggcgaacgagtcggcgagccacacggtgcggtcctccacaccgtgcgccttgaggagcgcgcgcatcaggatgcaggcgccgccgcgccacacgccggtctcgatgagatcaccgggaacatcgtcctggaggaccctctccacgcacctctggatgttgtccaggcgttgcaggccgatcatggtgtgcgcgaccgtcggcacgtccagtccttcggcccggtggtcggcgtcgaaggtcccgcgctcgacgtccgggccctccgcccccggacgcatgcggtccgccagttccttgtccaggacgttcatcccgaccgggaaggtcgggtgatccccgtagatggtgttcgacacgacgttcttcatgaggtccacgtagagctcgcgggtctgcacggtcgacttacctccggctagatatcgaacagagagagaatgtgcggggcgacggcgtccgcggacgcggtgggagccgtcgcggtcgggtcacctcaggaagccgcgcgcgtcctcccagccggccacgacatcggcctccagctggttgagccgcgccgccaccacctggtcgaacccgtccatgaagtactcgtcaccggcgtgcggcgctatcagacggccgtcgtccacgaaccgctcgacgacctccgtcagggaggtgcccggccccacccggcccgcgacgtaccggtccgctccggtgagatccgggaaccccgcctcccggtacaggtacacgtcacccagcaggtccacctgtacggacacctggggatgcgcggaccggcgcatggttccgggcctgatcctgagcagctcggcgtccgcgccggtcttcaggctgtgcaacgcgtagccgtagtcgatgttcagcgtgggggtccgccggctcaccgcctcctcgaacgcgagcagcccctcctggagttcggcgcgttcggcctcgaacagcctgccgtcctcccggccgctgtagtcctcgcgcacgttcaggaagtccaccggccggtccggcgcggcctcgttcaggtcggcgatgaagtcgacgaggtcgagcagccggtggacgcgcccgggcagcacgatgtagctgagcccgagccggatcgggctctcccgctcggaccgcagctgctggaagcgccgcaggttggcgcggacccttccgaaggcggcgcgcttgcccgtggtctcctcgtactcctcgtcgttcaagccgtacagcgaggtgcgcaccgcgtgcaggtcccacaccccgggctgcctgtccagcgtccgctcggtgagcgcgaacgagttcgtgtacacggtcggccgcagcccgcgagccgccgcacggctgctgagcgcgcccaggcccgggttggtgaggggctccagaccgcccgagaagtacatggcgtacgggttgccggccgggatctcgtcgatgacggacgcgaacatcgcgttgccggactccagcgccgacgggtcgtaccgcgcaccggtcacccggacgcagaagtggcagcggaacatgcagctcggccccgggtacaggcccacgacataggggaaggccggcttgtgcgcgagcgccgcgtcgaagaccccccgtcgctccagcggcagcagggtgttctgccagtacttgcccgccggcccgttctccacggcgtggcgcagctccgggacacggccgaagaggccgaggagccggccgaaggaggcccggtccacgtccagcatccggcgtgcctcctccaggggcgtgaaggggtggttcccgtagcgctcggccagccgcacgagccggcgcgcgaccgtcgggccctcgtcgacaccgacgcggccgtcggagaccagggcgcggtgcaccgtgtggaccgctgccggcaggtcggcgccggggcgggcgcacagggcgaccggatcggccgcggtggaggcgtgcgaggtcatccttcgtgtccaatcgtctgcgtccggagggtcggtccgttccccgggaaacgcggcggggcggtcaccgggacggcaccgtgacggatgtggtcagggacgtctcggccgcggacgggcccacgtgtacggccctcctgccggtgcccggcttccacgtgccggcggcggtgtcccagtagcgcagttgctgctcgtcgacggggacggtgacccgccgctgttctcccggccggagggtgaccttggtgaatccggcgagtttgcgcagcgcctggggggcctgggtgtccggacttgccccgaggtagacctggacggtctccctgcccgcccggtcaccggtgttgcggacggtgaccgtggcctccagcccccgggcggtgcgcgccacggacaggtcgctgtaggcgaaggtggtgtacgacaggccgtacccgaacgggaagagcggggccacgccggtccggtcgtaccaccggtagccgacatccagtccctcggaataggtctccttgccgtcgacacccgggtagcggtgcgggtcaccggccatgggatggccggtctcggtggccgggaaggtctgggtcagccggccgctgggattcgcgtcgccgaacagcagggcagcggtggcctccgcgccctcctgccccgggtaccacatctccagtacggcggccgtctcacgcagccagggcatgagcaccgaggaaccggtgttgaggacgacgatcgtgcgggggttgaccttggtgatcgccgcgatcagctcgtcctgtcggccgggcagggacagcgaggaccggtccacgccctcggcactgtcgtcgtgggcgaagacgacggcggtgcgggcctcggccgccgccgcgacggcgcggtcgaagtcgtcccgggcagcctcgggagtgacccagctcagctccaccgacagcggggtctgttcgaaggcccagccgttcatggtcaccgcgtgggtgccgcgggtcagccgcatcgtggtggtgacggtgccgaacgcctcggtgccgaggacagcggactgccccgcgatctgcaggttggcgaccccgccgacggcggtgaaggcgatcttgtagtcgccgtcggcggggacggtgagccgaccctcgtagaagacgccctggccgctgggatccagttgcttcccgctcgtgaaggcgggggtgagcgagctttcgggcacgggcacgccgaccaggtcctcaccgggctcgtggacgacccgggccttctcgccggcgcgccgtgcgagggcgtcgacgggtgcggtcgcccggtcgggtatgacgtgcgcgctgccgttgccggtgaccttggggtgcttggcgctgttgccgatgacggcgatgtccgtggcgtcctcaccggtcaggggcagcgtccggcgctcgttgcgcagcagcaccccgccgttctcggcgatggtgcgggccgccgcgcggccggcctcgggatcacgctgcgggcgctcggtcgccttgccgtcgagcaggccgaagcgctccatctggccgaggatgcggacgacggaccgatcgagcgtcgcctcggggacgctgccgtcccgcacggcggccctgagcgcggaggagaagtacttcgattccggcaccggctgccccagggtcagctcgacgcccagttcctggtcgaggcccctggtgatgtcgccggtggcgtgggtcgccagccagtcggacaccacccagccgcggaagtcccactgttcgcgcagcacctcctgcaacaggtgctcgttcccgcatgcgtgcgcgccgttgaccttgttgtacgagcacatcaccgaggcggcgcccgcccgcacggcgctgcggaaggccggcagctccacctcctggagcgtctgctcgtcgaccacggcgtcgatggtctcccgctggtactcctggttgttcgcggcgaagtgcttgaccgtggccatcaggccctggttctggatgccgcggacctcgtgggcggccatccgggacgacagcagaggatcctcgctgtaggtctcgaagttccgccccgcgtgcggcacccggatgacgttggtcatgggaccgaggacgatgtcctgtccgagggcccgtccctcccggcccaggacggtgccgtactcctcggcgaggcgttcgtcgaaggtggcggcgagcgccacgggggtgggcatggcggtggcggtaccgccgagcagacggatgccgaccgggccgtcggcggtgcgcagctcggggatgccgagccggggcacgcccggcagatagccgatgccggtcatggtcggcccgcccacgggcccggtggtccagtggacgaacgagatcttctcgtcgagggtcatcttcgccaccagcccgcgcacccgcgccgtcccgggctcggcggccggagcaccggcggcggagggggcggtgagcagaccgccgacgcacagggccgcgagcgcggatccgacggtgcgccggatgcggcgacccgtcctcgtcgtgccgaacagcatgctgacggacgtcctttctgccgaggttgccgtcctcatcggggcgggaacgtttctgtccgtgccgccatgatcaccagcccacgagcatcgtgcgcgggccccgcaccaccatctccgtcttccactcgacgggctgggcgaggtgcagcccggggagttcgtcgagcagggtccgcagcgcctcctggagctccagccgcgcgagggacgcgccgaggcagtgatgcggaccgtgtccgtaccccaggtgccggccggcgtcgtcgcgggtgatgtcgagcacgccgggcgagcggaagcgcagggcgtcccggttggcggcgttcatctggaccagcaccggatcccccgcccgcaccagcgtcccgcccacctcgacgtcctcggtggcgtagcgcgggaatcccgcctggctgccgagcggaacgaaccgcgtcagctcttccaccgcgttgtccagcaggtcaggacggtcccggaggagggccagctggtccggctgttcgagcaggaccaggacgaagttgctgatctggctggccgtggtctcgtgcccggcgaacaggaggaagacgatcaggtcgaccaactcctcctgcgacagccggccctgggcgtcacgggcctcgacgagcgccgagacgagatcgtctcgcggcgcggcgcggcgggccgtgatcaggtccgccagatagccggtcagctcgccggccagccgcacatggtcctcggcggtgagcgagctggtggacatcgctatctcgcaccagccgcggaacaggtcacggtcctcctcgggcacgcccagcaggccgcagatgaccgccacgggaagggggacggcgtagtggtccacgaggtcgacgggcgatccgagcgccgtcatgtcccggagcagcgacgccgtcatccttcggacgtgcggccgcatggcctccacgcggcgcggggtgaacgccttggtgaccagcccgcgcagcctggtgtggtccggcgcgtccatgccgatgatgccgttgggcgtccgcagctcccgcatgcgcggcgcgtcgttctccgcctccgcggcgtggctgaaccgccggtcgccgaggacgaaacgcgcgtcgtcgtaacgggagacgagccagcccggctccccgtacggcatgtggacccagaggagtccttcgctcttccgcgcctgctcgtacgcctcgtccaggtccagtccctccgggcggccgaagggataggagacgggtgcggtcttcgtgctggtcaaggggagccccatatcgcgtcgttcgggtgcttaatcgtcgtgggccggatcatgcggccgagccgccccaggtgacggggaccgcctcgggggtgcgcaggagggagccggcccgccagcgaagctcactcgcggggacggacagccggagctccggcaggcgctcgaccagggtgctcagcacgacctgcagttcgagccgggccaggggggcgcccatgcagtagtgggcgccgtgaccgaagccgaggtgcgggttctgggcacgggccaggtcgagggaggcggcaccgggaaacgccgtgtcgtcgaggttcgcggacgcgatcgaggtgatgaccgcctcgcccgcgcggatgagcgtgccgccgatctcgacgtcctccaaggcgatgcgggcgaacccgtccagggtgctcagcggggtgaagcgcagcagctcctcgacggcggagggaaccaggtcaggttcggtcaccaggcgctcgtagaggcggcggtcgtcgagcaggaggaggacgaaattgccgatctgggtggcggtcgtctcgtatccgctgatcagcaggccggagcccatcatgaggagctcggcctcggagagccggtcgccttcgtcacgggcctggaccatggtgctgaggaagtcgtcggtgggccgggcgcggcgatcggccacgaggtcggccatgtaggcggagaagtcctcggtcgcccgctgcacttcggcgggccccagggaggaggacaccagcgcctcggagaagtcccggaagaggtggcggtcgtcgtaggagacgcccagcagtccgcagatcacggagatcgacacgggcagcgccaggtcttccatgacctcgccggacggctcgtcgcgcgcgaccatgccgtcgagcagttcatcggtgaaccgtgagacccgcgggcgcagttcctccacccggcgggcggtgaacgccttcccggcgagccgccggagcctggtgtgtccgggcgggtccatggagatgagaccgccggcgggcagcgggtactcggtgggccggggcacgtcacgtccggccccggcctccatgctgaaccgcgggtctcccaggaccgccctcacgtcggcgtgtcgggtcagcagccaggcctcaccgccgtacggcatgcggatccgggacacgggctcgtgctcccgcagacggctgtagagcggatccagcgcgatgccctcgggcggggagaaggggtacgggcgagtccggtggaccggacagctagacgtcatggacactctctcggtcgtacggggcggcggcagagctcgaagggtgcgggcggccgacggcgagcccggtcgccgtccggatcgtccggcacaccgcttcggcctgctcggtgaggtagaagtggcctccggggaaggaacggaacgccgtctccccggtggtcagcccgtgccaggccagggctccgtcggtggggacgtgcggatccgcgcttcccgtcagcacggtgatcgggcaggcgagcggagctcccggacgccacgtgtaggtggcggcggcccggtagtcgttgcggatcgccggcagggccatccgcaggacctcctcgtcggccagcacccgggggtcggtgccttgcagttcggccagcttggcgaccagccggtcgtcgctcagcaggtgcgcggtggtccggtgggcgacggtcggggcgacgcggccggagacgaacagatgcgccagcctgctctccggcgcctgttccgcgagccggcgggcggtctcgaaggccaccagcgaccccaggctgtgaccgaagagcgccaccggccggtccagccaggggccgagcgctccggcgacgtgcgtcaccagagcgtcgacggagtccacgaacggctccagccgccggtcctgccgcccgggatactggacggcgagcacctcgacccggtccggcagcagacaggtgaaggggaggaaggcgctggcgctgccgccggcgtgcggaaggcacacgagccggagcgcggggtcggcgacgggccggtagcggcgcagccagaggtcgccggcgttggtggagccgggcgccgtcgtggcgggcccctcgttcatgcgtgatccgcttccggacgtcacggtgtcagcgccttccaccacgtgccgttgtcccggtaccagtcgacggtctccgcgagcccgcgatcgaaggcgacgcggggagcccatcccagctcctcggtgatcttccgggtgtccacgcagtagcgacggtcgtgcgcggcccggtccggcacccggtccaccacggaccagtcggcaccgaagacgccgagaagacgacgggtgaggtcgacgttggacagttccgtcccgccgccgatgttgtacacctctccggcccgcccccgggcggcgaccatggcgatgccccggcagtggtcgtccacgtgcagccagtcgcgccggtggaggccgtccccgtacaggggaaggcgcaggccctccagcagatgggtcacgaagagcgggacgaccttctcggggtgctggtgggggccgtagttgttggagcagcgcgtcacccggacgtcgaggccgtgggtacggtggaaggcgagcgccaggaggtcggaggaggccttggacgccgcgtagggggagttcgggtccagcggatcggcctcggtggaggagccctcggggatcgatccgtacacctcgtcggtggagacgtggacgaagcggctgacgcccgcttcgagcgccgaccgcagcagggtctgcgtccccagcacgttggtggtgacgaaggcggcggagtccgtgatcgagcggtccacgtgcgactcggcggcgaagtgcaccaccaggtcggtgccgcgcagggcctgggccaccgtgggcgggtcgcagatgtcgccgtgcaggaaggtgtgacgggggtggccggcgacctcggcgaggttctcggtgttcccggcgtacgtgagcttgtccagcgagaggacgtgggcgccggccagctccggataggagcccgacaggagctgtctgacgaagtgcgagccgatgaagcccgctgccccggtcaccaggacgcgcatccttgatccgcctttctgccgtgctgtgtgggggaggtgtggtcaaccggtgcgcgtcgccccgagtcccgaggcgacctccatgaggtagtccccgtagctggaggcgctcagctcctcacccagccggtaacaggtgtccgcgtcgatgaagcccatgcgcagcgcgatctcctcgagacaggcgatgcgtacgccctggcgctgctcgaggagctggacgtactgacccgcctggagcagggagtcgtgggttcccatgtcgagccaggcgaatccacgtccgagctgggtcagccgggcgcggccctgctccagataggacaggttgatgtcggtgatctccaactcgcctcgggctgagggcttgaggtccttcgccagctccaccacgtcgttgtcgtagaagtacagcccggtgacggcgaggttggagcgaggacggctcggcttctcctccagggacaacaggtgcccctgctcgtcgatctcggcgacgccgtagcgctcgggcgacttggacggatagccgaacagttcacatccgtcgagacgtcgcagggagtgcttcaggacggtggagaatccggggccgtggaagacgttgtcgcccagaatgagcgccacccggtcgttcccgatgtgcttgtcgccgaccaggaacgcgtcggcgacaccgcgcggctcgtcctggaccgcgtagtcgagctggataccgaggcgcgagccgtcgccgagcatgacctggaacgtctccacgtgctggtgcgaggagatgatgaggatgtcctggattcccgccagcatcagcaccgacagcggataatagatcatgggtttgtcataaacgggcaactgctgtttggaaagcgcaccggtcagcggctgcagtcgagtggcgcttcccccggcgaggattatccccctcactccggggcgtttcagcggtgtctcgaacacggttggtcctccgtggtcacatggccgatatggggggtgaagacactgtcctgagaggccggcggaccggctgtcgcctcgcggacacagcggcttaatgcattcaccccgccccgggaccgtcatccgagaagaaggaatgcggtgtcgtgggaaccgacgtccaggagttcctttcgggccgcggaacggcggcgcggagattctgaaccgcgggggattccagggcggtggcagggaagggaaccaccgccgcgccatctctcccggaacgttccgcaagcggcgggccgtgcttcggacggcctatctctgcgcctgttgctgttcctgccaggcctgataggtcggcagcagaccgagccgttcggccgtcgcgagggtcggggcgttctcgtccctgtcggagagcagcggctcgatgtcgtccggccacgcgattccgaggtcggggtcgagcggattgacggagtgctcgcgtgcgggagcgtatccggaggagcagaggtagacgagcgtggcgtcgtcggtgagggagaggaaggcacggcccagtcccgcggtcagatagacggcggtgttgcgttccgcgtccatgggcacgatctcccagcgcccgaaggtcggcgaaccgatgcggacgtccacgacgacgtcgaggccggctccccgcacgcacacgctgtacttggcctgacctggcgggatctcggtgtagtggatcccgcgcagcgcgccgcggtgcgacaccgcgacattgacctgggccaccgggaagtcatggccgaacgcctgacggaagctctcgccccggaaccactcgtgggacctcccgcggtggtcggagtggatgacgggttcctgcgaccaggccccctcgatgctgagtggatgcatcgcgccttctccttcggaccgatgggtggggtgcggggcgggccgggggcacggccgagccggtcagctggagcgtctccagtaggaaccctggcgatcgatctggtggatctcgtccgtgatgccgtgctcgtcccggaactcgtggacggcctgccggcacgccggaatgcagtagtcgtcgacgatgacgtacccgccgtccgacaccttgtggtagaggttggtgagaacctccctggtggctgcgtacgagtccccgtcgagcctcagcaccgcgagcctctcgatgggcgcggtgggcatcgtgtccttgaaccagcccgggaggaagcggacctggtcgtccaggagtccgtagcgcgcgaagttccccttcacggtctccacgtcgaccgggatgctgaggacgtcgttgtactggccgaggtcgatgtcgacgtccagctggtggtcgtcctcggtggtcttggggaaaccctggaaggagtccgcgacccacaccttccggtcccgcacgccgtgcgcccggaagacgccgcgggcgaagatgcaggccccgccccgccagaccccggtctccgcgaagtcccccggcacgccgtcgcgcagcacgtcctccaggcacttctgcaggttgtcgagccgcttgagaccgaccatcgagtgcgccacgcgcggaaagtcctcacccaccgagcgcagttccgcggaatacgagctgctggtgatgaggccggcgacattggtctggtcctcgtaaatcatgttcgtcacgaccttcttcagaaggtcgagatacaggtccgcttcggcagctatgacagtcattttcctcacttacgggtagcagtgcccagcgggcggctcgttcaggacgggggccgccggggctgaattccctgtgtccacacagatgaggtggatgaggtggatgaggtagccatctaaccccagtgatcagattcgggcaagggtcgaaaacgagccacgtcttatgtcgatcctgtccggaagcgaggggcatatggtgcagtggcgactgcggccgatctggctgatccttgctgcggcgctgaccgtgtgcttgcatgttggacgtagatcaccttctcccgattgcattcagggtgaggaaatccatgaaatcttcaaaagtcgttcacagccgtcctgcggaagcgggcgtcgcatggcccgtcgcgcgaacctgcccctttacgctccctgatcagtacgcagagaagcgcaagaacgagcccatatgccgggctcaggtctgggacgactccagaacctggctcatcaccaagcacgagcacgtacgagcccttctcgccgacccccgggtcaccgtcgacccggccaagctgcccaggctctccccctccgacggcgacggcggcggcttccggtccctgctgaccatggaccccccggaccacaacgccctccgccgcatgctcatatccgagttcagcgtgcaccgggtccgggagatgcgcccgggcatcgagcgcaccgtgcacgggctgctggacgggatcctcgaacggcgggggccggtggacctggtggccgaactcgcgctgccgatgtccaccctggtgatctgtcagctcctcggagtgccctacgaagaccgcgagttcttccaggaacgcagcgaacaggccacccgggtgggcgggagccaggaatcgctgaccgcgctcctggaactacgggactacctggaccggttggtcaccgcgaagatcgagacgccgggtgacgacctgctgtgccggctcatcgccagtcgactgcacaccggtgagatgcgacacgccgagatcgtggacaacgccgtgctgctgctcgccgccggccacgagaccagtgccgccatggtggcactgggcatcctgacactgttgcggcaccccggcgccctggcggagttgcggggcgacggtacgctgatgccgcagacggtcgacgaactcctgcgtttccactccatcgcggacggccttcgacgggcggtcacggaggacatcgaactcggcggcatcacgctgcgcgccggagacggcctcatcgtctcgctggcctccgccaaccgcgacgagagcgccttcgcctccccggacggcttcgacccgcaccatccggcgagccggcacgtcgccttcggctacggcccccaccagtgcctgggccagaacctggcccggctggagctggaggtcaccctgggcgcggtggtggagcgcattcccacgctcaggctggccggcgacgccgacgcactgcgcgtcaaacaggattcgaccatcttcgggctgcacgagctgccggtcgagtggtgacggaaggaggacacagcgtgcgggtgacagtcgaccagagccggtgcctgggagccggccagtgcgagcagctggcgccggaggtcttccgccaggacgaggaaggactgagccgggtcctcgtccccgagcccgatccggcgtcatggccgcgggtgctccagacggtggacctctgccccgtacaggccgtcctcatcgacgagggccccggtcccgcgccgcaggacaccaagtgaccgctgaccgctgggccggccgcacggtgctcgtcacgggagcactcgggttcatcggctcccacttcgtccgacagctggaggcgcgcggagccgaggtgctcgccctctaccgcaccgaacggccgcaattacaggccgagttggccgcgctcgaccgagtacgcctgatcaggacggagctgcgggacgagtcggacgtgcgaggggccttcaagtacctggcaccctccatcgacaccgtcgtccactgcgcggccatggacggcaacgcgcagttcaagctggagcgctcggccgagatcctcgacagcaaccagcggaccatctcccacctgctgaactgcgtacgggacttcggcgtcggcgaggccgtggtcatgagctcctccgagctgtactgcgcgccgcccaccgcggcggcccacgaggacgacgacttccgccgatccatgcggtacacggacaacggctacgtcctgtccaagacctacggcgagatcctggccaggctccaccgcgagcagttcggcaccaacgtcttcctggtgcgaccgggcaacgtctacgggccgggagacggctacgacccctcccggggccgggtgatccccagcatgctggccaaggccgacgccggcgaggagatagagatctggggggacggcagtcagacccggtccttcatccacgtcaccgacctggtacgggcctcactgcgcctgctggagaccggcaagtaccccgagatgaacgtggccggcgcggaacaggtctccatcctggagctcgcccggatggtgatggccgtcctgggacggcccgagcgcatccgcctcgaccccggccgccccgtcggcgccccgagcagacttctggatctgaccaggatgtcggaagtgatcgacttcgagccccagcccctgcggaccgggctggaagagaccgctcgctggttccgccaccacacgcgctgaacctcctctcatacccccctggaaggtaactcgtggtcacacacgccccgaactcgctgatcagtgacataatccgcgcctccggcgggcatgacgccgacctcaaggacctggccgcccgacacgatccggccgacatcgtccgcgtactcctggacgagatcacctcacgctgccccgctcccgtgaacgacgtccccgtcctcgtcgagctggccgtccgggcgggagaccgcctcttccccacctatctgtacgtcctcaagggcggcccggtgcgcctcgcggccaaggacgaggcgttcgtcgccatgcgcgtcgagtacgagctgggcgaactggcccgcgagctgttcggaccggtgcgggagaacgtcaccggcgtccgcggaacgactctcttcccctacgtcggggacacggcgtcggaaggcgaggaggactcgggtgccgagcacatcggcacgcacttcctggccgcgcagcagggctcccagaccgtgctcgccggctgccattcccacaagccggacctcagcgagctctcctcgcgctacctcaccccgaagtggggctcgctgcactggttcaccccccactacgaccgccacttcaggagctaccgggaccagcccgtacgcgttctggagatcggcatcggcggctacaagcaccccgagtggggcggcggctccctgcgcatgtggaagcacttcttccatcgcggcgagatctacggcctggacatcgtcgacaagtcgcacttcgacgcgccgcgcatcacgaccctgcgcggcgaccagagcgaccccgaccacctgcggtcgatcgccgagaagtacggaccgttcgacatcgtcatcgacgacggaagccacatcaacgaccacatccggacctcgttccaggcactgttcccgcatgtgcggcccggcggcctctacgtgatcgaggacctgtggaccgcctactggtccggcttcggcggcgacgaggacccgaagcggtacagcgggacgagcctgggcctgctcaagtccctcgtcgactcgatccagcacgaggaactgccggaggccggcgaccaccgtcccagttacgcggaccagcacgtggtcggcatgcacctctaccacaacctggcgttcatcgagaagggcaccaacgccgagggcggcatccccccgtggatcccacgcgacttcgagaccctcgtcgccgtctcctccgggggccacgcatgaggagccgtcggcaccagccacccgaacacacccgaccggaccgcaggaggcccgcatgcgcgtgaccctgctgagcgtcggatcccgaggcgacgtccagccgttcgtcgccctcggcatcggcctcaaggcccgcggccacgacgtcaccctggccgcccccgccacgctgcggccactggtcgagcgcgcgggactgacgtacaggctgtcccccggggatcccgacggattcttcaccatgcccgaggtcgtcgaagcgctgcggcgcggcccctcgttcaagaacatgctcgcggggatgcccgaggcgcccgagagctacacacagcaggtcgtcgacgcgatccacgacgccgccgagggcgccgacctcatagtgaacgcgcccctcaccctggccgccgcgtacgggcacccgcccgccccgtgggcctcggtgtcctggtggcccaacagcatgacctcggccttcccggccgtcgaatccgggcagcgccacctcggaccgctgacgtccctgtacaaccgctacacccatcgcagggcggcacgcgacgagtgggagtggcgacgccccgagatcgacggctaccgccgacgcctcggcctccggcccttcggcgacgagtccccgttcctccgactggggcacgaccgcccgtacctgttccccttcagccccagcgtgctgcccaagccgcgggactggccgcgccagagccacgtcaccggctactggttctgggaccagcacggggagccgcccgccgagctggagtcgttcctggaggacggggagcccccggtggcgctcaccttcggcagcacctggtcactccaccggcaggaggaggccctccaggccgccctcgacgccgtccgtggcgtcggacgccgactggtcatggtcgacggaccggacagcgacctgcccgacgacgtgctccgcctgcaccaggtggactacgccaccctcttccccaggatggccgcggtgatccaccacggcggcgccggcacgaccgccgaggtcctccgggccggagtgccccaggtcatcgtgccggtcttcgccgatcaccccttctgggcggctcgactgtccagaacaggcgtcgccgcccggccggtccccttcgcccgcttcagccgagaggcactggcgcagagcgtccgccaggcggtcaccgatcccgcgatggcgggccgggccaggcgactgggcgaacgggtctccaaggaacggggagtggacaccgcctgcgacatcctcgagaagtgggcggagacggcacgcgccacggcctgacacggccaccggcgggcgggcccggaagccgcacgcccgccggccgacgggtcccgggaccgcgccgctacgccgacaaccggtaggcggagagccgcacggagagcgtgacccgagtcggcgccggcagccgctggatcgtctccagatcccgctgcgcgtcacggtgccaggcattgggccccatgccgaccaggtgcgccagagcctggtggtcgagagccatggtccgggtcaactcctccgtggcgacggccgagaagtgcggagcgagctgctccgcgagacgcgactccttgccttcgtccacctgcagcaggccgagggcgccgatcacctcccgcaggtgatcgggcagaggagtgacaaccaggagaacgccacggggatggagaacgcgatgcagttcaggaccgttgcggggagcgaacgtgttgatcaccatgccggctgcggcatcccgcagcggaagcgtctgccaggcgtcggtcaccgcggccgcgatccgcggatgcgccttcgcggcacgccgcacggcgtacttggagatgtccagcagcaggccctgggcatcggggaacgcttccatgaccccggcgtgatagtggcccgtccccccaccgatgtcgaccacacagccgggcacggccggatcggccgtccgccgcgccagatcgaccagcgcatccatcacggggtcgtagtgacccgccgacaggaatgcgtcccgggcctccaccatttccttggtgtcggcccgcagcttcgtcgccctgggaagcagattcacataaccctgcttcgcgatgtcgaaggagtgtccggcggggcagaaaagtgcgcggtcgccctgagccagcgaagcaccgcagtgcgggcaggcgaggtagcgcacgatcctgttgagcatggggcaccgttccttcgggcgagtcggcagaccggccaaccctaacgcaggcgcccggccgcccaccgcccggcgcagggccgacgaaccaccgtgacgtgcaccagccgcgcgtgagaactcctcatgcgcgcaccctacggaaatcggcaggtcaaccggcgattcctgcgggaattccgagcgaaacggcctcactgtgtttccccgctgcatttcctcgctgaattcagcgaatcccggcagacgaccggctctgccggcgtgacagcccctatcgatcgaccaggagtttcgatggccccgaagagtggtgcgcagcgttcgagcgacatagccgtcgtcggcatgtcctgccgccttccgggggcaccgggcatcgatgagttctggcatctgctgaccaccggaggcagcgcgatcgagcgtcgcgccgacggcacctggcgcggctccctggacggagccgccgacttcgacgccgccttcttcgacatgaccccccgccaggccgccgccgccgacccgcagcaacgactcatgctggaactcggctggacggccctggagaacgccgggatcgtccccggcagcctcgccggcacggacaccggcgtcttcgtcggcatcgcggccgacgactacgcggcactcctgcaccggtccgccacccccgtcagcgggcacaccgcgacgggcctgagccggggcatggccgccaaccgcctctcctacctcctgggcctgcgcggtcccagcctcgcggtggacagcgcgcagtcctcctcgctcgtcgcggtccacctggcctgcgagagcctgcgccgcggcgagtccgacctcgcgatcgtcggcggcgtcagcctgatcctcgccgaggacagcacggcgggcatggagctcatgggcgcgctctcgccggacggccgctgccacaccttcgacgcacgcgccaacggctacgtacgcggtgagggcggagcctgcgtcgtcctcaagcccctggagcgggcactggccgacggggaccgcgtccactgcgtcgtccgaggaagcgcggtcaacaacgacggcggcggctccaccctgaccaccccccaccgcgaggcccaggccgccgtcctgcgggcggcgtacgaacgggccggggtcggcccggaccaggtgtcctacgtcgaactgcacggtacggggacgccggtcggcgaccccgtcgaggcggcggctctcggcgcggtcctcggcacggcccacggccgtaacgccccgctgtccgtgggatcggtcaagacgaacgtcggccacctggaggcggccgcgggcctcgtgggattcgtgaaggcagccctgtgcgtccgcgagggcgtggtgccgccgagcctgaaccacgcgacgcccaaccctgccatccccatggaccggctaaacctgcgcgtacccacgcgactggagccctggccgcacccggacgaccgagcgaccggccggctgcgactggccggcgtctcgtccttcggcatgggtgggacgaacgcgcatgtggtggtggaggaggcgccgcttccggaggccggggagccggtcggggccggtgtgcctttggctgtggtgccggtggtggtgtcgggtcgttctgcgggtgcggtggctgaactggcctcccgcttgaacgagtcggttcgttcggatcggttggtggatgtggggttgtcgtcggtggtgtcgcggtcggtgttcgagcatcggtccgtggttctggcgggggactctgccgagctgagtgccggtttggatgctctggccgctgacggagtgtctcctgtcctggtgtcgggtgtggcgtcggtcgggggtggccggtcggtgttcgtgttcccgggtgcgggggtgaagtgggcggggatggcgctcgggttgtgggcggagtctgctgtgtttgcggagtcgatggcgcggtgtgaggcggcgttcgccgggttggtggagtggcgtctggcggatgtgctgggtgatggggctgcgttggagcgtgaggacgtggtgcagccggcgtcgttcgcggtgatggtgtcgttggcggcgttgtggcggtcgttgggtgtggtgccggatgcggtggtggggcattcgcagggggagatcgctgctgcggtggtggcgggtggtctgtcgttggaggacggggcgcgtgtggtggtgttgcgtgcgcgggtggctgaggaggttttgtcgggtggggggattgcttcggtgcgtctttcgcgggccgaggtggaggagcggttggcgggtgggggtggtgggttgagtgtggcggtggtgaatgcgccgtcgtcgacggtggtggcgggtgagttgggggatttggatcggtttgttgcggcgtgtgaggcggagggggtgcgggcgcgtcggctggagttcgggtatgcgtcgcattcgaggttcgtggagccggtgcgtgagcggttgttggaggggttggccgatgtccgtccggtgagggggcggattccgttctattcgacggtggaggctgcggagttcgatacggctggtctggatgcggagtactggttcgggaatctgcgtcggccggttcgcttccaggagacggtcgagcggctgttggcggatggtttccgggtgttcgtggagtgcggcgcgcatccggtgctgaccggggcggtgcaggagaccgcggagactgcgggccgggagatctgctccgtcggatccctgcgtcgtgacgagggtggactgcgtcgcttcctgacctctgcggcggaggcgttcgtccagggggtggaggtgtcctggccggtgctgttcgacggcaccggcgcccggacggtcgacctgcccacctaccccttccaacgccggcaccactgggcacccgacggctccgcgtcggcggcgcccacacgggacatccgaccggacgagaccgccgcggttccagcggacacgatggacctcgcgggacaacttcgcgcggacgtggcgtcgttgcccaccaccgagcagatcgcgcggttgctcgaccaggtacgcgacggtgtcgccacggtcctcggactggacgcccgggacgaggtgcgcgcggaggcgacgttcaaggaactgggcgtcgaatcgctgaccggcgtcgagctgaagaaccacctccgtgcccggaccggactgcacgtccccacctcgctgatctacgactgcccgactcccctcgccgccgctcactacctccgcgacgagctcttgggccgacccgcggagcaggccgtcgtccccgccggcatcccggtcgacgaaccgatcgcgatcgtggggatggggtgccgtctgccgggtggggtgtcgtcgccggaggggttgtgggatctggtggcgtcgggggtggatgcggtttctccgttccccacggatcggggttgggatgtggggggtctgttcgatccggagccgggggtgccggggcgttcgtatgtgcgtgagggcgggttccttcatgaggcgggggagttcgacgcggggttcttcggtatctctccgcgtgaggcgttggcgatggatccgcagcagcggttgttgctggagacgtcgtgggaggcgttggagcgggcgggcatcgatccgcacacgcttcgcggttcacggaccggcgtctacgccggagtgatggcccaggaatacggcccccgactccacgaaggcgcagacggatacgagggctatctgctgaccggatcctccagcagtgtcgcctcgggtcgtatctcgtacgtgctgggtctggaagggccggcggtgacggtggacaccgcgtgctcgtcgtcgctggtcgcgctgcacctggccgtgcgggcgctgcgcagcggtgagtgcgacctcgccctcgccggcggcgcgaccgtcatggccgaacccggcatgttcgtggagttctcacggcagcgcgggctgtctgcccacggacggtgcaaggcgtactcggactcggccgatggcacgggctgggccgagggggcgggtgtgctgctcgtcgagcggttgtcggatgcggtacgtcatgggcgtcgggtgctggcggtcgtgcgtggttccgcggtcaaccaggacggtgcgagcaacggactgacggcgccgaacgggcggtcccagtcgcgtttgatccgtcaggcgttggcggatgcgcggttgggtgtggctgatgtggatgtggtggagggccacggcacggggacgcgtctgggtgatccgatcgaggcgcaggcgttgttggcgacgtatgggcagcgggatgcgggtcggcctctgcggcttggttcgctgaagtcgaacgtggggcatacgcaggcggctgccggtgtggcgggcgtgatcaagatggtcatggcgatgcggcacggtgtcctgccgaagacgctgcacgtggatgagccgacggcggaggtggactggtcggccggcgcggtgtccctgctgagggagcaggaggcgtggccgcgtggcgagcgtgtgcggcgggccggagtctcctcgttcggcgtgagtgggacgaacgcgcatgtggtggtcgaggaggcgccggttccggaggacggggaggcgatcgagggcggtgcgcctttggctgtggtgccggtggtggtgtcgggtcgttctgcgggtgcggtggcggagctggcgggccgggtcagcgaggtcgctgggtctggtcggttggtggatgtggggttgtcgtcggtggtgtcgcggtcggtgttcgagcaccggtccgtggtgctggcgggggactctgccgagctgaatgccggtctggatgctctggccgctgacggagtgtctcctgtcctggtgtcgggtgtggcgtcgggtgagggtggccggtcggtgttcgtgttccctggtcaggggacgcagtgggcggggatggcgctcgggttgtgggcggagtcggcggtgttcgcggagtcgatggcgcggtgtgaggcggcgttcgccgggttggtggagtggcgtctggcggatgtgctgggtgacgggtctgcgttggagcgggtcgatgtggtgcagccggcgtcgttcgcggtgatggtgtcgctggcggagttgtggcggtcgttgggtgtggtgccggatgcggtggtggggcattcgcagggggagatcgctgctgcggtggtggcgggtggtctctcgctggaggatggcgcgcgtgtggtggtgttgcgtgcgcggttgataggccgtgagctggccgggcgcggtgggatggcgtcggtggcgctgccggtcgcggtggtggaggagcgtctggcggggtgggcggggcgtctgggtgtggcggtggtcaacggaccctccgccacggtcgtcgcgggtgatgtggatgcggtgggggagtttgtgaccgcgtgcgaggtggagggggttcgggcgcgtgttctgccggtggactacgcctcgcactcggcgcacgtggaggacctgaaagccgagcttgaacagattctggccggcatcggcccggtgaccggtggcatcccgttctattcgacgtccgaagccgcgcagatcgacacggctggtctggacgcggggtactggttcgggaatctgcgtcggccggtgcggttccaggagacggtcgagcggttgctggcggatggtttccgggtgttcgtggagtgtggcgcgcatccggtgctgacgggggcggtgcaggagaccgcggaatccaccggtcgccaggtgtgtgcggtcggatccctgcgtcgtgacgagggaggtctgcgccgcttcctgacctctgcggcggaggcgttcgtccagggggtcggggtgtcctggccggcactgttcgacggcaccggcgcccgcacggtcgacctgcccacctatcccttccagcgtcggcgttactggctggagtcacgtcctcctgcggcggttgttccgtcgggggtccaggacggattgtcgtatgaggtggtgtggaagagcctgccggtacgggagtcgtcgcgtcttgacggccggtggctgctcgtcgtgcccgaaaccctggacgccgacggcacgcggatcgcccacgacctccagcacgccctcaccacccacggcgccacggtctcccgtgtgtcggtcgacgtgacgacgatcgaccgcgccgacctgtcggcgcggctcaccacgagcgcggccgaagaccaggaaccgcttgggcgagtggtgtccctcctggggtgggccgagggagtacgggcccatggcccgaacgtaccgacttccgtcgccgcctccctggcactcgtgcaggctgtcggcgatgccgggttcggtgttccggtgtgggcggtgacgcggggtgcggtgtccgtggtgcctggtgaggtgccggagacggcgggtgcgcaactgtgggcgctcggccgggtcgccggtctcgaacttcccgaccgttggggtggtctgatcgatcttccggcggatgccgatgcgcgtacggcggggcttgcggtgcgggccctggccgccgggatcgccgatggtgaggaccaggtggcggtgcgcccctcgggtgcctacggccggcgtgtagttcaggcagcccaccgggagccgtcgggagcgaagacggagtggcgaccgcgtggcaccgtgctcgtcaccgggggaatgggcgccatcggcactcgggtggcccgctggctggcccggaacggagccgaacacctggtgctgaccggccgccgcggtgccgggacccccggcgcggacgagctggcgggagagctgagggcgtccggagtccaggtcacgctcgccgcctgcgacgtgtccgatcgtgccgcgctggccgcgctgctcgacgcgcatccgccgaccgccgtcttccacacggccggtgtactgaacgacggaacggtcgacacgctcactcccgctcacctggacggggtcctgagccccaaggcgacggccgccgttcacctgcacgagctcaccgcccacctggacctggacgccttcgtcctcttcgcctccgtcaccggcgtatggggtaacggcggccaggccgggtacgccatggccaacgcggctctggacgcgctcgccgagcagcgccgtgccggcggacttgcggcgacctccatcagttggggcctctggggtggcggcggcatggccgagggtgacggcgaggtgagcctcaaccgtcgtggaatccgcgctcttgagcccgccaccggcatcgaggcgctgcagcggacgctcgaccagggcgccacctgccgcaccgtcgtcgacgtggactggggtcagttcgctcctcgtacggcggcgctgcggcgcgggcggctcttcgcggatctgcccgaggtgcggcgtgtcctggagtccgagggggttgcacgggaggacgccggaaccgtcgagcccggcgccgtgctcgccgagcgcctcgcatcgcgctccgaggccgaacagcgacgcatgctcgtcgagttggtacgagccgaagcggctgccgtcctgcgtcacgacacgacggacctcctggcgccgcgcaggtcgttcaaggacgccgggttcgactccttgaccgcgctggagctccgtaaccggctgaacaccgccaccggtgtcgtccttcccgtcaccgtcgtcttcgaccacccgaaccccggtgcactggcggacttcctgtacggcgaagcactgggcctgtccgcggccaggtcttccgcgagcgatacggccgacacgacccgcccggccgccgcccccgaagagccgatcgcgatcgtcggaatggcctgccgctacccgggcgaggcccgttcccccgaggaactgtggaagttgctcatcgacgaacgggacgtcatcggccccatgcccacggatcggggttgggatgtggggggtctgttcgatccggagccgggggtgccggggcgttcgtatgtgcgtgagggcgggttccttcatgaggcgggggagttcgacgcggggttcttcggtatttctccgcgtgaggcgttggcgatggatccgcagcagcggttgttgctggagacgtcgtgggaggcgttggagcgggcgggcatcgatccgcacacgctccgcggctcacagaccggcgtctacgcggggatgttccaccaggagtacgcgacccggctgcacgaggcacccgtggagttcgaaggccacttgctgacggggacgtccgggagtgtggcttcgggtcgtatctcgtatgtgctgggtctggaggggccggcggtgacggtggacacggcgtgttcgtcgtcgttggtggcgctgcatctggcggtgcgggcgttgcggagtggtgagtgtgaccttgctcttgcgggtggggtgacggtgatggcggagccgggtgtgttcgtggagttctcgcggcagcgggggttggctgcggacgggcggtgcaaggcgttcgcggctgctgccgatggcacgggctgggccgagggtgtgggcgtgctggcggtggagcggttgtcggatgcggtgcgtcatgggcgtcgggtgctggcggtggtgcgtggctcggcggtgaatcaggacggtgcgagcaatgggttgacggctccgaacggtccgtcgcagcagcgggtgattcgtcaggcgttggcggatgcgcggttgggtgtggctgatgtggatgtggtggaggggcatgggacggggacgcgtctgggtgatccgatcgaggcgcaggcgttgttggcgacgtatgggcagcgggatgcgggtcggcctctgcggcttggttcgctgaagtcgaatgtggggcatacgcaggcggctgccggtgtggcgggcgtgatcaagatggtcatggcgatgcggcacggggtcctgccgaagacgctgcacgtcgatgaggtctctccgcacgtcgactggtcggcgggcgcggtgtccctgctgacggagcaggagccgtggccccgtggtgagcgcgtacggcgggctggtgtctccgcgttcggtgtgagtgggacgaacgcgcatgtggtggtggaggaggcacctgcttcggaggcgccggtcgcggtggagccggtggagccgggggctgtggggcttcttccggtggtgcccgtggtggtgtcgggtcgttctgcgggtgcggtggctgaactggcctcccgcttgaacgagtcggttcgttcggatcggttggtggatgtggggttgtcgtcggtggtgtcgcggtcggtgttcgagcatcggtccgtggttctggcgggggactctgccgagctgagtgccggtttggatgctctggccgctgacggagtgtctcctgtcctggtgtcgggtgtggcgtcggtcgggggtggccggtcggtgttcgtgttcccgggtgcgggggtgaagtgggcggggatggcgctcgggttgtgggcggagtctgctgtgtttgcggagtcgatggcgcggtgtgaggcggcgttcgccgggttggtggagtggcgtctggcggatgtgctgggtgatggggctgcgttggagcgtgaggacgtggtgcagccggcgtcgttcgcggtgatggtgtcgttggcggcgttgtggcggtcgttgggtgtggtgccggatgcggtggtggggcattcgcagggggagatcgctgctgcggtggtggcgggtggtctgtcgttggaggacggggcgcgtgtggtggtgttgcgggcgcgggtggctgaggaggttttgtcgggtggggggattgcttcggtgcgtctttcgcgggccgaggtggaggagcggttggcgggtgggggtggtgggttgagtgtggcggtggtgaatgcgccgtcgtcgacggtggtggcgggtgagttgggggagttggatcggtttgttgcggcgtgtgaggcggagggggtgcgggcgcgtcggctggagttcgggtatgcgtcgcattcgaggttcgtggagccggtgcgtgagcggttgttggaggggttggccgatgtccgtccggtgagggggcggattccgttctattcgacggtggaggctggggagttcgatacggctggtctggatgcggagtactggttcgggaatctgcgtcggccggttcggttccaggagacggtcgagcggttgttggcggatggtttccgggtgttcgtggagtgtggtgcgcatccggtgctgaccggggcggtgcaggagaccgcggagactgcgggccgggaggtgtgtgcggttggttcgctgcgtcgtgacgagggaggtctccgtcgcttcctgacctctgcggcggaggcgttcgtccagggggtggaggtgtcctggccggtgctgttcgacggcaccggcgcccgcacggtcgacctgcccacctaccccttccaacgccgccactactgggcacagtcctcgcccgccggcgccggcagctctgctgcggcccgcttcggtatgacctgggaggagcatccgctcctcggcggcgcgctgccgctcgcggactccggcgagctgctgctcgtcgggaggatctccccggcctcccactcctggatcgccgaccacaccgtggccgggaccgccctgctgcccgggacggccttcgtcgacatggcactgcacgccgccgcggtcgcgggctgtgcgggtgtggaggagctgagcatcgaggccccgctgccggtgcacggcgggatccggctccaggtggtgatcgacgagcccgactcctccgcgcggcgccgcgtgagcgtgttcgcgaggccggaagaggaagacggggacgccggccgctggacccgacacgccaccggcgtgctgacccccgacgtcgcccccgagccgggccggccgcagtggtgccggcaggcctggccgccgagcggctccgtccgggtggaggcgtcggagctctacgaccggttctccgcgctgggatacgactacggcgaggtcttcgccggggtcgaggccgtctggctgcgcgagggcgaggccttcgccgaggtccgcctgcccacgggcgcggcgcccgacgccgagcggttcggggtgcaccccggcctcctcgacgcggctctgcacccctggctgctgggcgacttcctgtcgcggcccgacggcggatccgtactgctgccgttcgcgtggcgcggcatcacgctccacacggccggtgccgacgcgctgcgggtccgtctcggaccggccggagaaggcgctctgtcgctcgaagccgtcgacctctccggtgccccggtgctgtcgatggacgcactggtgctgcgtccgctcgcccaggaccgcctggcggaactggtcggcggcacgacctccaccccgctgtaccgcgtggactggcagcggagcccgatcgcgaggacggcgccgtcggccacggggctcttcggctccctcccgtccggtgccgtccgccgctgggcggtcgtcgggcagggcggtgtcgccgcacggtacgcgacggcggaacccggcacggggtgcgtcggggtcttccccgacctggacgcactgcgtacgacgctggactccggagcggacggccccgacatcgtcctcgccgacttcggggcccggccaggcgacgccgcgccgcacgggacggatccggccgacggcgcacgcgacacggtccggcgggggctcgccctcatacagggctggctgtccgacgagcgcttcgccgccgcgcgtctcgccgtgctcaccgagcacgccgtcgccaccgaggcggacacccgccggacggacctcgcgggctcggcactgtgggggctgatgcgttcggcgcagaccgagcaccccgaccgcttcgtcctcgtcgaccacgacgggcaggacgcctcgtaccggacgctgcccaccgcgctcgacagcgaaatcccgcaactggcgctccgagccggggagacgctggcccccgaactggcggtcctgccgagtccggccgacggggggcccgcgacaagcgcggcgttcgatcccgaaggcacggtactcgtcaccggagccaccggcaccctcggcagcctgctggcccggcacctggtcacggcacacggcgtacggcatctgctgctgctcagccgcagcggacgcgaagccgcgggggccgccgaactggagcgtgaactccgtcaacggggagccgagttccagctcctctcctgtgacgcgacggaccgggcagcgctgaaggaggccctcgccaccgtccccgccgcccacccgctgaccgcggtgatccacacggccggcgtcctcgacgacggcgtcgtcgaggcgctgacccccgaacggctggaccgcgtgctgcgccccaaggcggacgccgcgctgaacctgcacgacctgaccgagggaatgccgctgaaggcgttcgtcctgtactccggggcggtcggactgctgggtggagcgggccaggccaactacgcggcagccaacgcgttcctggacggcctggcccaacaccggcacgcgcaagggctgcccgcggtgtccctggcatggggactctggagcgccaccagcacgttcaccgaccatctcggcgaggtggacctgcggcgcatggagcggtccggcatcacgccgctcacggacgagcagggccttgacctgttcgaccgggccctcggcgccgcggtggacgcgccgcagctctgcgtgatggggctggacacggcggcgctgcgccggcaggcggccgagcacgggccgacttcgatgcctcctctgctgcgtacgctggcggcgcctcccgtacggcgcggcgcggggcgctccggccggggcggacgggcggcgtccgccacggacgcgccgagccgggcgcaggccctgcgcgagcgactgacgggcctggacgcggcggcacggcgggacgaactcctggtcctgtcgcaggcgcagttggccgatgtgctgggcttcgccgacaagaccgcggtggaccccgttcgttccttccgcgagatcggtctggactcgctgaccgccgtcgagctgcgcaaccggctcggtgtcgtcaccggactgcggctgccgccggcgctggtcttcgaccaccccaacctcgacgcgctcgcggcccacctggcggagctcctcgcggctgagggccgggacgacgcgggcgccgcggcgctctcgggaatcgacgccctggaccgggcggtccgggagatggcggccgacgacacgcgccgtgacgccgtccgccgacgcctcgcggaactgctggcggtggtcggcgacgccccgcgggacggcggccgcgccccacgggcggccgccgacgcgggaggccgcgacgctcaagcggaccccgacctgctgggccggctggactccgcctccgacgacgatctgttcgcgttcatcgaagaccagctgtgagcgggacgccgcgcgcgttccccacccgtccctaagcgccgcatcaggcgcactcgcaccgacacgagcacgcaggccaggagggtccggtcgatgacggccaacgatgacaagatccgcgactacctgaagcgggtcgtcgccgagctgcacagcacgcggcaacggctcaacgccttggagcacgacgcccgcgagcccatcgccatcgtggggatgagctgccggctgcccggcggggtgaccacccccgagagcctgtggaggctggtcgactccggcaccgacgccgcctcgccgttccccgacgaccggggctgggacctggacgcgctccaccatccggagtcgggagccgtccactcccgcgagggcggattcctccacgacagcgcggacttcgacgcggagttcttcggcatcagcccgcgagaggccctggccatggacccgcagcagcggctgctgctggagaccgcctgggaggtgttcgagcgcgccggcatcgacccggtctccgcccgcggctcccgcacgggggtgtacgcgggcgtcatgtaccacgactacggcgcccggctgaacgagatcccgccgggcctcgagggctacctggtcaacggcagcgcgggcagcatcgcctcgggccgggtggcctacaccctcggtctggagggccccgccgtcaccgtcgacacggcctgctcctcgtcactggtcgccgtgcacctggcggcacaggcactgcggcggcgggagtgcgacatggcgctcgcgggcggcgcgaccgtcctgtccacccccgacctgttcatcgacttcgcgcgactcggcggcctggcctccgacgggcgctgcaaggcgttctccgacgccgccgacggcaccagcttcgccgagggcgccggcctgctgctcctcatgcggctgtcggacgcggtggccgagggccacaccgtcctggcggtcgtccgaggctccgccgtcaaccaggacggggcgagcaacggcctgacggcccccaacggcctcgcccagcaacgcgtgatccgcgaggcgctcgccgacgcggacctggaccccgaccagatcgacgcggtggaggcgcacggcaccggaaccaggctcggcgaccccatcgaggcgcaggccctgctgcacacgtacggcacgagccgcagccccgaacgacccctgtggctcggttcgctgaagtccaacatcggccacacccaggccgccgccggagtggccggagtcatcaagacggtgctggcgatgcgccacggacggctgccccgcacactgcacgtcacccgcccctccagccgggtggaatggtcggcgggcgcggtggaactgctcacgcgggcacaggactggcccggccaggggaacgccccgcgccgcgccggagtgtcgtccttcggtgccagcggcaccaacgcacacctgatcctggaaggcgtcccggacggcgacatcacggtcgcggagacccgaccggccaccggcggcggcgcctggccgctcgcgggccggaccgaagcggccctgcgcgcacaggcccggcggctccacgaccacctcgccgcccgcccccacgtctcacccgccgcggtcgggcggacgctggtccgctcccgcacggcgttcgagcaccgggccgtcgtcctcggacaggacaccgccgacctcctgagcggcctcgcggagctggcgtccggcggcgctcacggacccggcgtgatcaccggccgggccgcgcgcggccgccgtaccgcactgctcttcaccggacagggcagccagcggccgggtgccggacggcatctctacgagcggtacgaggtgttcgcccgcgccctggacgagacggccgcggcactcgaccggcacctcgaccgcccgctgcgcgacgtgatgttcgcggagccgggcggcgcgacggccggactcctcgaccgcaccgagtacacccagcccgcactgttcgccctggaagtcgccctcttccgactggtgaccgccgggggcctgcgccccgacgcactcctcgggcactccgtcggcgaactggccgccgcccacgtcgccggagtgttcaccctgcccgacgccgcccgcctggtgacggcgcgaggccgactgatgggcgagctgccggccggtggcgccatgatggcgatccaggcgagcggcccggagatcgaggagacgatcacggcgctcgcggcccaccggtcggcgcgcgtcgccgtcgccgcactcaacggtcccgacgccaccgtgatctcgggcgacgaggacgtggtcgccgaactcgccacgctgtggcgggagcggggccgccgcaccagggcgctgcccgtcagccacgccttccactcgccgcacatggacgccgcactggaaccgttcgcccggatcgcgcgcgacgtctcctacgccgaaccgcgcatcccggtggtctccaacctgaccggcggcatcgcgtcggccacgacgctgtgcgcccccgagtactgggtgcgccacgcgcgcgaggccgtgcgcttcagcgacggcttccgcgccctgcgcgaccaggggatcgacaccttcatcgagctcggaccggacggcgtgctgtccgccctgggccgcgactgcctccgcgaggaggaaggagacgccccacgccaggacggctcggcggaccccgacacgaccggctcccgcgccgacggggggcggcgacccgtgctgacggtgccgctgctgcgccgggaccgcgacgagacgaccacctgcctcggggccctggccaccgtccacacccacggcgtccccgtcgacctcgcggccgtgcacggcgcccccgaggggcccgccgtcgagctccccacctacgccttccaacgcacgcgctactggctggacgccccggcccccgccgccggccccaccgccaccggtctggaggcgacggaccagcccctgctcccggccgtcatcgacctgcccgacggggaaggcaccgtacggaccggactgctctccctgcgcacccatccgtggatcgccgaccaccgcgtccgcgaccacgccgtggtgcccggagcggccctgctggacgtcgccgcctgggcgggcaccgaagcgggctgcccccgggtcgccgaactgaccttcgccacgcccctcgtcctgcccgagaacggagaaggagtccgactgcgcgtgacggtcagcggccccgacgcggaaggcatccgttcgctacgcatcgactcccggcccgccgacacggtccgtaccgccgatgccccctccgactggacccgccacgcgtccggcaccctcgtccccgcacccgaggaggccggcgacggcaccggcgtgccgaccgaactgctcggcgcctggcccccggccgacgcgaccccggtcgccctcgacgcggacgccgtcgcggccgagtatcagcgcctcgcggccggcggcgtgacgtacggccccgcgttccgggcactgcgcgccgtctggcgccgcggcgcagaggtcttcgccgaggtccggcttcccggccaggcggccgccgacgcctcgcggtacggcatgcacccggccctgctggacgccctgacgcacgccaccgggttcggcgagcggtccaccgaggcccgcggcctggtcccgttcgcctggagtgacgtccggatccacgtccgcggcgccgactccctgcgcgtacgcatcgcgccggccggccccgacgccgtgaccgtcgccgcggtcgacccgacgggccgcccggtcctcgccgcccgctcgctcacgctgcgccccctggcggagagccggttccaggacccggaggcggacagcacgccgctgtaccgactggagtggacaccggcccccggttccgtgaccgggcacgccggtccaaggcaggcggcagcggagtggggcgtcctcggcgacccggtccaggccctcctcgacgccgtgcgcgacggggcggaggcacccgtgcgaacccatgacgacctgctcgcgctcgcggcctccgacacggccccgcccgaccatgtgctggcgctgctgggccacgacggggacgctctcgccacgggcgcccacgacctggccgcacgcgccctggccctggtacagggctggctgacccacgcccgcttcgccgacgcgcgactggtcgtgctgacgcagggggcggtgacggccggcacgagccccgtccacccggccgcggccgccgcctgggggctgctgcgcagcgcacagtccgagcacccgggccgcttcgtcctcgtcgacgcggaccccgccgacccggccgcctcgtaccgttccctgccacgggccgtcgcctccggggcgtcccaactcgccctgcgcggcgccgagatcctcgtcccccggctcgcccggggaacggaccgacaggccaccgtgcccggacaccccggcgacgtcaccgcaccggagacgaccgctgcccccgagccggccccgtccggcaccccctccggcccctggcccgcggacggcaccgtgctcgtcacgggcggaaccgggaccctgggcaaggccgtggcccggcacctcgtgaccaagcacggtgtccggcacctgatcctggccggtcggcgaggcgcggacacccccggggcggccgacctggccaccgaactgaccggcctgggcgccaccgtgaacatcgtccgctgcgatgccgccgaccgctcggcgctcgaaggcgtcctggccgccgtccccgccgcgcacccgctcaccgccgtcgtgcacaccgccggagtgctcgacgacggcatcgtcacggcgcagacgccccgacgcctctcggcggtcctgcgcgccaaggcggacgcggtcagccacctgcacgaactgacccgcgacctggacctgtccgccttcgtcctcttctcatcggccgccggaaccctcggcagccccggccagtccggctacgcggccgccaacagcttcctcgacgcgttcgccgcctggcggcgagcgcagggcctccccgccgtgtccctggcatggggactgtggggtgacggcggtgacggtcgtgacggcggtggctcggcggccgacggcatgggagcgagcctggccgccgccgacctggcacggctgcgccgttccggcatcctcccgctcgacccggccgaagcgctgcgcctgttcgacgaggcctgcgaccccgccaggaccgaggccgtactgctgccgatccgcctcgacctgaccggcctgcgcgcccgttccgcccgcggcgccgtacacgcgagcgtggtgcccgaagtgctgcacaccttggtgcccccgcccgccggtgccggatccccggccggtgccgacgcgtcggatcccgcggcgggccaggcgcccccggccccggcgtccgacaccctggccgaacggctcgccgggaagccccgaggcgaacggctcaccgccctcaccgaactggtacgcaccgagatcgcctcggtactcgggcaccccgactccggccgcgtccagctccagtcctccttcaaggagtccggcttcgactcgctcaccgccgtcgaactccgcaaccggctcaccgcggccaccggaacgaagcttcccgccaccctcgtcttcgaccatccgacacccgcggcactcgtcgaccacctggaacaggaactgccgaaggcagcacaggagatcccggcggacctcccggccgttctcgacgcactcgaccggatccgggacggactcgccaccgccgccaccgacgacagcagccgcgaccacatcgcggagcgtctccaggcgttgctcggcacgctcacctcggctgcgggcgtcagccgcccgaccggcagcccgggcgagcacgaccggcagggccccgatgagctgtcgctcggccaacgactcgccgccagcagcgacgacgaactcttcgacctcttcgacagcgacttccgatcgacgtagcccagggccacccttccgcctccgccgcccgccccacacccctggagaacagcacgatgtcttccacatcgcccgccaccaacgaagagaagctccgcgactacctccgccgcgccatgaccgacctgcacgaggcccgcgagcagatccgccggaccgagtcggccaggcacgagccgatcgcgatcgtggggatggggtgccgtctgccgggtggggtgtcgtcgccggaggggttgtgggatctggtggcgtcgggggtggatgcggtttctccgttccccacggatcggggttgggatgtggggggtctgttcgatccggagccgggggtgccggggcgttcgtatgtgcgtgagggcgggttccttcatgaggcgggggagttcgacgcggggttcttcggtatctctccgcgtgaggcgttggcgatggatccgcagcagcggttgctgttggagacgtcgtgggaggcgttggagcgggcgggcatcgatccgcacacgctccgcggctcacggaccggcgtctacgccggagtgatgtaccacgactacggcagcaccgcgaccgtctccgtcgcctccgacgacgagaccgccggattcctcggcacggggacgtccgggagtgtggcttcgggtcgtatctcgtatgtgctggggctggaggggccggcggtgacggtggacacggcgtgttcgtcgtcgttggtggcgctgcatctggcggtgcgggcgctgcggagtggtgagtgtgaccttgctcttgcgggtggggtgacggtgatggcggagccgggtgtgttcgtggagttctcgcggcagcgggggttggctgcggacgggcggtgcaaggcgttcgcggctgctgccgatggcacgggctgggccgagggtgtgggcgtgctggcggtggagcggttgtcggatgcggtgcgtcacgggcgccgggtcctggcggtcgtgcgtggttccgcggtcaaccaggacggtgcgagcaatgggttgacggctccgaacggtccgtcgcagcagcgggtgattcgtcaggcgttggcggatgcgcggttgggtgtggctgatgtggatgtggtggaggggcatgggacggggacgcgtctgggtgatccgatcgaggcgcaggcgttgttggcgacgtatgggcagcgggatgcgggtcgggctttgcggcttggttcgctgaagtcgaacgtggggcatacgcaggcggctgccggtgtggcgggcgtgatcaagatggtcatggcgatgcggcacggtgtcctgccgaagacgctgcacgtggatgagccgacggcggaggtggactggtcggcgggcgcggtgtccctgctgagggagcaggaggcgtggcctgaggtggggcgtctgcgtagggctgcggtgtcttcgttcggtgtgagtgggacgaacgcgcatgtggtggtggaggaggcacctgcttcggaggcgccggtcgcgggggagccggtggagccggtggagccgggggctgtggggcttcttccggtggtgccggtggtggtgtcgggtcgttctgcgggtgcggtggcggagctggcctcccgcttgaacgagtcggttcgttcggatcggttggtggatgtggggttgtcgtcggtggtgtcgcggtcggtgttcgagcaccggtccgtggttctggcggaggactctgccgagctgcataccggtctggttgctgtcgggactggggtgccgtcgcctggcgtggtgtcgggtgtggcgtcggtcgagggtggccggtcggtgttcgtgttccctggtcaggggacgcagtgggcggggatggcgctcgggttgtgggcggagtcggcggtgttcgcggagtcgatggcgcggtgtgaggcggcgttcgccgggttggtggactggcgtctggcggatgtgctgggtgacaggtctgcgttggagcgggtcgatgtggtgcagccggcgtcgttcgcggtgatggtgtcgctggccgagctgtggcggtcgctgggtgtggtgcccgatgcggtggtggggcattcgcagggggagatcgctgctgcggtggtggcgggtggtctctcgctggaggacggcgcgcgtgtggtggtgttgcgtgcgcggttgataggccgtgagctggccgggcacggtgggatggcgtcggtggcgctgccggtcgcggtggtggaggagcgtctggcggcgtgggcggggcgtctgggtgtggcggtggtcaacgcaccctccgccacggtcgtcgcgggtgatgtggacgcggtggcggagtttgtgaccgcgtgcgaggtggagggggttcgggcgcgtgttctgccggtggactacgcctcgcactcggcgcacgtggaggagctgagggccgagcttgaacagattctggccggcatcgacccggtggccggtgagacccccctgtactccacggtggaggcgggtgtcgtggatacggcgtcgatggatgcggggtactggttcaggaatctgcgtcggccggttcgtttccaggagacggtcgagcggttgctggcggatggtttccgggtgttcgtggagtgcggcgcgcatccggtgctgacgggggcggtgcaggagaccgcggaatccaccggtcgccaggtgtgtgcggtcggatccctgcgtcgtgacgagggtggtctgcgacgcttcctgacctctgcggcggaggcgttcgtccagggggtggaggtgtcctggccggtgctgttcgatggcaccggcgcccgcacggtcgacctgcccacctaccccttccagcgtcggcgttactggctggagtcacgtcctcctgcggcggttgttccgtcgggggtccaggacggattgtcgtatgaggtggtgtggaagagcctgccggtacgggagtcgtcgcgtcttgacggccggtggctgctcgtcgtgcccgaaaccctggacgccgacggcacgcggatcgcccacgacctccagcacgccctcaccacccacggcgccaccgtgcacactcttgctcttgaccccagcgcggcgcacttcgacggtctctttgacgggatactccaggaagaaacagatgtcacgggcatcttctctctcctcggactggcatcgggcccgcacccggatcacggcgaggtggagctcgcgggagccgcgtcgctgacgttgatgcgccaagcccagcgagacggcttccgtgctccggtgtgggcggtgacgcggggtgcggtgtccgtggtgcctggtgaggtgccggagaccgcgggtgcgcaactgtgggcgctcggccgggtcgccggtctcgaactccccgaccgttggggtggtctgatcgatctcccggcggatgccgatgcgcgtacggcggggcttgcggtgcgggccctggccgccgggatcgccgatggtgaggaccagctggcggtgcgcccctcaggtgcctacggccggcgcctcgtacgagccaccgcgcgccggggacggaaggactggcgcccgcagggtacggtgctgctcgccgggcacctcgacgccgtcggtgaaccactggcccgatggctgctcaccggcggcgcggaccacgtcgtccttgcggatcccgccctgaccgaactcccggccaccctcgcggatctggcccagaccgtgacgaccgctgcggcacccgaccttgccgaccgtgcagtcctcgccgccctggtcaccgagtacgtacccgccaccgtggtcgtcgttccgcccgcggcggagctcgctccgctggcgagtatcagcccggccgacctcgcggcggccgtcaccgccaagtccgcgaccgcggcgcacttcgacgcgctgctcgacggaccccacgcaccggagctggtgctgatctcctcggtcgcggggatctggggtggtgtccggcagggtgcgtacgccgtcggtgccgctcacctcgatgccctggccgcccgccgcagggcccgcggtctgtcggcggcctccgtcgcgtggacgccctgggcgggttccgtcaccgcggacggctccgccgccgagtcgctgcggcagtacggcatcgctccgctggagccgcaggcggcgctcgcggagctggaccgggcgctgaaccagcagctgcacggcggcgggggcgacgcggcggtggccgacatcgactgggagcggttcctcgcgtcgttcacctccgtacgtcccagcgttctcttcgacgagctgcccgaggtacgccgtctccgcgaggcggaggcggcggccatggcggaccaggccgccgcccggacgggagcgcccggcggaacggagctggcgcgctctctgcgggccaagtccctgaacgcccagcgaactgcgctcctggaattggtcactgcccacgtggcggccgtgctgggagagagcgttcccgaggcgatcgaccggagccgggcgttcaaggacatcggcttcacctccatgaccgcgatggaactgcgcaaccggctcaaggaggccaccgggctcgcccttcctgcctccctcgtcttcgaccacccccaccccggcgcactcgccgaccacctgcgcgaggaactcctgggcgaggacggtgcggcgggcgccgactccgcggcggaggaaccgagcgctacctctccgacggtccaggacgagccgatcgccatcatcggcatggcctgccgcctccctggtgacgtcggaacacccgacgaactctgggagctgctggaaaccggccgcgacgcgatgtcggacctgcccgtcaaccgcgggtgggacgtggcggggctctacgacccggatccagacgcggcggggcgttcctacgtccgggagggcgggttcctccacgacgcgggggagttcgacgcggagttcttcggcatctcgccgcgtgaggcgctggcgatggacccgcagcagcgcatcgtcctcgaactcgcctgggaatcgttcgaacgtgcgggcctggacccggccggccgccgcggcagccgtaccggcgtgttcatgggaaccaacggccagcactacatgccgctgctgcagaacggcaacgacagcttcgacggctacctcggcaccgggaactcggccagtgtcatgtcgggccgcatctcgtacaccctcggcttggaggggccggcgctgacggtggacacggcgtgttcgtcgtcgctggtcgcgctgcatctggcggtgcgggcgctgcgcaacggtgagtgcgacctcgccctcgccggcggcgcgaccgtgatgtcgacgccggaagtcctggtggagttctcccggcagcgtgcagtctccgcagacggtcgctgcaaggcgttctccgcctcggccgacggcttcggaccggccgagggtgcgggcgtgttgctcgtcgagcggttgtcggatgcggtgcgtcatgggcgtcgggtgttggcggtcgtgcgtggttcggcggtgaatcaggacggtgcgagtaatgggttgacggctccgaacggtccgtcgcagcagcgggtgattcgtcaggcgttggcggatgcgcggttgggtgtggctgatgtggatgtggtggaggggcatgggacggggacgcgtctgggtgatccgatcgaggcgcaggcgttgttggcgacgtatgggcagcgggatgcgggtcggccgttgcggcttggttcgttgaagtcgaacgtggggcatacgcaggcggctgccggtgtggcgggcgtgatcaagatggtcatggcgatgcggcacggtgtcctgccgaagacgctgcacgtcgatgaggtctctccgcacgtcgactggtcggcgggtgcggtgtccctgctgacggagcaggagccgtggccggaggtggggcgccctcgcagggctgcggtctcttcgttcgggctcagcgggacgaatgcgcatgtggtggtcgaggaggcgccggtcggggaggcggggcaggccgccggggatgctcggttggctgtggtgccggtggtggtgtcgggccggtctgcgggtgcggttgctgaactggcctcccgcttgaacgagtcgattcgttcggatcggttggtggatgtggggttgtcgtcggtggtgtcgcggtcggtgttcgagcaccggtccgtgctactggcgggggactctggcgagctgcataccggtctggttgctgtcgggactggtgtgccgtcgcctggtgtggtgtcgggtgtggcgtcggtcgggggtggccggtcggtgttcgtgttccctggtcaggggacgcagtgggcggggatggcgctcgggttgtgggcggagtcgtcggtgttcgcggagtcgatggcgcggtgtgaggcggcgttcgaggggttggtggactggagtctggcggatgtgctgggtgacgggtccgcgttggagcgggtcgatgtggtgcagccggcgtcgttcgcggtgatggtgtcgcttgctgagctgtggcggtcgttgggtgtggtgccggatgcggtggtggggcattcgcagggggagatcgctgctgcggtggtggcgggtggtctgtcgttggaagacggggcgcgtgtggtggtgttgcgtgcgcggttgatcggccgtgagctggccgggcgcggtgggatggcgtcggtggcgctgccggtcgcggtggtggaggagcgtctggcggggtgggcggggcgtctgggtgtggcggtggtcaacggaccgtccgccacggtcgtcgcgggtgatgtggatgcggtggcggagtttgtgaccgcgtgcgaggtggagggggttcgggcgcgtgttctgccggtggactacgcctcgcactcggcgcacgtggaggacctgaaggccgagcttgaagaggtgctggccggcatcggcccggtgaccggtgggatcccgttctattcgacgtccgaagccgcgcagatcgacacggctggtctggacgcggggtactggttcgggaatctgcgtcggccggtgcggttccaggagacggtcgagcggttgttggcggatggtttccgggtgttcgtggagtgtggtgcgcatccggtgctgacgggggcggtgcaggagaccgcggaatccaccggtcgccaggtgtgtgcggtcggatccctgcgtcgtgacgagggaggtctgcgccgcttcctcacctctgcggcggaggcgttcgtccagggggtcggggtgttctggccggcactgttcgacggcaccggcgcccgtatcgtcgacctgcccacctaccccttccaacgacggcactactggtacaacgaccctgcccgccgcacgggcgatgccacctccttcggtatggcgcaggccggtcatcccttgctcgatgccggtacggagctccctgaatcaggcgagcacctctacaccgctcggctcgccgccgactcgcatccttggctgctggaacacaccctgctgggtgcgccgttgctgcccggtgcggcgttcgtcgacctcgtcctgtgggccggcggggaggtcggatgcgacctgatcgaagagctgacgctgacctcgccgctgctgttgtccgacagcgctgcccttcaactgcggctggtcgtgggcacggcggacgccgagggacgtcgtacgatcaccgtccactcgcggccggacggagacccgcgtacgacgcgcacacctgcggcatcgtccgagaccagcccggacgcggagtcggacacggagatccgtagggacacgtccgcctggacgaagcacgctcaggcgacggtcgcccccgcccctgacgtccccccttccggggtggacgcggaaggggacgccgttcgccccgcagtggaatggagcgtggcggcgacggagtcggatgccttccaggccgaggacttctacgcgtccttcgccgcacacggctatggctacggcccgctgttccagggcgtacggtcaggccgtcaggacgggaccgacgtctacgccgaagtcgccctggatcacgaccgcttgccgtctgccgagcagttcggcctgcaccccgcgctgctcgacgcggcgttccagacgatgcgtctgggatcgttcttccccgacgacggacaggcacgtgtgccgtacaccttccgggggattcgtctctacgccccgggagccgcgcgcctgcgggtccgtgtctcggcggtcggggccgatgccgtacgcgtggagtgcgccgatgagcgggggcggctcgtctgtgagatcgacgccctcgtcgtcagcacggtctccccggaccagttgcggccggccggacaggacgcgacccaggacatgctgcaccggatcgagtggcccgtcctctccccgccgaccggcagcgccacctcccctgctccgccccgctggatcgtggtcgggggcgaggacgagggcctcgggctcgggggccttcgactcgacggtccgaggcttgacggtcccgggcttgcggaagcgctgtccgaagccggtatggggaccgagcgtcaccggaacctggccgacgcgctgtcggccgtacggacgccggtggacacggcaggctccgctgccgccgccggcacgacctccctcatagccgtccccgtaccgcagtcgcccaccatggacgccggtgccgtgcgccacgccgtccaccgagccctggagctggtgcagggctgggtggcggcggacgaggcggcggaagagggcgggagcgacggtgccgcggccgaccggcggctggtgctggtcacgagcggagcggtgtccacgggtgacgccgacccgctgcgcgacccggtggccgcggccgtctggggtctgatcaagtccgcccagtcggagcagcccggccgcatcgtcctcgttgacctcgacgagggagccgtggacggggcggccttggcagccgcgatctcgaccggcgaaccacaactcgccctgcgcgacggcgatgtgcacgtgcccaggctggcacccctgtccgtgcgggactcgcagacgctgctgccgcccgccggtacgcgcgcctggcatctggtcggcgccggcaccggaaccctttcggacctcgcgctcgtaccggcgcagaccgacaccgtcgcgctcgcacctgggcaggtgcggatcgcggtgcgagccgccggactcaacttccgggacacgctcatcgcgctcggtatgtatccgggcgagggcgtgatgggcgccgagggcgccggagtgatcaccgaggtcggcccggacgtggtgagcctcgccgtcggggaccgcgtcctgggcatgtggaccgacgggttcgggccgtacgtcgtggccgaccaccgcatggtggccccgatgccgcgcgactggtcctacgcggaggccgcttcggtacccgccgtcttcctcagtgcctactacggactcaggcacctggccggtctgcgcgccggccagtcggtgctggtgcacgcggcggcgggcggtgtgggcatggctgccgtccaactcgcccggcacttcggggccgaggtcttcggcacggccggcacggccaaatgggacgcactgcgggcacagggcctggacgaccggcacatcgccggttcacggacgctggacttcgcggaccggtttctcgacgcgaccgaggggcgcggcgtggacgtcgtcctgaactcgctggcgggcgacttcgtcgacgcctccctgcgactgctgccgcgcggagggcggttcgtggaactgggcaaggcggacgtacgcgacgccgcgcaggtcgccgccgaccggccgggaaccgtctaccgggccttcgagctgatggaggccgggccggagctgatcggccgcatgctgaacgaactgctggaactgttcgagtccggggcgctgcgcctgctgcccgtcaccccgtacgacatccggcgggcacccgacgccttccgcacgctcagccaggccggtcacgtcggcaaactggtcctgacgatgccaccggccttcgaaccccacggcacggtcctgatcaccggcggcaccgggaacctgggcggaacactcgcccgccatctcgtgaccgaacacggagtgcgccacctgctcctggccggacgtagggggcccgaggccgaaggcgccgccgaactcgtacgggaactgcacgacctgggcgcctccgtcacggtcgccgcctgtgacgtggccgaccgagcggcgctccggaaactcctcggcggcataccgccggagcgcccgctcaccggagtcgtccacgcggcgggcgttctcgacgacggcgtggtcacgtcattgaccccggacagggtcgacggcgtcctgcggcccaaggtggatgccgctctcaacctccacgaagcggccctcgatcccgaactcggtctcgacatcaccgcgttcgtcctgttctcgtccgtcgctgccctgctcggcggctcgggtcagggaagctacgccgccgccaacggcttcctcgacggactcgcccagtaccggcgtggccgctcgctgcccgcgctctccctcggctggggcctggccgggagcggccggatgacatcccacctggacagccgggccctgctccggcgcatggccaggggcggtgtcctgccgctgtccccggcggagagcatggcactgttcgacgcggcccagggcttcgacgaggcgctccaggtgccggcgcgcttccacaccgccgcactgggcgccgacggcaacgtcccgccgctcttcaacggactgatccggggcgggacggcgcatgccgaggcccggcgcaggacggtcggcgcgtcgcccgcgggtggtcctgccggaggcgagccggtgaacctcgccgaccggctgtccggactgacggaggacgaacagcgggcgctgctcctcgacacggtgcgcacgcacgcggctctcgtcctgggccacacgggcacggacggcatccaggccgaccgggcgttcaaggacctgggattcgactcgctgacggccgtcgagatgcgcaaccggctcaccgccgccacggggctgcacctcgcggcgacgctggtcttcgaccaccccgctccggcggacctcgccgagcacctccgctctcgactcgtccccgaggggacggacgtaccgccgctcctcgcggaactcggtcggctcgaaacggcgttcaagaagctgaccaccgcggacctcgcctcggtcgtgcccgacgacatcgcccgcgacgagatcgccgtacgtctcgccgccctcggttccctgtggaacgggctccatggcaacggcctcagcggagacgcggcgcagaagcacggcgactcgatcgtcgaggacatcgactccgccgacgacgacgagatcttcgccttcctcgacgagagcttcggcgactcctgaccgcaggcacctccgtacggaccgacgactctgcagacgggtgatgagagatggcgaccgaacacgagcagaagctccgcgactacctcaagcgggccaccaccgaactccacaaggccacggaacggctgaaggaggtcgaacaacgcgctcacgagccggttgcgatcgtggggatgggatgccggttcccgggcggggcgtcctcgcctgaggagttgtgggacctggtggcggcggagacggacgcggtctcccccttcccggtggaccgggggtgggacgtgacggggctgtacgacccggatccggacgcggcagggcgtgcctacgtccgcgagggcgggttcctccacgacgcgggggagttcgacgcggggttcttcggaatctctccgcgtgaggcgttggcgatggatccgcagcagcggttgctgctggagacgtcgtgggaggcgttggagcgggcgggcatcgatccgcacacgctgcgcggcacgcggaccggggtctacatgggtgcctggaacggcggatacgccgaggggattccccaacccacggcggaactggaggcccagctcctcaccggcggcgtggtgagcttcacctcgggccgtgtgtcctacctcctgggtctggaggggccggcggtgacggtggacacggcgtgttcgtcgtcgctggtcgcgctgcacctggcggtgcgggcgctgcgcagtggtgagtgcgacctcgccctcgccggcggcgcgacggtgatgtcgacgcccgacgtgttcgtgcgcttctcccggcagcgaggagtggccgcggacggtcgctgcaaggcgttctccgcgtcggccgacggattcggaccggctgagggtgtgggcgtgctggcggtggagcggttgtcggatgcggtgcgtcatgggcgtcgggtgctggcggtcgtgcgtggttccgcggtcaaccaggacggtgcgagcaacggactgacggcgccgagcggacgagctcaggcccttctgattcgtcgagcgttggcggatgcgcggttgggtgtggctgatgtggatgtggtggaggggcatgggacggggacgcgtctgggtgatccgatcgaggcgcaggcgttgttggcgacgtatgggcagcgggatgcgggtcggccgttgcggcttggttcgttgaagtcgaatgtggggcatacgcaggcggctgccggtgtggcgggcgtgatcaagatggtcatggcgatgcggcacggtgtcctgccgaagacgctgcacgtggatgagccgacggcggaggtggactggtcggccggcgcggtgtctttgctgagggagcaggaggcgtggcctgaggtggggcgtctgcgtagggctgcggtgtcttcgttcggtgtgagtgggacgaacgcgcatgtggtggtggaggaggcgccggttccggaggacggggaggcggtcgggggcggtgtgcctttggctgtggtgccggtggtggtgtcgggtcgttctgcgggtgcggtggcggagctggcgggccgggtcagcgaggtggctgcgtctggtcggttggtggatgtggggttgtcgtcggtggtgtcgcggtcggtgttcgagcaccggtccgtggtactggcgggggactctgccgagctgaatgccggtttggatgctgtggccggtggtgtgccgtcgcctggtgtggtgtcgggtgtggcgtcgggtgagggtggccggtcggtgttcgtgttccctggtcaggggacgcagtgggcggggatggcgctcgggttgtgggcggagtcgtcggtgttcgcggagtcgatggcgcggtgtgaggcggcgttcgtcggcttggtggactggcgcttgtcgcaggttttgagcgatgggtcggcgctggagcgggtggaggtggtgcagccggcgtcgttcgcggtgatggtgtcgcttgctgagctgtggcggtcgttgggtgtggtgccggatgcggtggtggggcattcgcagggggagatcgctgctgcggtggtggcgggtggtttgtcgctggaggacggggcgcgtgtggtggtgttgcgtgcgcggttgatcggtcgtgagctggccgggcgcggtgggatggcgtcggtggcgctgccggtcgcggtggtggaggagcgtctggcggggtgggcggggcgtctgggtgtggcggtggtcaacggaccgtccgccacggtcgtcgcgggtgatgtggatgcggtggcggagttcgtggccgcgtgcgaggtggagggggttcgggcgcgtgttctgccggtggactacgcctcgcactcggcgcacgtggaggacctgaaagccgagcttgaacagattctggccggcatcggcccggtgaccggtgggatcccgttctattcgacgtccgaagccgcgcagatcgacacggctggtctggacgcggggtactggttcgggaatctgcgtcggccggtgcggttccaggagacggtcgagcggttgttggcggatggtttccgggtgttcgtggagtgtggcgcgcatccggtgctgacgggggcggtgcaggagaccgcggaatccaccggtcgccaggtgtgtgcggtcggatccctgcgtcgtgacgagggaggtctgcgccgcttcctcacctcggccgcggaggcattcgtccaaggcgtggaggtgtcctggccggcactgttcgaaggcaccggcgcccgcacggtcgacctgcccacctaccccttccaacgtcggcgctactggctggagtcgcgccctcccgcggcgccgatcgagactgccgcagcctctggcatcgagagctggcgctaccgcgtggcgtggaagagcctgtcgctgtcggagtcgtcgcgtcttgacggccggtggctgctcgtcgtgcccgaaaccctggacgccgacggcacgcggatcgcccacgacatccagcacgccctcaccacccacggcgccacggtctcccgtctgacggtcgacgtgacgacgaccgaccgcgccgacctgtcggcgcggctcaccaccaccgcggccgaagaccaggggcctctccggggcgtcctctccctcctgtccaccgatgaacggcagcacccggatcatcccggtgtcgaccgtgccacggcgggcacgatgctgctcgcccaggcgtgcggggatctggtcgtggcccggggcgtggagccgaggctgtgggtcgtgacccgcggggcggtcgcggtgtcccccgccgagcgtccgtcgtcagccggcgcccaggtctggggcctggggcgctgcgcggcgctcgaacttcccactcggtggggtgggatggtcgaccttcccccggcggcccgggatgctggaaggcacgtacggcggctcgtgcgtctgctgtcggagacctgtgcggaggaccaggtggcgctgcgtgcgtcgggtgcgtacggccgcaggctgctgcccgcgtccagcccctccgtatccgtcccccggaccgcgaagagcggctaccagccgcgcggcacggtgctggtgaccggcggaaccggtgccctcggtggccacttggcacggtggctggcccgcaacggcgccgagcacatcgttctggccgggcgtcggggcgagggtgctccaggagccgcggaactgtcggcggagctcaaggagctgggtgcggaggtcaccgtcgcggcctgcgacgtggcggaccggaacgcgttgcgtgacatgctggaatccctgccggccgaccggccgctgtcgggggtgttccacgctgccggtgtcccgcactcggcgccgctggccgagacggatgtggcggggctcgccgccgtgctcccggggaaggtcgtcggggcacggcacctgcacgaactcaccagggagaaggaactggacgcgttcgtgctgtacgcgtcgggcgccggggtgtgggggagcggcgggcagagcgcgtacggagccgccaacgccgcactggacgcgctggccgaacagcgccgggctgagggactgcccgccacttcggtctcctggggcctgtgggacggcggaggcatggccggcgagcgaggcgaggagttcctcaccgccctcggcctgcgggccatggagcccgagtcggctgtcgccgccctggaggaggccctggatcgtggggacacctgcgtgagcgtggtcgacgtcgactggtcccggttcgccgagtcgttcaccgccttccggcccagcccgctgatcggggagctccccggggtacgtgccgtgcccgacggatcggcgggcggaccgtcggacgacctcgcggacgctgcgcggcacggcggggcagccgaccggggtgtgcctgcagggctcgcccgggcgacgggcgacgaccggcaggacatcctgctcgatctcgtacgccgccatgccgccgccgtcctcggtcacccgggaccgcagcacatcgagcccgacgccggtttccggaccctggggttcagttcggtcaccgcggtggaactggccaacaagctcggtgcggccgtgggaacgaagatccccgccaccttcgcgttcgaccaccccaacgcccgtgccgcggcgtcccgcctcgacgtcctgttggcggcgtcgagcgatgagaccgcgcaggaggcggagatccggcaggcactgcggactgtgccgctggcccggctgcgggctgcggggctcctcgacggcctgctcgaactcgccgggctggaagcggagcccggcctgccgggcgacgtaccggatcgcggtgcggccacgccggacgaggagtccgccctggcggaagtcgacggcctggacgccgaagcactggtcgacctcgtcctcaaccagtccgactcctgaccgccggcggcggcgccgcggcccgccgtgccgtcgccgccctcggccgtacgaagaaccccacagacctgaccgggtcacggcccggtgctcagcaaggagaccactcatggctctgtcccaagagaaggtactggaggcactgcgcacctccgtcaaggacgccgaacggctgcgcaagcgcaaccgcgaactcctcgcggcccgccacgagcccatcgccgtcgtcggcatggcctgccgctatcccggcggggtccgttcgcccgaggacctctgggaactcgtcgtgtcgggcacggacgcggtgggtccctttcccgaggaccgtggctgggacgtggagcggatctacgaccaggacccgtccgtcccgggcaccacgtactgccgcgagggcggattcctttacgatgcgggggacttcgacgcggctttcttcgggatagggccgcgcgaggccaccgtgatggacccccagcagcgccagctgctggaggcgtcctgggaagccctggagcaggccgggctggacccccgggcgctccgcggcagccaggggggtgtgttcgtcggcgcggcgaaccagggctacgtaccgggtgacgcggcggcgtcgggacgtctgccggagggttccgacggctatctgctcaccggcaacgccgacgccgtcctgtcgggccggatcagctacttcctgggcctggaaggcccgtccatgaccgtcgagacggcctgctcctcctccctggtggcactgcacctggcggtgcaggcgctgcgccgtgaggagtgcgagttcgccctggccggaggggtcgccgtgctcgccaacccggccgcctacgtggagttcgcccggcagcggggactcgccccggacgggcgctgcaaggcgttcgacgacgcggcggacggtacgggctgggccgagggcgtcggcgtcctggtggtggagcggctgtcggacgcggtacgcaaggggcaccgggtcctcgccgtcgtgcggggcacggcggtgaaccaggacggtgccagcagcggtctgtccgtgcccaacgggccctcccagcagcgggtcatccgccgagcgctggccgacgcccggctggaggccggccagatcgacgcggtggaggcccacggcaccggcactcggctgggggaccccatcgaggcgcaagccctgctggacacgtacggagaggagcggagccccgaacgccctctgtgggtcgggtcgttgaagtcgaacttcggtcacgcacaggcggcagccggagtcggcggcgtcatcaagacggtgatggcgctccggcacggcctgcttccccgcacgctccatgtgaccagcccgacgcggcacgtcgactggggcgacggacaggtgcggctgctgaccgagccggtcgactggccgcggaccggcgccccccggcgggccgcggtctcggcgttcggcgtgagcggcaccaacgggcacatcatcctcgaggaggcgccgccgcccacccggcccgaagcggtccggcaggccggggagcggcggccggtcctggtcccgtggacgctgtccggccgtacgaggccggcgctgtgccggcaggccgcgcgcctggcggcgcacctcgaacagcacccggacctcgacccgctggacgtcgggttctcgctcgccacgacgcgcacccacttcgagcaccgggccgtgctgctcgcggacgccgccaccgagggcggctcccgtgccgacgcgctcggggcgttgcgggcgatcgcggaggaccgcgacccgggcggggcggtacgggacaccgcgcggggcgaagggcgtatcgccttcctgttctgcgggcagggcagccagcggcccggcatggcggagcagctgtacgcgcagtacccggcgttcgcgcgggaactggacacgatcgcgacgcatctggacgcccatctggaccgtccgttggcgacggtgatgttcgcgccggccggtacggcggaggccgcgctgctcgacggcacgcagtacgcccaggcggccctgttcgccgtagaggtcgcgttgttccggctcttcgagggctgggggctgcgccccgacgtactgctgggccattccgtgggcgagctggccgccgcccacgtggccggggtgttcgggccggcggacgcctgctcgctggtcgccgcacgcggccggctcatgcaggagctgccggccggcggcgcgatgctctcggtccgtgccgccgagcacgaggtgcgggagctgatcgccgggcaggaggaccgcatcgcggtggcggccgtcaacgggccccgctccgtggtcgtatccggggacgaggacgcggtctcggcgctcgccgaggagctgaccgaatacggcgtgcgcaccaagcgcctcaacgtcagccacgccttccactccccacgtctggactccatgctggagacgttccgccgggtcgcggagacggtggagtaccgcgagccgacgctcgacgtgatcagcggcctgaccggccgcccggccgacgccggggaactcgccaccgccgactactgggtccggcaggcgagggagaccgtccggttccacgacggggtgcgcgccgcgcacgcgcgcggcgtcagcaccttcgtggagctggggccggacggcgtgctgtgcggcctggccctggagaccctggcggaggagaccgacggggaagcggccgccgagacgcccggccgggcgcgggcggcgctggtgcccgtgatgcgtcgggagcggccggagggcagtaccctcctgacggcgctcgccacggcccacgcgcgcggggcggaggtggactggtcccggttctacgccgacaccggcgcccgccacaccacactgcccacctatgccttccagcgccagcggttctggctggagacggcggcccccgccgcgcccgcggcgggccagggggccggaccggccgacccgcaggacagcaccggtccggccgcccggcccacgctgacggaacaggacctcctcctgctcgtgcggacggaagcggcggccgcactcggccacgccgaactggaggacgtaccggccgacagcctcttcggcgacatcggcttcgactcgctcgcggccatcgaactgggcgccgccctgaccggcgccaccgggctggaagtgccgtcgtccctcgtcctcgaccaccccacgcccagggagctggccgcgcacctggcagccgcccggacggccgccgacagcgacgacacgtcccccgaaggcccggacacggccggtgagagcagcctgtcggcgatgtaccggcgggccgtgcggctcggccgggccgagccgttcatcggcacactcgccgaactcgccgccttccggcccgtcttccccgccgatcacaccctcgcggacggcgagaccgtcggacaggcggccgccgcctggcagccggctccggtgcgcctggccaccacggacggtgagggaccggagctgatctgctgcgcgggtacggcggtggcgtcgggaccggaggagttcaccgcgctggccgcggccctgggcgaccggctgaccgtgtcggcactgcgccagcccggcttccgcgcgaacgagttgctgcccggctccctggacgggctgctcgacgcgcaggcggacgcggtgctgcggcacacgggtgacaggccctacgccctcctcggccactcggcgggcggggcgctggcgcacgcgctggcctgccgactggaggagctcggcgcgggtcccgcggcgctggtcctggccgacgtctatctgcccagctcgccgggggcgatgggggtgtggcgcaacgagatgctcgactgggtcatgcggcgttccgtggtgtccatcgacgatgcccggttgacggccatgggcgcctacaaccagatgctcctggagtggacaccgcggcccacgaaggcgccggtcctgttcctgcgcgcgacggagccggtgaggccgtggtccggagaaccggagagctggcgggcgcactgggacggcggcgaccacaccgccgtcgacgtgcccggcacccacctgacgctgatgaccgagcacgcccgccacctcgcggcgaccctccacacctggctcggcaccctgtgaaccacgcccggggcggcttcgccgcgcgtaggactgccgcctcccccgacttccgtacaccgcgacaccttggaggactcccgtgacaacgcagtggaccaccccgtccgtgctcggccgcagactgcaacgcacctacgtggggcactggttcgcaggaacgcagggagacccctacgcgctgatcctgcgcgcccagcgggacgacaccaccccctacgaggaggacgtccgcgcacgcggaccggtgttccacagcgaggtgctcgacacctgggtgatcacggacggcgctctcgcccggtccgtcctgaccgacgcccgcttcggcgggctgacgcgcgcgggaggacggtatcgcgcggagcttctccctccggcgggccccgaggtcggtccggcccgcgcaggggtacgcggcggcgtgcgggccgacgccgatccggcggtgtcggcgcaggacgaggtggtggtggaggccctcgccgagcagctctcacgcaccctcctgggcggactcggcgacgacttcgacctcgtcgccgcctttgcgcgacgcctgccggcacaggtcctggcggaattcctcgggctgcccgcagccgcgcgcagccggttcgaggaactgctggccggctgcgcccacagcctcgacagccggctctgtccgcagacgctcgacatcacacggaccggcctcggagcggcggccgagctccgggaactgctcgcgcgccacctcggcgggagcggaccacgctccgctcaagcggcagtctccctggcagtcgaggtggccgcacccgccggcgcgctcatctgcaacgcggtcgaggcgctgagcagctctcccgggcagtggaacgccctccgccagaacccggagaaggccgacgccgtcgtggcggagacctggtggcgacgaccgccggtgcgggtggagagccggatcgcccaggaggacgtcgacgtggccggagtgcccgtccccgcggacgggcacgtggcgatcctcgtcgccgccgcccagcgcgacccggcgatcaccccggccccgacgaaggacgacaccggcacccccggacagggcgactgcggcgtgcccctggggctcgtcggcgacgcgcacgccacctccgccgcccggacggtccgcgccctctgccgcggtgcgctgcgagcgctcgcgcaggaggcaccgggcctgcggccgaacgggaccccggtgcgcctcaggcgggcacccgtcacgctcggccacgcccgcttccccgtcgcccggacgggccgggggacaccgaccgacgcgggcgcggcatgagcacccgcgacgaccaccgactgccgaacggggagacgagccgatgcgcgtcctgatgacgtcgatcgcccacaacacgcactactaccacctggtgccgctcgcctgggccctgaaggccgcgggccacgaggtgcgcgtcgccggccagccccgcgtcacggacatcatcaccgggtccggactgaccgccgtgccggtcggtgacgacgaggacatgatggagctgttcgccgagatcggcggagacatcaccccctatcaggagggactggacttcgccgaggagcggcccgaggcacggtcctgggaacatctgctcggacagcagaccgttctgacctcgctgtgcttcgcaccgctcaacggcgactcgacgatggacgacatcgtcgcgctggcccgctcctggcagccggacctggtgatctgggaacccttcaccttcgccggagcggtcgccgcccacgccgtgggcgcggcgcacgcccgcgtcctgtggggtcccgatgtcatcggccgggcccgggaacggttcgtggaggccaaggcacagcaggctcccgaacaccgggaggacccgatggccgagtggctcggctggaccctggagaggctgggcctcccggccgccggagacgggatggaggagttgctgaacggccagtgggtcatcgacccgggcccggagagcgtccggctcgaccttcgcgagccgatcctgcccatgcgtttcgttccctacaacggacctgccgtcgtccccggatggctgtccgagaagccgaagcgaccgcgcgtctgcctcacccagggagtgtcgggacgcgagacccacggcaaggacgccgtccgcttccaggacctgctcgcggcgctcggcgacctcgacatcgagatcgtcgccaccctggacagcacccagcgggagaacctgacggaggtccccgacaacgtccggatcgtcgacttcgtctcgatggacgtgctgctgccgagttgcgccatgatcatctaccacggtggcgccggcacctcggcgacggccctcctgcacggcgttccgcaggtcgtcatcggagcgcactgggacgtgccggtcagggcacggcagctcgacgacctgggcgccggcatcttcatccggcccgaggacctcgacgccgccacactgcgcgcggcggttcagcgcgtgctcaccgagccctccctccagcgggccgcggaccggctgcgggccgagatgcgctccaaccccacgccggccgagaccgtcacggtgctggagcggctctcccggagccaccgacagccccgctgaccacacgcggtacacggtgcgggcccacgtgccgggggctccaccgtcgccggcggtcgtcggatcgccgtccggccatgtcccggcacccaaggacggagcagagcagaacatggaattcgaaggtcaggtcgcgctcgtcaccggggccggcagggggatcggccgtgcgacggtcgtccgcctcgcggaggccggatgtgacatcgccctccactacaaccaagcgaaagcgcaggccgaggaagtcgccgagcgcatcgccgcactgggccgcacggtcgaactgttcccgggcgacctctcccgccccgagaccgggcgacagctcgtggccgcggtgcagcagaagttcgaccggatcgacatcctggtgaacagcgcgggcatcacacgggacaaactcctgctgtccatggaggcggacgacatccaccaggtcatcgccaccaacctcgtcggcccgatgttcctcacccaggcggtcgcgctcaccatgctgcgtcaacgctccgggcgcatcgtcaacatctcctccgccgccgcgagcaggcccggaaagggccagtccaactacgccgcgtccaaggccggtctggaggccttcaccagggccatggcggtggaactcggatcccgcggaattctcgtcaacgcggtcgctcccggcatcgtcaagaccggcctgaccgaggctctccgcgagggggcggagcccgaactcctggcccggcaggtgatcggttccttcgccgaacccgaggcggtggcggaggcggtggcctacttggcgagcccgcgcaacacgcacacgacgggcacggtcctcaccgtcgacggcgggctcaagatggtgtgaggcccaccgggcatcggacagccggtggatccgccggaggcggagacccgcgcaccgcgggcgtcgacccgcgcaccgcgggcgcgcgtgggggcgcccgcggtgcgcgaaggccgcctctggctcgcgtcaccggaagaagcccgattccccagcgggcgaccgtagccggagcgttggaccgccctgctccgcacgtcgagccccaccgtggtagcggcggacatgcccagggggcgcacgcccggatcctgccgcacgaccaggcgcaccagggtttcggcgaacacggcggccgacgccgcggggttgccgccgcggcacggccgacgcgcgctggtcgcacggggtgagcccgcgggagcggagggcgccggttcgctcaggacgccgggatctccgccgggacgacggactcgggggcgccgtcggggccgggcctcgtgccaccgtcggcgtcgagctgcggcggatcggagagctgctcgcgcacgtacccccacaccacggcgacgagggcggcgacgggcacggcgaggaggctgccgacgatgcccgccagactgccgcccagggtgacggcgagcaggacgacggcggcgtgcagtccgagtccgcggctctggatcatgggctggaagacgttcccctccagctgctgcaccaccacgatgatcgcgagcacgatcagggcgtcggtcaggccgttggagaccagggcgatgagcacggcgacgaatcctgctaacagcgcgccgatgatcggcacgaaggcgctgacgaaggtgagtacggcgagcggcaggacgagcgggacgccgaggatccacaggccgatgccgatgaggacggcgtcgaggaggcccaccgcggcctgggagcggacgaaggcgccgagggtgtcccagccgcgttcggcgatcgtggtggcgtcggtcgcgaggcggccggggagctgacgggcgagccagggcaggaagcgcggcccgtccttgaggaagaagaacatcaggaagagcgcgaggacggccgtgacgaccccgttcaccaccgtgccgacgccggtggcgagggtggtcagcagggatccgacgctgttctggagacggtcggtcgcggtgtccagggcgccggtgatctggtcgtcgccgatgttcaggggcggacccgcggtccactcgcggagacgctggatgccctcgacgacaccgtcggccagttcgccggactgcgaggcgacgggcacggcgatgagcgcgacggtgccggcggtgacggcgaggaagaggacggtgaccaccgaggcggcgagcgccggcggccagccgagacggcgcaacaagcgggcgaagggccaggtcagcgtggtgatcagcagaccgatgacgagtggccacacgatcgaccacatccggccgagcagccagatcaccgccgccgtgcccaagagcaccagcagaagctccgtcgatatgcgggccgaggcccgtagcgcggcacgtgttctcgcaggactgagagaggcagacatggcgatcaccctagagcggcccgggcggcccgctgcccccgtgccccgatccttcgcgccggggtgacgcgcatcgggggttccggcactgcctgagcgcctgcacggacggggcgggttacgccgaggggaagcagccggctccggatcgacaggagtgcgggtgacggtcgtacaccggcgctacgtcgcccactccgcggcgcacaagggcggtcgccgtctggcccctccccgcgtccgacgaccgcccacccctcgtcagggagcggggtccggggcgaggtggatacgggccgctaccgggaggtggtcgctggccgtcgcgggaagggtccatgccgaggcggcccggacgcccccgagaaggatctggtcgatccggacgaccggcaggcgcgccggccaggtgaagccgaagcccgcgcccgccgcggcctgtgcggagacgagacggtcggtgagggggcgcagtgcgcggtcgtccgtggagccgttgaggtcgcccaggaggacgacgcgccggacgggctcggcccgcacctccgccgccagcagccccagcgcctcgtcgcgcgccccggcggtgaatccgcccgggcccacgcggacggacggcagatgggcgacatagacggccagcggcccgccgggcgcgtccaccgtggcccgcatcgcccgcgtccacggcatgatcggcacagcccgcgcgtcgctcagcggatgcacgctccacaagcccaccgtgccctcgtagaagtggtacgggtacgactccgcaagggcccgctcgtaggcgggcgccgtggccgggctcagctcctccaacgccagtacgtccgccccggcggcgagcaggctccgcaccgttccggcggggtcggggttggcctgctcgacgttgtgactgaccagcgtgaggtccccaccgggcgtcgtcttgtcggtcagcgctccgccgaaggccgtcagccaggccacggccggcaccaccagggccaccaccgccgccacccgcgcgcgccacagcagggccgcggtcaccagcacgggcacggccagcgcgctccagggcagcaaggtctctgccagactgctcagccgtccgggcagtccggggagccgcccatggcccgcgacgctcaccgcgaggaagacggaacagcccgtgaccgccgccccgcggcgtcgccaccaccgccggcgcggaccggcgtcccccacgccgaccgcggctccgggagcgcggcccctgccgtccgccgtgccgtccgcagccagggagcagacggtctgttccgccctgccgtccgccgcagtggagggggcgctctccgctgccccgctgtccggcgccgcgggggaggggacagtctgtgccgccctgccgtccggcgccgcgaggaagacggaacagcccgtgaccgccgccccgcggcgtcgccaccaccgccggcgcggaccggcgtcccccacgccgaccgcggctccgggagcgcggcccctgccgtccgccgtgccgtccgcagccagggagcagacggtctgttccgccctgccgtccgccgcagtggagggggcgctctccgctgccccgctgtccggcgccgcgggggaggggacagtctgtgccgccctgccgtccggcgccgcgggggaggggacgggctccgccgtcccgccgtccggcaccgcggccggatcccggcgtgtcgtcgccgtcatcgttccccgccctgggttccggcggcggccagccgctcgcggacggcggtgagcaggccacgggccgcctcgaccgcggcccggagcccctcggcgggcgtcgtgttcgcccgctggtgcaggacggcgcccaccagcagccggggcgcgccgcccacgcgcacctcgtaggcccacaggaggttcccgcccgccggggtgctggagcccgtcttcacaccgaggacgcccggggtgtccagcaggggattggtgttggtgatcgtcccgaggcccgggacggtggtctcgcgggtggcgacgaccgcccggaagacagggtcctccatcgcggcccgcgtcagccgcacctggtcggccgcggtgcttgtggtcgtcggctcgatgccgctcgcccccgtgtagaccgtgtccttcatcccgagccgtacggcggcgcgccgcatcttcgtcacgaaggccgcctggctgccggagtcccagcgggccagcagacgggcgacgttgttgccggaggggatgaggagcagttccaggagccggcgctgggagtggcgttcgccggaccgtaccgggacggtcgactcgccgccgacccccgcctcgtgcgcggccgtccggtccacttcgatcagggggccgtcctcgtcgggcctcagcggatgctcttcgaggatgacgtaggcggtcatcaccttcgtcaggctcgcgatcggtacgggccgccgctcgccccgctcgcccagcgagccggttccttcgagctcgacggcgctctggccgtcctgcggccaggggagcggcccgatgtcggagaccggtagtcgctcgcctcccgccgccggaggcgcgccggagggcgaggcgacggtgatgcccagggccgtcagcagggccacggacagggcaccgccgacgaggcggtgacggggggtgcggggcagggacacgggccgcctccaggggctgcggtacgggatcggtacggcagcaagactccgggggctacgtctccgcctcacggtcgggagagcgcggcccgcgctcgggaacccatcggtcgtgtatcggcggggcggcggcgaccggccgccgggcgacgcggagggagcgcctgaggggcggggcgtaccgacaggcgaccgtctggggtggggaggcccgcgggctgtcccggggaccggttcacgcctcggacgtctgcccgtcctcgggcaggctcagggtcgcgaccgctcctccgtcccgggcgttggcgaaggccagggtcgcgccgatcaccctcgcctggccggacgcgatcgtcaggccaaggccgtgcccgtgcccccgttcggccgagcccgtgcggaaccgctgggggccgtgggacagcaggtcggcggggaagccggggccgtggtcccggacggtcacggtccggccggcgaccgtgacctccacccgacccgcgccgtgccggtgggcgttgacgacgaggttggagacgatgcggtccaggcgccgggggtccgactcgaccactgccgccccctgtcgcgtcacctgcgccgcgaggcccgtccgcgccaccgagtcccggacgagggcgcccaggtcgaccggtccctgctgggccgtctcggcgcccgcgtcgagccgggagacctccaggaggtcctccaccaggtcgcgcagcacccgtacccggctctggaccatgtccgtcacctcgccctcgggcagcagttccgccgaggtgaccaggcccatcagcggggtgcgcagctcgtgtgcgacgtcggcggtgaagcgctgctcggtgtcgatccgctgctggaggctgtcggccatcgagtcgaccacggccgagatctcggcgacctcgtcgcctccgcggaccgttcccgtccgggcgtcgaggtcgcccgccgtgatgcgccgggccgtgcgggcgacccggcgcagccgccgggcggggagttccgtcgccagggccgtggcggggacgacgacgcccagggtgagcagcgagtacttccacatgtgacggtccagggcctgccgggtcagcaggtcggcggtcatgtcgacctcgaccgcgtacagcttcccgccctcccggcgggccgcccggaagacgggggccgggggcccgtcctcgtagagggtggcctcgccgccgtgctcgatctgcctcagcagggcctcgggcagttcctcgggggacacccggggcccttcctcacctgctgcgtcggcgtcctccaaggccgttgccagcgccacgtgggccctgcccgcgccctcgtgcagggagcgccgcagcaccgagtcgtgcaccagcactccgacggtcagcgccatcgaggcacaggcgagcgccaccaggaggacgatcttccagcgcagcgagcgggagcgcggtaccaggcccgtgacggctccccggggggcccggctcaccgcttccacttgtagccgaagccccggaccgtctcgacgcgctcggcgccgatcttcttgcgcagccgctgcacgcacaggtcgacgacccgggtgtccccgtcccagccgtagtcccacacctcgcgcaggagggtctgacggtccagcacgatcccggggtgcgcggcgaactggagcagcagccgcagctcggtcggggcgagcgcgatccgctcgccgccccggcggacctccagggccgcggggtcgagggagaggtcgccgaagagcagcgggccggccggcgtcgcggggtcggcgggtccgggggcgggggacacgaaggcggcccggcgcagcagcgaacggatgcgcgccaccaggacggcggtgtcgacgggcttcaccacgtagtcgtcggccccggcctccaggccggacaccacgtcgagggcgtcaccgcgcgccgacatcatcaggatcgggtccgtggccgtctcccggatgcgccggcacagtccgatgccgtccaggcccggcagcatcacgtcgagcagcaccaggtcgtgccgtccctcccggaagagttcgagcccggtcagtccgtcggccgcgacgcgcacgcggtagccgtagcgctccagggacatcgcgacggaccgccggatcacctcgtcgtcctcgaccagcaggacggtcacggtcgcaggcgcgggcgccgacacggaatcagacatgtccatctctcgggcacggggcggggcgggccgacgcgcggcccggccctccatcatgcctcactcgcgcgcctcccccggcgccgccggagagccgtcggcacgcctctgagctggtctgatacctgactgatacatcatggcgcggtgaccgacacacgcccgagcggggacggacacggaggcggcgcgccggacacaagatgccgcgggagccgatccggcgcccaccgaggccgtgtgaacgacgccgtccggaaccgcacgcccgcgggggctcgtggcgggtgtcagtggtgtgcggcaggcgcgggcagcgtctccggagcggggtcggtcctgcggccgaggtggttgaaggcgaggttgaggaggacggcgaccacacatccggtgctgatgcctgagtcgaggacgatgcgggcgccttcggggaaggcgtggtagaagtccggggcggcgatggggatgatgccggcgcccagggagatggcgacgatgaggacgttgtcgccgcgttcgagggcggctccggcgagggtctggatgccgctggcggcgacggtgccgaagagggcgatgcccactccgccgaggacgggctgggggatgagcgcgacgacggaggcgagcagcgggcagaggccgagcaggaggaggatgccgccggccgcggctacgacgaagcggctgcggaccttggtgatcgcgacgaggccgacgttctgggcgaaggcgctggcggcgaagccgttgaagagcgggctgagggcggtgccgaggccgtcggcgcggagcgcggcggccagggtcttctcgtcggccggccgctcgacgatctcaccgagggcgaggacgtcggcggtggactcggtcatcgacacgagcatcacgatgcacatggagatgatcgccgcggccgcgaactgcggagcgccgaagtggaacggggtggggagtccgatgacgtcggcgtcgcctacggcgctgaagtcggcgacgcccagcggcagcgagaggagcgtgccggcgacgaggcccagcaggatggagatctgcttgaggaagcccgtcaggacgcggcgcaggaccacggtgatcagcagggtggcggtggcgaggccgatgtacgtgaggctgccgtagtccggtgcctgcgcgttgccgccctgggcccagttgaacgcgacgggcagcagggagacaccgatgagggtgatgaccgtgcccgtgaccacgggcgggaagaagcggatcagtttgcagaagaagggggcgaggaggaatccgaagacgccggcgacgatcacggcgccgtagatgacgggcagcgcgtcgtcggggccttcggccttcgctatcgcgagcatcggtgcgacgccggcgaaggagacgccgttgacgaacgggagccgtgctccgaccttccagaagccgagcgtctgcaggagcgtggcgatgcccgaggtgaacaggctggcgctcatgaggaacgcgatgtccgcggtggacaggccgacgccgatgccgacgaccagcggcggcgcgacgacgccggcgtacatggcggcgacgtgctgcagaccggcgctgaagagcttcagcgggggcagcatctggtcgaccggatgggtgtcgcccggtcttgcgggggtgtcgggacgagcgtcggttccgtcctcggtcttcgtcggttccggggcggggcgggattccggcatggcgtgtctcctggccgcggcggtctcgtgggagcgcgggcagccctgtgggagagccggcgtcgctgacgcacggctctgcggatgggggtggtgacggggtggggaggatgcggcaccgggcgtgggacgagaggccgtcgtgcctgggtgccgcggccggagagcggggagtggggcgccggagaacgggcgggagggattcgtgtcccggggttcccgcggtcctccggccggccggcgcgcagctgtccgtgatgcgcgtcgtgccactccgtccggcgaagtcggaacagttcctcacggggcggcgtgggtgtcaagaggcgggacgggccgggttgaggacctcccggacgtggtgccagaggtgcaggtcgatgtgttcgaggaagtccgccgcccgggcctccgcgcgggccgcctgacggggcgggccgccggccgcgccctcccattgctggtaggtgaggtggaggcgttcgagggcgtggccgagtacggcggggtcgagcggcacgatgcgggaaagggcccccacgtaggggggccaccaggtggtggcggcctctctgagcaggacgcggagccggccggtcctgccgcacgcggcgacgaagcaggccggtggcgggcagaggactctcaggagggcgtccgtctgggcgggggtcccctgccagcggttccggcgttcggcgtcctcccactggccgcgggtgaggccgagctcggtcgcggtctcgttgacggtgagtccggcgacggtacggcactgggcgagggtgccgggctcttcgacgagatcgaccggagagcaccagagggccgccgcgagggccttcacctgggtgttgtcgggggtggcggtgccggcttcccacgcctcgacgaggccggggtgcgcggggtgtccgcagtaggcggtgacggcccaggcgacctggccgagcgtcaggccgagctgggcgcggacggccgcggcacgggtgggatcgaagcggggcagggcatacggatcgtctgcgggtggcatagcgggacaccgtagggaccccgcggtccacggccaagggccggaacggtctcgatgtcggctccgccgcccggcgttcggcttcttcgtcggatctcctgagcgggcagaccgtcgatgacctgctctttcatgggaggaggcgggcaagcggagagaagaggaggctgaggcgactgtcggttcctccagaatccgcgaggaactggcgccttctcttgggggagttgacgaggttcaggccgggccataaagtcccgtcccgtccacaacggaaaacttcttccgtcatgcgaaacctgtggtgacagccgcccacccccgcggctcgcccccatcggccatgctcccggttcgggccgtcccccggcaccggtcactctgcccacacgcccccggcagtccgctgcccgcgggcggtgtctcaaggacctgccttgctgccccgcgccactgaccacgcatcgccgaccccaccccatccggtcgacgagatccttcccgcccgccgcatgctgcccgccgccctccaacacgtggcgagcatgtacgccggcctgaccgcaccaccgctgatcatcagcagcgccctggggctgaccccggcccagctctccgccctcctggccgccgcgctgctgatcgccgggctcggcacgatcgcccagaccctcggcgtctacggcgtcggcgccgggttgcccctcgtcaacggcgtctcgttcgccgtcgtgtcaccggcgctcgccaccgccgccacccaggggcgcgacggcgccctcccggcgatcttcggggccaccctcgtggcgggactcctctgcctgctcctcgctcccgtcttctgccgactggtcaggttcttcccaccggtcgtcagcggctgcgtcatcaccctggtcggcatctccctcctgccggtcgccggcacctgggcccggggcggagacgccgaagccgccggcttcggctcccccgccgacctggccctggcggcgacgaccctcgtcatcaccctgaccgtgcaccgcatgctctcgggccgcttcctcgggcgggtcgccatcctcatcggcatgctcgcgggcaccctgatcgcgatcccgctgggcaaggtcgacctcgaccccctcgcccaggcgcccctcttcgccctgcccacgcccttcggcttcggcaccccgcagttcgtccccaccgtgatcgccaccgccgcggtcgtgatgatcgtgtccatgatggagtccaccgccgcgctgctggcgctgggcgcggtcgccgaacggccggtccgggaccggaccatcgccggaagcctccgcgccctcggcctcgccacggtcctcggcggcgtcctcggctcgttcaccagcacgtcgtacgcgcagaacgtcggcctggtctccctcagccggatccgcagccgctatgtggtcacgctctgcggcgccgtcctcgtcctgatgggcttcgtgcccgtcctgggctcgttcgtcgccctcgtccctctgcccgtcctcggcggtgcgggggtggtcttcttcggctccgtcgccgtcacgggcatccgtacgctggccaaggccgccctcggcaccggacacaacgctgtgatcgtctccgtcaccctcgccttcggtctcttccccgtcctggacccggacttctacgcccgtcttcccgccccggtggcgaccgtgctcggctcggggatcaccgccggctgcctggtcgcggtcctcctcaactacctcctgaaccacctgggccgcggcaccgaggccgaccccgacgcgatctccgcggaacaggtcaccgccctcgacaccgcggacaccgtcctcgggccgaagcgttcctccgactggacgcccttccagccctccggcagcccctccggcacccctgaccacggccgtcacaccaggggcacggcacggcccgctcccgcctggccctacgtgaccggccccgtggaccccaccgacaccgggcggcaccaccggccgcacgaggttccggcgccgccccaccgaccagacgaggtgcccccgccgctccacccctctgccgctcacgaaggcgaacccccgcccgccgtcaccgagaacgcggtctttcccggaccgctccacccgctccacccgctccacccccggcccaccggtcgtcccgaccgtccccggcaacggcacagtgcggaggccgacccctggcagcatccgcagaccccctccgcatccggcgacagccagtagagacgacctcccccgacctcttcgcagagcccggcatcgcacaggcccggcggagaggtggcgccgacccacccgaccaccgccggaagcgcccccggggacccgtgtcccacggattccccggcgacaagacgaggtagccccgatgaccaccgtttccgccgcccgccaccgtgcgggcggctccccgcgcggcggcacgtcccgcccgggccccgacgagagaatcgcccaggtcgtggccgaggccctcggatccgcccggacggtcctcgacccggatgcgctgcccggcctcggcaccacgcgactcccgttcggcgacgggaggttcgacgcggcgatgatgctctgcaacgcccccggcgtccccgacgcgctctcgcggctcggggaactgcgccgcgtgacacggggccccgtcgtggtcctcgcgaccgacccctcgcgcgtccgctcgttctggctggaccggtacgcccccgaggtcctggccgtcgaagcgcggcgtcatccgccgatcgccgatctgaccgccgtcctcgggggttccgccgaggtgcggagcgtccccgttcccctcgactgcaccgacaccttcgacgaggcgtactacggaagacccgagaagctcctggacccgtcggcccgccaggcggggtcggcctggagcttcgtggacgaccgggtccgcgaggagttcgacacgaccctgcgccgcgaactccggtcgggggagtgggacgagcgcttcggccacctccgccgccggcccgtctacgagggatcactggtgatcgtccgtgccgtcccctgacgtcctcccggggacgcgccacccccggggtcgcgtcagcggtctccggccaggtgccccgagagctcgcgcatcagcgtctcgtgctgctggagcaggtagaagtggccgccgggcaggacccgtacccggaagccctcgggtgcgacatccgcccaggcgtccatgtcccctaccgccacgttcggatccgtgtcgccgatccaggcgtgcaccggacagccgacggcggtggggacgcgggggccgtaggtgctcaccacggtgaagtccgcccggaccgcgggcagcacgagctgtcggatgtccgggtcgtcaagcagggcggtatcggtcccgccgagcccgcgcagtacggcgaccagctcgtcgtcccccttccggtgcaggtcgagcggggtcagccggtgcggggccttgcggctggagacgtggagggcggcgggcgtgacgcggtgccgctcctccaggcgcagcgccacctcgtaggccagggacgcgcccatgctgtggccgaagagcgtcaggggcacgtccgcgagcggcagcagtgccgccgtgacccggtcggcgagcacgtccatccggtcgacgaacggctcgttgaaccgctcctggcgtccggggtactgcgccaccaggacctcggtgtcgccgccgaaggcgctgccccaggcgtggaagaagctcgcggaaccgcccgcgtgcggcaggaccgccagccgccgccggggcgcgggagtgctggagtacctgcggaaccacgtcgtgctgtccgtgccggtcgtcatgtgtgcgtacaccccgtcctcgggttcttggggtgccagtgtccccgcagggcccggtgtccggacgcggtgggggtccggtggcgagccgcttacgtgtcccggcgcttccgggaccggcggccgcacacgtgtcggcccccacgaacaccagggtgcgtgggggccgatgcgtgtttcgagtcctggtctgacgatttcaggccgaaagatatgtcggactttacagctgcgatcgaagccgatcgataatgccgtggacgggtaacgtcggaatcactcggtgctcttgagcgcaccactcacgttgacgacctcgtggcactcccgcgcctgctgtcccgtcgcgggcgtaccgggcttcgtgcaggtcacgtcgatggtcacggtgtccttcgcgccgatcctgatgggagtcacccagtggtagtcctggttgcggaacgtctccagcgcgatcgtggtgatcttgcggtccccgaaggtgatcgtcatcaccccttcgtcaccctggaagttcgcgacaacgatgtccgtgatcccgaacacggtcttctcggggaccgtgtacgtcccggtcctggactgtcccgctcccgacctcaggtcgatggtggccgagctctgccgtcccccgccggtcgcggtgccgtcgcccgtgccggccgaaccgtcgtccccgccgcccgctccaccgcccgatcccggcttctggctgctgccgccggacggccggcccgggccgggggaggagccgtccgagccgctctccgtcgggaccgggcggggctgcaccgcctgggtggccgcctcctcggcggcgctgcgcacggccggccggaccagcgtgaaccaggcgatcagcagcgcgatcagcgccgcgagcagcaggagcagccacttggggaagaccggtatctggacgaactccgcgtccagcgtcggcgccgtgtggggctcctcggcgcggtcctcggtctgctccggctcgccggtctcgcgggcgtccacggtgaacggccagaccacggggtcgccgaaccacaccgggctcgcggtgcggacccgcagccggagttccttcgactcgcccggctccagcgccggctccgccggcgtgaaggcgaaccggagctcctcgcccgcctgcccgggcgtgaaccccacccgtaccggggtgttgccctggttgcggacggccagcagatagcggccccggagccagccgcgccggcggcgcggcgagaggtcggtccgcagctcgtggaacgcgccgacgcgcaccacggtctccaggaccttgaccgactcgggctgctcgttcgggaggatccgtacaccgaggggcagctcgccggcccgtgtctccggcgagcgcggcggtgccagacggagcgtcaccgtctcggacgtgccgggatagagggagagccgctcgggctcgacggtggtccattcggcaccgtcaccgacgaccttcaggtcgtacgcctcgacgatgtcactgtcgttgcggacggtcagggtggtggtggcgatgtcgcccggcgtcacggacacggccgggatgtcgaggccgggcgcaccgggaccggaggaggctgcggaaggcgtcacccgccccaccgtaggagacctgacagatccgtacgaggcacgcgagggcaatgtccgggcagctcggctgcccggcaagcacaagtcaactctccggtaacaatggatttctagtctggagagccgccttcggcacaccaccggcccgtggtcggctcgtgtcgtgtccgccttccccccaccgacccaggaaaacaggtatccgatgttccgcaccgaggagaagaggccggtcgcgaccggcactacggcgcatgacgccgtccggggccacccggacgcccatgccgccggcttcggccgcccgcgccgcgtcaccgtggcggtctacgccgccgaccccgtgctgcgggtcggcgtcgtccaacagctccgccagcgccccgagaccgagctcgtcgacgacgcggacgcggagaacgcgcaggtctccctggtcgtcgtcgacgccctcgacgacgacgtgaccgccctgctgacccggctgagctacaacggcgccacccgcgcgggactcgtgatcggcaccctcggcgtcggggcgctccaacgcgtcgtcgagtgcggggtgtcggcggtgctgcgccgcgccgaggccgaccaggaccagctcgtccagctggtcctggcggtggccaacggcgagggcgtgctcccgggcgacctgctcggcgagttactgggacacgtcggcagcctgcgccgcgcggccctcgaccccggcgccctgcccctctccaccctcaccagcagggaggcggagatgctgcgcctggtctcggagggcctggacaccgcggcgatcgcccgcaagacctcgtactccgagcggaccgtgaagaacgtcctgcacgagatcaccacccgcctccaactgcgcaaccgcgcccacgccgtgggctacgcgctccgcaacgggctgatatgaccgtcccgtccggaccgcggcccggcggccggcgcgacagccggagggaaggcggcgctgccccaaagtgcatcccgcccttcccccgggtgcggcccccgggccctcccgccgcgtgcgccgccgccgcacgatgacggggggcacctcccggtgccgcaccggacggagaagggcaccgtgatgaagaccgctggccccggtggacggcaccgccgggggagactcgcctcggcgctcctgctgctcgtccccctgctgggcgcgacgggcgtggccgggccggacgacccccggaccgcggcggccgcggcggacgccgccgagaccacccgcatcgcctacgcgggcaccggccaccgcagcctcggcgaaccggcctccaccgactccagcaccccgctgttcggagcgggacccacccactacgacaccgacccgtccgccctcggcgaccggctggtcttcgcgagccgccgcgacgagaagcacccccagatctatctgcggggcgccgacggcggagtcctgcggctcaccagcggcctggacgcggcccgtccccggctcaccccggacggcgggtcggtgctcttcgacgccgccgacccggccggcggctcccagcgcgacctgtggctggtgcgcaccgacggcaccgggctgacccggctgacggacacgcccgccagcgaggaggacccggcggtctcccccgacggcgcccggatcgcctactccagcgacgccgaccccctggccgggcggcagatctacgtccgcgccctcacgggcggcatccccacccggctcaccgacccggcccgcggcacggcctccgagcccgcctggaaccccgtcgacgacgacgtcaaccgcgcgtggatcgcgtacacgtcgaccacgaccgaggacgggcggaccaggcagcggctgcggatcaccgacggcaccaccgacgagaccctgttcaccggcgcgtacgcgaactggcagggccacggggcggcatggctgcccgacggggacgggatcgtgttcctcagccccgagaccacctgcacctgcaggaccccctacgaccacgtcttccggtcggtcgtgcacgccgaccgggaaccctccctggtgctcgacgaggaccgcgacgtcctctcgcccacctggatcggcaccgccgagggcggccacgcgatcgtcgagcgcagctcggcggcgaccgcgcacacggcgaccctccaggacatccgcgcggacggttccgacccgcgcgacctgcagcggaagatcctgcgcgaggacccccaggccgacaccaacaccgaccccgccaaggatccgctcttccagcccgcgcccccgttcgacccgtggaccgaacggcagaactacacccccgacgggcgccgcctcgtcctcacccgcttcgagggccccgacgacgcgcggatcgagcggatctggacggccgacgccgacggtacgaacgaggcgccgatgcccctcgacgggcgcggcgcgcgggactgggacaccgacccgacgttctccccggacggcacccgcctggccttcacccgcacctcgcccggcggggtcggcgaggccgcgggagacagccgcatcctcctcgccgaggtcgccaccggccggatcaccggagagatcgtgcccccggccggtgaactccgcggcggggacgcccagccgacctggtcctccgacggcaccaccctggccttcacccgcgcccggcagatcgccgggggcggcggcagcaagcacgtgtggaccgcgtccacggctgacctgacccggcagcgcgacctgagcgcgacgcactgcccgcgcgactgcgacgtcatcgacgacagccccgccttctcgcccgacggacgctccctcgccttcaaccgcaagaacggcggcgggcggatcgacgagcgcaacggactgctcctgaccaccctgtccggcgacgcctgccaggtcctgctgcccaccgccgcccgcggccaggacggcgcgtgcgagcgggaactgccggacaccacgctcaccggtccgcaccagccgcgcgacgccgcctggaccgccgacggcaagaggctggtcttcagctcccgggccgcggccgcggtcaacagcccggagaagctgaacgtcctggacgtcggctccggtgacatcaccccgctcaccgccgagctcgccggacgccagaaggaacccaccgtccagcagtccgtggacctcgccgtcgaggcacccgccacgacgcccgacgtcaccgtcggcgcgtccggcacggtcaccgtccacgtggtcaaccacggtcccgccgcctcgcccggcacccggctcaccgtcgtcccgccgtccggtgtgcggatcaccgggatcgagtggcccggcggcacctgcgacgccgcctccctccagtgcgacctgggcgtcgtcgaggccggagcccaggtccccgtggacgtcacgctcaccggcgtcaccgccggcgacgcacccgtcgactggtcggtcaccggcgccgtcctcgacccccggcccggcgacaacgacggccggagcgtgatccccgtacgcgaggcacccccgacgccgacccccacgccgacgccgacccccacgcccaccccgaccccgactccgacgccgacccccacccggaccccgacgcccaccccgactccgacccggcccccgcagccccccgcgccgaaggccggacccggggtgcggatcaccgtccagcccgagcccggctacgtcggcggacgcgtcgtcgtcacgtacagcgtccgcaacggccgcaacgcgctcgccaccggactccggctcaggatcggactgcccgccggggtgccccacggcggacttccggcgggctgcgaccggaacggcgcgtgcgcgctgcccgacctcaccccgggcacgaccgccgtcctgcgggtcgtcctcagcccgaagaaggcgatgaccgcccgcgtcacggccgtgctcgacaccaccggcacggacgccgaccgcagcgacaacaccgcccgggagcggctgcgcgtcctccagccgcgcatcgtcgccgtgcccgacatcggcaagcccggattcgtcacctccgtccgaggcgtggacttcccgcccggcgtcccggtgcgcttcagctggaaccccgggatcaccgccgccgcctcgccgaccttcccggaggccgacggcacgttcatcggacagctcctcatcctcgccaaggaccagaccgggccgcgcaccatcacggcctcgggccccggattctccccggtgaagaccgacttcctggtcgtcagcggcaccgtccagccgccggacggggtgactcgccggtgatcc

SEQ ID NO: 2 - protein, 343 aa, Streptomyces bikiniensis
MADVLDIRQYRSPAATALMSLAVAVLYYAAGRLGLMGRLVVEGVVVTPIWPPTGVAVAALLLLGARVWPGIALGSFLVIASLTTPGPTTVVTVVSNTVAPLCAFLLLTRAGFRLDMARLRDGLSLVFLGGFGAMLISATAGVGLQVAKSSLDTTEFWPVWLAWWVGDTMGVLLVAPLLLVLAGPAGRFRVRRWKEAALLGLATLILMPMAVLSPVGMLFLVFPLLIWAALRFQLTGSMLCALFASVLATFEANAGRGGFLHLSDVEIMAKLQAFNGSAALTALLLASVITEQRATRRSVRRACRELAEVLEHLAAGEPAPGPPGTAESRAAPSGGPRDPFSEG

SEQ ID NO: 3 - protein, 279 aa, Streptomyces bikiniensis
MFRRRPADSSEDLLVRLGSLTARARELAETQRSRVELAVALQRGMLPSDLPSLPGARLAVRYEPANHGLNVGGDWYDAFPLPGGRIGMSIGDVQGHNIEAAAFMGQVRVALRALASVTADPGELLGRTNDLLVSLGADLFATCTFLRLDPAAGTLECARAGHIPHIWATADGRSGIDDSEGGPPLGVLRGADYPATRHRLTADGVFVLLTDGVVEGPSLHIDEGLDRVTRLAGITAVAGLDVDALATAVMRLAATVGHEDDAAVLVVGHDGGPPRPATG

SEQ ID NO: 4 - protein, 293 aa, Streptomyces bikiniensis
MAQPGRTTFPLGGDLSVHRLGFGALRLTGPGYWGPPEDREAAISTARLAVDLGVSFIDTADSYGLGSNEELLAEALRPYPDGLVIATKAGQCRPRPDQWVPCGRPEYLRQQAELSLRRLRLDRIDLFQLHRIDPKVPAAEQFGALGELQDEGKIRHIGLSKVTVREVEAAREHITVASVQNVYNLTDRRNDEVVEYCEKHEIAFIAWLPIARGALAAGGSPVADIATELEVSAGQVALAWLLHRSPAVIPIPGTSSPAHLRENVAASGIALSDGQLARLNALGRTHDDATVPV

SEQ ID NO: 5 - protein, 405 aa, Streptomyces bikiniensis
MTMKRNLGDLAVFGGPSAFLQPLHVGRPNVGDRARFLDRVNWALDHQWLTNGGPLAREFEQRVAELAGVRYCVSTCNATVALQLLARAADLSGEVIMPSLTFPATAQAFRWLGLTPVFVDIDPATNCLDPDLIEAAITPRTSAVVPVHLWGRPCAVDRLAKVAADHGLRLFYDAAHGLGCTSEGQPIGGFGQAEVFSFHATKVVNAFEGGAVVTDDERLAQRVRAMHNFGFTQGRVSTETGTNGKMTEAAAAMGLTSLDAFEETVRRNEANHDDYRSELAGLAGLKVMEFDRAERNNYHYLVVEVDAAVTGVHRDLLDEVLRAENIMCQRYFSPGCHEMEPYRSERPVSLPHTERLARKVLALPTGPTVSREDIRRVCDIVRLTLERGHEVTRRADERVPSTTAP

SEQ ID NO: 6 - protein, 259 aa, Streptomyces bikiniensis
MKNVVSNTIYGDHPTFPVGMNVLDKELADRMRPGAEGPDVERGTFDADHRAEGLDVPTVAHTMIGLQRLDNIQRCVERVLQDDVPGDLIETGVWRGGACILMRALLKAHGVEDRTVWLADSFAGVPVTSEDSHPLDRAMEFHHLNWVLSCSEEQVRENFARYGLLDEQVRFLPGMFADTLPTAPIDRLAVLRVDGDLYESTRDALVNLYPKLSVGGFVIVDDYLIPACKQAVHDYRSEHGIDEPIETVDVTGVYWRREH

SEQ ID NO: 7 - protein, 485 aa, Streptomyces bikiniensis
MTSHASTAADPVALCARPGADLPAAVHTVHRALVSDGRVGVDEGPTVARRLVRLAERYGNHPFTPLEEARRMLDVDRASFGRLLGLFGRVPELRHAVENGPAGKYWQNTLLPLERRGVFDAALAHKPAFPYVVGLYPGPSCMFRCHFCVRVTGARYDPSALESGNAMFASVIDEIPAGNPYAMYFSGGLEPLTNPGLGALSSRAAARGLRPTVYTNSFALTERTLDRQPGVWDLHAVRTSLYGLNDEEYEETTGKRAAFGRVRANLRRFQQLRSERESPIRLGLSYIVLPGRVHRLLDLVDFIADLNEAAPDRPVDFLNVREDYSGREDGRLFEAERAELQEGLLAFEEAVSRRTPTLNIDYGYALHSLKTGADAELLRIRPGTMRRSAHPQVSVQVDLLGDVYLYREAGFPDLTGADRYVAGRVGPGTSLTEVVERFVDDGRLIAPHAGDEYFMDGFDQVVAARLNQLEADVVAGWEDARGFLR

SEQ ID NO: 8 - protein, 836 aa, Streptomyces bikiniensis
MRTATSAERTSVSMLFGTTRTGRRIRRTVGSALAALCVGGLLTAPSAAGAPAAEPGTARVRGLVAKMTLDEKISFVHWTTGPVGGPTMTGIGYLPGVPRLGIPELRTADGPVGIRLLGGTATAMPTPVALAATFDERLAEEYGTVLGREGRALGQDIVLGPMTNVIRVPHAGRNFETYSEDPLLSSRMAAHEVRGIQNQGLMATVKHFAANNQEYQRETIDAVVDEQTLQEVELPAFRSAVRAGAASVMCSYNKVNGAHACGNEHLLQEVLREQWDFRGWVVSDWLATHATGDITRGLDQELGVELTLGQPVPESKYFSSALRAAVRDGSVPEATLDRSVVRILGQMERFGLLDGKATERPQRDPEAGRAAARTIAENGGVLLRNERRTLPLTGEDATDIAVIGNSAKHPKVTGNGSAHVIPDRATAPVDALARRAGEKARVVHEPGEDLVGVPVPESSLTPAFTSGKQLDPSGQGVFYEGRLTVPADGDYKIAFTAVGGVANLQIAGQSAVLGTEAFGTVTTTMRLTRGTHAVTMNGWAFEQTPLSVELSWVTPEAARDDFDRAVAAAAEARTAVVFAHDDSAEGVDRSSLSLPGRQDELIAAITKVNPRTIVVLNTGSSVLMPWLRETAAVLEMWYPGQEGAEATAALLFGDANPSGRLTQTFPATETGHPMAGDPHRYPGVDGKETYSEGLDVGYRWYDRTGVAPLFPFGYGLSYTTFAYSDLSVARTARGLEATVTVRNTGDRAGRETVQVYLGASPDTQAPQALRKLAGFTKVTLRPGEQRRVTVPVDEQQLRYWDTAAGTWKPGTGRRAVHVGPSAAETSLTTSVTVPSR

SEQ ID NO: 9 - protein, 401 aa, Streptomyces bikiniensis
MGLPLTSTKTAPVSYPFGRPEGLDLDEAYEQARKSEGLLWVHMPYGEPGWLVSRYDDARFVLGDRRFSHAAEAENDAPRMRELRTPNGIIGMDAPDHTRLRGLVTKAFTPRRVEAMRPHVRRMTASLLRDMTALGSPVDLVDHYAVPLPVAVICGLLGVPEEDRDLFRGWCEIAMSTSSLTAEDHVRLAGELTGYLADLITARRAAPRDDLVSALVEARDAQGRLSQEELVDLIVFLLFAGHETTASQISNFVLVLLEQPDQLALLRDRPDLLDNAVEELTRFVPLGSQAGFPRYATEDVEVGGTLVRAGDPVLVQMNAANRDALRFRSPGVLDITRDDAGRHLGYGHGPHHCLGASLARLELQEALRTLLDELPGLHLAQPVEWKTEMVVRGPRTMLVGW

SEQ ID NO: 10 - protein, 407 aa, Streptomyces bikiniensis
MTSSCPVHRTRPYPFSPPEGIALDPLYSRLREHEPVSRIRMPYGGEAWLLTRHADVRAVLGDPRFSMEAGAGRDVPRPTEYPLPAGGLISMDPPGHTRLRRLAGKAFTARRVEELRPRVSRFTDELLDGMVARDEPSGEVMEDLALPVSISVICGLLGVSYDDRHLFRDFSEALVSSSLGPAEVQRATEDFSAYMADLVADRRARPTDDFLSTMVQARDEGDRLSEAELLMMGSGLLISGYETTATQIGNFVLLLLDDRRLYERLVTEPDLVPSAVEELLRFTPLSTLDGFARIALEDVEIGGTLIRAGEAVITSIASANLDDTAFPGAASLDLARAQNPHLGFGHGAHYCMGAPLARLELQVVLSTLVERLPELRLSVPASELRWRAGSLLRTPEAVPVTWGGSAA

SEQ ID NO: 11 - protein, 282 aa, Streptomyces bikiniensis
MNEGPATTAPGSTNAGDLWLRRYRPVADPALRLVCLPHAGGSASAFLPFTCLLPDRVEVLAVQYPGRQDRRLEPFVDSVDALVTHVAGALGPWLDRPVALFGHSLGSLVAFETARRLAEQAPESRLAHLFVSGRVAPTVAHRTTAHLLSDDRLVAKLAELQGTDPRVLADEEVLRMALPAIRNDYRAAATYTWRPGAPLACPITVLTGSADPHVPTDGALAWHGLTTGETAFRSFPGGHFYLTEQAEAVCRTIRTATGLAVGRPHPSSSAAAPYDRESVHDV

SEQ ID NO: 12 - protein, 323 aa, Streptomyces bikiniensis
MRVLVTGAAGFIGSHFVRQLLSGSYPELAGAHVLSLDKLTYAGNTENLAEVAGHPRHTFLHGDICDPPTVAQALRGTDLVVHFAAESHVDRSITDSAAFVTTNVLGTQTLLRSALEAGVSRFVHVSTDEVYGSIPEGSSTEADPLDPNSPYAASKASSDLLALAFHRTHGLDVRVTRCSNNYGPHQHPEKVVPLFVTHLLEGLRLPLYGDGLHRRDWLHVDDHCRGIAMVAARGRAGEVYNIGGGTELSNVDLTRRLLGVFGADWSVVDRVPDRAAHDRRYCVDTRKITEELGWAPRVAFDRGLAETVDWYRDNGTWWKALTP

SEQ ID NO: 13 - protein, 305 aa, Streptomyces bikiniensis
VFETPLKRPGVRGIILAGGSATRLQPLTGALSKQQLPVYDKPMIYYPLSVLMLAGIQDILIISSHQHVETFQVMLGDGSRLGIQLDYAVQDEPRGVADAFLVGDKHIGNDRVALILGDNVFHGPGFSTVLKHSLRRLDGCELFGYPSKSPERYGVAEIDEQGHLLSLEEKPSRPRSNLAVTGLYFYDNDVVELAKDLKPSARGELEITDINLSYLEQGRARLTQLGRGFAWLDMGTHDSLLQAGQYVQLLEQRQGVRIACLEEIALRMGFIDADTCYRLGEELSASSYGDYLMEVASGLGATRTG

SEQ ID NO: 14 - protein, 196 aa, Streptomyces bikiniensis
MHPLSIEGAWSQEPVIHSDHRGRSHEWFRGESFRQAFGHDFPVAQVNVAVSHRGALRGIHYTEIPPGQAKYSVCVRGAGLDVVVDVRIGSPTFGRWEIVPMDAERNTAVYLTAGLGRAFLSLTDDATLVYLCSSGYAPAREHSVNPLDPDLGIAWPDDIEPLLSDRDENAPTLATAERLGLLPTYQAWQEQQQAQR

SEQ ID NO: 15 - protein, 255 aa, Streptomyces bikiniensis
MTVIAAEADLYLDLLKKVVTNMIYEDQTNVAGLITSSSYSAELRSVGEDFPRVAHSMVGLKRLDNLQKCLEDVLRDGVPGDFAETGVWRGGACIFARGVFRAHGVRDRKVWVADSFQGFPKTTEDDHQLDVDIDLGQYNDVLSIPVDVETVKGNFARYGLLDDQVRFLPGWFKDTMPTAPIERLAVLRLDGDSYAATREVLTNLYHKVSDGGYVIVDDYCIPACRQAVHEFRDEHGITDEIHQIDRQGSYWRRSS

SEQ ID NO: 16 - protein, 420 aa, Streptomyces bikiniensis
MLDVDHLLPIAFRVRKSMKSSKVVHSRPAEAGVAWPVARTCPFTLPDQYAEKRKNEPICRAQVWDDSRTWLITKHEHVRALLADPRVTVDPAKLPRLSPSDGDGGGFRSLLTMDPPDHNALRRMLISEFSVHRVREMRPGIERTVHGLLDGILERRGPVDLVAELALPMSTLVICQLLGVPYEDREFFQERSEQATRVGGSQESLTALLELRDYLDRLVTAKIETPGDDLLCRLIASRLHTGEMRHAEIVDNAVLLLAAGHETSAAMVALGILTLLRHPGALAELRGDGTLMPQTVDELLRFHSIADGLRRAVTEDIELGGITLRAGDGLIVSLASANRDESAFASPDGFDPHHPASRHVAFGYGPHQCLGQNLARLELEVTLGAVVERIPTLRLAGDADALRVKQDSTIFGLHELPVEW

SEQ ID NO: 17 - protein, 73 aa, Streptomyces bikiniensis
VRVTVDQSRCLGAGQCEQLAPEVFRQDEEGLSRVLVPEPDPASWPRVLQTVDLCPVQAVLIDEGPGPAPQDTK

SEQ ID NO: 18 - protein, 356 aa, Streptomyces bikiniensis
MAAGAPDGGPLPRTGRPHRRGPRSRAAGHQVTADRWAGRTVLVTGALGFIGSHFVRQLEARGAEVLALYRTERPQLQAELAALDRVRLIRTELRDESDVRGAFKYLAPSIDTVVHCAAMDGNAQFKLERSAEILDSNQRTISHLLNCVRDFGVGEAVVMSSSELYCAPPTAAAHEDDDFRRSMRYTDNGYVLSKTYGEILARLHREQFGTNVFLVRPGNVYGPGDGYDPSRGRVIPSMLAKADAGEEIEIWGDGSQTRSFIHVTDLVRASLRLLETGKYPEMNVAGAEQVSILELARMVMAVLGRPERIRLDPGRPVGAPSRLLDLTRMSEVIDFEPQPLRTGLEETARWFRHHTR

SEQ ID NO: 19 - protein, 326 aa, Streptomyces Bikiniensis
VTADRWAGRTVLVTGALGFIGSHFVRQLEARGAEVLALYRTERPQLQAELAALDRVRLIRTELRDESDVRGAFKYLAPSIDTVVHCAAMDGNAQFKLERSAEILDSNQRTISHLLNCVRDFGVGEAVVMSSSELYCAPPTAAAHEDDDFRRSMRYTDNGYVLSKTYGEILARLHREQFGTNVFLVRPGNVYGPGDGYDPSRGRVIPSMLAKADAGEEIEIWGDGSQTRSFIHVTDLVRASLRLLETGKYPEMNVAGAEQVSILELARMVMAVLGRPERIRLDPGRPVGAPSRLLDLTRMSEVIDFEPQPLRTGLEETARWFRHHTR

SEQ ID NO: 20 - protein, 403 aa, Streptomyces bikiniensis
VVTHAPNSLISDIIRASGGHDADLKDLAARHDPADIVRVLLDEITSRCPAPVNDVPVLVELAVRAGDRLFPTYLYVLKGGPVRLAAKDEAFVAMRVEYELGELARELFGPVRENVTGVRGTTLFPYVGDTASEGEEDSGAEHIGTHFLAAQQGSQTVLAGCHSHKPDLSELSSRYLTPKWGSLHWFTPHYDRHFRSYRDQPVRVLEIGIGGYKHPEWGGGSLRMWKHFFHRGEIYGLDIVDKSHFDAPRITTLRGDQSDPDHLRSIAEKYGPFDIVIDDGSHINDHIRTSFQALFPHVRPGGLYVIEDLWTAYWSGFGGDEDPKRYSGTSLGLLKSLVDSIQHEELPEAGDHRPSYADQHVVGMHLYHNLAFIEKGTNAEGGIPPWIPRDFETLVAVSSGGHA

SEQ ID NO: 21 - protein, 418 aa, Streptomyces bikiniensis
MRVTLLSVGSRGDVQPFVALGIGLKARGHDVTLAAPATLRPLVERAGLTYRLSPGDPDGFFTMPEVVEALRRGPSFKNMLAGMPEAPESYTQQVVDAIHDAAEGADLIVNAPLTLAAAYGHPPAPWASVSWWPNSMTSAFPAVESGQRHLGPLTSLYNRYTHRRAARDEWEWRRPEIDGYRRRLGLRPFGDESPFLRLGHDRPYLFPFSPSVLPKPRDWPRQSHVTGYWFWDQHGEPPAELESFLEDGEPPVALTFGSTWSLHRQEEALQAALDAVRGVGRRLVMVDGPDSDLPDDVLRLHQVDYATLFPRMAAVIHHGGAGTTAEVLRAGVPQVIVPVFADHPFWAARLSRTGVAARPVPFARFSREALAQSVRQAVTDPAMAGRARRLGERVSKERGVDTACDILEKWAETARATA

SEQ ID NO: 22 - protein, 280 aa, Streptomyces bikiniensis
MLNRIVRYLACPHCGASLAQGDRALFCPAGHSFDIAKQGYVNLLPRATKLRADTKEMVEARDAFLSAGHYDPVMDALVDLARRTADPAVPGCVVDIGGGTGHYHAGVMEAFPDAQGLLLDISKYAVRRAAKAHPRIAAAVTDAWQTLPLRDAAAGMVINTFAPRNGPELHRVLHPRGVLLVVTPLPDHLREVIGALGLLQVDEGKESRLAEQLAPHFSAVATEELTRTMALDHQALAHLVGMGPNAWHRDAQRDLETIQRLPAPTRVTLSVRLSAYRLSA

SEQ ID NO: 23 - protein, 4441 aa, Streptomyces bikiniensis
MRAPYGNRQVNRRFLREFRAKRPHCVSPLHFLAEFSESRQTTGSAGVTAPIDRPGVSMAPKSGAQRSSDIAVVGMSCRLPGAPGIDEFWHLLTTGGSAIERRADGTWRGSLDGAADFDAAFFDMTPRQAAAADPQQRLMLELGWTALENAGIVPGSLAGTDTGVFVGIAADDYAALLHRSATPVSGHTATGLSRGMAANRLSYLLGLRGPSLAVDSAQSSSLVAVHLACESLRRGESDLAIVGGVSLILAEDSTAGMELMGALSPDGRCHTFDARANGYVRGEGGACVVLKPLERALADGDRVHCVVRGSAVNNDGGGSTLTTPHREAQAAVLRAAYERAGVGPDQVSYVELHGTGTPVGDPVEAAALGAVLGTAHGRNAPLSVGSVKTNVGHLEAAAGLVGFVKAALCVREGVVPPSLNHATPNPAIPMDRLNLRVPTRLEPWPHPDDRATGRLRLAGVSSFGMGGTNAHVVVEEAPLPEAGEPVGAGVPLAVVPVVVSGRSAGAVAELASRLNESVRSDRLVDVGLSSVVSRSVFEHRSVVLAGDSAELSAGLDALAADGVSPVLVSGVASVGGGRSVFVFPGAGVKWAGMALGLWAESAVFAESMARCEAAFAGLVEWRLADVLGDGAALEREDVVQPASFAVMVSLAALWRSLGVVPDAVVGHSQGEIAAAVVAGGLSLEDGARVVVLRARVAEEVLSGGGIASVRLSRAEVEERLAGGGGGLSVAVVNAPSSTVVAGELGDLDRFVAACEAEGVRARRLEFGYASHSRFVEPVRERLLEGLADVRPVRGRIPFYSTVEAAEFDTAGLDAEYWFGNLRRPVRFQETVERLLADGFRVFVECGAHPVLTGAVQETAETAGREICSVGSLRRDEGGLRRFLTSAAEAFVQGVEVSWPVLFDGTGARTVDLPTYPFQRRHHWAPDGSASAAPTRDIRPDETAAVPADTMDLAGQLRADVASLPTTEQIARLLDQVRDGVATVLGLDARDEVRAEATFKELGVESLTGVELKNHLRARTGLHVPTSLIYDCPTPLAAAHYLRDELLGRPAEQAVVPAGIPVDEPIAIVGMGCRLPGGVSSPEGLWDLVASGVDAVSPFPTDRGWDVGGLFDPEPGVPGRSYVREGGFLHEAGEFDAGFFGISPREALAMDPQQRLLLETSWEALERAGIDPHTLRGSRTGVYAGVMAQEYGPRLHEGADGYEGYLLTGSSSSVASGRISYVLGLEGPAVTVDTACSSSLVALHLAVRALRSGECDLALAGGATVMAEPGMFVEFSRQRGLSAHGRCKAYSDSADGTGWAEGAGVLLVERLSDAVRHGRRVLAVVRGSAVNQDGASNGLTAPNGRSQSRLIRQALADARLGVADVDVVEGHGTGTRLGDPIEAQALLATYGQRDAGRPLRLGSLKSNVGHTQAAAGVAGVIKMVMAMRHGVLPKTLHVDEPTAEVDWSAGAVSLLREQEAWPRGERVRRAGVSSFGVSGTNAHVVVEEAPVPEDGEAIEGGAPLAVVPVVVSGRSAGAVAELAGRVSEVAGSGRLVDVGLSSVVSRSVFEHRSVVLAGDSAELNAGLDALAADGVSPVLVSGVASGEGGRSVFVFPGQGTQWAGMALGLWAESAVFAESMARCEAAFAGLVEWRLADVLGDGSALERVDVVQPASFAVMVSLAELWRSLGVVPDAVVGHSQGEIAAAVVAGGLSLEDGARVVVLRARLIGRELAGRGGMASVALPVAVVEERLAGWAGRLGVAVVNGPSATVVAGDVDAVGEFVTACEVEGVRARVLPVDYASHSAHVEDLKAELEQILAGIGPVTGGIPFYSTSEAAQIDTAGLDAGYWFGNLRRPVRFQETVERLLADGFRVFVECGAHPVLTGAVQETAESTGRQVCAVGSLRRDEGGLRRFLTSAAEAFVQGVGVSWPALFDGTGARTVDLPTYPFQRRRYWLESRPPAAVVPSGVQDGLSYEVVWKSLPVRESSRLDGRWLLVVPETLDADGTRIAHDLQHALTTHGATVSRVSVDVTTIDRADLSARLTTSAAEDQEPLGRVVSLLGWAEGVRAHGPNVPTSVAASLALVQAVGDAGFGVPVWAVTRGAVSVVPGEVPETAGAQLWALGRVAGLELPDRWGGLIDLPADADARTAGLAVRALAAGIADGEDQVAVRPSGAYGRRVVQAAHREPSGAKTEWRPRGTVLVTGGMGAIGTRVARWLARNGAEHLVLTGRRGAGTPGADELAGELRASGVQVTLAACDVSDRAALAALLDAHPPTAVFHTAGVLNDGTVDTLTPAHLDGVLSPKATAAVHLHELTAHLDLDAFVLFASVTGVWGNGGQAGYAMANAALDALAEQRRAGGLAATSISWGLWGGGGMAEGDGEVSLNRRGIRALEPATGIEALQRTLDQGATCRTVVDVDWGQFAPRTAALRRGRLFADLPEVRRVLESEGVAREDAGTVEPGAVLAERLASRSEAEQRRMLVELVRAEAAAVLRHDTTDLLAPRRSFKDAGFDSLTALELRNRLNTATGVVLPVTVVFDHPNPGALADFLYGEALGLSAARSSASDTADTTRPAAAPEEPIAIVGMACRYPGEARSPEELWKLLIDERDVIGPMPTDRGWDVGGLFDPEPGVPGRSYVREGGFLHEAGEFDAGFFGISPREALAMDPQQRLLLETSWEALERAGIDPHTLRGSQTGVYAGMFHQEYATRLHEAPVEFEGHLLTGTSGSVASGRISYVLGLEGPAVTVDTACSSSLVALHLAVRALRSGECDLALAGGVTVMAEPGVFVEFSRQRGLAADGRCKAFAAAADGTGWAEGVGVLAVERLSDAVRHGRRVLAVVRGSAVNQDGASNGLTAPNGPSQQRVIRQALADARLGVADVDVVEGHGTGTRLGDPIEAQALLATYGQRDAGRPLRLGSLKSNVGHTQAAAGVAGVIKMVMAMRHGVLPKTLHVDEVSPHVDWSAGAVSLLTEQEPWPRGERVRRAGVSAFGVSGTNAHVVVEEAPASEAPVAVEPVEPGAVGLLPVVPVVVSGRSAGAVAELASRLNESVRSDRLVDVGLSSVVSRSVFEHRSVVLAGDSAELSAGLDALAADGVSPVLVSGVASVGGGRSVFVFPGAGVKWAGMALGLWAESAVFAESMARCEAAFAGLVEWRLADVLGDGAALEREDVVQPASFAVMVSLAALWRSLGVVPDAVVGHSQGEIAAAVVAGGLSLEDGARVVVLRARVAEEVLSGGGIASVRLSRAEVEERLAGGGGGLSVAVVNAPSSTVVAGELGELDRFVAACEAEGVRARRLEFGYASHSRFVEPVRERLLEGLADVRPVRGRIPFYSTVEAGEFDTAGLDAEYWFGNLRRPVRFQETVERLLADGFRVFVECGAHPVLTGAVQETAETAGREVCAVGSLRRDEGGLRRFLTSAAEAFVQGVEVSWPVLFDGTGARTVDLPTYPFQRRHYWAQSSPAGAGSSAAARFGMTWEEHPLLGGALPLADSGELLLVGRISPASHSWIADHTVAGTALLPGTAFVDMALHAAAVAGCAGVEELSIEAPLPVHGGIRLQVVIDEPDSSARRRVSVFARPEEEDGDAGRWTRHATGVLTPDVAPEPGRPQWCRQAWPPSGSVRVEASELYDRFSALGYDYGEVFAGVEAVWLREGEAFAEVRLPTGAAPDAERFGVHPGLLDAALHPWLLGDFLSRPDGGSVLLPFAWRGITLHTAGADALRVRLGPAGEGALSLEAVDLSGAPVLSMDALVLRPLAQDRLAELVGGTTSTPLYRVDWQRSPIARTAPSATGLFGSLPSGAVRRWAVVGQGGVAARYATAEPGTGCVGVFPDLDALRTTLDSGADGPDIVLADFGARPGDAAPHGTDPADGARDTVRRGLALIQGWLSDERFAAARLAVLTEHAVATEADTRRTDLAGSALWGLMRSAQTEHPDRFVLVDHDGQDASYRTLPTALDSEIPQLALRAGETLAPELAVLPSPADGGPATSAAFDPEGTVLVTGATGTLGSLLARHLVTAHGVRHLLLLSRSGREAAGAAELERELRQRGAEFQLLSCDATDRAALKEALATVPAAHPLTAVIHTAGVLDDGVVEALTPERLDRVLRPKADAALNLHDLTEGMPLKAFVLYSGAVGLLGGAGQANYAAANAFLDGLAQHRHAQGLPAVSLAWGLWSATSTFTDHLGEVDLRRMERSGITPLTDEQGLDLFDRALGAAVDAPQLCVMGLDTAALRRQAAEHGPTSMPPLLRTLAAPPVRRGAGRSGRGGRAASATDAPSRAQALRERLTGLDAAARRDELLVLSQAQLADVLGFADKTAVDPVRSFREIGLDSLTAVELRNRLGVVTGLRLPPALVFDHPNLDALAAHLAELLAAEGRDDAGAAALSGIDALDRAVREMAADDTRRDAVRRRLAELLAVVGDAPRDGGRAPRAAADAGGRDAQADPDLLGRLDSASDDDLFAFIEDQL

SEQ ID NO: 24 - protein, 1974 aa, Streptomyces bikiniensis
MTANDDKIRDYLKRVVAELHSTRQRLNALEHDAREPIAIVGMSCRLPGGVTTPESLWRLVDSGTDAASPFPDDRGWDLDALHHPESGAVHSREGGFLHDSADFDAEFFGISPREALAMDPQQRLLLETAWEVFERAGIDPVSARGSRTGVYAGVMYHDYGARLNEIPPGLEGYLVNGSAGSIASGRVAYTLGLEGPAVTVDTACSSSLVAVHLAAQALRRRECDMALAGGATVLSTPDLFIDFARLGGLASDGRCKAFSDAADGTSFAEGAGLLLLMRLSDAVAEGHTVLAVVRGSAVNQDGASNGLTAPNGLAQQRVIREALADADLDPDQIDAVEAHGTGTRLGDPIEAQALLHTYGTSRSPERPLWLGSLKSNIGHTQAAAGVAGVIKTVLAMRHGRLPRTLHVTRPSSRVEWSAGAVELLTRAQDWPGQGNAPRRAGVSSFGASGTNAHLILEGVPDGDITVAETRPATGGGAWPLAGRTEAALRAQARRLHDHLAARPHVSPAAVGRTLVRSRTAFEHRAVVLGQDTADLLSGLAELASGGAHGPGVITGRAARGRRTALLFTGQGSQRPGAGRHLYERYEVFARALDETAAALDRHLDRPLRDVMFAEPGGATAGLLDRTEYTQPALFALEVALFRLVTAGGLRPDALLGHSVGELAAAHVAGVFTLPDAARLVTARGRLMGELPAGGAMMAIQASGPEIEETITALAAHRSARVAVAALNGPDATVISGDEDVVAELATLWRERGRRTRALPVSHAFHSPHMDAALEPFARIARDVSYAEPRIPVVSNLTGGIASATTLCAPEYWVRHAREAVRFSDGFRALRDQGIDTFIELGPDGVLSALGRDCLREEEGDAPRQDGSADPDTTGSRADGGRRPVLTVPLLRRDRDETTTCLGALATVHTHGVPVDLAAVHGAPEGPAVELPTYAFQRTRYWLDAPAPAAGPTATGLEATDQPLLPAVIDLPDGEGTVRTGLLSLRTHPWIADHRVRDHAVVPGAALLDVAAWAGTEAGCPRVAELTFATPLVLPENGEGVRLRVTVSGPDAEGIRSLRIDSRPADTVRTADAPSDWTRHASGTLVPAPEEAGDGTGVPTELLGAWPPADATPVALDADAVAAEYQRLAAGGVTYGPAFRALRAVWRRGAEVFAEVRLPGQAAADASRYGMHPALLDALTHATGFGERSTEARGLVPFAWSDVRIHVRGADSLRVRIAPAGPDAVTVAAVDPTGRPVLAARSLTLRPLAESRFQDPEADSTPLYRLEWTPAPGSVTGHAGPRQAAAEWGVLGDPVQALLDAVRDGAEAPVRTHDDLLALAASDTAPPDHVLALLGHDGDALATGAHDLAARALALVQGWLTHARFADARLVVLTQGAVTAGTSPVHPAAAAAWGLLRSAQSEHPGRFVLVDADPADPAASYRSLPRAVASGASQLALRGAEILVPRLARGTDRQATVPGHPGDVTAPETTAAPEPAPSGTPSGPWPADGTVLVTGGTGTLGKAVARHLVTKHGVRHLILAGRRGADTPGAADLATELTGLGATVNIVRCDAADRSALEGVLAAVPAAHPLTAVVHTAGVLDDGIVTAQTPRRLSAVLRAKADAVSHLHELTRDLDLSAFVLFSSAAGTLGSPGQSGYAAANSFLDAFAAWRRAQGLPAVSLAWGLWGDGGDGRDGGGSAADGMGASLAAADLARLRRSGILPLDPAEALRLFDEACDPARTEAVLLPIRLDLTGLRARSARGAVHASVVPEVLHTLVPPPAGAGSPAGADASDPAAGQAPPAPASDTLAERLAGKPRGERLTALTELVRTEIASVLGHPDSGRVQLQSSFKESGFDSLTAVELRNRLTAATGTKLPATLVFDHPTPAALVDHLEQELPKAAQEIPADLPAVLDALDRIRDGLATAATDDSSRDHIAERLQALLGTLTSAAGVSRPTGSPGEHDRQGPDELSLGQRLAASSDDELFDLFDSDFRST

SEQ ID NO: 25 - protein, 3788 aa, Streptomyces bikiniensis
MSSTSPATNEEKLRDYLRRAMTDLHEAREQIRRTESARHEPIAIVGMGCRLPGGVSSPEGLWDLVASGVDAVSPFPTDRGWDVGGLFDPEPGVPGRSYVREGGFLHEAGEFDAGFFGISPREALAMDPQQRLLLETSWEALERAGIDPHTLRGSRTGVYAGVMYHDYGSTATVSVASDDETAGFLGTGTSGSVASGRISYVLGLEGPAVTVDTACSSSLVALHLAVRALRSGECDLALAGGVTVMAEPGVFVEFSRQRGLAADGRCKAFAAAADGTGWAEGVGVLAVERLSDAVRHGRRVLAVVRGSAVNQDGASNGLTAPNGPSQQRVIRQALADARLGVADVDVVEGHGTGTRLGDPIEAQALLATYGQRDAGRALRLGSLKSNVGHTQAAAGVAGVIKMVMAMRHGVLPKTLHVDEPTAEVDWSAGAVSLLREQEAWPEVGRLRRAAVSSFGVSGTNAHVVVEEAPASEAPVAGEPVEPVEPGAVGLLPVVPVVVSGRSAGAVAELASRLNESVRSDRLVDVGLSSVVSRSVFEHRSVVLAEDSAELHTGLVAVGTGVPSPGVVSGVASVEGGRSVFVFPGQGTQWAGMALGLWAESAVFAESMARCEAAFAGLVDWRLADVLGDRSALERVDVVQPASFAVMVSLAELWRSLGVVPDAVVGHSQGEIAAAVVAGGLSLEDGARVVVLRARLIGRELAGHGGMASVALPVAVVEERLAAWAGRLGVAVVNAPSATVVAGDVDAVAEFVTACEVEGVRARVLPVDYASHSAHVEELRAELEQILAGIDPVAGETPLYSTVEAGVVDTASMDAGYWFRNLRRPVRFQETVERLLADGFRVFVECGAHPVLTGAVQETAESTGRQVCAVGSLRRDEGGLRRFLTSAAEAFVQGVEVSWPVLFDGTGARTVDLPTYPFQRRRYWLESRPPAAVVPSGVQDGLSYEVVWKSLPVRESSRLDGRWLLVVPETLDADGTRIAHDLQHALTTHGATVHTLALDPSAAHFDGLFDGILQEETDVTGIFSLLGLASGPHPDHGEVELAGAASLTLMRQAQRDGFRAPVWAVTRGAVSVVPGEVPETAGAQLWALGRVAGLELPDRWGGLIDLPADADARTAGLAVRALAAGIADGEDQLAVRPSGAYGRRLVRATARRGRKDWRPQGTVLLAGHLDAVGEPLARWLLTGGADHVVLADPALTELPATLADLAQTVTTAAAPDLADRAVLAALVTEYVPATVVVVPPAAELAPLASISPADLAAAVTAKSATAAHFDALLDGPHAPELVLISSVAGIWGGVRQGAYAVGAAHLDALAARRRARGLSAASVAWTPWAGSVTADGSAAESLRQYGIAPLEPQAALAELDRALNQQLHGGGGDAAVADIDWERFLASFTSVRPSVLFDELPEVRRLREAEAAAMADQAAARTGAPGGTELARSLRAKSLNAQRTALLELVTAHVAAVLGESVPEAIDRSRAFKDIGFTSMTAMELRNRLKEATGLALPASLVFDHPHPGALADHLREELLGEDGAAGADSAAEEPSATSPTVQDEPIAIIGMACRLPGDVGTPDELWELLETGRDAMSDLPVNRGWDVAGLYDPDPDAAGRSYVREGGFLHDAGEFDAEFFGISPREALAMDPQQRIVLELAWESFERAGLDPAGRRGSRTGVFMGTNGQHYMPLLQNGNDSFDGYLGTGNSASVMSGRISYTLGLEGPALTVDTACSSSLVALHLAVRALRNGECDLALAGGATVMSTPEVLVEFSRQRAVSADGRCKAFSASADGFGPAEGAGVLLVERLSDAVRHGRRVLAVVRGSAVNQDGASNGLTAPNGPSQQRVIRQALADARLGVADVDVVEGHGTGTRLGDPIEAQALLATYGQRDAGRPLRLGSLKSNVGHTQAAAGVAGVIKMVMAMRHGVLPKTLHVDEVSPHVDWSAGAVSLLTEQEPWPEVGRPRRAAVSSFGLSGTNAHVVVEEAPVGEAGQAAGDARLAVVPVVVSGRSAGAVAELASRLNESIRSDRLVDVGLSSVVSRSVFEHRSVLLAGDSGELHTGLVAVGTGVPSPGVVSGVASVGGGRSVFVFPGQGTQWAGMALGLWAESSVFAESMARCEAAFEGLVDWSLADVLGDGSALERVDVVQPASFAVMVSLAELWRSLGVVPDAVVGHSQGEIAAAVVAGGLSLEDGARVVVLRARLIGRELAGRGGMASVALPVAVVEERLAGWAGRLGVAVVNGPSATVVAGDVDAVAEFVTACEVEGVRARVLPVDYASHSAHVEDLKAELEEVLAGIGPVTGGIPFYSTSEAAQIDTAGLDAGYWFGNLRRPVRFQETVERLLADGFRVFVECGAHPVLTGAVQETAESTGRQVCAVGSLRRDEGGLRRFLTSAAEAFVQGVGVFWPALFDGTGARIVDLPTYPFQRRHYWYNDPARRTGDATSFGMAQAGHPLLDAGTELPESGEHLYTARLAADSHPWLLEHTLLGAPLLPGAAFVDLVLWAGGEVGCDLIEELTLTSPLLLSDSAALQLRLVVGTADAEGRRTITVHSRPDGDPRTTRTPAASSETSPDAESDTEIRRDTSAWTKHAQATVAPAPDVPPSGVDAEGDAVRPAVEWSVAATESDAFQAEDFYASFAAHGYGYGPLFQGVRSGRQDGTDVYAEVALDHDRLPSAEQFGLHPALLDAAFQTMRLGSFFPDDGQARVPYTFRGIRLYAPGAARLRVRVSAVGADAVRVECADERGRLVCEIDALVVSTVSPDQLRPAGQDATQDMLHRIEWPVLSPPTGSATSPAPPRWIVVGGEDEGLGLGGLRLDGPRLDGPGLAEALSEAGMGTERHRNLADALSAVRTPVDTAGSAAAAGTTSLIAVPVPQSPTMDAGAVRHAVHRALELVQGWVAADEAAEEGGSDGAAADRRLVLVTSGAVSTGDADPLRDPVAAAVWGLIKSAQSEQPGRIVLVDLDEGAVDGAALAAAISTGEPQLALRDGDVHVPRLAPLSVRDSQTLLPPAGTRAWHLVGAGTGTLSDLALVPAQTDTVALAPGQVRIAVRAAGLNFRDTLIALGMYPGEGVMGAEGAGVITEVGPDVVSLAVGDRVLGMWTDGFGPYVVADHRMVAPMPRDWSYAEAASVPAVFLSAYYGLRHLAGLRAGQSVLVHAAAGGVGMAAVQLARHFGAEVFGTAGTAKWDALRAQGLDDRHIAGSRTLDFADRFLDATEGRGVDVVLNSLAGDFVDASLRLLPRGGRFVELGKADVRDAAQVAADRPGTVYRAFELMEAGPELIGRMLNELLELFESGALRLLPVTPYDIRRAPDAFRTLSQAGHVGKLVLTMPPAFEPHGTVLITGGTGNLGGTLARHLVTEHGVRHLLLAGRRGPEAEGAAELVRELHDLGASVTVAACDVADRAALRKLLGGIPPERPLTGVVHAAGVLDDGVVTSLTPDRVDGVLRPKVDAALNLHEAALDPELGLDITAFVLFSSVAALLGGSGQGSYAAANGFLDGLAQYRRGRSLPALSLGWGLAGSGRMTSHLDSRALLRRMARGGVLPLSPAESMALFDAAQGFDEALQVPARFHTAALGADGNVPPLFNGLIRGGTAHAEARRRTVGASPAGGPAGGEPVNLADRLSGLTEDEQRALLLDTVRTHAALVLGHTGTDGIQADRAFKDLGFDSLTAVEMRNRLTAATGLHLAATLVFDHPAPADLAEHLRSRLVPEGTDVPPLLAELGRLETAFKKLTTADLASVVPDDIARDEIAVRLAALGSLWNGLHGNGLSGDAAQKHGDSIVEDIDSADDDEIFAFLDESFGDS

SEQ ID NO: 26 - protein, 1612 aa, Streptomyces bikiniensis
MATEHEQKLRDYLKRATTELHKATERLKEVEQRAHEPVAIVGMGCRFPGGASSPEELWDLVAAETDAVSPFPVDRGWDVTGLYDPDPDAAGRAYVREGGFLHDAGEFDAGFFGISPREALAMDPQQRLLLETSWEALERAGIDPHTLRGTRTGVYMGAWNGGYAEGIPQPTAELEAQLLTGGVVSFTSGRVSYLLGLEGPAVTVDTACSSSLVALHLAVRALRSGECDLALAGGATVMSTPDVFVRFSRQRGVAADGRCKAFSASADGFGPAEGVGVLAVERLSDAVRHGRRVLAVVRGSAVNQDGASNGLTAPSGRAQALLIRRALADARLGVADVDVVEGHGTGTRLGDPIEAQALLATYGQRDAGRPLRLGSLKSNVGHTQAAAGVAGVIKMVMAMRHGVLPKTLHVDEPTAEVDWSAGAVSLLREQEAWPEVGRLRRAAVSSFGVSGTNAHVVVEEAPVPEDGEAVGGGVPLAVVPVVVSGRSAGAVAELAGRVSEVAASGRLVDVGLSSVVSRSVFEHRSVVLAGDSAELNAGLDAVAGGVPSPGVVSGVASGEGGRSVFVFPGQGTQWAGMALGLWAESSVFAESMARCEAAFVGLVDWRLSQVLSDGSALERVEVVQPASFAVMVSLAELWRSLGVVPDAVVGHSQGEIAAAVVAGGLSLEDGARVVVLRARLIGRELAGRGGMASVALPVAVVEERLAGWAGRLGVAVVNGPSATVVAGDVDAVAEFVAACEVEGVRARVLPVDYASHSAHVEDLKAELEQILAGIGPVTGGIPFYSTSEAAQIDTAGLDAGYWFGNLRRPVRFQETVERLLADGFRVFVECGAHPVLTGAVQETAESTGRQVCAVGSLRRDEGGLRRFLTSAAEAFVQGVEVSWPALFEGTGARTVDLPTYPFQRRRYWLESRPPAAPIETAAASGIESWRYRVAWKSLSLSESSRLDGRWLLVVPETLDADGTRIAHDIQHALTTHGATVSRLTVDVTTTDRADLSARLTTTAAEDQGPLRGVLSLLSTDERQHPDHPGVDRATAGTMLLAQACGDLVVARGVEPRLWVVTRGAVAVSPAERPSSAGAQVWGLGRCAALELPTRWGGMVDLPPAARDAGRHVRRLVRLLSETCAEDQVALRASGAYGRRLLPASSPSVSVPRTAKSGYQPRGTVLVTGGTGALGGHLARWLARNGAEHIVLAGRRGEGAPGAAELSAELKELGAEVTVAACDVADRNALRDMLESLPADRPLSGVFHAAGVPHSAPLAETDVAGLAAVLPGKVVGARHLHELTREKELDAFVLYASGAGVWGSGGQSAYGAANAALDALAEQRRAEGLPATSVSWGLWDGGGMAGERGEEFLTALGLRAMEPESAVAALEEALDRGDTCVSVVDVDWSRFAESFTAFRPSPLIGELPGVRAVPDGSAGGPSDDLADAARHGGAADRGVPAGLARATGDDRQDILLDLVRRHAAAVLGHPGPQHIEPDAGFRTLGFSSVTAVELANKLGAAVGTKIPATFAFDHPNARAAASRLDVLLAASSDETAQEAEIRQALRTVPLARLRAAGLLDGLLELAGLEAEPGLPGDVPDRGAATPDEESALAEVDGLDAEALVDLVLNQSDS

SEQ ID NO: 27 - protein, 1350 aa, Streptomyces bikiniensis
MALSQEKVLEALRTSVKDAERLRKRNRELLAARHEPIAVVGMACRYPGGVRSPEDLWELVVSGTDAVGPFPEDRGWDVERIYDQDPSVPGTTYCREGGFLYDAGDFDAAFFGIGPREATVMDPQQRQLLEASWEALEQAGLDPRALRGSQGGVFVGAANQGYVPGDAAASGRLPEGSDGYLLTGNADAVLSGRISYFLGLEGPSMTVETACSSSLVALHLAVQALRREECEFALAGGVAVLANPAAYVEFARQRGLAPDGRCKAFDDAADGTGWAEGVGVLVVERLSDAVRKGHRVLAVVRGTAVNQDGASSGLSVPNGPSQQRVIRRALADARLEAGQIDAVEAHGTGTRLGDPIEAQALLDTYGEERSPERPLWVGSLKSNFGHAQAAAGVGGVIKTVMALRHGLLPRTLHVTSPTRHVDWGDGQVRLLTEPVDWPRTGAPRRAAVSAFGVSGTNGHIILEEAPPPTRPEAVRQAGERRPVLVPWTLSGRTRPALCRQAARLAAHLEQHPDLDPLDVGFSLATTRTHFEHRAVLLADAATEGGSRADALGALRAIAEDRDPGGAVRDTARGEGRIAFLFCGQGSQRPGMAEQLYAQYPAFARELDTIATHLDAHLDRPLATVMFAPAGTAEAALLDGTQYAQAALFAVEVALFRLFEGWGLRPDVLLGHSVGELAAAHVAGVFGPADACSLVAARGRLMQELPAGGAMLSVRAAEHEVRELIAGQEDRIAVAAVNGPRSVVVSGDEDAVSALAEELTEYGVRTKRLNVSHAFHSPRLDSMLETFRRVAETVEYREPTLDVISGLTGRPADAGELATADYWVRQARETVRFHDGVRAAHARGVSTFVELGPDGVLCGLALETLAEETDGEAAAETPGRARAALVPVMRRERPEGSTLLTALATAHARGAEVDWSRFYADTGARHTTLPTYAFQRQRFWLETAAPAAPAAGQGAGPADPQDSTGPAARPTLTEQDLLLLVRTEAAAALGHAELEDVPADSLFGDIGFDSLAAIELGAALTGATGLEVPSSLVLDHPTPRELAAHLAAARTAADSDDTSPEGPDTAGESSLSAMYRRAVRLGRAEPFIGTLAELAAFRPVFPADHTLADGETVGQAAAAWQPAPVRLATTDGEGPELICCAGTAVASGPEEFTALAAALGDRLTVSALRQPGFRANELLPGSLDGLLDAQADAVLRHTGDRPYALLGHSAGGALAHALACRLEELGAGPAALVLADVYLPSSPGAMGVWRNEMLDWVMRRSVVSIDDARLTAMGAYNQMLLEWTPRPTKAPVLFLRATEPVRPWSGEPESWRAHWDGGDHTAVDVPGTHLTLMTEHARHLAATLHTWLGTL

SEQ ID NO: 28 - protein, 412 aa, Streptomyces bikiniensis
VTTQWTTPSVLGRRLQRTYVGHWFAGTQGDPYALILRAQRDDTTPYEEDVRARGPVFHSEVLDTWVITDGALARSVLTDARFGGLTRAGGRYRAELLPPAGPEVGPARAGVRGGVRADADPAVSAQDEVVVEALAEQLSRTLLGGLGDDFDLVAAFARRLPAQVLAEFLGLPAAARSRFEELLAGCAHSLDSRLCPQTLDITRTGLGAAAELRELLARHLGGSGPRSAQAAVSLAVEVAAPAGALICNAVEALSSSPGQWNALRQNPEKADAVVAETWWRRPPVRVESRIAQEDVDVAGVPVPADGHVAILVAAAQRDPAITPAPTKDDTGTPGQGDCGVPLGLVGDAHATSAARTVRALCRGALRALAQEAPGLRPNGTPVRLRRAPVTLGHARFPVARTGRGTPTDAGAA

SEQ ID NO: 29 - protein, 425 aa, Streptomyces bikiniensis
MRVLMTSIAHNTHYYHLVPLAWALKAAGHEVRVAGQPRVTDIITGSGLTAVPVGDDEDMMELFAEIGGDITPYQEGLDFAEERPEARSWEHLLGQQTVLTSLCFAPLNGDSTMDDIVALARSWQPDLVIWEPFTFAGAVAAHAVGAAHARVLWGPDVIGRARERFVEAKAQQAPEHREDPMAEWLGWTLERLGLPAAGDGMEELLNGQWVIDPGPESVRLDLREPILPMRFVPYNGPAVVPGWLSEKPKRPRVCLTQGVSGRETHGKDAVRFQDLLAALGDLDIEIVATLDSTQRENLTEVPDNVRIVDFVSMDVLLPSCAMIIYHGGAGTSATALLHGVPQVVIGAHWDVPVRARQLDDLGAGIFIRPEDLDAATLRAAVQRVLTEPSLQRAADRLRAEMRSNPTPAETVTVLERLSRSHRQPR

SEQ ID NO: 30 - protein, 248 aa, Streptomyces bikiniensis
MEFEGQVALVTGAGRGIGRATVVRLAEAGCDIALHYNQAKAQAEEVAERIAALGRTVELFPGDLSRPETGRQLVAAVQQKFDRIDILVNSAGITRDKLLLSMEADDIHQVIATNLVGPMFLTQAVALTMLRQRSGRIVNISSAAASRPGKGQSNYAASKAGLEAFTRAMAVELGSRGILVNAVAPGIVKTGLTEALREGAEPELLARQVIGSFAEPEAVAEAVAYLASPRNTHTTGTVLTVDGGLKMV

SEQ ID NO: 31 - protein, 382 aa, Streptomyces bikiniensis
MSASLSPARTRAALRASARISTELLLVLLGTAAVIWLLGRMWSIVWPLVIGLLITTLTWPFARLLRRLGWPPALAASVVTVLFLAVTAGTVALIAVPVASQSGELADGVVEGIQRLREWTAGPPLNIGDDQITGALDTATDRLQNSVGSLLTTLATGVGTVVNGVVTAVLALFLMFFFLKDGPRFLPWLARQLPGRLATDATTIAERGWDTLGAFVRSQAAVGLLDAVLIGIGLWILGVPLVLPLAVLTFVSAFVPIIGALLAGFVAVLIALVSNGLTDALIVLAIIVVVQQLEGNVFQPMIQSRGLGLHAAVVLLAVTLGGSLAGIVGSLLAVPVAALVAVVWGYVREQLSDPPQLDADGGTRPGPDGAPESVVPAEIPAS

SEQ ID NO: 32 - protein, 487 aa, Streptomyces bikiniensis
MTATTRRDPAAVPDGGTAEPVPSPAAPDGRAAQTVPSPAAPDSGAAESAPSTAADGRAEQTVCSLAADGTADGRGRAPGAAVGVGDAGPRRRWWRRRGAAVTGCSVFLAAPDGRAAQTVPSPAAPDSGAAESAPSTAADGRAEQTVCSLAADGTADGRGRAPGAAVGVGDAGPRRRWWRRRGAAVTGCSVFLAVSVAGHGRLPGLPGRLSSLAETLLPWSALAVPVLVTAALLWRARVAAVVALVVPAVAWLTAFGGALTDKTTPGGDLTLVSHNVEQANPDPAGTVRSLLAAGADVLALEELSPATAPAYERALAESYPYHFYEGTVGLWSVHPLSDARAVPIMPWTRAMRATVDAPGGPLAVYVAHLPSVRVGPGGFTAGARDEALGLLAAEVRAEPVRRVVLLGDLNGSTDDRALRPLTDRLVSAQAAAGAGFGFTWPARLPVVRIDQILLGGVRAASAWTLPATASDHLPVAARIHLAPDPAP

SEQ ID NO: 33 - protein, 231 aa, Streptomyces bikiniensis
MTAYVILEEHPLRPDEDGPLIEVDRTAAHEAGVGGESTVPVRSGERHSQRRLLELLLIPSGNNVARLLARWDSGSQAAFVTKMRRAAVRLGMKDTVYTGASGIEPTTTSTAADQVRLTRAAMEDPVFRAVVATRETTVPGLGTITNTNPLLDTPGVLGVKTGSSTPAGGNLLWAYEVRVGGAPRLLVGAVLHQRANTTPAEGLRAAVEAARGLLTAVRERLAAAGTQGGER

SEQ ID NO: 34 - protein, 386 aa, Streptomyces bikiniensis
MALTVGVLVHDSVLRRSLHEGAGRAHVALATALEDADAAGEEGPRVSPEELPEALLRQIEHGGEATLYEDGPPAPVFRAARREGGKLYAVEVDMTADLLTRQALDRHMWKYSLLTLGVVVPATALATELPARRLRRVARTARRITAGDLDARTGTVRGGDEVAEISAVVDSMADSLQQRIDTEQRFTADVAHELRTPLMGLVTSAELLPEGEVTDMVQSRVRVLRDLVEDLLEVSRLDAGAETAQQGPVDLGALVRDSVARTGLAAQVTRQGAAVVESDPRRLDRIVSNLVVNAHRHGAGRVEVTVAGRTVTVRDHGPGFPADLLSHGPQRFRTGSAERGHGHGLGLTIASGQARVIGATLAFANARDGGAVATLSLPEDGQTSEA

SEQ ID NO: 35 - protein, 228 aa, Streptomyces bikiniensis
VTVLLVEDDEVIRRSVAMSLERYGYRVRVAADGLTGLELFREGRHDLVLLDVMLPGLDGIGLCRRIRETATDPILMMSARGDALDVVSGLEAGADDYVVKPVDTAVLVARIRSLLRRAAFVSPAPGPADPATPAGPLLFGDLSLDPAALEVRRGGERIALAPTELRLLLQFAAHPGIVLDRQTLLREVWDYGWDGDTRVVDLCVQRLRKKIGAERVETVRGFGYKWKR

SEQ ID NO: 36 - protein, 476 aa, Streptomyces bikiniensis
MPESRPAPEPTKTEDGTDARPDTPARPGDTHPVDQMLPPLKLFSAGLQHVAAMYAGVVAPPLVVGIGVGLSTADIAFLMSASLFTSGIATLLQTLGFWKVGARLPFVNGVSFAGVAPMLAIAKAEGPDDALPVIYGAVIVAGVFGFLLAPFFCKLIRFFPPVVTGTVITLIGVSLLPVAFNWAQGGNAQAPDYGSLTYIGLATATLLITVVLRRVLTGFLKQISILLGLVAGTLLSLPLGVADFSAVGDADVIGLPTPFHFGAPQFAAAAIISMCIVMLVSMTESTADVLALGEIVERPADEKTLAAALRADGLGTALSPLFNGFAASAFAQNVGLVAITKVRSRFVVAAAGGILLLLGLCPLLASVVALIPQPVLGGVGIALFGTVAASGIQTLAGAALERGDNVLIVAISLGAGIIPIAAPDFYHAFPEGARIVLDSGISTGCVVAVLLNLAFNHLGRRTDPAPETLPAPAAHH

SEQ ID NO: 37 - protein, 238 aa, Streptomyces bikiniensis
MPPADDPYALPRFDPTRAAAVRAQLGLTLGQVAWAVTAYCGHPAHPGLVEAWEAGTATPDNTQVKALAAALWCSPVDLVEEPGTLAQCRTVAGLTVNETATELGLTRGQWEDAERRNRWQGTPAQTDALLRVLCPPPACFVAACGRTGRLRVLLREAATTWWPPYVGALSRIVPLDPAVLGHALERLHLTYQQWEGAAGGPPRQAARAEARAADFLEHIDLHLWHHVREVLNPARPAS

SEQ ID NO: 38 - protein, 612 aa, Streptomyces bikiniensis
VSQGPALLPRATDHASPTPPHPVDEILPARRMLPAALQHVASMYAGLTAPPLIISSALGLTPAQLSALLAAALLIAGLGTIAQTLGVYGVGAGLPLVNGVSFAVVSPALATAATQGRDGALPAIFGATLVAGLLCLLLAPVFCRLVRFFPPVVSGCVITLVGISLLPVAGTWARGGDAEAAGFGSPADLALAATTLVITLTVHRMLSGRFLGRVAILIGMLAGTLIAIPLGKVDLDPLAQAPLFALPTPFGFGTPQFVPTVIATAAVVMIVSMMESTAALLALGAVAERPVRDRTIAGSLRALGLATVLGGVLGSFTSTSYAQNVGLVSLSRIRSRYVVTLCGAVLVLMGFVPVLGSFVALVPLPVLGGAGVVFFGSVAVTGIRTLAKAALGTGHNAVIVSVTLAFGLFPVLDPDFYARLPAPVATVLGSGITAGCLVAVLLNYLLNHLGRGTEADPDAISAEQVTALDTADTVLGPKRSSDWTPFQPSGSPSGTPDHGRHTRGTARPAPAWPYVTGPVDPTDTGRHHRPHEVPAPPHRPDEVPPPLHPSAAHEGEPPPAVTENAVFPGPLHPLHPLHPRPTGRPDRPRQRHSAEADPWQHPQTPSASGDSQ

SEQ ID NO: 39 - protein, 223 aa, Streptomyces bikiniensis
MTTVSAARHRAGGSPRGGTSRPGPDERIAQVVAEALGSARTVLDPDALPGLGTTRLPFGDGRFDAAMMLCNAPGVPDALSRLGELRRVTRGPVVVLATDPSRVRSFWLDRYAPEVLAVEARRHPPIADLTAVLGGSAEVRSVPVPLDCTDTFDEAYYGRPEKLLDPSARQAGSAWSFVDDRVREEFDTTLRRELRSGEWDERFGHLRRRPVYEGSLVIVRAVP

SEQ ID NO: 40 - protein, 251 aa, Streptomyces bikiniensis
MTTGTDSTTWFRRYSSTPAPRRRLAVLPHAGGSASFFHAWGSAFGGDTEVLVAQYPGRQERFNEPFVDRMDVLADRVTAALLPLADVPLTLFGHSMGASLAYEVALRLEERHRVTPAALHVSSRKAPHRLTPLDLHRKGDDELVAVLRGLGGTDTALLDDPDIRQLVLPAVRADFTVVSTYGPRVPTAVGCPVHAWIGDTDPNVAVGDMDAWADVAPEGFRVRVLPGGHFYLLQQHETLMRELSGHLAGDR

SEQ ID NO: 41 - protein, 316 aa, Streptomyces bikiniensis
MDHRRARAALPLSRHVRDGDAPSGTAALAGDTGRRAAPRCTDPPERAARVGQGPGDRGARRRVPRAADRPLAAPPARLAPGPLSAGRPQPGQHPGTGGVHARAGGRGAPVRLHAGGAGAGAGRVEGTPAAGPHREPGVVRRPRGLAVHRGRPRDRRAGADRGPRRGAPHGADAGRGVRPDTGLPQVAAPAARGADRAADRLVHAGPAGRAQRRRGGGHPGGAAPPGPDGERLGRLLPRPGPAVRRQQPEAGIGRWSGRRGRRFGRHGRRHRDRRGTAELGHHRPEVGSGTVQDRDVHGPREDRVRDHGHRCRELPG

SEQ ID NO: 42 - protein, 240 aa, Streptomyces bikiniensis
MFRTEEKRPVATGTTAHDAVRGHPDAHAAGFGRPRRVTVAVYAADPVLRVGVVQQLRQRPETELVDDADAENAQVSLVVVDALDDDVTALLTRLSYNGATRAGLVIGTLGVGALQRVVECGVSAVLRRAEADQDQLVQLVLAVANGEGVLPGDLLGELLGHVGSLRRAALDPGALPLSTLTSREAEMLRLVSEGLDTAAIARKTSYSERTVKNVLHEITTRLQLRNRAHAVGYALRNGLI

SEQ ID NO: 43 - protein, 1066 aa, Streptomyces bikiniensis
MKTAGPGGRHRRGRLASALLLLVPLLGATGVAGPDDPRTAAAAADAAETTRIAYAGTGHRSLGEPASTDSSTPLFGAGPTHYDTDPSALGDRLVFASRRDEKHPQIYLRGADGGVLRLTSGLDAARPRLTPDGGSVLFDAADPAGGSQRDLWLVRTDGTGLTRLTDTPASEEDPAVSPDGARIAYSSDADPLAGRQIYVRALTGGIPTRLTDPARGTASEPAWNPVDDDVNRAWIAYTSTTTEDGRTRQRLRITDGTTDETLFTGAYANWQGHGAAWLPDGDGIVFLSPETTCTCRTPYDHVFRSVVHADREPSLVLDEDRDVLSPTWIGTAEGGHAIVERSSAATAHTATLQDIRADGSDPRDLQRKILREDPQADTNTDPAKDPLFQPAPPFDPWTERQNYTPDGRRLVLTRFEGPDDARIERIWTADADGTNEAPMPLDGRGARDWDTDPTFSPDGTRLAFTRTSPGGVGEAAGDSRILLAEVATGRITGEIVPPAGELRGGDAQPTWSSDGTTLAFTRARQIAGGGGSKHVWTASTADLTRQRDLSATHCPRDCDVIDDSPAFSPDGRSLAFNRKNGGGRIDERNGLLLTTLSGDACQVLLPTAARGQDGACERELPDTTLTGPHQPRDAAWTADGKRLVFSSRAAAAVNSPEKLNVLDVGSGDITPLTAELAGRQKEPTVQQSVDLAVEAPATTPDVTVGASGTVTVHVVNHGPAASPGTRLTVVPPSGVRITGIEWPGGTCDAASLQCDLGVVEAGAQVPVDVTLTGVTAGDAPVDWSVTGAVLDPRPGDNDGRSVIPVREAPPTPTPTPTPTPTPTPTPTPTPTPTRTPTPTPTPTRPPQPPAPKAGPGVRITVQPEPGYVGGRVVVTYSVRNGRNALATGLRLRIGLPAGVPHGGLPAGCDRNGACALPDLTPGTTAVLRVVLSPKKAMTARVTAVLDTTGTDADRSDNTARERLRVLQPRIVAVPDIGKPGFVTSVRGVDFPPGVPVRFSWNPGITAAASPTFPEADGTFIGQLLILAKDQTGPRTITASGPGFSPVKTDFLVVSGTVQPPDGVTRR

SEQ ID NO: 44 - protein, 22 aa, Artificial Sequence
  Synthetic Construct
TTCGAYSCSGVSTTCTTCGSAT

SEQ ID NO: 45 - protein, 23 aa, Artificial Sequence
  Synthetic Construct
GCSATGGAYCCSCARCARCGSVT

SEQ ID NO: 46 - protein, 22 aa, Artificial Sequence
  Synthetic Construct
SSCTSGTSGCSMTSCAYCWSGC

SEQ ID NO: 47 - protein, 23 aa, Artificial Sequence
  Synthetic Construct
GTSCCSGTSCCRTGSSCYTCSAC

SEQ ID NO: 48 - protein, 22 aa, Artificial Sequence
  Synthetic Construct
ASRTGSGCRTTSGTSCCSSWSA

SEQ ID NO: 49 - DNA, 21 bp, Artificial Sequence
  Synthetic Construct
cgtcagcctgatcctcgccga

SEQ ID NO: 50 - DNA, 21 bp, Artificial Sequence
  Synthetic Construct
tccaggtggccgacgttcgtc

SEQ ID NO: 51 - DNA, 21 bp, Artificial Sequence
  Synthetic Construct
aacgagatcccgccgggcctc

SEQ ID NO: 52 - DNA, 17 bp, Artificial Sequence
  Synthetic Construct
cgcgttgctgggcgagg

SEQ ID NO: 53 - DNA, 21 bp, Artificial Sequence
  Synthetic Construct
ggacgtctgccggagggttcc

SEQ ID NO: 54 - DNA, 21 bp, Artificial Sequence
  Synthetic Construct
ggcccgttgggcacggacaga

SEQ ID NO: 55 - DNA, 30 bp, Artificial Sequence
  Synthetic Construct
tttgcatgcgatgttgacgatctcctcgtc

SEQ ID NO: 56 - DNA, 30 bp, Artificial Sequence
  Synthetic Construct
ggaagcttcatatgttctctccggaatgtg

SEQ ID NO: 57 - DNA, 31 bp, Artificial Sequence
  Synthetic Construct
ttaagctttctagagaggagaggccgtgaac

SEQ ID NO: 58 - DNA, 31 bp, Artificial Sequence
  Synthetic Construct
aaagaattcgaactcgagcacggactcgttg

SEQ ID NO: 59 - DNA, 21 bp, Artificial Sequence
  Synthetic Construct
gacacggccggtgagagcagc

SEQ ID NO: 60 - DNA, 23 bp, Artificial Sequence
  Synthetic Construct
cttctagatgtcgcggtgtacgg

SEQ ID NO: 61 - DNA, 42 bp, Artificial Sequence
  Synthetic Construct
cttaagggttaattaaggaggacacatatgtccggagaattc

SEQ ID NO: 62 - protein, 5 aa, Artificial Sequence
  Synthetic Construct
MSGEF

(19) United States
(12) Patent Application Publication
(10) Pub. No.: US 2004/0132055 A1
(43) Pub. Date: Jul. 8, 2004

(54) Recombinant chalcomycin polyketide synthase and modifying genes

(76) Inventors: Leonard Katz, Oakland, CA (US); Ralph C. Reid, San Rafael, CA (US); Zhihao Hu, Castro Valley, CA (US); Andreas Schirmer, Hayward, CA (US); Shannon L. Ward, Pleasanton, CA (US); Christopher Reeves, Orinda, CA (US)

Correspondence Address: MORRISON & FOERSTER LLP, 755 PAGE MILL RD, PALO ALTO, CA, 94304-1018, US

(21) Appl. No.: 10/647,196

(22) Filed: Aug. 21, 2003

Related U.S. Application Data
(60) Provisional application No. 60/420,994, filed on Oct. 24, 2002.
(60) Provisional application No. 60/493,966, filed on Aug. 8, 2003.

(51) Int. Cl. C12Q 1/68; C12P 19/60; C07H 21/04; C12N 9/10; C12N 1/21

(52) U.S. Cl. 435/6; 435/69.1; 435/75; 435/193; 435/252.3; 435/320.1; 536/23.2

(57) ABSTRACT

Domains of chalcomycin polyketide synthases and modification enzymes and polynucleotides encoding them are provided. Methods to prepare chalcomycin in pharmaceutically useful quantities are described, as are methods to prepare chalcomycin analogs and other polyketides using the polynucleotides encoding chalcomycin polyketide synthase domains or modifying enzymes.

### CROSS-REFERENCE TO RELATED APPLICATIONS

The present application claims benefit of U.S. provisional patent application Nos. 60/405,194 (filed Aug. 21, 2002); 60/420,994 (filed Oct. 24, 2002); and 60/493,966 (filed Aug. 8, 2003) each of which is incorporated herein by reference in its entirety.

### STATEMENT OF GOVERNMENT INTEREST

Subject matter disclosed in this application was made, in part, with government support under NIH Grant No. R43 CA AI50305. As such, the United States government may have certain rights in this invention.

### FIELD OF THE INVENTION

The invention relates to recombinant polynucleotides that encode polypeptides or domains of the chalcomycin polyketide synthase gene cluster. Accordingly, the present invention is directed to the production of chalcomycin PKS enzymes, to polynucleotides that encode such enzymes, and to host cells that contain such polynucleotides. Further enhancements in the biological activities of chalcomycin and other polyketides, through production of derivatives thereof, is also made possible according to the practice of the invention by providing P450 hydroxylases that provide attachment points on the polyketide molecule for further modifications. Thus the present invention relates to the fields of molecular biology, chemistry, recombinant DNA technology, medicine, animal health, and agriculture.

### BACKGROUND OF THE INVENTION

Polyketides represent a large family of diverse compounds synthesized from 2 carbon units through a series of condensations and subsequent modifications. Polyketides occur in many types of organisms including fungi and mycelial bacteria, in particular the actinomycetes. An appreciation for the wide variety of polyketide structures and for their biological activities, may be gained upon review of the extensive art, for example, published PCT Patent Publication WO 95/08548 and U.S. Pat. Nos. 5,672,491 and 6,303,342

Polyketides are synthesized in nature by polyketide synthases (“PKS”). The Type I or modular PKS comprise a set of separate catalytic active sites; each active site is termed a “domain”, and a set thereof is termed a “module”. One module exists for each cycle of carbon chain elongation and modification. FIG. 9 of aforementioned WO95/08548 depicts a typical Type I PKS, in this case 6-deoxyerythronolide B synthase (“DEBS”), which is involved in the production of erythromycin. Six separate modules, each catalyzing a round of condensation and modification of a 2-carbon unit, are present in DEBS. The number and type of catalytic domains that are present in each module varies based on the needed chemistry, and the total of 6 modules is provided on 3 separate polypeptides (designated DEBS-1, DEBS-2, and DEBS-3, with 2 modules per each polypeptide). Each of the DEBS polypeptides is encoded by a separate open reading frame (gene), see Caffrey et al., FEBS Letters, 304, pp. 205, 1992. DEBS provides a representative example of a Type I PKS. In DEBS, modules 1 and 2 reside on DEBS-1, modules 3 and 4 on DEBS-2, and modules 5 and 6 on DEBS-3, wherein module 1 is defined as the first module to act on the growing polyketide backbone, and module 6 the last.

The minimal PKS module is typified by module 3 of DEBS which contains a ketosynthase (“KS”) domain, an acyltransferase (“AT”) domain, and an acyl carrier protein (“ACP”) domain. These three enzyme activities are sufficient to activate a 2-carbon extender unit and attach it to the growing polyketide molecule. Additional domains that may be included in a module relate to reactions other than the actual condensation, and include domains for a ketoreductase activity (“KR”), a dehydratase activity (“DH”), and an enoylreductase activity (“ER”). With respect to DEBS-1, the first module thereof also contains an additional set of the AT and ACP activities because that module catalyzes initial condensation, and so begins with a “loading domain” (sometimes referred to as a loading module) that contains an AT and ACP, that bind the starter unit. The “finishing” of the 6-deoxyerythronolide molecule is regulated by a thioesterase activity (“TE”) in module 6 that catalyzes cyclization of the macrolide ring during release of the product of the PKS.

PKS genes can be engineered in a variety of ways to achieve biosynthesis of polyketides. For instance, PKS genes can be inserted into a heterologous host to make a polyketide in a host that does not make it naturally. Polyketides can also be made by hybrid or otherwise altered PKSs or polyketide biosynthetic gene clusters. Also, polyketides can be overproduced by increasing the pools of available starting polyketide biosynthetic precursors and by other means. See U.S. Pat. Nos. 5,672,491; 5,962,290; 6,080,555; 6,391,594; and 6,221,641 and PCT Patent Publications 00/47724, 01/27306, and 01/31035.

Chalcomycin is a 16-membered macrolide antibiotic produced by some strains of Streptomyces bikiniensis and possesses a broad spectrum of antimicrobial activity. Certain naturally occurring derivatives of chalcomycin produced by other Streptomyces organisms also have antimicrobial activity. For instance, the 8-deoxy chalcomycin derivative produced by Streptomycin hirsutus has antimicrobial activity against gram-positive bacteria. Chalcomycin has two modifying sugar molecules, D-mycinose and D-chalcose, the former being subject to post-glycosylation modification by O-methylation at two positions. For additional information regarding chalcomycin, see Woo, P. W. K. et al., J.A.C.S., 1962, 84, 1512; 1964, 86, 2724; 2726; Celmer, W. D., J. A. C. S., 1965, 87, 1801; Omura, S. et al., J. A. C. S., 1975, 97, 4001; Neszmelyi, A. et al., Chem. Comm., 1976, 97; Jardim, M. E. et al., Int. J. Mass Spectrom. Ion Phys., 1983, 48, 189; Hauske, J. R. et al., J. O. C., 1986, 51, 2808; Kim, S. D. et al., J. Antibiot., 1996, 49, 955; Woo, P. W. K. et al., Tetrahedron, 1996, 52, 3857 and Goo, Y. M. et al., J. Antibiot., 1997, 50, 85.

The chemical structure of chalcomycin, shown without stereochemistry, is provided by formula I below.

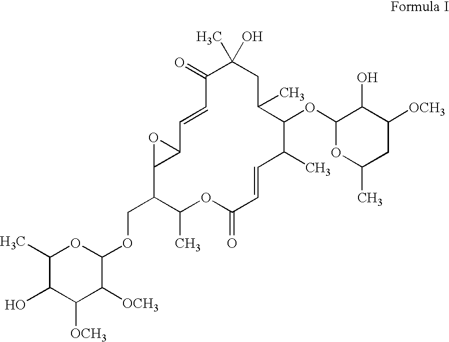

Chalcomycin is synthesized by a Type I or modular PKS and modification enzymes. Post-PKS modification reactions include P450 oxidation at three sites to add hydroxyl groups and glycosylation at the C5 hydroxyl to add D-chalcose, and at the C20 hydroxyl to add allose, which is then methylated at two positions to yield D-mycinose.

There is a need for recombinant nucleic acids, host cells, and methods of using those host cells to produce polyketides including but not limited to chalcomycin and chalcomycin analogs. These and other needs are met by the materials and methods provided by the present invention.

### SUMMARY OF THE INVENTION

The present invention provides recombinant nucleic acids encoding polyketide synthases and polyketide modification enzymes. The recombinant nucleic acids of the invention are useful in the production of polyketides, including but not limited to chalcomycin and chalcomycin analogs and derivatives in recombinant host cells. The biosynthesis of chalcomycin is performed by a modular PKS and polyketide modification enzymes. The chalcomycin synthase is made up of several proteins, each having one or more modules. The modules have domains with specific synthetic functions.

The present invention also provides domains and modules of the chalcomycin PKS and corresponding nucleic acid sequences encoding them and/or parts thereof. Such compounds are useful in the production of hybrid PKS enzymes and the recombinant genes that encode them.

The present invention also provides modifying genes of chalcomycin biosynthetic gene cluster in recombinant form, including but not limited to isolated form and incorporated into a vector or the chromosomal DNA of a host cell. The present invention also provides recombinant P450 hydroxylases that provide hydroxyl attachment points useful for further chemical modification. The P450 hydroxylases of the present invention include ChmHI, ChmPI and ChmPII hydroxylases.

The present invention also provides recombinant host cells that contain the nucleic acids of the invention. In one embodiment, the host cell provided by the invention is a Streptomyces host cell that produces a chalcomycin modification enzyme and/or a domain, module, or protein of the chalcomycin PKS. Methods for the genetic manipulation of Streptomyces are described in Kieser et al, “Practical Streptomyces Genetics,” The John Innes Foundation, Norwich (2000), which is incorporated herein by reference in its entirety.

Accordingly, there is provided a recombinant PKS wherein at least 10, 15, 20, or more consecutive amino acids in one or more domains of one or more modules thereof are derived from one or more domains of one or more modules of chalcomycin polyketide synthase. Preferabiy at least an entire domain of a module of chalcomycin synthase is included. Representative chalcomycin PKS domains useful in this aspect of the invention include, for example, KR, DH, ER, AT, ACP and KS domains. In one embodiment of the invention, the PKS is assembled from polypeptides encoded by DNA molecules that comprise coding sequences for PKS domains, wherein at least one encoded domain corresponds to a domain of chalcomycin PKS. In such DNA molecules, the coding sequences are operably linked to control sequences so that expression therefrom in host cells is effective. In this manner, chalcomycin PKS coding sequences or modules and/or domains can be made to encode PKS to biosynthesize compounds having antibiotic or other useful bioactivity other than chalcomycin.

These and other aspects of the present invention are described in more detail in the Detailed Description of the Invention, below.

### BRIEF DESCRIPTION OF THE DRAWINGS

FIG. 1 illustrates the structure of the chalcomycin PKS biosynthetic gene cluster, and cosmids pKOS146.185.1 and pKOS146.185.10, which contain insert DNA encompassing the chalcomycin PKS gene cluster and associated modification enzyme genes. Abbreviations: ACP, acyl carrier protein; chm, chalcomycin gene; Orf, open reading frame.

FIG. 2 shows proposed pathways for post-PKS modification of the chalcomycin-spiramycin hybrid PKS macrolide product.

### DETAILED DESCRIPTION OF THE INVENTION

The invention provides recombinant materials for the production of polyketides. In an aspect, the present invention provides recombinant nucleic acids encoding polyketide synthases that contain all or a portion of the chalcomycin PKS. The biosynthesis of chalcomycin is performed by a modular PKS and modification enzymes. The chalcomycin synthase is made up of five proteins, each having one or more modules, each module comprising domains with specific synthetic functions. Thus, the present invention also provides the domains and modules of the chalcomycin PKS and corresponding nucleic acid sequences encoding them in recombinant form.

Modifying genes of the chalcomycin biosynthetic gene cluster are also provided, including but not limited to the genes for the ChmHI, ChmPI and ChmPII P450 hydroxylases that provide hydroxyl attachment points useful for further chemical modification.

Methods and host cells for using these genes to produce or modify a polyketide in recombinant host cells are also provided.

The nucleotide sequences encoding chalcomycin PKS and modifying proteins of the present invention were isolated from Streptomyces bikiniensis NRRL 2737 (obtained from the Agricultural Research Service Culture Collection, National Center for Agricultural Utilization Research, Peoria, Ill. USA). The chalcomycin PKS gene cluster and modifying genes are contained in cosmids pKOS 146.185.1 and pKOS146.185.10. The cloning and characterization of the chalcomycin PKS gene cluster is described in Example 1, infra. pKOS146-185.1 was deposited under the terms of the Budapest Treaty with the American Type Culture Collection, 10801 University Blvd., Manassas, Va., 20110-2209, on Feb. 19, 2003, with accession number PTA-4961. pKOS146-185.10 was deposited under the terms of the Budapest Treaty with the American Type Culture Collection, 10801 University Blvd., Manassas, Va., 20110-2209, on Feb. 19, 2003, with accession number PTA-4962.

Given the valuable properties of chalcomycin and its modifying enzymes, means to produce useful quantities thereof and derivatives or analogs of chalcomycin are valuable. Further, the chalcomycin modifying enzymes can also be used to modify other polyketides and produce derivatives thereof with enhanced solubility and/or bioactivity, for instance as antibiotics, and/or sites for further enzymatic or chemical modification. The nucleotide sequences of the chalcomycin biosynthetic gene cluster encoding chalcomycin PKS and modifying enzymes, and domains and/or modules of the PKS can be used, for example, to make antibiotics or other useful compounds in addition to chalcomycin, and in host cells in addition to Streptomyces bikiniensis.

There is a need for recombinant nucleic acids, host cells, and methods of expressing those nucleic acids in host cells resulting in production of chalcomycin and or its analogs or derivatives, and modifying enzymes, such as the cytochrome P450 hydroxylases that specifically attach hydroxyl groups on the resulting aglycone (which can then be used as attachment points for further modifications). The modifying P450's from the chalcomycin PKS cluster of the present invention can be used to make compounds in a host that does not naturally produce such compounds. These and other needs are met by the materials and methods of the present invention

In one aspect of the invention, purified and isolated DNA molecules are provided that comprise one or more coding sequences for one or more domains or modules of chalcomycin synthase. Examples of such encoded domains include chalcomycin synthase KR, DH, ER, AT, ACP, and KS domains. In one aspect, the invention provides DNA molecules in which the complete set of chalcomycin PKS-encoding sequences are operably linked to expression control sequences that are effective in suitable host cells to produce chalcomycin and/or its analogs or derivatives. In one aspect, the invention provides polypeptides comprising a portion of the coding sequences for the proteins of the chalcomycin synthase.

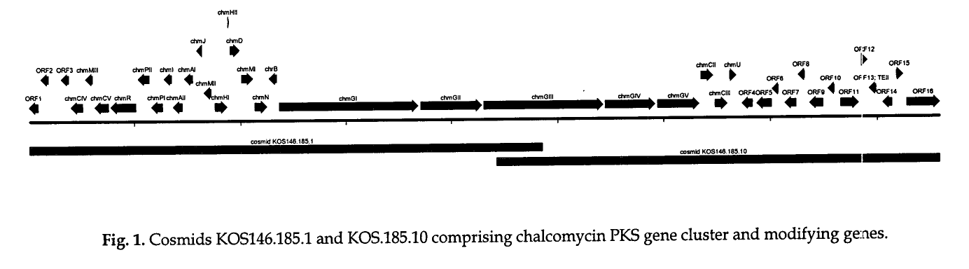

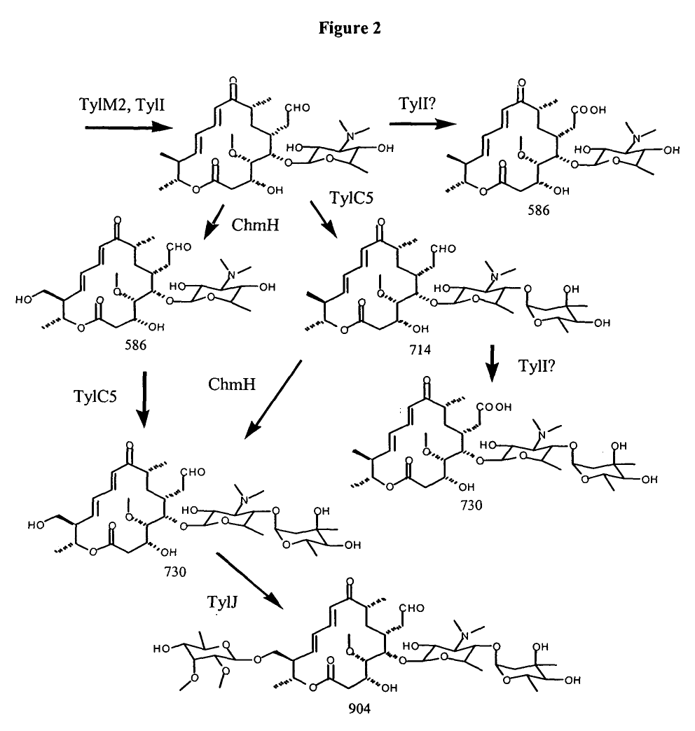

Table 2 in Example 1 provides a description of genes in the chalcomycin PKS gene (i.e., SEQ ID NO:1 and subsequences encoding modules, domains and ORFs, e.g., as indicated), as well as encoded proteins (including SEQ ID. NOS: 2-43) or domains. It will be apparent from Table 2, and FIGS. 1 and 2, which DNA strand comprises the coding sequence for a protein (i.e., the strand having the sequence of SEQ ID NO:1, or its complement.

In one aspect, the invention provides an isolated or recombinant DNA molecule comprising a nucleotide sequence that encodes at least one polypeptide, alternatively at least one module, alternatively at least one domain, involved in the biosynthesis of a chalcomycin. In one aspect, the invention provides the present invention provides an isolated or recombinant DNA molecule comprising a nucleotide sequence that encodes at least one polypeptide, alternatively at least one module, alternatively at least one domain, involved in the biosynthesis of a chalcomycin. The invention also provides polypeptides comprising PKS interpolypeptide linker sequences, and polynucleotides encoding such linker sequences. Also provided by the invention are polypeptides comprising intrapolypeptide linker sequences, and polynucleotides encoding such linkers.

In one aspect, the invention provides an isolated or recombinant DNA molecule comprising a sequence identical or substantially similar to at least one subsequence of SEQ ID NO:1 or its complement. In an embodiment the subsequence comprises a sequence encoding a chalcomycin PKS domain or module. In an aspect, the invention provides a recombinant DNA molecule that encodes a polypeptide, module or domain derived from a chalcomycin polyketide synthase (PKS) gene cluster. In this context, a polypeptide, module or domain is derived from a chalcomycin polyketide synthase (PKS) gene cluster when it is encoded by a DNA with substantial sequence identity to the corresponding coding region of the S. bikiniensis chalcomycin gene cluster. For example, in an embodiment, the DNA encoding sequence of the polypeptide, module or domain hybridizes under stringent conditions to a nucleic acid having the sequence of SEQ ID NO:1 (or its complement). Generally, such a polypeptide, module or domain is biologically active, i.e., has at least one enzymatic activity chracteristic of the polypeptide, module or domain encoded exactly by corresponding sequence of SEQ ID NO:1 or its complement. The biological activity of a polypeptide of the invention can be measured by methods well known to the art.

In one aspect, the invention provides the present invention provides an isolated or recombinant DNA molecule comprising a nucleotide sequence that encodes an open reading frame, module or domain having an amino acid sequence identical or substantially similar to an ORF, module or domain encoded by SEQ ID NO:1 or its complement. Generally, a polypeptide, module or domain having a sequence substantially similar to a reference sequence has substantially the same activity as the reference protein, module or domain (e.g., when integrated into an appropriate PKS framework using methods known in the art). In certain embodiments, one or more activities of a substantially similar polypeptide, module or domain are modified or inactivated as described below.

In one aspect, the invention provides an isolated or recombinant DNA molecule comprising a nucleotide sequence that encodes at least one polypeptide, module or domain encoded by SEQ ID NO:1, e.g., a polypeptide, module or domain involved in the biosynthesis of a chalcomycin, wherein said nucleotide sequence comprises at least 20, 25, 30, 35, 40, 45, or 50 contiguous base pairs identical to a sequence of SEQ ID NO:1 or its complement. In one aspect, the invention provides an isolated or recombinant DNA molecule comprising a nucleotide sequence that encodes at least one polypeptide, module or domain encoded by SEQ ID NO:1, e.g., a polypeptide, module or domain involved in the biosynthesis of a chalcomycin, wherein said polypeptide, module or domain comprises at least 10, 15, 20, 30, or 40 contiguous residues of a corresponding polypeptide, module or domain encoded by SEQ ID NO:1 or its complement.

In a related aspect, the invention provides a recombinant DNA molecule, comprising a sequence of at least about 200, optionally at least about 500, basepairs with a sequence identical or substantially identical to a protein encoding region of SEQ ID NO:1. In an embodiment, the DNA molecule encodes a polypeptide, module or domain derived from a chalcomycin polyketide synthase (PKS) gene cluster.

It will be understood that SEQ ID NO:1 was determined using the inserts of pKOS 146.185.1 and pKOS146-185.10. Accordingly, the invention provides an isolated or recombinant DNA molecule comprising a sequence identical or substantially similar to a ORF encoding sequence of the insert of pKOS 146.185.1 or pKOS146-185.10.

Those of skill will recognize that, due to the degeneracy of the genetic code, a large number of DNA sequences encode the amino acid sequences of the domains, modules, and proteins of the chalcomycin PKS, the enzymes involved in chalcomycin modification and other polypeptides encoded by the genes of the chalcomycin biosynthetic gene cluster. The present invention contemplates all such DNAs. For example, it may be advantageous to optimize sequence to account for the codon preference of a host organism. The invention also contemplates naturally occurring genes encoding the chalcomycin PKS and tailoring enzymes that are polymorphic or other variants. In addition, it will be appreciated that polypeptide, modules and domains of the invention may comprise one or more conservative amino acid substitutions relative to the polypeptides encoded by SEQ ID NO: 1, such as, for example, conservative substitutions include aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids.

As used herein, the terms “substantial identity,” “substantial sequence identity,” or “substantial similarity” in the context of nucleic acids, refers to a measure of sequence similarity between two polynucleotides. Substantial sequence identity can be determined by hybridization under stringent conditions, by direct comparison, or other means. For example, two polynucleotides can be identified as having substantial sequence identity if they are capable of specifically hybridizing to each other under stringent hybridization conditions. Other degrees of sequence identity (e.g., less than “substantial”) can be characterized by hybridization under different conditions of stringency. “Stringent hybridization conditions” refers to conditions in a range from about 5° C. to about 20° C. or 25° C. below the melting temperature (Tm) of the target sequence and a probe with exact or nearly exact complementarity to the target. As used herein, the melting temperature is the temperature at which a population of double-stranded nucleic acid molecules becomes half-dissociated into single strands. Methods for calculating the Tm of nucleic acids are well known in the art (see, e.g., Berger and Kimmel, 1987, Methods In Enzymology, Vol. 152: Guide To Molecular Cloning Techniques, San Diego: Academic Press, Inc. and Sambrook et al., 1989, Molecular Cloning: A Laboratory Manual, 2nd Ed., Vols. 1-3, Cold Spring Harbor Laboratory). Typically, stringent hybridization conditions for probes greater than 50 nucleotides are salt concentrations less than about 1.0 M sodium ion, typically about 0.01 to 1.0 M sodium ion at pH 7.0 to 8.3, and temperatures at least about 50° C., preferably at least about 60° C. As noted, stringent conditions may also be achieved with the addition of destabilizing agents such as formamide, in which case lower temperatures may be employed. Exemplary conditions include hybridization at 7% sodium dodecyl sulfate (SDS), 0.5 M NaPO₄ pH 7.0, 1 mM EDTA at 50° C. (or alternatively 65° C.); wash with 2×SSC, 1% SDS, at 50° C. (or alternatively 0.1-0.2 ×SSC, 1% SDS, at 50° C. or 65° C.). Other exemplary conditions for hybridization include (1) high stringency: 0.1×SSPE, 0.1% SDS, 65° C.; (2) medium stringency: 0.2×SSPE, 0.1% SDS, 50° C.; and (3) low stringency: 1.0×SSPE, 0.1% SDS, 50° C. Equivalent stringencies may be achieved using alternative buffers, salts and temperatures.

Alternatively, substantial sequence identity can be described as a percentage identity between two nucleotide or amino acid sequences. Two nucleic acid sequences are considered substantially identical when they are at least about 70% identical, at least about 75% identical, or at least about 80% identical, or at least about 85% identical, or at least about 90% identical, or at least about 95% or 98% identical. Two amino acid sequences are considered substantially identical when they are at least about 60%, sequence identical, more often at least about 70%, at least about 80%, or at least about 90% sequence identity to the reference sequence. Percentage sequence (nucleotide or amino acid) identity is typically calculated using art known means to determine the optimal alignment between two sequences and comparing the two sequences. Optimal alignment of sequences may be conducted using the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. U.S.A. 85: 2444, by the BLAST algorithm of Altschul (1990) J. Mol. Biol. 215: 403-410; and Shpaer (1996) Genomics 38:179-191, or by the Needleham et al. (1970) J. Mol. Biol. 48: 443-453; and Sankoff et al., 1983, Time Warps, String Edits, and Macromolecules, The Theory and Practice of Sequence Comparison, Chapter One, Addison-Wesley, Reading, Mass.; generally by computerized implementations of these algorithms (GAP, BESTFIT, FASTA, and TFASTA in the Wisconsin Genetics Software Package, Genetics Computer Group, 575 Science Dr., Madison, Wis.; BLAST from the National Center for Biotechnology Information (http:// www.ncbi.nlm.nih.gov/). In each case default parameters are used (for example the BLAST program uses as defaults a wordlength (W) of 11, the BLOSUM62 scoring matrix (see Henikoff (1992) Proc. Natl. Acad. Sci. USA 89: 10915-10919) alignments (B) of 50, expectation (E) of 10, M=5, N=−4, and a comparison of both strands).

As used herein the term “recombinant” has its usual meaning in the art and refers to a polynucleotide synthesized or otherwise manipulated in vitro, or to methods of using recombinant polynucleotides to produce gene products in cells or other biological systems. Thus, a “recombinant” polynucleotide is defined either by its method of production or its structure. In reference to its method of production, the process is use of recombinant nucleic acid techniques, e.g., involving human intervention in the nucleotide sequence, typically selection or production. Alternatively, a recombinant polynucleotide can be a polynucleotide made by generating a sequence comprising fusion of two fragments which are not naturally contiguous to each other, but is meant to exclude products of nature. Thus, for example, products made by transforming cells with any non-naturally occurring vector is encompassed, as are polynucleotides comprising sequence derived using any synthetic oligonucleotide process, as are polynucleotides from which a region has been deleted. A recombinant polynucleotide can also be a coding sequence that has been modified in vivo using a recombinant oligo or polynucleotide (such as a PKS in which a domain is inactivated by homologous recombination using a recombinant polynucleotide). A “recombinant” polypeptide is one expressed from a recombinant polynucleotide.

It will be immediately recognized by those of skill that recombinant polypeptides of the invention have a variety of uses, some of which are described in detail below, including but not limited to use as enzymes, or componants of enzymes, useful for the synthesis or modification of polyketides. Recombinant polypeptides encoded by the chalcomycin PKS gene cluster are also useful as antigens for production of antibodies. Such antibodies find use for purification of bacterial (e.g., Streptomyces bikiniensis) proteins, detection and typing of bacteria, and particularly, as tools for strain improvement (e.g., to assay PKS protein levels to identify “up-regulated” strains in which levels of polyketide producing or modifying proteins are elevated) or assessment of efficiency of expression of recombinant proteins. Polyclonal and monoclonal antibodies can be made by well known and routine methods (see, e.g., Harlow and Lane, 1988, ANTIBODIES: A LABORATORY MANUAL, COLD SPRING HARBOR LABORATORY, New York; Koehler and Milstein 1075, Nature 256:495). In selecting polypeptide sequences for antibody induction, it is not to retain biological activity; however, the protein fragment must be immunogenic, and preferably antigenic (as can be determined by routine methods). Generally the protein fragment is produced by recombinant expression of a DNA comprising at least about 60, more often at least about 200, or even at least about 500 or more base pairs of protein coding sequence, such as a polypeptide, module or domain derived from a chalcomycin polyketide synthase (PKS) gene cluster. Methods for expression of recombinant proteins are well known. (See, e.g., Ausubel et al., 2002, Current Protocols In Molecular Biology, Greene Publishing and Wiley-Interscience, New York.)

Further aspects of the invention include chimeric PKSs comprising a portion (in one embodiment at least a domain, optionally at least a module, or alternatively at least one polypeptide) from the chalcomycin PKS, and a portion (in one embodiment at least a domain, optionally at least a module, or alternatively at least a polypeptide) from one or more non-chalcomycin PKSs. For example, the invention provides (1) encoding DNA for a chimeric PKS that is substantially patterned on a non-chalcomycin producing enzyme, but which includes one or more functional domains or modules of chalcomycin PKS; (2) encoding DNA for a chimeric PKS that is substantially patterned on the chalcomycin PKS, but which includes one or more functional domains or modules of another PKS or NRPS; and (3) methods for making chalcomycin analogs and derivatives. With respect to item (1) above, examples include chimeric PKS enzymes wherein the genes for the erythromycin PKS, rapamycin PKS, tylosin PKS, and spiramycin PKS, or another PKS function as accepting genes, and one or more of the above-identified coding sequences for chalcomycin domains or modules are inserted as replacements for domains or modules of comparable function. With respect to item (2) above, examples include chimeric PKS enzymes wherein the chalcomycin PKS serves as an accepting gene, and genes for the erythromycin PKS, rapamycin PKS, tylosin PKS, and spiramycin PKS, or another PKS function as accepting genes, and one or more of the above-identified coding sequences for chalcomycin domains or modules are inserted as replacements for domains or modules of comparable function. A partial list of sources of PKS sequences for use in making chimeric molecules, for illustration and not limitation, includes Avermectin (U.S. Pat. No. 5,252,474; MacNeil et al., 1993, Industrial Microorganisms: Basic and Applied Molecular Genetics, Raltz. Hegeman, & Skatrud, eds. (ASM), pp. 245-256; MacNeil et al., 1992, Gene 115: 119-25); Candicidin (FRO008) (Hu et al., 1994, Mol. Microbiol. 14: 163-72); Epothilone (U.S. Pat. No. 6,303,342); Erythromycin (WO 93/13663; U.S. Pat. No. 5,824,513; Donadio et al., 1991, Science 252:675-79; Cortes et al., 1990, Nature 348:176-8); FK-506 (Motamedi et al., 1998, Eur. J. Biochem. 256:528-34; Motamedi et al., 1997, Eur. J. Biochem. 244:74-80); FK-520 (U.S. Pat. No. 6,503,737; see also Nielsen et al., 1991, Biochem. 30:5789-96); Lovastatin (U.S. Pat. No. 5,744,350); Nemadectin (MacNeil et al., 1993, supra); Niddamycin (Kakavas et al., 1997, J. Bacteriol. 179:7515-22); Oleandomycin (Swan et al., 1994, Mol. Gen. Genet. 242:358-62; U.S. Pat. No. 6,388,099; Olano et al., 1998, Mol. Gen. Genet. 259:299-308); Platenolide (EP Pat. App. 791,656 ); Rapamycin (Schwecke et al., 1995, Proc. Natl. Acad. Sci. USA 92:7839-43); Aparicio et al., 1996, Gene 169:9-16); Rifamycin (August et al., 1998, Chemistry & Biology, 5: 69-79); Soraphen (U.S. Pat. No. 5,716,849; Schupp et al., 1995, J. Bacteriology 177: 3673-79); Spiramycin (U.S. Pat. No. 5,098,837); Tylosin (EP 0 791,655; Kuhstoss et al., 1996, Gene 183:231-36; U.S. Pat. No. 5,876,991). Additional suitable PKS coding sequences remain to be discovered and characterized, but will be available to those of skill (e.g., by reference to GenBank).

Construction of such chimeric enzymes is most effectively achieved by construction of appropriate encoding polynucleotides. In preparing modified and chimeric proteins, it is not necessary, although it may be most efficient, to replace or substitute one or more entire domains or modules of one PKS (e.g., the chalcomycin PKS or another PKS) with an entire domain or module of a different PKS (e.g., the chalcomycin PKS or another PKS). Rather, peptide subsequences of a PKS domain or module that correspond to a peptide subsequence in an accepting domain or module, or which otherwise provide useful function, may be used as replacements. Accordingly, appropriate encoding DNAs for construction of such chimeric PKS include those that encode at least 10, 15, 20 or more amino acids of a selected chalcomycin domain or module. Recombinant methods for manipulating modular PKS genes to make chimeric PKS enzymes are described in U.S. Pat. Nos. 5,672,491; 5,843,718; 5,830,750; and 5,712,146; and in PCT publication Nos. 98/49315 and 97/02358. A number of genetic engineering strategies have been used with DEBS to demonstrate that the structures of polyketides can be manipulated to produce novel natural products, primarily analogs of the erythromycins (see the patent publications referenced supra and Hutchinson, 1998, Curr Opin Microbiol. 1:319-329, and Baltz, 1998, Trends Microbiol. 6:76-83).

The invention methods may be directed to the preparation of an individual polyketide. The polyketide may or may not be novel, but the method of preparation permits a more convenient or alternative method of preparing it. The resulting polyketides may be further modified to convert them to other useful compounds. Examples of chemical structures of sixteen-membered macrolides that can be made using the materials and methods of the present invention are described in PCT Patent Publication WO 02/32916; U.S. Patent Application US20020128213A (app. Ser. No. 09/969,177); and copending U.S. provisional patent application No. 60/493,966.

The recombinant DNAs and DNA vectors of the inventions can also be used to make “libraries” of polyketides. Generally, members of these polyketide libraries may themselves be novel compounds, and the invention further includes novel polyketide members of these libraries. Regardless of the naturally occurring PKS gene used as an acceptor, the invention provides libraries of polyketides by generating modifications in, or using a portion of, the chalcomycin PKS so that the protein complexes produced have altered activities in one or more respects, and thus produce polyketides other than the natural product of the PKS. Novel polyketides may thus be prepared, or polyketides in general prepared more readily, using this method. By providing a large number of different genes or gene clusters derived from a naturally occurring PKS gene cluster, each of which has been modified in a different way from the native cluster, an effectively combinatorial library of polyketides can be produced as a result of the multiple variations in these activities.

As noted, in one aspect the invention provides recombinant PKS wherein at least 10, 15, 20, or more consecutive amino acids in one or more domains of one or more modules thereof are derived from one or more domains of one or more modules of chalcomycin polyketide synthase. A polyketide synthase “derived from” a naturally occurring PKS contains the scaffolding encoded by all the portion employed of the naturally occurring synthase gene, contains at least two modules that are functional, and contains mutations, deletions, or replacements of one or more of the activities of these functional modules so that the nature of the resulting polyketide is altered. This definition applies both at the protein and genetic levels. Particular embodiments include those wherein a KS, AT, KR, DH, or ER has been deleted or replaced by a version of the activity from a different PKS or from another location within the same PKS, and derivatives where at least one noncondensation cycle enzymatic activity (KR, DH, or ER) has been deleted or wherein any of these activities has been added or mutated so as to change the ultimate polyketide synthesized.

There are at least five degrees of freedom for constructing a polyketide synthase in terms of the polyketide that will be produced. First, the polyketide chain length will be determined by the number of modules in the PKS. Second, the nature of the carbon skeleton of the PKS will be determined by the specificities of the acyl transferases which determine the nature of the extender units at each position—e.g., malonyl, methyl malonyl, methoxy malonyl, or ethyl malonyl, etc. Third, the loading domain specificity will also have an effect on the resulting carbon skeleton of the polyketide. Fourth, the oxidation state at various positions of the polyketide will be determined by the dehydratase and reductase portions of the modules. This will determine the presence and location of ketone, alcohol, alkene or alkane substituents at particular locations in the polyketide. Fifth, the stereochemistry of the resulting polyketide is a function of three aspects of the synthase. The first aspect is related to the AT/KS specificity associated with substituted malonyls as extender units, which affects stereochemistry only when the reductive cycle is missing or when it contains only a ketoreductase since the dehydratase would abolish chirality. Also, the specificity of the ketoreductase will determine the chirality of the corresponding hydroxyl group. Also, the enoyl reductase specificity for substituted malonyls as extender units will influence the result when there is a complete KR/DH/ER available.

As can be appreciated by those skilled in the art, polyketide biosynthesis can be manipulated to make a product other than the product of a naturally occurring PKS biosynthetic cluster. For example, AT domains can be altered or replaced to change specificity. For example, and not limitation, the AT domain of chalcomycin module 0 (loading domain) can be replaced by an AT with specificity for methylmalonyl-CoA to produce chalcomycin derivatives with a C-15 ethyl group in place of the C-15 methyl group. The variable domains within a module can be deleted and or inactivated or replaced with other variable domains found in other modules of the same PKS or from another PKS. See e.g., Katz & McDaniel, Med Res Rev 19: 543-558 (1999) and WO 98/49315. Similarly, entire modules can be deleted and/or replaced with other modules from the same PKS or another PKS. See e.g., Gokhale et al., Science 284:482 (1999) and WO 00/47724. For example, and not limitation, 3-hydroxy derivatives of chalcomycin can be produced using a modified chalcomycin PKS in which module 7 of the chalcomycin PKS is replaced by module 7 of the tylosin PKS (optionally with appropriate linker modifications). Similarly, protein subunits of different PKSs also can be mixed and matched to make compounds having the desired backbone and modifications. For example, subunits of 1 and 2 (encoding modules 1-4) of the pikromycin PKS were combined with the DEBS3 subunit to make a hybrid PKS product (see Tang et al., Science, 287: 640 (2001), WO 00/26349 and WO 99/6159). Also see Examples, below.

Mutations can be introduced into PKS genes such that polypeptides with altered activity are encoded. Polypeptides with “altered activity” include those in which one or more domains are inactivated or deleted, or in which a mutation changes the substrate specificity of a domain, as well as other alterations in activity. Mutations can be made to the native sequences using conventional techniques. The substrates for mutation can be an entire cluster of genes or only one or two of them; the substrate for mutation may also be portions of one or more of these genes. Techniques for mutation include preparing synthetic oligonucleotides including the mutations and inserting the mutated sequence into the gene encoding a PKS subunit using restriction endonuclease digestion. (See, e.g., Kunkel, T. A. Proc Natl Acad Sci USA (1985) 82:448; Geisselsoder et al. BioTechniques (1987) 5:786.) Alternatively, the mutations can be effected using a mismatched primer (generally 10-20 nucleotides in length) that hybridizes to the native nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. (See Zoller and Smith, Methods in Enzymology (1983) 100:468). Primer extension is effected using DNA polymerase. The product of the extension reaction is cloned, and those clones containing the mutated DNA are selected. Selection can be accomplished using the mutant primer as a hybridization probe. The technique is also applicable for generating multiple point mutations. (See, e.g., Dalbie-McFarland et al. Proc Natl Acad Sci USA (1982) 79:6409). PCR mutagenesis can also be used for effecting the desired mutations.

Random mutagenesis of selected portions of the nucleotide sequences encoding enzymatic activities can be accomplished by several different techniques known in the art, e.g., by inserting an oligonucleotide linker randomly into a plasmid, by irradiation with X-rays or ultraviolet light, by incorporating incorrect nucleotides during in vitro DNA synthesis, by error-prone PCR mutagenesis, and by preparing synthetic mutants or by damaging plasmid DNA in vitro with chemicals. Chemical mutagens include, for example, sodium bisulfite, nitrous acid, hydroxylamine, agents which damage or remove bases thereby preventing normal base-pairing such as hydrazine or formic acid, analogues of nucleotide precursors such as nitrosoguanidine, 5-bromouracil, 2-aminopurine, or acridine intercalating agents such as proflavine, acriflavine, quinacrine, and the like. Generally, plasmid DNA or DNA fragments are treated with chemicals, transformed into E. coli and propagated as a pool or library of mutant plasmids.

In addition to providing mutated forms of regions encoding enzymatic activity, regions encoding corresponding activities from different PKS synthases or from different locations in the same PKS synthase can be recovered, for example, using PCR techniques with appropriate primers. By “corresponding” activity encoding regions is meant those regions encoding the same general type of activity—e.g., a ketoreductase activity in one location of a gene cluster would “correspond” to a ketoreductase-encoding activity in another location in the gene cluster or in a different gene cluster; similarly, a complete reductase cycle could be considered corresponding—e.g., KR/DH/ER could correspond to KR alone.

If replacement of a particular target region in a host polyketide synthase is to be made, this replacement can be conducted in vitro using suitable restriction enzymes or can be effected in vivo using recombinant techniques involving homologous sequences framing the replacement gene. One such system involving plasmids of differing temperature sensitivities is described in PCT application WO 96/40968. Another useful method for modifying a PKS gene (e.g., making domain substitutions or “swaps”) is a RED/ET cloning procedure developed for constructing domain swaps or modifications in an expression plasmid without first introducing restriction sites. The method is related to ET cloning methods (see, Datansko & Wanner, 2000, Proc. Natl. Acad. Sci. U.S.A. 97, 6640-45; Muyrers et al, 2000, Genetic Engineering 22:77-98). The RED/ET cloning procedure is used to introduce a unique restriction site in the recipient plasmid at the location of the targeted domain. This restriction site is used to subsequently linearize the recipient plasmid in a subsequent ET cloning step to introduce the modification. This linearization step is necessary in the absence of a selectable marker, which cannot be used for domain substitutions. An advantage of using this method for PKS engineering is that restriction sites do not have to be introduced in the recipient plasmid in order to construct the swap, which makes it faster and more powerful because boundary junctions can be altered more easily.

In one embodiment, the invention provides a chimeric PKS in which one of more polypeptides are derived from a chalcomycin PKS polypeptide, and one or more peptides are derived from one or more non-chalcomycin PKS(s) that, like the chalcomysin PKS, produces a 16-membered macrolide. Examples of PKS(s) that produces a 16-membered macrolide include, for example, the tylosin PKS, the spiramycin PKS, the niddamycin PKS, and the mycinamicin PKS. All the currently known PKSs for 16-membered macrolides consists of five large polypeptides encoded by colinear genes in a single operon. The arrangement of modules on these polypeptides is conserved. Thus, for known 16-membered macrolide PKSs, the first polypeptide has a loading module and two extender modules, the second a single extender module, the third two extender modules, the fourth a single extender module, and the fifth a single extender module followed by a thioesterase domain. The different aglycone core structures produced by different 16-membered macrolide PKSs is due to differences in the catalytic domains within each of these modules.

As is illustrated in the examples, below, new hybrid 16-membered macrolides can be made by expressing combinations of PKS polypeptides from different sources in a suitable host. The hybrid PKS produces hybrid polyketides that, optionally can be further modified by the post-PKS tailoring enzymes present within the host. See Examples, infra.

By expressing particular combinations of these PKS polypeptides one can produce molecules with desired combinations of structural features based on, for example, the macrolactone structural features specified by each of the five polypeptides of different 16-membered macrolide PKSs as shown in Table 1, below. As noted, by expressing particular combinations of these PKS polypeptides one can produce molecules with desired combinations of structural features. Although, as described in the Examples and Table 1, selection of particular combinations of polypeptides provides a level of predictability as to the products formed by the hybrid PKS, the invention is not limited to any particular combinations or structures “predicted” by the table.

TABLE 1

| PKS | 1 | 2 | 3 | 4 | 5 |
|---|---|---|---|---|---|
| TylG | 15-ethyl | 10,11-ene | 9-keto | 5-hydroxy | 3-hydroxy |
| | 14-methyl | 10-H | 8-methyl | 4-methyl | 2-H |
| | 12,13-ene | | 7-methylene | | |
| | 12-methyl | | 6-ethyl | | |
| SrmG | 15-methyl | 10,11-ene | 9-keto | 5-hydroxy | 3-hydroxy |
| | 14-H | 10-H | 8-methyl | 4-methoxy | 2-H |
| | 12,13-ene | | 7-methylene | | |
| | 12-H | | 6-ethyl | | |
| ChmG | 15-methyl | 10,11-ene | 9-keto | 5-hydroxy | 3-keto |
| | 14-methyl | 10-H | 8-methyl | 4-methyl | 2-H |
| | 12,13-ene | | 7-methylene | | |
| | 12-H | | 6-methyl | | |

In one embodiment, the components of the chimeric PKS are arranged onto polypeptides having interpolypeptide linkers that direct the assembly of the polypeptides into the functional PKS protein, such that it is not required that the PKS have the same arrangement of modules in the polypeptides as observed in natural PKSs. Suitable interpolypeptide linkers to join polypeptides and intrapolypeptide linkers to join modules within a polypeptide are described in PCT publication WO 00/47724.

In one embodiment of the invention, the components of the PKS are arranged into five polypeptides similarly to natural PKS proteins involved in the biosynthesis of tylactone, platenolide, and the like. Thus, for example, the first polypeptide comprises the loading domain, first and second extender modules, and a C-terminal interpolypeptide linker region suitable for interaction with the second polypeptide. The second polypeptide comprises an N-terminal interpolypeptide linker region suitable for interaction with the first polypeptide, the third extender module, and a C-terminal interpolypeptide linker region suitable for interaction with the third polypeptide. The third polypeptide comprises an N-terminal interpolypeptide linker region suitable for interaction with the second polypeptide, the fourth and fifth extender modules, and a C-terminal interpolypeptide linker region suitable for interaction with the fourth polypeptide. The fourth polypeptide comprises an N-terminal interpolypeptide linker region suitable for interaction with the third polypeptide, the sixth extender module, and a C-terminal interpolypeptide linker region suitable for interaction with the fifth polypeptide. The fifth polypeptide comprises an N-terminal interpolypeptide linker region suitable for interaction with the fourth polypeptide, the seventh extender module, and the terminal thioesterase domain.

In other embodiments of the invention, the components of the PKS residing on any given polypeptide are derived from the same source, and are naturally contiguous in that source, but the intrapolypeptide linkers are changed to allow proper assembly across heterologous polypeptide junctions to form a functional PKS. For example, in one embodiment of the invention, the first polypeptide is the intact first polypeptide of the chalcomycin PKS, encoded by chmGI, and comprises the loading domain and first and second extender modules from the chalcomycin PKS together with the native C-terminal interpolypeptide linker region that directs interaction of the first polypeptide with the second polypeptide of the chalcomycin PKS. The second polypeptide comprises the N-terminal interpolypeptide linker and module 3 of the chalcomycin PKS, encoded by chmGII, but with the C-terminal interpolypeptide linker replaced by the C-terminal interpolypeptide linker from the second polypeptide of the spiramycin PKS, encoded by srmG2. This replaced C-terminal interpolypeptide linker directs the second polypeptide to interact with the third polypeptide, taken from the spiramycin PKS and encoded by the srmG3 gene. The remaining polypeptides are the third, fourth, and fifth polypeptides of the spiramycin PKS, encoded by srmG3, srmG4, and srmG5, respectively. In another embodiment of the invention, the first polypeptide comprises the loading domain and first, second and third extender modules from the chalcomycin PKS, together with a C-terminal interpolypeptide linker region derived from the C-terminus of the first polypeptide of the tylosin PKS. The remaining polypeptides are the third, fourth, and fifth polypeptides of the tylosin PKS. The use of the appropriate interpolypeptide linkers directs the proper assembly of the PKS, thereby improving the catalytic activity of the resulting hybrid PKS.

As noted above, the DNA compounds of the invention can be expressed in host cells for production of known and novel compounds. Preferred hosts include fungal systems such as yeast and procaryotic hosts, but single cell cultures of, for example, mammalian cells could also be used. A variety of methods for heterologous expression of PKS genes and host cells suitable for expression of these genes and production of polyketides are described, for example, in U.S. Pat. Nos. 5,843,718 and 5,830,750; WO 01/31035, WO 01/27306, and WO 02/068613; and U.S. patent application Ser. Nos. 10/087,451 (published as US2002000087451); 60/355,211; and 60/396,513 (corresponding to published application 20020045220).

Appropriate host cells for the expression of the hybrid PKS genes include those organisms capable of producing the needed precursors, such as malonyl-CoA, methylmalonyl-CoA, ethylmalonyl-CoA, and methoxymalonyl-ACP, and having phosphopantotheinylation systems capable of activating the ACP domains of modular PKSs. See, for example, U.S. Pat. No. 6,579,695. However, as disclosed in U.S. Pat. No. 6,033,883, a wide variety of hosts can be used, even tnough some hosts natively do not contain the appropriate post-translational mechanisms to activate the acyl carrier proteins of the synthases. Also see WO 97/13845 and WO 98/27203. The host cell may natively produce none, some, or all of the required polyketide precursors, and may be genetically engineered so as to produce the required polyketide precursors. Such hosts can be modified with the appropriate recombinant enzymes to effect these modifications. Suitable host cells include Streptomyces, E. coli, yeast, and other procaryotic hosts which use control sequences compatible with Streptomyces spp. Examples of suitable hosts that either natively produce modular polyketides or have been engineered so as to produce modular polyketides include but are not limited to actinomycetes such as Streptomyces coelicolor, Streptomyces venezuelae, Streptomyces fradiae, Streptomyces ambofaciens, and Saccharopolyspora erythraea, eubacteria such as Escherichia coli, myxobacteria such as Myxococcus xanthus, and yeasts such as Saccharomyces cerevisiae.

In one embodiment, any native modular PKS genes in the host cell have been deleted to produce a “clean host,” as described in U.S. Pat. No. 5,672,491, incorporated herein by reference. The construction of the clean host S. fradiae K159-1, and the clean host S. fradiae K159-1/244-17a that produces methoxymalonyl-ACP are described below in Examples 2 and 3. Other organisms can be engineered using similar methods.

In some embodiments, the host cell expresses, or is engineered to express, a polyketide “tailoring” or “modifying” enzyme. Once a PKS product is released, it is subject to post-PKS tailoring reactions. These reactions are important for biological activity and for the diversity seen among 16-membered macrolides. Tailoring enzymes normally associated with polyketide biosynthesis include oxygenases, glycosyl- and methyltransferases, acyltransferases, halogenases, cyclases, aminotransferases, and hydroxylases. Tailoring enzymes for modification of a product of the chalcomycin PKS, a non-chalcomycin PKS, or a chimeric PKS, can be those normally associated with chalcomycin biosynthesis (including, but not limited to, proteins described in Table 2) or “heterologous” tailoring enzymes. As noted above, the P450 hydrolases encoded by the chmHI, chmPI and chmPII genes are of particular interest for production of polyketides having hydroxy groups well suited for subsequent chemical modification.

For purposes of the present invention, tailoring enzymes can be expressed in the organism in which they are naturally produced, or as recombinant proteins in heterologous hosts. For example, as shown in Examples 6 and 7, a hybrid PKSs having elements from the chalcomycin and spiramycin PKSs, or from the tylosin and chalcomysin PKSs were expressed in an engineered host derived from a tylosin producing strain of S. fradiae in which all or most of the post-PKS tailoring reactions of the tylosin biosynthetic pathway (see Baltz and Seno, 1988, “Genetics of Streptomyces fradiae and tylosin biosynthesis” Annu Rev Microbiol. 42:547-74) were expressed and which modified the polyketide product.

In some cases, the structure produced by the heterologous or hybrid PKS may be modified with different efficiencies by post-PKS tailoring enzymes from different sources. In such cases, post-PKS tailoring enzymes can be recruited from other pathways to obtain the desired compound. For example, as discussed in Example 6, a chmH gene has been used to modify the product of a chalcomycin-spiramycin hybrid PKS. Similarly, host cells can be selected, or engineered, for expression of a glycosylatation apparatus (discussed below), amide synthases, (see, for example, U.S. patent publication 20020045220 “Biosynthesis of Polyketide Synthase Substrates”). For example and not limitation, the host cell can contain the desosamine, megosamine, and/or mycarose biosynthetic genes, corresponding glycosyl transferase genes, and hydroxylase genes (e.g., picK, megK, eryK, megF, and/or eryF). Methods for glycosylating polyketides are generally known in the art and can be applied in accordance with the methods of the present invention; the glycosylation may be effected intracellularly by providing the appropriate glycosylation enzymes or may be effected in vitro using chemical synthetic means as described herein and in WO 98/49315, incorporated herein by reference. Glycosylation with desosamine, mycarose, and/or megosamine is effected in accordance with the methods of the invention in recombinant host cells provided by the invention. Alternatively and as noted, glycosylation may be effected intracellularly using endogenous or recombinantly produced intracellular glycosylases. In addition, synthetic chemical methods may be employed.

Alternatively, the aglycone compounds can be produced in the recombinant host cell, and the desired modification (e.g., glycosylation and hydroxylation) steps carried out in vitro (e.g., using purified enzymes, isolated from native sources or recombinantly produced) or in vivo in a converting cell different from the host cell (e.g., by supplying the converting cell with the aglycone).

It will be apparent to the reader that a variety of recombinant vectors can be utilized in the practice of aspects of the invention. As used herein, “vector” refers to polynucleotide elements that are used to introduce recombinant nucleic acid into cells for either expression or replication. Selection and use of such vehicles is routine in the art. An “expression vector” includes vectors capable of expressing DNAs that are operatively linked with regulatory sequences, such as promoter regions. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

The vectors used to perform the various operations to replace the enzymatic activity in the host PKS genes or to support mutations in these regions of the host PKS genes may be chosen to contain control sequences operably linked to the resulting coding sequences in a manner that expression of the coding sequences may be effected in an appropriate host. Suitable control sequences include those which function in eucaryotic and procaryotic host cells. If the cloning vectors employed to obtain PKS genes encoding derived PKS lack control sequences for expression operably linked to the encoding nucleotide sequences, the nucleotide sequences are inserted into appropriate expression vectors. This can be done individually, or using a pool of isolated encoding nucleotide sequences, which can be inserted into host vectors, the resulting vectors transformed or transfected into host cells, and the resulting cells plated out into individual colonies.

Suitable control sequences for single cell cultures of various types of organisms are well known in the art. Control systems for expression in yeast are widely available and are routinely used. Control elements include promoters, optionally containing operator sequences, and other elements depending on the nature of the host, such as ribosome binding sites. Particularly useful promoters for procaryotic hosts include those from PKS gene clusters which result in the production of polyketides as secondary metabolites, including those from Type I or aromatic (Type II) PKS gene clusters. Examples are act promoters, tcm promoters, spiramycin promoters, tylosin promoter (e.g., tylGIp, see Rodriguez et al., “Rapid engineering of polyketide overproduction by gene transfer to industrially optimized strains” J Ind Microbiol Biotechnol. Apr. 16, 2003; and DeHoff et al., “Streptomyces fradiae tylactone synthase, starter module and modules 1-7, (tylG) gene, complete cds” Genbank Accession No. U78289), and other promoters. However, other bacterial promoters, such as those derived from sugar metabolizing enzymes, such as galactose, lactose (lac) and maltose, are also useful. Additional examples include promoters derived from biosynthetic enzymes such as for tryptophan (trp), the β-lactamase (bla), bacteriophage lambda PL, and T5. In addition, synthetic promoters, such as the tac promoter (U.S. Pat. No. 4,551,433), can be used.

As noted, particularly useful control sequences are those which themselves, or with suitable regulatory systems, activate expression during transition from growth to stationary phase in the vegetative mycelium. The system contained in the plasmid identified as pCK7, i.e., the actI/actIII promoter pair and the actII-ORF4 (an activator gene), is particularly preferred. Particularly preferred hosts are those which lack their own means for producing polyketides so that a cleaner result is obtained. Illustrative control sequences, vectors, and host cells of these types include the modified S. coelicolor CH999 and vectors described in PCT publication WO 96/40968 and similar strains of S. lividans. See U.S. Pat. Nos. 5,672,491; 5,830,750, 5,843,718; and 6,177,262, each of which is incorporated herein by reference.

Other regulatory sequences may also be desirable which allow for regulation of expression of the PKS sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

Selectable markers can also be included in the recombinant expression vectors. A variety of markers are known which are useful in selecting for transformed cell lines and generally comprise a gene whose expression confers a selectable phenotype on transformed cells when the cells are grown in an appropriate selective medium. Such markers include, for example, genes which confer antibiotic resistance or sensitivity to the plasmid. Alternatively, several polyketides are naturally colored, and this characteristic provides a built-in marker for screening cells successfully transformed by the present constructs.

The various PKS nucleotide sequences, or a mixture of such sequences, can be cloned into one or more recombinant vectors as individual cassettes, with separate control elements or under the control of a single promoter. The PKS subunits or components can include flanking restriction sites to allow for the easy deletion and insertion of other PKS subunits so that hybrid or chimeric PKSs can be generated. The design of such restriction sites is known to those of skill in the art and can be accomplished using the techniques described above, such as site-directed mutagenesis and PCR. Methods for introducing the recombinant vectors of the present invention into suitable hosts are known to those of skill in the art and typically include the use of CaCl₂ or other agents, such as divalent cations, lipofection, DMSO, protoplast transformation, conjugation, and electroporation.

Expression vectors containing nucleotide sequences encoding a variety of PKS systems for the production of different polyketides can be transformed into the appropriate host cells to construct a polyketide library. In one approach, a mixture of such vectors is transformed into the selected host cells and the resulting cells plated into individual colonies and selected for successful transformants. Each individual colony has the ability to produce a particular PKS and ultimately a particular polyketide. Typically, there will be duplications in some of the colonies; the subset of the transformed colonies that contains a different PKS in each member colony can be considered the library. Alternatively, the expression vectors can be used individually to transform hosts, which transformed hosts are then assembled into a library. A variety of strategies might be devised to obtain a multiplicity of colonies each containing a PKS gene cluster derived from the naturally occurring host gene cluster so that each colony in the library produces a different PKS and ultimately a different polyketide. The number of different polyketides that are produced by the library is typically at least four, more typically at least ten, and preferably at least 20, more preferably at least 50, reflecting similar numbers of different altered PKS gene clusters and PKS gene products. The number of members in the library is arbitrarily chosen; however, the degrees of freedom outlined above with respect to the variation of starter, extender units, stereochemistry, oxidation state, and chain length is quite large. The polyketide producing colonies can be identified and isolated using known techniques and the produced polyketides further characterized. The polyketides produced by these colonies can be used collectively in a panel to represent a library or may be assessed individually for activity.

The libraries can thus be considered at four levels: (1) a multiplicity of colonies each with a different PKS encoding sequence encoding a different PKS cluster but all derived from a naturally occurring PKS cluster; (2) colonies which contain the proteins that are members of the PKS produced by the coding sequences; (3) the polyketides produced; and (4) compounds derived from the polyketides. Of course, combination libraries can also be constructed wherein members of a library derived, for example, from the erythromycin PKS can be considered as a part of the same library as those derived from, for example, the rapamycin PKS cluster.

Colonies in the library are induced to produce the relevant synthases and thus to produce the relevant polyketides to obtain a library of candidate polyketides. The polyketides secreted into the media can be screened for binding to desired targets, such as receptors, signaling proteins, and the like. The supernatants per se can be used for screening, or partial or complete purification of the polyketides can first be effected. Typically, such screening methods involve detecting the binding of each member of the library to receptor or other target ligand. Binding can be detected either directly or through a competition assay. Means to screen such libraries for binding are well known in the art. Alternatively, individual polyketide members of the library can be tested against a desired target. In this event, screens wherein the biological response of the target is measured can be included.

Thus, the present invention provides recombinant DNA molecules and vectors comprising those recombinant DNA molecules that encode all or a portion of the chalcomycin PKS and/or chalcomycin modification enzymes and that, when transformed into a host cell and the host cell is cultured under conditions that lead to the expression of said chalcomycin PKS and/or modification enzymes, results in the production of polyketides including but not limited to chalcomycin and/or analogs or derivatives thereof in useful quantities. The present invention also provides recombinant host cells comprising those recombinant vectors.

Suitable culture conditions for production of polyketides using the cells of the invention will vary according to the host cell and the nature of the polyketide being produced, but will be know to those of skill in the art. See, for example, the examples below and WO 98/27203 “Production of Polyketides in Bacteria and Yeast” and WO 01/83803 “Overproduction Hosts for Biosynthesis of Polyketides.”

The polyketide product produced by host cells of the invention can be recovered (i.e., separated from the producing cells and at least partially purified) using routine techniques (e.g., extraction from broth followed by chromatography).

The compositions, cells and methods of the invention may be directed to the preparation of an individual polyketide or a number of polyketides. The polyketide may or may not be novel, but the method of preparation permits a more convenient or alternative method of preparing it. It will be understood that the resulting polyketides may be further modified to convert them to other useful compounds. For example, an ester linkage may be added to produce a “pharmaceutically acceptable ester” (i.e., an ester that hydrolyzes under physiologically relevant conditions to produce a compound or a salt thereof). Illustrative examples of suitable ester groups include but are not limited to formates, acetates, propionates, butyrates, succinates, and ethylsuccinates.

The polyketide product produced by recombinant cells can be chemically modified in a variety of ways. For example , for example by addition of a protecting group, for example to produce prodrug forms. A variety of protecting groups are disclosed, for example, in T. H. Greene and P. G. M. Wuts, Protective Groups in Organic Synthesis, Third Edition, John Wiley & Sons, New York (1999). Prodrugs are in general functional derivatives of the compounds that are readily convertible in vivo into the required compound. Conventional procedures for the selection and preparation of suitable prodrug derivatives are described, for example, in “Design of Prodrugs,” H. Bundgaard ed., Elsevier, 1985.

Similarly, improvements in water solubility of a polyketide compound can be achieved by addition of groups containing solubilizing functionalities to the compound or by removal of hydrophobic groups from the compound, so as to decrease the lipophilicity of the compound. Typical groups containing solubilizing functionalities include, but are not limited to: 2-(dimethylaminoethyl)amino, piperidinyl, N-alkylpiperidinyl, hexahydropyranyl, furfuryl, tetrahydro furfuryl, pyrrolidinyl, N-alkylpyrrolidinyl, piperazinylamino, N-alkylpiperazinyl, morpholinyl, N-alkylaziridinylmethyl, (1-azabicyclo[1.3.0]hex-1-yl)ethyl, 2-(N-methylpyrrolidin-2-yl) ethyl, 2-(4-imidazolyl)ethyl, 2-(1-methyl-4-imidazolyl)ethyl, 2-(1-methyl-5-imidazolyl)ethyl, 2-(4-pyridyl)ethyl, and 3-(4-morpholino)-1-propyl. In the case of geldanamycin analogs, solubilizing groups can be added by reaction with amines, which results in the displacement of the 17-methoxy group by the amine (see, Schnur et al., 1995, “Inhibition of the Oncogene Product p185^{erbB-2} in Vitro and in Vivo by Geldanamycin and Dihydrogeldanamycin Derivatives,”, J. Med. Chem. 38, 3806-3812; Schnur et al., 1995 “erbB-2 Oncogene Inhibition by Geldanamycin Derivatives: Synthesis, Mechanism of Action, and Structure-Activity relationships,” J. Med. Chem. 38, 3813-3820; Schnur et al., “Ansamycin Derivatives as Antioncogene and Anticancer Agents,” U.S. Pat. No. 5,932,655; all of which are incorporated herein by reference). Typical amines containing solubilizing functionalities include 2-(dimethylamino)-ethylamine, 4-aminopiperidine, 4-amino-1-methylpiperidine, 4-aminohexahydropyran, furfurylamine, tetrahydrofurfurylamine, 3-(aminomethyl)-tetrahydrofuran, 2-(amino-methyl)pyrrolidone, 2-(aminomethyl)-1-methylpyrrolidine, 1-methylpiperazine, morpholine, 1-methyl-2(aminomethyl)aziridine, 1-(2-aminoethyl)-1-azabicyclo-[1.3.0]hexane, 1-(2-aminoethyl)piperazine, 4-(2-aminoethyl)morpholine, 1-(2-amino-ethyl) pyrrolidine, 2-(2-aminoethyl)pyridine, 2-fluoroethylamine, 2,2-difluoroethylamine, and the like.

In addition to post synthesis chemical or biosynthetic modifications, various polyketide forms or compositions can be produced, including but not limited to mixtures of polyketides, enantiomers, diastereomers, geometrical isomers, polymorphic crystalline forms and solvates, and combinations and mixtures thereof can be produced

Many other modifications of polyketides produced according to the invention will be apparent to those of skill, and can be accomplished using techniques of pharmaceutical chemistry.

Prior to use the PKS product (whether modified or not) can be formulated for storage, stability or administration. For example, the polyketide products can be formulated as a “pharmaceutically acceptable salt.” Suitable pharmaceutically acceptable salts of compounds include acid addition salts which may, for example, be formed by mixing a solution of the compound with a solution of a pharmaceutically acceptable acid such as hydrochloric acid, hydrobromic acid, sulfuric acid, fumaric acid, maleic acid, succinic acid, benzoic acid, acetic acid, citric acid, tartaric acid, phosphoric acid, carbonic acid, or the like. Where the compounds carry one or more acidic moieties, pharmaceutically acceptable salts may be formed by treatment of a solution of the compound with a solution of a pharmaceutically acceptable base, such as lithium hydroxide, sodium hydroxide, potassium hydroxide, tetraalkylammonium hydroxide, lithium carbonate, sodium carbonate, potassium carbonate, ammonia, alkylamines, or the like.

Prior to administration to a mammal the PKS product will be formulated as a pharmaceutical composition according to methods well known in the art, e.g., combination with a pharmaceutically acceptable carrier. The term “pharmaceutically acceptable carrier” refers to a medium that is used to prepare a desired dosage form of a compound. A pharmaceutically acceptable carrier can include one or more solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), disclose various carriers used in formulating pharmaceutical compositions and known techniques for the preparation thereof.

The composition may be administered in any suitable form such as solid, semisolid, or liquid form. See Pharmaceutical Dosage Forms and Drug Delivery Systems, 5^{th} edition, Lippicott Williams & Wilkins (1991). In an embodiment, for illustration and not limitation, the polyketide is combined in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. The carriers that can be used include water, glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semi-solid, or liquified form. In addition, auxiliary stabilizing, thickening, and coloring agents and perfumes may be used.

It will be apparent from the forgoing that the invention provides may useful compositions and methods of using them. Without intending to limit its scope, in one aspect, the invention provides a recombinant DNA molecule that encodes a polypeptide, module or domain derived from a chalcomycin polyketide synthase (PKS) gene cluster. In an embodiment, the DNA molecule (or its complement) has substantial sequence identity to SEQ ID NO:1. In an embodiment, the DNA molecule hybridizes under stringent conditions to a nucleic acid having the sequence of SEQ ID NO:1 or its complement. In a related aspect, the invention provides a recombinant DNA molecule, comprising a sequence of at least about 200, optionally at least about 500, basepairs with a sequence identical or substantially identical to a protein encoding region of SEQ ID NO:1. In an embodiment, the DNA molecule encodes a polypeptide, module or domain derived from a chalcomycin polyketide synthase (PKS) gene cluster.

In one embodiment, the recombinant DNA molecule comprises a sequence encoding at least one module of a chalcomycin polyketide synthase. In an embodiment, the recombinant DNA molecule encodes a chalcomycin polyketide synthase polypeptide selected from the group consisting of ChmGI, ChmGII, ChmGIII, ChmGIV, and ChmV.

In one aspect, the recombinant DNA molecule includes a coding sequence for a chalcomycin modifying enzyme, such as a chalcomycin P450 hydrolase enzyme selected from the group consisting of ChmHI, ChmPI, and ChmPII.

The invention also provides vector that comprise the recombinant DNA molecules of the invention. In an aspect, the invention provides a recombinant host cell comprising the vector. In a related aspect, the invention provides a recombinant host cell comprising a DNA molecule of the invention integrated into the cell chromosomal DNA.

Also provided is a chimeric PKS that comprises at least one domain of a chalcomycin PKS, and a cell containing such a chimeric PKS. In a related aspect, the invention provides a modified functional chalcomycin PKS that differs from the S. bikiniensis chalcomycin PKS by the inactivation of at least one domain of the chalcomycin PKS and/or addition of at least one domain of a non-chalcomycin PKS (for example, a loading domain, a thioesterase domain, an AT domain, a KS domain, an ACP domain, a KR domain, a DH domain, and an ER domain). The invention provides a cell comprising a modified functional PKS. The invention also provides a method to prepare an chalcomycin derivative which method comprises providing substrates including extender units to the cell.

In an aspect, the invention provides a recombinant expression system capable of producing a chalcomycin synthase domain in a host cell, said system comprising an encoding sequence for a chalcomycin polyketide synthase domain, and said encoding sequence being operably linked to control sequences effective in said cell to produce RNA that is translated into said domain, and a host cell modified to contain the recombinant expression system.

The invention provides an isolated polypeptide encoded by a recombinant chalcomycin polyketide synthase (PKS) gene, and a recombinant host cell containing or expressing such a polypeptide.

The invention also provides a recombinant S. bikiniensis cell in which a chmGI, chmGII, chmGIII, chmGIV, or chmV is disrupted so as to reduce or eliminate production of chalcomycin.

The invention also provides a recombinant DNA molecule encoding a first protein comprising one or more modules of a chalcomycin PKS and a second protein comprising one or more modules of a tylosin PKS or spiramycin PKS, optionally one or more polypeptides of a chalcomycin PKS and one or more polypeptides of a tylosin PKS or spiramycin PKS. In a related aspect, the invention provides a recombinant host cell comprising a hybrid polyketide synthase comprising one or more modules of a chalcomycin PKS and one or more modules of a tylosin PKS or spiramycin PKS.

### EXAMPLES

The following examples are provided to illustrate, but are not intended to limit, the present invention.

### Example 1

### Isolation and Characterization of Chalcomycin PKS Cluster From Streptomyces bikiniensis NRRL 2737

Growth of organism and extraction of genomic DNA. For genomic DNA extraction, a spore stock of Streptomyces bikiniensis NRRL 2737 was used to prepare a seed culture. Spores were stored as a suspension in 25% (v/v) glycerol at −80° C. and used to inoculate 5 ml of unbuffered Trypticase Soy Broth (TSB) liquid media. The entire seed culture was transferred into 50 ml of the same growth medium in a 250 ml baffled Erlenmeyer flask and incubated with shaking for 24 h at 28° C. A 10 ml portion of the cell suspension was centrifuged (10,000×g) and the resulting pellet was washed once with 10 ml buffer 1 (Tris, 50 mM, pH7.5; 20 mM EDTA). The pellet was suspended in 3.5 ml of buffer 1 containing 150 μg/ml RNase (Sigma-Aldrich) and 1 mg/ml lysozyme. After incubation of the mixture at 37° C. for 30 min, the salt concentration was adjusted by adding 850 μl 5 M NaCl solution, then the mixture was extracted two times with phenol:chloroform:isoamylaclohol (25:24:1, vol/vol) with gentle agitation followed by centrifugation for 10 min at 3500×g. After precipitation with 1 vol of isopropanol, the genomic DNA knot was spooled on a glass rod and redissolved in 200 μl of water. This method yielded about 0.5 mg total DNA. Standard agarose gel electrophoresis using 0.7% Seakem® LE-Agarose (Bio Whiaker Molecular Applications, Rockland, Me.) at a voltage of 50 mV overnight revealed that the sample contained mainly high molecular weight DNA of 50 kb or greater.

PKS Probe design. Five degenerate PCR primers were designed (degKS1F 5′-TTCGAY SCSGVSTTCTTCGSAT-3′[SEQ ID NO:44]; degKS2F 5′-GCSATGGAYCCSCARCARCGSVT-3′[SEQ ID NO:45]; degKS3F 5′-SSCTSGTSGCSMTSCAYCWSGC-3′[SEQ ID NO:46]; degKS5R 5′-GTSCCSGTSCCRTGSSCYTCSAC-3′[SEQ ID NO:47]; degKS7R 5′-ASRTGSGCRTTSGTSCCSSWSA-3′[SEQ ID NO:48]) based on conserved regions of ketosynthase (KS) domains of type I PKS genes and codon bias of high G+C organisms. The primers were used in the following combinations: degKS1F/degKS5R, degKS2F/degKS5R and degKS3F/degKS7R. The PCR conditions for the amplification of KS domains were as follows: A total reaction volume of 50 μl contained 100 ng of S. bikiniensis total DNA, 200 pmol of each primer, 0.2 mM dNTP, 10% DMSO and 2.5 U Taq DNA polymerase (Roche Applied Science, Indianapolis, Ind.). Thirty-five cycles of PCR were performed using the following steps: denaturation (94° C.; 40 sec); annealing (55° C.; 30 sec); extension (72° C.; 60 sec), 35 cycles. The resulting PCR reactions were subjected to electrophoresis on 1% agarose gels and the PCR products of approximately 700 bp were extracted from the gels using the gel extraction kit from Quiagen (Valencia, Calif.) according to manufacturer's protocol. The fragments were treated with Pfu DNA Polymerase (Stratagene, La Jolla, Calif.) to remove the A overhangs and cloned into the plasmid vector pLitmus28 (New England Biolabs, Beverley, Mass.) cut with EcRV. Thirty-two “amplimers” (the ca. 700 bp PCR-amplified segment) for each primer pair were sequenced using standard protocols. Of the 96 inserts sequenced, 81 were found to be KS amplimers. Employing the sequence comparison program ClustalW, 15 of the 81 KS amplimers were found to be unique and were compared with the 8 KS sequences of the related tylosin PKS cluster of Streptomyces fradiae using the program ClustalW. Each KS amplimer was thus assigned to a particular KS within the putative chalcomycin PKS cluster.

Genomic library preparation. Approximately 10 μg of genomic DNA was partially digested with Sau3A1 (1 hr incubation using dilutions of the enzyme) and the digested DNA was run on an agarose gel with DNA standards. One of the conditions used was found to have generated fragments of size 35-47 kb. The DNA from this digestion was ligated with pSuperKos plasmid, a derivative of pSuperCos (Stratagene) digested with AfeI and self-ligated to eliminate the neo marker, pre-linearized with BamHI and XbaI and the ligation mixture was packaged using a Gigapack XIII (Stragene) in vitro packaging Kit and the mixture was subsequently used for infection of Escherichia coli DH5α employing protocols supplied by the manufacturer. Approximately 2000 of E. coli transductants were probed by in-situ colony hybridization with DIG labeled Sb3/7-31 (KS^{q}), Sb1/5-75 (KS3) and and Sb1/5-78 (KS7). Plasmids from 15 colonies, which showed strong hybridization signals were isolated, digested with BamHI and subjected to Southern blotting employing the KS^{q} or KS7 amplimers as probes. Ten plasmids showed strong hybridization with one or both amplimers. The ends of the insert in each of the 10 plasmids were sequenced using convergent primers for each (T7 promoter and T3 promoter). Two cosmids, pKOS146.185.1 and pKOS146.185.10 were found to possess high homology at one end with a segment of the PKS from the tylosin biosynthesis cluster. These two plasmids also each gave rise to DNA fragments of ca. 1 kb and 1.2 kb after BamHI-digestion.

Identification of chalcomycin biosynthetic gene cluster. Further verification that cosmids pKOS146.185.1 and pKOS146.185.10 contained the chalcomycin biosynthesis cluster was performed by PCR. Specific primer pairs were designed for the chalcomycin KSq (Sb3/7-31 forward 5′-CGTCAGCCTGATCCTCGCCGA-3′[SEQ ID NO:49]; reverse 5′-TCCAGGTGGCCGACGTTC GTC)[SEQ ID NO:50], KS3 (Sb1/5-75 forward 5′-AACGAGATCCCGCCGGG CCTC-3′[SEQ ID NO:51]; reverse 5′-ATCA CGCGTTGCTGGGCGAGG-3′[SEQ ID NO:52]) and KS7 (Sb1/5-78 forward 5′-GGACGTCTGCCGGAGG GTTCC-3′[SEQ ID NO:53]; reverse 5′-GGCCCGTTGGGCACGGACAGA-3′[SEQ ID NO:54]) amplimers and used in PCR reactions with each of the 8 KS amplimers using the following conditions: total reaction volume of 50 μl contained 20-100 ng of plasmid DNA containing an amplimer, 100 pmol of each primer, 0.2 mM dNTP,10% DMSO and 2.5 U Taq DNA polymerase. Cycle steps were as follows: denaturation (94° C.; 40 sec), annealing (55° C. for KSq and KS3 specific primers, 65° C. for KS5 specific primers; 30 sec), extension (72° C.; 60 sec), 25 cycles. Each primer set was found to amplify its cognate amplimer exclusively, with the exception of the primer set for KS7, which was also seen to give a small amount of amplification of non-cognate amplimers. Each primer set was then used for PCR with cosmids pKOS146.185.1, pKOS146.185.10 and pKOS146.185.11 employing the same conditions as described above but using cosmid DNA in place of the plasmid-containing amplimer DNA. pKOS146.185.1 gave correctly sized amplimers with KSq and KS3 primers but not with KS7 specific primers, whereas pKOS 146-185.10 gave a correctly sized amplimer with KS7 but not with KSq and KS3 specific primers, indicating that pKOS146.185.1 contained the 5′ region of the chalcomycin PKS genes.

The sequence of the insert of pKOS146.185.1 corresponds to bases 1 to 48,595 of SEQ ID No.1 and the sequence of the insert of PKOS146.185.10 corresponds to bases 44,218 to 85,915 of SEQ ID No.1. Table 2 below provides open reading frame (ORF) boundaries corresponding to the nucleotide position in SEQ ID NO:1 (Table 3) of the chalcomycin PKS as well as the nucleotide sequences encoding enzymes involved in precursor synthesis and chalcomycin modification. In addition to the ORFs listed in Table 2, SEQ ID NO:1 includes additional open reading frames of genes encoding proteins or domains thereof that may be useful in the biosynthesis of chalcomycin and/or analogs thereof in certain host cells. The various open reading frames, module-coding sequences, and domain encoding sequences shown in Table 2 and the figures are sometimes referred to as “subsequences.” Those of skill in the art will recognize, upon consideration of the sequence shown in Example 1, that the actual start locations of several of the genes could differ from the start locations shown in the table, for example due to the presence in-frame of codons utilizable by the initiator methionine tRNA in close proximity to the codon indicated as the start codon. The actual start codon can be confirmed by amino acid sequencing of the proteins expressed from the genes.

TABLE 2
Chalcomycin PKS and modifying gene cluster ORFs of SEQ ID NO: 1
* “C” encoded by complement of SEQ ID NO: 1

| ORF boundaries | Name | Proposed Function (homology) | Strand* | No. residues |
|---|---|---|---|---|
| 1-1009 | Orf1 | Orf1 complement | C | 343 [SEQ ID NO: 2] |
| | | integral membrane protein (homolog of S. coelicolor | | |
| | | SCF51A.28c) | | |
| 1121-1963 | Orf 2 | Orf2 complement | C | 280 [SEQ ID NO: 3] |
| | | (homolog of S. coelicolor SCF51A.29c) | | |
| 3008-3889 | Orf3 | Orf 3 complement | C | 293 [SEQ ID NO: 4] |
| | | Oxidoreductase (homolog of M. tuberculosis | | |
| | | MLCL581.18c) | | |
| 3991-5208 | chmCIV | 3,4-dehydratase, D-chalcose pathway (EryCIV | C | 405 [SEQ ID NO: 5] |
| | | homolog) | | |
| 5339-6118 | chmMIII | D-chalcose O-methytransferase (SpnH homolog) | C | 259 [SEQ ID NO: 6] |
| 6239-7696 | chmCV | D-chalcose pathway (EryCV homolog) | C | 485 [SEQ ID NO: 7] |
| 7761-10271 | chmR | beta-glucosidase, extracellular reactivator of | C | 836 [SEQ ID NO: 8] |
| | | chalcomycin (OleR, DesR, EryBI homolog) | | |
| 10306-11511 | chmPII | P450 C12, C13-epoxidase (MycG homolog) | C | 401 [SEQ ID NO: 9] |
| 11549-12772 | chmPI | P450 C8-hydroxylase (OleP homolog) | C | 407 [SEQ ID NO: 10] |
| 12762-13610 | chmI | TEII (homolog of TEII of tylosin cluster [the predicted | C | 282 [SEQ ID NO: 11] |
| | | product of ORF5 of GenBank accession # AF145042]) | | |
| 13631-14602 | chmAII | TDP-glucose 4,6-dehydratase | C | 323 [SEQ ID NO: 12] |
| 14648-15562 | chmAI | TDP-glucose synthase | C | 305 [SEQ ID NO: 13] |
| 15869-16459 | chmJ | 3-epimerase; D-allose pathway (TylJ homolog) | C | 196 [SEQ ID NO: 14] |
| 16523-17290 | chmMII | D-mycinose 3′ OH-MT (TylF homolog) | C | 255 [SEQ ID NO: 15] |
| 17551-18810 | chmHI | P450 C20-hydroxylase (TylHI homolog) | | 420 [SEQ ID NO: 16] |
| 18831-19052 | chmHII | Ferredoxin (TylHII homolog) | | 73 [SEQ ID NO: 17] |
| 18959-20029 | chmD | D-allose pathway 4-KR (TylD homolog) | | 326 [SEQ ID NO: 18] |
| 19049-20029 | chmD | Alternate N-terminus | | [SEQ ID NO: 19] |
| 20062-21273 | chmMI | D-mycinose pathway 2′ OH-MT(TylE homolog) | | 403 [SEQ ID NO: 20] |
| 21329-22576 | chmN | D-allose glycosyltransferase (TylN homolog) | | 418 [SEQ ID NO: 21] |
| 22653-23495 | chrB | Resistance determinant; 23S-rRNA N1- | C | 280 [SEQ ID NO: 22] |
| | | methyltransferase (TlrB homolog) | | |
| 23622-36947 | chmGI | PKS, Modules 0-2 | | 4441 [SEQ ID NO: 23] |
| 23823-25046 | KSOq | PKS Ketosynthase 0q loading domain | | |
| 25353-26396 | AT0 | PKS Acyltransferase loading domain | | |
| 26499-26756 | ACP0 | PKS Acyl carrier protein loading domain | | |
| 26808-28079 | KS1 | PKS Ketosynthase 1 domain | | |
| 28386-29432 | AT1 | PKS Acyl transferase 1 domain | | |
| 30099-30794 | KR1 | PKS Ketoreductase 1 domain | | |
| 30966-31220 | ACP1 | PKS Acyl carrier protein domain | | |
| 31296-32567 | KS2 | PKS Ketosynthase 2 domain | | |
| 32889-33932 | AT2 | PKS Acyl transferase 2 domain | | |
| 33975-34574 | DH2 | PKS Dehydrogenase 2 domain | | |
| 35472-36257 | KR2 | PKS Ketoreductase 2 domain | | |
| 36402-36659 | ACP2 | PKS Acyl carrier protein 2 domain | | |
| 37041-42965 | chmGII | PKS, Module 3 | | 1974 [SEQ ID NO: 24] |
| 37143-38414 | KS3 | PKS Ketosynthase 3 domain | | |
| 38724-39869 | AT3 | PKS Acyl transferase 3 domain | | |
| 39903-40544 | DH3 | PKS Dehydrogenase 3 domain | | |
| 41442-42281 | KR3 | PKS Ketoreductase 3 domain | | |
| 42411-42668 | ACP3 | PKS Acyl carrier protein 3 domain | | |
| 43022-54388 | chmGIII | PKS, Modules 4 and 5 | | 3788 [SEQ ID NO: 25] |
| 43139-44422 | KS4 | PKS Ketosynthase 4 domain | | |
| 44750-45796 | AT4 | PKS Acyl transferase 4 domain | | |
| 46436-47248 | KR4 | PKS Ketoreductase 4 domain | | |
| 47318-47575 | ACP4 | PKS Acyl carrier protein 4 domain | | |
| 47651-48925 | KS5 | PKS Ketosynthase 5 domain | | |
| 49226-50272 | AT5 | PKS Acyl transferase 5 domain | | |
| 50309-51001 | DH5 | PKS Dehydrogenase 5 domain | | |
| 52085-52957 | ER5 | PKS Enoylreductase 5 domain | | |
| 52925-53728 | KR5 | PKS Ketoreductase 5 domain | | |
| 54439-59277 | chmGIV | PKS, Module 6 | | 1612 [SEQ ID NO: 26] |
| 54544-55818 | KS6 | PKS Ketosynthase 6 domain | | |
| 56122-57168 | AT6 | PKS Acyl transferase 6 domain | | |
| 57844-58707 | KR6 | PKS Ketoreductase 6 domain | | |
| 58753-59019 | ACP6 | PKS Acyl carrier protein 6 domain | | |
| 59387-63439 | chmGV | PKS, Module 7 and TE | | 1350 [SEQ ID NO: 27] |
| 59489-60778 | KS7 | PKS Ketosynthase 7 domain | | |
| 61112-62209 | AT7 | PKS Acyl transferase 7 domain | | |
| 62276-62533 | ACP7 | PKS Acyl carrier protein 7 domain | | |
| 62549-63436 | TE | Thioesterase | | |
| 63522-64760 | ChmCII | NDP - hexose 3,4-isomerase; D-chalcose pathway | | 412 [SEQ ID NO: 28] |
| | | component (EryCII homolog) | | |
| 64804-66081 | ChmCIII | Chalcose glycosyltransferase (EryCIII homolog) | | 425 [SEQ ID NO: 29] |
| 66194-66940 | ChmU | Post PKS Ketoreductase (SimJ2, NovJ homolog) | | 248 [SEQ ID NO: 30] |
| 67323-68471 | Orf4 | Permease homolog (SCF6.09 homolog) | C | 382 [SEQ ID NO: 31] |
| 68733-70196 | Orf5 | Membrane protein homolog (SC66T3.03 homolog) | C | 487 [SEQ ID NO: 32] |
| 70193-70888 | Orf6 | D-alanyl-D-alanine carboxypeptidase homolog | C | 231 [SEQ ID NO: 33] |
| | | (SCD6.17c homolog) | | |
| 71382-72542 | Orf7 | Sensory histidine kinase homolog (SCE94.10 | C | 386 [SEQ ID NO: 34] |
| | | homolog) | | |
| 72638-73324 | Orf8 | Two-component syst. response regulator homolog | C | 228 [SEQ ID NO: 35] |
| | | (SCE94.09 homolog) | | |
| 73651-75081 | Orf9 | permease (xanthine/uracil permease type) (SC9G1.02, | C | 476 [SEQ ID NO: 36] |
| | | SC9G1.04 homolog) | | |
| 75401-76117 | Orf10 | SC6A11.03c Homolog | C | [SEQ ID NO: 37] |
| 76537-78375 | Orf11 | Permease homolog (SC9G1.02, SC9G1.04 homolog) | | 612 [SEQ ID NO: 38] |
| 78521-79192 | Orf12 | MerR-family transcriptional regulator (SC1A4.06c | | 223 [SEQ ID NO: 39] |
| | | homolog) | | |
| 79228-79983 | Orf13 | Type-II thioesterase (SanP homolog) | C | 251 [SEQ ID NO: 40] |
| 80489-81439 | Orf14 | Open reading frame | C | [SEQ ID NO: 41] |
| 81806-82528 | Orf15 | Response regulator homolog (SCD49.02c homolog) | | 240 [SEQ ID NO: 42] |
| 82712-85912 | Orf16 | Open reading frame | | [SEQ ID NO: 43] |

Genes listed in Table 2 that encode proteins with post-PKS polyketide-modifying activities include: chmPI, chmPII, chmHI (P450 homologs), chmN, chnCIII (glycosyltransferases) and chmU (polyketide ketoreductase).

Genes listed in Table 2 that encode proteins predicted to participate in the biosynthesis of sugar residue subunits of chalcomycin or modification of sugar residues after their addition to the polyketide include: chmCIV, chmMIII, chmCV, chmAII, chmAI, chmJ, chmMII, chmD, chmMI, and chmCII. Of these, three are predicted to participate in D-chalcose residue biosynthesis (ChmCII, ChmCIV and ChmCV), two are predicted to participate in D-biosynthesis allose residue biosynthesis (ChmD and ChmJ) two are predicted to participate in conversion of the D-allose residue to D-mycinose residue after covalent linkage of the D-allose to the polyketide (ChmMI and ChmMII), two are predicted to provide precursors for both the allose and chalcose pathways (ChmAI and ChmAII), and one is predicted to O-methylate the chalcose residue (ChmMIII).

As noted above, the invention also provides inter-polypeptide linker sequences, which can be identified by the skilled reader (e.g., comprinsing the sequences between the N-terminus of the polyptide and the beginning of the first KS domain; or between the C-terminue of the polypeptide and the beginning of the last ACP domain) and polynucleotides encoding such linkers.

TABLE 3

Chalcomyin PKS cluster from Streptomyces bikiniensis NRRL 2737
(SEQ ID NO:1)

| | | | | | | |
|---|---|---|---|---|---|---|
| 1 | GGGGCCCGCC | GGACGGGGCT | GCCCGGCTCT | CGGCGGTGCC | CGGTGGGCCG | GGTGCGGGCT |
| 61 | CGCCCGCGGC | GAGATGCTCC | AGGACCTCCG | CCAGTTCCCG | GCAGGCGCGG | CGTACCGAGC |
| 121 | GGCGGGTCGC | ACGCTGCTCC | GTGATGACGG | AGGCGAGAAG | CAGGGCGGTC | AGTGCGGCGG |
| 181 | AACCGTTGAA | CGCCTGGAGC | TTGGCCATGA | TCTCGACGTC | CGAGAGGTGG | AGGAACCCTC |
| 241 | CCCGTCCGGC | GTTCGCCTCG | AAGGTGGCGA | GCACGGAGGC | GAAGAGCGCG | CAGAGCATGC |
| 301 | TTCCGGTGAG | CTGGAAGCGG | AGCGCCGCCC | AGATCAGCAG | GGGGAAGACG | AGGAAGAGCA |
| 361 | TGCCCACCGG | GCTGAGCACG | GCCATGGGCA | TGAGGATCAG | GGTCGCGAGA | CCCAGCAGGG |
| 421 | CCGCCTCCTT | CCAGCGCCGT | ACGCGGAACC | GTCCGGCCGG | CCCCGCGAGG | ACGAGGAGGA |
| 481 | GCGGGGCGAC | GAGCAGCACC | CCCATCGTGT | CGCCCACCCA | CCAGGCCAGC | CAGACGGGCC |
| 541 | AGAACTCGGT | CGTGTCCAGG | GAGCTCTTCG | CCACCTGCAG | TCCGACCCCG | GCGGTCGCGC |
| 601 | TGATCAGCAT | GGCGCCGAAC | CCGCCGAGGA | AGACCAGGGA | GAGTCCGTCC | CGCAGCCGTG |
| 661 | CCATGTCGAG | CCGGAAGCCG | GCCCGTGTCA | GCAGCAGGAA | GGCGCAGAGC | GGCGCGACGG |
| 721 | TGTTGCTGAC | CACGGTGACC | ACGGTGGTGG | GCCCCGGCGT | GGTGAGGGAG | GCGATGACGA |
| 781 | GGAAGGAGCC | GAGGGCGATC | CCGGGCCAGA | CGCGCGCGCC | GAGCAGCAGC | AGGGCGGCGA |
| 841 | CGGCGACGCC | GGTGGGAGGC | CAGATGGGGG | TGACCACCAC | GCCTTCGACG | ACGAGGCGGC |
| 901 | CCATCAGGCC | GAGTCGTCCG | GCCGCGTAGT | AGAGCACCGC | CACGGCCAGC | GACATCAGCG |
| 961 | CCGTCGCCGC | GGGGGACCGG | TACTGCCGAA | TATCCAACAC | GTCTGCCATC | AGACACCGAC |
| 1021 | CGGGTTGCCG | CGCCCCTGCA | TGGCCGCCTG | CGTGTCGGGG | ACACGCCGCC | CGGCCCGGAC |
| 1081 | CGGGCCGCGG | GGAGGACCGG | CCCGAGGCGG | GTCCCCTCGC | TCACTCCCCG | GTCGCCGGCC |
| 1141 | GGGGCGGACC | GCCGTCGTGG | CCGACGACGA | GGACGGCCGC | GTCGTCCTCG | TGCCCCACCG |
| 1201 | TGGCCGCCAG | TCGCATGACG | GCGGTGGCGA | GCGCGTCGAC | GTCCAGCCCG | GCGACGGCCG |
| 1261 | TGATCCCGGC | GAGTCTCGTC | ACCCGGTCGA | GACCCTCGTC | GATGTGGAGC | GAGGGCCCCT |
| 1321 | CCACGACCCC | GTCGGTCAGG | AGGACGAAGA | CGCCGTCGGC | GGTGAGCCGG | TGGCGCGTGG |
| 1381 | CCGGGTAGTC | GGCGCCGCGC | AGCACGCCGA | GAGGAGGCCC | GCCCTCGCTG | TCCTCGATGC |
| 1441 | CGGAGCGGCC | GTCGGCGGTG | GCCCAGATGT | GGGGGATGTG | GCCGGCCCGG | GCGCATTCCA |
| 1501 | GGGTGCCGGC | GGCGGGGTCG | AGCCGCAGGA | AGGTGCAGGT | GGCGAAGAGG | TCGGCGCCGA |
| 1561 | GGGAGACGAG | CAGGTCGTTG | GTGCGGCCGA | GCAGCTCTCC | CGGGTCCGCG | GTGACGGAGG |
| 1621 | CCAGCGCGCG | CAGTGCCACG | CGGACCTGTC | CCATGAAGGC | GGCGGCCTCG | ATGTTGTGTC |
| 1681 | CCTGGACGTC | GCCGATGGAC | ATGCCGATCC | GCCCGCCAGG | CAGGGGGAAG | GCGTCGTACC |
| 1741 | AGTCGCCGCC | GACGTTGAGC | CCGTGGTTGG | CGGGCTCGTA | CCGGACGGCC | AGCCGGGCAC |
| 1801 | CCGGAAGACT | GGGCAGGTCC | GAGGGCAGCA | TGCCGCGCTG | CAGGGCCACG | GCGAGCTCGA |
| 1861 | CACGGGACCG | CTGCGTCTCG | GCCAGCTCCC | GCGCCCTGGC | GGTCAGCGAC | CCGAGCCGGA |
| 1921 | CCAGAAGGTC | CTCGCTGCTG | TCGGCCGGGC | GTCTGCGGAA | CATCGATCAC | TCCGACGTCA |
| 1981 | CGACAATCCT | CGCATCACTC | CGTCCCGTCT | CCAGCACGCG | GGACCACAGG | GGACCACCCC |
| 2041 | GGTACGAACA | GGTCCTTCCC | ACTGTGCCCG | GAGGGGGCGG | GGTCCGCATC | TCATCGGCGG |
| 2101 | GAGAGCGCGG | TGGATCCCAG | GGGGCCCGCT | CAGGTCACCG | AAAACGAGCA | AACGTTCGAT |
| 2161 | AATGTGGTCG | CGCCGGTCTG | TGCGGCCGTT | CAGCGTTCGA | CGGTCACTGC | GGCGGCCGCG |
| 2221 | ATGCCGTCGC | GCACCAGCCA | CCGGCCGGTG | AACCCGCCCA | GCTGCCGTCC | GTCGACCGTC |
| 2281 | GGCCCGGGCA | CGAGCAGCCG | GGCCCTGAAA | CGGCCGCCCG | GCTGGAAGTC | GACGGCCGCC |
| 2341 | TCCCCGAAGT | CGAGCCGGCT | CCGGGTGAGC | GGGAACCACG | CCTTGTAGAC | GGCCTCCTTG |
| 2401 | GCGCAGAACA | GCAGCCTGTC | CCACGGGATG | CCGGGCCGGG | CCAGTGCCAG | AGCGGCCAGC |
| 2461 | GGCCCGCGCT | CATCCGCGGT | CGTCACCGCC | TCGAAGACGC | CGTGCGGCAG | CGGAAGGGCG |
| 2521 | GGCTCGGCGT | CGATGCCCAG | AGACGCCACG | TCCGCCTCGG | ACGCCACCGT | CGCGGCGCGG |
| 2581 | TAGCCGGCGC | AGTGGGTCAT | GCTGCCGACG | ACACCGGGCG | GCCATCCCGG | CGCACCCCGC |
| 2641 | TCACCGGGCA | CGAGGGCCAC | CGGCCCCCAC | CCCAGACCGG | CCAGCGCGCG | TCGCGCGCAG |
| 2701 | AAGCGCACGG | AGGTGAACTC | CCTGCGTCGC | TTGTCGACCG | CGCGCCCGAT | CGCCTTCTCC |
| 2761 | TCGTCGGCGA | ACAGCCGCGC | CTCGGGGTAC | CGGTCGGCAT | CCAGGACGTC | GCCGAAGACG |
| 2821 | TCCACCGACA | CCGCGGCGGG | CGGCAGCAGT | GCGCAGATCA | GCCCGGCCCA | CCGCGGCACC |
| 2881 | GACACGGCGG | CGTCGGCCTC | GGCACGGTCG | TGCACGCACG | CCGACGCCGC | GGCGTCGGCG |
| 2941 | AGGGGGCCCG | TGCGGCTGCC | GTCCCCGGAC | CGCCGCGCCG | AGGACGCCGC | CGGCGGGAAG |
| 3001 | CCGTCCCTCA | CACGGGCACC | GTGGCGTCGT | CGTGCGTGCG | TCCGAGCGCG | TTCAGCCGGG |
| 3061 | CCAGCTGGCC | ATCGGAGAGA | GCGATGCCCG | ACGCCGCGAC | GTTCTCCCTC | AGATGCGCGG |
| 3121 | GCGAGCTGGT | GCCGGGGATG | GGAATGACCG | CCGGCGAGCG | GTGCAGCAGC | CACGCGAGCG |
| 3181 | CCACCTGACC | GGCCGACACC | TCCAGCTCGG | TGGCGATGTC | GGCCACCGGG | CTCCCCCCGG |
| 3241 | CGGCGAGCGC | ACCGCGGGCG | ATCGGCAGCC | AGGCGATGAA | CGCGATCTCG | TGCTTCTCGC |
| 3301 | AGTACTCGAC | CACCTCGTCG | TTGCGCCGGT | CGGTCAGGTT | GTACACGTTC | TGCACGCTGG |
| 3361 | CCACGGTGAT | GTGCTCACGG | GCGGCCTCCA | CTTCCCGGAC | GGTGACCTTG | GACAGCCCGA |
| 3421 | TGTGCCGGAT | CTTCCCTTCG | TCCTGCAGTT | CCCCGAGCGC | ACCGAACTGC | TCGGCCGCCG |
| 3481 | GCACCTTCGG | ATCGATCCGG | TGCAGCTGGA | AGAGGTCGAT | GCGGTCGAGT | CGCAGCCTGC |
| 3541 | GCAGGCTCAG | CTCGGCCTGC | TGGCGGAGGT | ACTCCGGTCG | GCCGCACGGC | ACCCACTGGT |
| 3601 | CGGGACGGGG | CCGGCACTGT | CCGGCCTTCG | TCGCGATCAC | GAGGCCGTCG | GGGTACGGGC |
| 3661 | GCAGTGCTTC | GGCCAGCAGC | TCCTCGTTGC | TTCCCAGCCC | GTAGGAATCG | GCCGTGTCGA |
| 3721 | TGAAGGACAC | ACCGAGGTCC | ACGGCCAGCC | GGGCCGTGCT | GATCGCAGCC | TCCCGGTCCT |
| 3781 | CCGGCGGGCC | CCAGTACCCC | GGACCGGTCA | GCCGGAGGGC | GCCGAAACCC | AACCGGTGTA |
| 3841 | CCGAGAGGTC | GCCTCCCAGA | GGGAAGGTGG | TCCTGCCGGG | CTGTGCCATG | CGTTCCTCCT |
| 3901 | GGACGACGTC | CGTGCACTCG | GGTGGGGCGC | GTGGTGACCT | CGGTGGGGGC | GGGCCGATGC |
| 3961 | CGACCGTCCG | AACACGGTGG | ATCCCCCGGT | TCAGGGAGCC | GTCGTGGAGG | GCACCCGCTC |
| 4021 | GTCGGCCCGT | CGGGTCACCT | CGTGTCCCCG | CTCCAGAGTG | AGCCGCACGA | TGTCGCAGAC |
| 4081 | CCGACGGATG | TCCTCGCGAG | AGACGGTGGG | GCCGGTGGGC | AGGGCGAGGA | CCTTCCGCGC |
| 4141 | GAGCCGTTCG | GTGTGGGGCA | GGGAGACCGG | CCGCTCCGAG | CGGTAGGGCT | CCATCTCGTG |
| 4201 | GCATCCGGGG | GAGAAGTACC | GCTGGCACAT | GATGTTCTCC | GCCCTCAGCA | CCTCGTCCAG |
| 4261 | CAGGTCCCGG | TGGACCCCGG | TCACCGCGGC | GTCGACCTCC | ACGACCAGAT | AGTGGTAGTT |
| 4321 | GTTGCGCTCG | GCACGGTCGA | ACTCCATGAC | CTTCAGCCCG | GCGAGCCCGG | CGAGTTCCGA |
| 4381 | CCGGTAGTCG | TCGTGGTTGG | CCTCGTTCCG | GCGAACCGTC | TCCTCGAAGG | CGTCGAGCGA |
| 4441 | GGTGAGTCCC | ATCGCGGCGG | CGGCTTCGGT | CATCTTCCCG | TTGGTGCCGG | TCTCCGTGGA |
| 4501 | CACCCGCCCC | TGGGTGAATC | CGAAGTTGTG | CATCGCGCGG | ACCCGCTGGG | CCAGCCTCTC |
| 4561 | GTCATCGGTC | ACCACCGCGC | CGCCCTCGAA | GGCGTTGACC | ACCTTGGTGG | CGTGGAAGCT |
| 4621 | GAACACCTCG | GCTTGTCCGA | AGCCGCCGAT | CGGCTGCCCT | TCCGACGTGC | AGCCGAGTCC |
| 4681 | GTGCGCGGCA | TCGTAGAAGA | GCCTGAGACC | GTGATCGGCC | GCGACCTTCG | CCAGCCGGTC |
| 4741 | GACCGCGCAG | GGCCGACCCC | ACAGATGGAC | CGGCACGACC | GCACTCGTAC | GGGGGGTGAT |
| 4801 | CGCCGCCTCG | ATCAGATCCG | GGTCCAGGCA | GTTGGTGGCC | GGGTCGATGT | CCACGAAGAC |
| 4861 | CGGGGTCAGA | CCCAGCCAGC | GGAACGCCTG | GGCGGTCGCC | GGGAAGGTCA | GCGACGGCAT |
| 4921 | GATGACCTCG | CCCGACAGAT | CGGCGGCGCG | GGCCAGCAAC | TGGAGGGCGA | CGGTCGCGTT |
| 4981 | GCAGGTCGAC | ACGCAGTAGC | GCACGCCGGC | GAGTTCCGCC | ACGCGCTGCT | CGAACTCCCG |
| 5041 | GGCCAGCGGC | CCGCCGTTGG | TCAGCCACTG | GTGGTCCAGC | GCCCAGTTCA | CACGGTCCAG |
| 5101 | GAACCGCGCT | CGGTCGCCCA | CGTTGGGCCG | CCCCACGTGA | AGTGGTTGCA | GGAACGCCGA |
| 5161 | CGGGCCGCCG | AACACGGCGA | GATCACCGAG | ATTGCGTTTC | ATGGTCATAC | CTCCCCGGTG |
| 5221 | CACGGGACGG | TGCACCCGGC | AGCAGCCGGC | ACAGGACGTG | GACGTACCGG | AGGGACAGGG |
| 5281 | GCCGCAGCGC | ACGACGGCGT | CTCCGTCGTG | CGTACGCGAA | AGGGGCGCAC | GGAACGGATC |
| 5341 | AGTGCTCGCG | GCGCCAGTAG | ACGCCCGTCA | CGTCCACCGT | CTCGATCGGC | TCGTCGATGC |
| 5401 | CGTGTTCGCT | CCGGTAGTCG | TGGACGGCCT | GCTTGCACGC | CGGGATCAGG | TAGTCGTCCA |
| 5461 | CGATCACGAA | CCCGCCCACG | GACAGCTTGG | GGTAGAGGTT | GACCAGCGCG | TCCCTCGTCG |
| 5521 | ACTCGTACAG | GTCGCCGTCC | ACCCGCAGCA | CCGCGAGCCG | GTCGATCGGC | GCGGTCGGCA |
| 5581 | GCGTGTCGGC | GAACATGCCG | GGCAGGAACC | TGACCTGCTC | GTCCAGCAGG | CCGTAGCGGG |
| 5641 | CGAAGTTCTC | CCTCACCTGC | TCCTCGGAAC | AGCTCAGTAC | CCAGTTCAGG | TGGTGGAACT |
| 5701 | CCATCGCCCG | GTCGAGCGGG | TGGCTGTCCT | CGGACGTCAC | CGGGACACCG | GCGAACGAGT |
| 5761 | CGGCGAGCCA | CACGGTGCGG | TCCTCCACAC | CGTGCGCCTT | GAGGAGCGCG | CGCATCAGGA |
| 5821 | TGCAGGCGCC | GCCGCGCCAC | ACGCCGGTCT | CGATGAGATC | ACCGGGAACA | TCGTCCTGGA |
| 5881 | GGACCCTCTC | CACGCACCTC | TGGATGTTGT | CCAGGCGTTG | CAGGCCGATC | ATGGTGTGCG |
| 5941 | CGACCGTCGG | CACGTCCAGT | CCTTCGGCCC | GGTGGTCGGC | GTCGAAGGTC | CCGCGCTCGA |
| 6001 | CGTCCGGGCC | CTCCGCCCCC | GGACGCATGC | GGTCCGCCAG | TTCCTTGTCC | AGGACGTTCA |
| 6061 | TCCCGACCGG | GAAGGTCGGG | TGATCCCCGT | AGATGGTGTT | CGACACGACG | TTCTTCATGA |
| 6121 | GGTCCACGTA | GAGCTCGCGG | GTCTGCACGG | TCGACTTACC | TCCGGCTAGA | TATCGAACAG |
| 6181 | AGAGAGAATG | TGCGGGGCGA | CGGCGTCCGC | GGACGCGGTG | GGAGCCGTCG | CGGTCGGGTC |
| 6241 | ACCTCAGGAA | GCCGCGCGCG | TCCTCCCAGC | CGGCCACGAC | ATCGGCCTCC | AGCTGGTTGA |
| 6301 | GCCGCGCCGC | CACCACCTGG | TCGAACCCGT | CCATGAAGTA | CTCGTCACCG | GCGTGCGGCG |
| 6361 | CTATCAGACG | GCCGTCGTCC | ACGAACCGCT | CGACGACCTC | CGTCAGGGAG | GTGCCCGGCC |
| 6421 | CCACCCGGCC | CGCGACGTAC | CGGTCCGCTC | CGGTGAGATC | CGGGAACCCC | GCCTCCCGGT |
| 6481 | ACAGGTACAC | GTCACCCAGC | AGGTCCACCT | GTACGGACAC | CTGGGGATGC | GCGGACCGGC |
| 6541 | GCATGGTTCC | GGGCCTGATC | CTGAGCAGCT | CGGCGTCCGC | GCCGGTCTTC | AGGCTGTGCA |
| 6601 | ACGCGTAGCC | GTAGTCGATG | TTCAGCGTGG | GGGTCCGCCG | GCTCACCGCC | TCCTCGAACG |
| 6661 | CGAGCAGCCC | CTCCTGGAGT | TCGGCGCGTT | CGGCCTCGAA | CAGCCTGCCG | TCCTCCCGGC |
| 6721 | CGCTGTAGTC | CTCGCGCACG | TTCAGGAAGT | CCACCGGCCG | GTCCGGCGCG | GCCTCGTTCA |
| 6781 | GGTCGGCGAT | GAAGTCGACG | AGGTCGAGCA | GCCGGTGGAC | GCGCCCGGGC | AGCACGATGT |
| 6841 | AGCTGAGCCC | GAGCCGGATC | GGGCTCTCCC | GCTCGGACCG | CAGCTGCTGG | AAGCGCCGCA |
| 6901 | GGTTGGCGCG | GACCCTTCCG | AAGGCGGCGC | GCTTGCCCGT | GGTCTCCTCG | TACTCCTCGT |
| 6961 | CGTTCAAGCC | GTACAGCGAG | GTGCGCACCG | CGTGCAGGTC | CCACACCCCG | GGCTGCCTGT |
| 7021 | CCAGCGTCCG | CTCGGTGAGC | GCGAACGAGT | TCGTGTACAC | GGTCGGCCGC | AGCCCGCGAG |
| 7081 | CCGCCGCACG | GCTGCTGAGC | GCGCCCAGGC | CCGGGTTGGT | GAGGGGCTCC | AGACCGCCCG |
| 7141 | AGAAGTACAT | GGCGTACGGG | TTGCCGGCCG | GGATCTCGTC | GATGACGGAC | GCGAACATCG |
| 7201 | CGTTGCCGGA | CTCCAGCGCC | GACGGGTCGT | ACCGCGCACC | GGTCACCCGG | ACGCAGAAGT |
| 7261 | GGCAGCGGAA | CATGCAGCTC | GGCCCCGGGT | ACAGGCCCAC | GACATAGGGG | AAGGCCGGCT |
| 7321 | TGTGCGCGAG | CGCCGCGTCG | AAGACCCCCC | GTCGCTCCAG | CGGCAGCAGG | GTGTTCTGCC |
| 7381 | AGTACTTGCC | CGCCGGCCCG | TTCTCCACGG | CGTGGCGCAG | CTCCGGGACA | CGGCCGAAGA |
| 7441 | GGCCGAGGAG | CCGGCCGAAG | GAGGCCCGGT | CCACGTCCAG | CATCCGGCGT | GCCTCCTCCA |
| 7501 | GGGGCGTGAA | GGGGTGGTTC | CCGTAGCGCT | CGGCCAGCCG | CACGAGCCGG | CGCGCGACCG |
| 7561 | TCGGGCCCTC | GTCGACACCG | ACGCGGCCGT | CGGAGACCAG | GGCGCGGTGC | ACCGTGTGGA |
| 7621 | CCGCTGCCGG | CAGGTCGGCG | CCGGGGCGGG | CGCACAGGGC | GACCGGATCG | GCCGCGGTGG |
| 7681 | AGGCGTGCGA | GGTCATCCTT | CGTGTCCAAT | CGTCTGCGTC | CGGAGGGTCG | GTCCGTTCCC |
| 7741 | CGGGAAACGC | GGCGGGGCGG | TCACCGGGAC | GGCACCGTGA | CGGATGTGGT | CAGGGACGTC |
| 7801 | TCGGCCGCGG | ACGGGCCCAC | GTGTACGGCC | CTCCTGCCGG | TGCCCGGCTT | CCACGTGCCG |
| 7861 | GCGGCGGTGT | CCCAGTAGCG | CAGTTGCTGC | TCGTCGACGG | GGACGGTGAC | CCGCCGCTGT |
| 7921 | TCTCCCGGCC | GGAGGGTGAC | CTTGGTGAAT | CCGGCGAGTT | TGCGCAGCGC | CTGGGGGGCC |
| 7981 | TGGGTGTCCG | GACTTGCCCC | GAGGTAGACC | TGGACGGTCT | CCCTGCCCGC | CCGGTCACCG |
| 8041 | GTGTTGCGGA | CGGTGACCGT | GGCCTCCAGC | CCCCGGGCGG | TGCGCGCCAC | GGACAGGTCG |
| 8101 | CTGTAGGCGA | AGGTGGTGTA | CGACAGGCCG | TACCCGAACG | GGAAGAGCGG | GGCCACGCCG |
| 8161 | GTCCGGTCGT | ACCACCGGTA | GCCGACATCC | AGTCCCTCGG | AATAGGTCTC | CTTGCCGTCG |
| 8221 | ACACCCGGGT | AGCGGTGCGG | GTCACCGGCC | ATGGGATGGC | CGGTCTCGGT | CCCCGGGAAG |
| 8281 | GTCTGGGTCA | GCCGGCCGCT | GGGATTCGCG | TCGCCGAACA | GCAGGGCAGC | GGTGGCCTCC |
| 8341 | GCGCCCTCCT | GCCCCGGGTA | CCACATCTCC | AGTACGGCGG | CCGTCTCACG | CAGCCAGGGC |
| 8401 | ATGAGCACCG | AGGAACCGGT | GTTGAGGACG | ACGATCGTGC | GGGGGTTGAC | CTTGGTGATC |
| 8461 | GCCGCGATCA | GCTCGTCCTG | TCGGCCGGGC | AGGGACAGCG | AGGACCGGTC | CACGCCCTCG |
| 8521 | GCACTGTCGT | CGTGGGCGAA | GACGACGGCG | GTGCGGGCCT | CGGCCGCCGC | CGCGACGGCG |
| 8581 | CGGTCGAAGT | CGTCCCGGGC | AGCCTCGGGA | GTGACCCAGC | TCAGCTCCAC | CGACAGCGGG |
| 8641 | GTCTGTTCGA | AGGCCCAGCC | GTTCATGGTC | ACCGCGTGGG | TGCCGCGGGT | CAGCCGCATC |
| 8701 | GTGGTGGTGA | CGGTGCCGAA | CGCCTCGGTG | CCGAGGACAG | CGGACTGCCC | CGCGATCTGC |
| 8761 | AGGTTGGCGA | CCCCGCCGAC | GGCGGTGAAG | GCGATCTTGT | AGTCGCCGTC | GGCGGGGACG |
| 8821 | GTGAGCCGAC | CCTCGTAGAA | GACGCCCTGG | CCGCTGGGAT | CCAGTTGCTT | CCCGCTCGTG |
| 8881 | AAGGCGGGGG | TGAGCGAGCT | TTCGGGCACG | GGCACGCCGA | CCAGGTCCTC | ACCGGGCTCG |
| 8941 | TGGACGACCC | GGGCCTTCTC | GCCGGCGCGC | CGTGCGAGGG | CGTCGACGGG | TGCGGTCGCC |
| 9001 | CGGTCGGGTA | TGACGTGCGC | GCTGCCGTTG | CCGGTGACCT | TGGGGTGCTT | GGCGCTGTTG |
| 9061 | CCGATGACGG | CGATGTCCGT | GGCGTCCTCA | CCGGTCAGGG | GCAGCGTCCG | GCGCTCGTTG |
| 9121 | CGCAGCAGCA | CCCCGCCGTT | CTCGGCGATG | GTGCGGGCCG | CCGCGCGGCC | GGCCTCGGGA |
| 9181 | TCACGCTGCG | GGCGCTCGGT | CGCCTTGCCG | TCGAGCAGGC | CGAAGCGCTC | CATCTGGCCG |
| 9241 | AGGATGCGGA | CGACGGACCG | ATCGAGCGTC | GCCTCGGGGA | CGCTGCCGTC | CCGCACGGCG |
| 9301 | GCCCTGAGCG | CGGAGGAGAA | GTACTTCGAT | TCCGGCACCG | GCTGCCCCAG | GGTCAGCTCG |
| 9361 | ACGCCCAGTT | CCTGGTCGAG | GCCCCTGGTG | ATGTCGCCGG | TGGCGTGGGT | CGCCAGCCAG |
| 9421 | TCGGACACCA | CCCAGCCGCG | GAAGTCCCAC | TGTTCGCGCA | GCACCTCCTG | CAACAGGTGC |
| 9481 | TCGTTCCCGC | ATGCGTGCGC | GCCGTTGACC | TTGTTGTACG | AGCACATCAC | CGAGGCGGCG |
| 9541 | CCCGCCCGCA | CGGCGCTGCG | GAAGGCCGGC | AGCTCCACCT | CCTGGAGCGT | CTGCTCGTCG |
| 9601 | ACCACGGCGT | CGATGGTCTC | CCGCTGGTAC | TCCTGGTTGT | TCGCGGCGAA | GTGCTTGACC |
| 9661 | GTGGCCATCA | GGCCCTGGTT | CTGGATGCCG | CGGACCTCGT | GGGCGGCCAT | CCGGGACGAC |
| 9721 | AGCAGAGGAT | CCTCGCTGTA | GGTCTCGAAG | TTCCGCCCCG | CGTGCGGCAC | CCGGATGACG |
| 9781 | TTGGTCATGG | GACCGAGGAC | GATGTCCTGT | CCGAGGGCCC | GTCCCTCCCG | GCCCAGGACG |
| 9841 | GTGCCGTACT | CCTCGGCGAG | GCGTTCGTCG | AAGGTGGCGG | CGAGCGCCAC | GGGGGTGGGC |
| 9901 | ATGGCGGTGG | CGGTACCGCC | GAGCAGACGG | ATGCCGACCG | GGCCGTCGGC | GGTGCGCAGC |
| 9961 | TCGGGGATGC | CGAGCCGGGG | CACGCCCGGC | AGATAGCCGA | TGCCGGTCAT | GGTCGGCCCG |
| 10021 | CCCACGGGCC | CGGTGGTCCA | GTGGACGAAC | GAGATCTTCT | CGTCGAGGGT | CATCTTCGCC |
| 10081 | ACCAGCCCGC | GCACCCGCGC | CGTCCCGGGC | TCGGCGGCCG | GAGCACCGGC | GGCGGAGGGG |
| 10141 | GCGGTGAGCA | GACCGCCGAC | GCACAGGGCC | GCGAGCGCGG | ATCCGACGGT | GCGCCGGATG |
| 10201 | CGGCGACCCG | TCCTCGTCGT | GCCGAACAGC | ATGCTGACGG | ACGTCCTTTC | TGCCGAGGTT |
| 10261 | GCCGTCCTCA | TCGGGGCGGG | AACGTTTCTG | TCCGTGCCGC | CATGATCACC | AGCCCACGAG |
| 10321 | CATCGTGCGC | GGGCCCCGCA | CCACCATCTC | CGTCTTCCAC | TCGACGGGCT | GGGCGAGGTG |
| 10381 | CAGCCCGGGG | AGTTCGTCGA | GCAGGGTCCG | CAGCGCCTCC | TGGAGCTCCA | GCCGCGCGAG |
| 10441 | GGACGCGCCG | AGGCAGTGAT | GCGGACCGTG | TCCGTACCCC | AGGTGCCGGC | CGGCGTCGTC |
| 10501 | GCGGGTGATG | TCGAGCACGC | CGGGCGAGCG | GAAGCGCAGG | GCGTCCCGGT | TGGCGGCGTT |
| 10561 | CATCTGGACC | AGCACCGGAT | CCCCCGCCCG | CACCAGCGTC | CCGCCCACCT | CGACGTCCTC |
| 10621 | GGTGGCGTAG | CGCGGGAATC | CCGCCTGGCT | GCCGAGCGGA | ACGAACCGCG | TCAGCTCTTC |
| 10681 | CACCGCGTTG | TCCAGCAGGT | CAGGACGGTC | CCGGAGGAGG | GCCAGCTGGT | CCGGCTGTTC |
| 10741 | GAGCAGGACC | AGGACGAAGT | TGCTGATCTG | GCTGGCCGTG | GTCTCGTGCC | CGGCGAACAG |
| 10801 | GAGGAAGACG | ATCAGGTCGA | CCAACTCCTC | CTGCGACAGC | CGGCCCTGGG | CGTCACGGGC |
| 10861 | CTCGACGAGC | GCCGAGACGA | GATCGTCTCG | CGGCGCGGCG | CGGCGGGCCG | TGATCAGGTC |
| 10921 | CGCCAGATAG | CCGGTCAGCT | CGCCGGCCAG | CCGCACATGG | TCCTCGGCGG | TGAGCGAGCT |
| 10981 | GGTGGACATC | GCTATCTCGC | ACCAGCCGCG | GAACAGGTCA | CGGTCCTCCT | CGGGCACGCC |
| 11041 | CAGCAGGCCG | CAGATGACCG | CCACGGGAAG | GGGGACGGCG | TAGTGGTCCA | CGAGGTCGAC |
| 11101 | GGGCGATCCG | AGCGCCGTCA | TGTCCCGGAG | CAGCGACGCC | GTCATCCTTC | GGACGTGCGG |
| 11161 | CCGCATGGCC | TCCACGCGGC | GCGGGGTGAA | CGCCTTGGTG | ACCAGCCCGC | GCAGCCTGGT |
| 11221 | GTGGTCCGGC | GCGTCCATGC | CGATGATGCC | GTTGGGCGTC | CGCAGCTCCC | GCATGCGCGG |
| 11281 | CGCGTCGTTC | TCCGCCTCCG | CGGCGTGGCT | GAACCGCCGG | TCGCCGAGGA | CGAAACGCGC |
| 11341 | GTCGTCGTAA | CGGGAGACGA | GCCAGCCCGG | CTCCCCGTAC | GGCATGTGGA | CCCAGAGGAG |
| 11401 | TCCTTCGCTC | TTCCGCGCCT | GCTCGTACGC | CTCGTCCAGG | TCCAGTCCCT | CCGGGCGGCC |
| 11461 | GAAGGGATAG | GAGACGGGTG | CGGTCTTCGT | GCTGGTCAAG | GGGAGCCCCA | TATCGCGTCG |
| 11521 | TTCGGGTGCT | TAATCGTCGT | GGGCCGGATC | ATGCGGCCGA | GCCGCCCCAG | GTGACGGGGA |
| 11581 | CCGCCTCGGG | GGTGCGCAGG | AGGGAGCCGG | CCCGCCAGCG | AAGCTCACTC | GCGGGGACGG |
| 11641 | ACAGCCGGAG | CTCCGGCAGG | CGCTCGACCA | GGGTGCTCAG | CACGACCTGC | AGTTCGAGCG |
| 11701 | GGGCCAGGGG | GGCGCCCATG | CAGTAGTGGG | CGCCGTGACC | GAAGCCGAGG | TGCGGGTTCT |
| 11761 | GGGCACGGGC | CAGGTCGAGG | GAGGCGGCAC | CGGGAAACGC | CGTGTCGTCG | AGGTTCGCGG |
| 11821 | ACGCGATCGA | GGTGATGACC | GCCTCGCCCG | CGCGGATGAG | CGTGCCGCCG | ATCTCGACGT |
| 11881 | CCTCCAAGGC | GATGCGGGCG | AACCCGTCCA | GGGTGCTCAG | CGGGGTGAAG | CGCAGCAGCT |
| 11941 | CCTCGACGGC | GGAGGGAACC | AGGTCAGGTT | CGGTCACCAG | GCGCTCGTAG | AGGCGGCGGT |
| 12001 | CGTCGAGCAG | GAGGAGGACG | AAATTGCCGA | TCTGGGTGGC | GGTCGTCTCG | TATCCGCTGA |
| 12061 | TCAGCAGGCC | GGAGCCCATC | ATGAGGAGCT | CGGCCTCGGA | GAGCCGGTCG | CCTTCGTCAC |
| 12121 | GGGCCTGGAC | CATGGTGCTG | AGGAAGTCGT | CGGTGGGCCG | GGCGCGGCGA | TCGGCCACGA |
| 12181 | GGTCGGCCAT | GTAGGCGGAG | AAGTCCTCGG | TCGCCCGCTG | CACTTCGGCG | GGCCCCAGGG |
| 12241 | AGGAGGACAC | CAGCGCCTCG | GAGAAGTCCC | GGAAGAGGTG | GCGGTCGTCG | TAGGAGACGC |
| 12301 | CCAGCAGTCC | GCAGATCACG | GAGATCGACA | CGGGCAGCGC | CAGGTCTTCC | ATGACCTCGC |
| 12361 | CGGACGGCTC | GTCGCGCGCG | ACCATGCCGT | CGAGCAGTTC | ATCGGTGAAC | CGTGAGACCC |
| 12421 | GCGGGCGCAG | TTCCTCCACC | CGGCGGGCGG | TGAACGCCTT | CCCGGCGAGC | CGCCGGAGCC |
| 12481 | TGGTGTGTCC | GGGCGGGTCC | ATGGAGATGA | GACCGCCGGC | GGGCAGCGGG | TACTCGGTGG |
| 12541 | GCCGGGGCAC | GTCACGTCCG | GCCCCGGCCT | CCATGCTGAA | CCGCGGGTCT | CCCAGGACCG |
| 12601 | CCCTCACGTC | GGCGTGTCGG | GTCAGCAGCC | AGGCCTCACC | GCCGTACGGC | ATGCGGATCC |
| 12661 | GGGACACGGG | CTCGTGCTCC | CGCAGACGGC | TGTAGAGCGG | ATCCAGCGCG | ATGCCCTCGG |
| 12721 | GCGGGGAGAA | GGGGTACGGG | CGAGTCCGGT | GGACCGGACA | GCTAGACGTC | ATGGACACTC |
| 12781 | TCTCGGTCGT | ACGGGGCGGC | GGCAGAGCTC | GAAGGGTGCG | GGCGGCCGAC | GGCGAGCCCG |
| 12841 | GTCGCCGTCC | GGATCGTCCG | GCACACCGCT | TCGGCCTGCT | CGGTGAGGTA | GAAGTGGCCT |
| 12901 | CCGGGGAAGG | AACGGAACGC | CGTCTCCCCG | GTGGTCAGCC | CGTGCCAGGC | CAGGGCTCCG |
| 12961 | TCGGTGGGGA | CGTGCGGATC | CGCGCTTCCC | GTCAGCACGG | TGATCGGGCA | GGCGAGCGGA |
| 13021 | GCTCCCGGAC | GCCACGTGTA | GGTGGCGGCG | GCCCGGTAGT | CGTTGCGGAT | CGCCGGCAGG |
| 13081 | GCCATCCGCA | GGACCTCCTC | GTCGGCCAGC | ACCCGGGGGT | CGGTGCCTTG | CAGTTCGGCC |
| 13141 | AGCTTGGCGA | CCAGCCGGTC | GTCGCTCAGC | AGGTGCGCGG | TGGTCCGGTG | GGCGACGGTC |
| 13201 | GGGGCGACGC | GGCCGGAGAC | GAACAGATGC | GCCAGCCTGC | TCTCCGGCGC | CTGTTCCGCG |
| 13261 | AGCCGGCGGG | CGGTCTCGAA | GGCCACCAGC | GACCCCAGGC | TGTGACCGAA | GAGCGCCACC |
| 13321 | GGCCGGTCCA | GCCAGGGGCC | GAGCGCTCCG | GCGACGTGCG | TCACCAGAGC | GTCGACGGAG |
| 13381 | TCCACGAACG | GCTCCAGCCG | CCGGTCCTGC | CGCCCGGGAT | ACTGGACGGC | GAGCACCTCG |
| 13441 | ACCCGGTCCG | GCAGCAGACA | GGTGAAGGGG | AGGAAGGCGC | TGGCGCTGCC | GCCGGCGTGC |
| 13501 | GGAAGGCACA | CGAGCCGGAG | CGCGGGGTCG | GCGACGGGCC | GGTAGCGGCG | CAGCCAGAGG |
| 13561 | TCGCCGGCGT | TGGTGGAGCC | GGGCGCCGTC | GTGGCGGGCC | CCTCGTTCAT | GCGTGATCCG |
| 13621 | CTTCCGGACG | TCACGGTGTC | AGCGCCTTCC | ACCACGTGCC | GTTGTCCCGG | TACCAGTCGA |
| 13681 | CGGTCTCCGC | GAGCCCGCGA | TCGAAGGCGA | CGCGGGGAGC | CCATCCCAGC | TCCTCGGTGA |
| 13741 | TCTTCCGGGT | GTCCACGCAG | TAGCGACGGT | CGTGCGCGGC | CCGGTCCGGC | ACCCGGTCCA |
| 13801 | CCACGGACCA | GTCGGCACCG | AAGACGCCGA | GAAGACGACG | GGTGAGGTCG | ACGTTGGACA |
| 13861 | GTTCCGTCCC | GCCGCCGATG | TTGTACACCT | CTCCGGCCCG | CCCCCGGGCG | GCGACCATGG |
| 13921 | CGATGCCCCG | GCAGTGGTCG | TCCACGTGCA | GCCAGTCGCG | CCGGTGGAGG | CCGTCCCCGT |
| 13981 | ACAGGGGAAG | GCGCAGGCCC | TCCAGCAGAT | GGGTCACGAA | GAGCGGGACG | ACCTTCTCGG |
| 14041 | GGTGCTGGTG | GGGGCCGTAG | TTGTTGGAGC | AGCGCGTCAC | CCGGACGTCG | AGGCCGTGGG |
| 14101 | TACGGTGGAA | GGCGAGCGCC | AGGAGGTCGG | AGGAGGCCTT | GGACGCCGCG | TAGGGGGAGT |
| 14161 | TCGGGTCCAG | CGGATCGGCC | TCGGTGGAGG | AGCCCTCGGG | GATCGATCCG | TACACCTCGT |
| 14221 | CGGTGGAGAC | GTGGACGAAG | CGGCTGACGC | CCGCTTCGAG | CGCCGACCGC | AGCAGGGTCT |
| 14281 | GCGTCCCCAG | CACGTTGGTG | GTGACGAAGG | CGGCGGAGTC | CGTGATCGAG | CGGTCCACGT |
| 14341 | GCGACTCGGC | GGCGAAGTGC | ACCACCAGGT | CGGTGCCGCG | CAGGGCCTGG | GCCACCGTGG |
| 14401 | GCGGGTCGCA | GATGTCGCCG | TGCAGGAAGG | TGTGACGGGG | GTGGCCGGCG | ACCTCGGCGA |
| 14461 | GGTTCTCGGT | GTTCCCGGCG | TACGTGAGCT | TGTCCAGCGA | GAGGACGTGG | GCGCCGGCCA |
| 14521 | GCTCCGGATA | GGAGCCCGAC | AGGAGCTGTC | TGACGAAGTG | CGAGCCGATG | AAGCCCGCTG |
| 14581 | CCCCGGTCAC | CAGGACGCGC | ATCCTTGATC | CGCCTTTCTG | CCGTGCTGTG | TGGGGGAGGT |
| 14641 | GTGGTCAACC | GGTGCGCGTC | GCCCCGAGTC | CCGAGGCGAC | CTCCATGAGG | TAGTCCCCGT |
| 14701 | AGCTGGAGGC | GCTCAGCTCC | TCACCCAGCC | GGTAACAGGT | GTCCGCGTCG | ATGAAGCCCA |
| 14761 | TGCGCAGCGC | GATCTCCTCG | AGACAGGCGA | TGCGTACGCC | CTGGCGCTGC | TCGAGGAGCT |
| 14821 | GGACGTACTG | ACCCGCCTGG | AGCAGGGAGT | CGTGGGTTCC | CATGTCGAGC | CAGGCGAATC |
| 14881 | CACGTCCGAG | CTGGGTCAGC | CGGGCGCGGC | CCTGCTCCAG | ATAGGACAGG | TTGATGTCGG |
| 14941 | TGATCTCCAA | CTCGCCTCGG | GCTGAGGGCT | TGAGGTCCTT | CGCCAGCTCC | ACCACGTCGT |
| 15001 | TGTCGTAGAA | GTACAGCCCG | GTGACGGCGA | GGTTGGAGCG | AGGACGGCTC | GGCTTCTCCT |
| 15061 | CCAGGGACAA | CAGGTGCCCC | TGCTCGTCGA | TCTCGGCGAC | GCCGTAGCGC | TCGGGCGACT |
| 15121 | TGGACGGATA | GCCGAACAGT | TCACATCCGT | CGAGACGTCG | CAGGGAGTGC | TTCAGGACGG |
| 15181 | TGGAGAATCC | GGGGCCGTGG | AAGACGTTGT | CGCCCAGAAT | GAGCGCCACC | CGGTCGTTCC |
| 15241 | CGATGTGCTT | GTCGCCGACC | AGGAACGCGT | CGGCGACACC | GCGCGGCTCG | TCCTGGACCG |
| 15301 | CGTAGTCGAG | CTGGATACCG | AGGCGCGAGC | CGTCGCCGAG | CATGACCTGG | AACGTCTCCA |
| 15361 | CGTGCTGGTG | CGAGGAGATG | ATGAGGATGT | CCTGGATTCC | CGCCAGCATC | AGCACCGACA |
| 15421 | GCGGATAATA | GATCATGGGT | TTGTCATAAA | CGGGCAACTG | CTGTTTGGAA | AGCGCACCGG |
| 15481 | TCAGCGGCTG | CAGTCGAGTG | GCGCTTCCCC | CGGCGAGGAT | TATCCCCCTC | ACTCCGGGGC |
| 15541 | GTTTCAGCGG | TGTCTCGAAC | ACGGTTGGTC | CTCCGTGGTC | ACATGGCCGA | TATGGGGGGT |
| 15601 | GAAGACACTG | TCCTGAGAGG | CCGGCGGACC | GGCTGTCGCC | TCGCGGACAC | AGCGGCTTAA |
| 15661 | TGCATTCACC | CCGCCCCGGG | ACCGTCATCC | GAGAAGAAGG | AATGCGGTGT | CGTGGGAACC |
| 15721 | GACGTCCAGG | AGTTCCTTTC | GGGCCGCGGA | ACGGCGGCGC | GGAGATTCTG | AACCGCGGGG |
| 15781 | GATTCCAGGG | CGGTGGCAGG | GAAGGGAACC | ACCGCCGCGC | CATCTCTCCC | GGAACGTTCC |
| 15841 | GCAAGCGGCG | GGCCGTGCTT | CGGACGGCCT | ATCTCTGCGC | CTGTTGCTGT | TCCTGCCAGG |
| 15901 | CCTGATAGGT | CGGCAGCAGA | CCGAGCCGTT | CGGCCGTCGC | GAGGGTCGGG | GCGTTCTCGT |
| 15961 | CCCTGTCGGA | GAGCAGCGGC | TCGATGTCGT | CCGGCCACGC | GATTCCGAGG | TCGGGGTCGA |
| 16021 | GCGGATTGAC | GGAGTGCTCG | CGTGCGGGAG | CGTATCCGGA | GGAGCAGAGG | TAGACGAGCG |
| 16081 | TGGCGTCGTC | GGTGAGGGAG | AGGAAGGCAC | GGCCCAGTCC | CGCGGTCAGA | TAGACGGCGG |
| 16141 | TGTTGCGTTC | CGCGTCCATG | GGCACGATCT | CCCAGCGCCC | GAAGGTCGGC | GAACCGATGC |
| 16201 | GGACGTCCAC | GACGACGTCG | AGGCCGGCTC | CCCGCACGCA | CACGCTGTAC | TTGGCCTGAC |
| 16261 | CTGGCGGGAT | CTCGGTGTAG | TGGATCCCGC | GCAGCGCGCC | GCGGTGCGAC | ACCGCGACAT |
| 16321 | TGACCTGGGC | CACCGGGAAG | TCATGGCCGA | ACGCCTGACG | GAAGCTCTCG | CCCCGGAACC |
| 16381 | ACTCGTGGGA | CCTCCCGCGG | TGGTCGGAGT | GGATGACGGG | TTCCTGCGAC | CAGGCCCCCT |
| 16441 | CGATGCTGAG | TGGATGCATC | GCGCCTTCTC | CTTCGGACCG | ATGGGTGGGG | TGCGGGGCGG |
| 16501 | GCCGGGGGCA | CGGCCGAGCC | GGTCAGCTGG | AGCGTCTCCA | GTAGGAACCC | TGGCGATCGA |
| 16561 | TCTGGTGGAT | CTCGTCCGTG | ATGCCGTGCT | CGTCCCGGAA | CTCGTGGACG | GCCTGCCGGC |
| 16621 | ACGCCGGAAT | GCAGTAGTCG | TCGACGATGA | CGTACCCGCC | GTCCGACACC | TTGTGGTAGA |
| 16681 | GGTTGGTGAG | AACCTCCCTG | GTGGCTGCGT | ACGAGTCCCC | GTCGAGCCTC | AGCACCGCGA |
| 16741 | GCCTCTCGAT | GGGCGCGGTG | GGCATCGTGT | CCTTGAACCA | GCCCGGGAGG | AAGCGGACCT |
| 16801 | GGTCGTCCAG | GAGTCCGTAG | CGCGCGAAGT | TCCCCTTCAC | GGTCTCCACG | TCGACCGGGA |
| 16861 | TGCTGAGGAC | GTCGTTGTAC | TGGCCGAGGT | CGATGTCGAC | GTCCAGCTGG | TGGTCGTCCT |
| 16921 | CGGTGGTCTT | GGGGAAACCC | TGGAAGGAGT | CCGCGACCCA | CACCTTCCGG | TCCCGCACGC |
| 16981 | CGTGCGCCCG | GAAGACGCCG | CGGGCGAAGA | TGCAGGCCCC | GCCCCGCCAG | ACCCCGGTCT |
| 17041 | CCGCGAAGTC | CCCCGGCACG | CCGTCGCGCA | GCACGTCCTC | CAGGCACTTC | TGCAGGTTGT |
| 17101 | CGAGCCGCTT | GAGACCGACC | ATCGAGTGCG | CCACGCGCGG | AAAGTCCTCA | CCCACCGAGC |
| 17161 | GCAGTTCCGC | GGAATACGAG | CTGCTGGTGA | TGAGGCCGGC | GACATTGGTC | TGGTCCTCGT |
| 17221 | AAATCATGTT | CGTCACGACC | TTCTTCAGAA | GGTCGAGATA | CAGGTCCGCT | TCGGCAGCTA |
| 17281 | TGACAGTCAT | TTTCCTCACT | TACGGGTAGC | AGTGCCCAGC | GGGCGGCTCG | TTCAGGACGG |
| 17341 | GGGCCGCCGG | GGCTGAATTC | CCTGTGTCCA | CACAGATGAG | GTGGATGAGG | TGGATGAGGT |
| 17401 | AGCCATCTAA | CCCCAGTGAT | CAGATTCGGG | CAAGGGTCGA | AAACGAGCCA | CGTCTTATGT |
| 17461 | CGATCCTGTC | CGGAAGCGAG | GGGCATATGG | TGCAGTGGCG | ACTGCGGCCG | ATCTGGCTGA |
| 17521 | TCCTTGCTGC | GGCGCTGACC | GTGTGCTTGC | ATGTTGGACG | TAGATCACCT | TCTCCCGATT |
| 17581 | GCATTCAGGG | TGAGGAAATC | CATGAAATCT | TCAAAAGTCG | TTCACAGCCG | TCCTGCGGAA |
| 17641 | GCGGGCGTCG | CATGGCCCGT | CGCGCGAACC | TGCCCCTTTA | CGCTCCCTGA | TCAGTACGCA |
| 17701 | GAGAAGCGCA | AGAACGAGCC | CATATGCCGG | GCTCAGGTCT | GGGACGACTC | CAGAACCTGG |
| 17761 | CTCATCACCA | AGCACGAGCA | CGTACGAGCC | CTTCTCGCCG | ACCCCCGGGT | CACCGTCGAC |
| 17821 | CCGGCCAAGC | TGCCCAGGCT | CTCCCCCTCC | GACGGCGACG | GCGGCGGCTT | CCGGTCCCTG |
| 17881 | CTGACCATGG | ACCCCCCGGA | CCACAACGCC | CTCCGCCGCA | TGCTCATATC | CGAGTTCAGC |
| 17941 | GTGCACCGGG | TCCGGGAGAT | GCGCCCGGGC | ATCGAGCGCA | CCGTGCACGG | GCTGCTGGAC |
| 18001 | GGGATCCTCG | AACGGCGGGG | GCCGGTGGAC | CTGGTGGCCG | AACTCGCGCT | GCCGATGTCC |
| 18061 | ACCCTGGTGA | TCTGTCAGCT | CCTCGGAGTG | CCCTACGAAG | ACCGCGAGTT | CTTCCAGGAA |
| 18121 | CGCAGCGAAC | AGGCCACCCG | GGTGGGCGGG | AGCCAGGAAT | CGCTGACCGC | GCTCCTGGAA |
| 18181 | CTACGGGACT | ACCTGGACCG | GTTGGTCACC | GCGAAGATCG | AGACGCCGGG | TGACGACCTG |
| 18241 | CTGTGCCGGC | TCATCGCCAG | TCGACTGCAC | ACCGGTGAGA | TGCGACACGC | CGAGATCGTG |
| 18301 | GACAACGCCG | TGCTGCTGCT | CGCCGCCGGC | CACGAGACCA | GTGCCGCCAT | GGTGGCACTG |
| 18361 | GGCATCCTGA | CACTGTTGCG | GCACCCCGGC | GCCCTGGCGG | AGTTGCGGGG | CGACGGTACG |
| 18421 | CTGATGCCGC | AGACGGTCGA | CGAACTCCTG | CGTTTCCACT | CCATCGCGGA | CGGCCTTCGA |
| 18481 | CGGGCGGTCA | CGGAGGACAT | CGAACTCGGC | GGCATCACGC | TGCGCGCCCC | AGACGGCCTC |
| 18541 | ATCGTCTCGC | TGGCCTCCGC | CAACCGCGAC | GAGAGCGCCT | TCGCCTCCCC | GGACGGCTTC |
| 18601 | GACCCGCACC | ATCCGGCGAG | CCGGCACGTC | GCCTTCGGCT | ACGGCCCCCA | CCAGTGCCTG |
| 18661 | GGCCAGAACC | TGGCCCGGCT | GGAGCTGGAG | GTCACCCTGG | GCGCGGTGGT | GGAGCGCATT |
| 18721 | CCCACGCTCA | GGCTGGCCGG | CGACGCCGAC | GCACTGCGCG | TCAAACAGGA | TTCGACCATC |
| 18781 | TTCGGGCTGC | ACGAGCTGCC | GGTCGAGTGG | TGACGGAAGG | AGGACACAGC | GTGCGGGTGA |
| 18841 | CAGTCGACCA | GAGCCGGTGC | CTGGGAGCCG | GCCAGTGCGA | GCAGCTGGCG | CCGGAGGTCT |
| 18901 | TCCGCCAGGA | CGAGGAAGGA | CTGAGCCGGG | TCCTCGTCCC | CGAGCCCGAT | CCGGCGTCAT |
| 18961 | GGCCGCGGGT | GCTCCAGACG | GTGGACCTCT | GCCCCGTACA | GGCCGTCCTC | ATCGACGAGG |
| 19021 | GCCCCGGTCC | CGCGCCGCAG | GACACCAAGT | GACCGCTGAC | CGCTGGGCCG | GCCGCACGGT |
| 19081 | GCTCGTCACG | GGAGCACTCG | GGTTCATCGG | CTCCCACTTC | GTCCGACAGC | TGGAGGCGCG |
| 19141 | CGGAGCCGAG | GTGCTCGCCC | TCTACCGCAC | CGAACGGCCG | CAATTACAGG | CCGAGTTGGC |
| 19201 | CGCGCTCGAC | CGAGTACGCC | TGATCAGGAC | GGAGCTGCGG | GACGAGTCGG | ACGTGCGAGG |
| 19261 | GGCCTTCAAG | TACCTGGCAC | CCTCCATCGA | CACCGTCGTC | CACTGCGCGG | CCATGGACGG |
| 19321 | CAACGCGCAG | TTCAAGCTGG | AGCGCTCGGC | CGAGATCCTC | GACAGCAACC | AGCGGACCAT |
| 19381 | CTCCCACCTG | CTGAACTGCG | TACGGGACTT | CGGCGTCGGC | GAGGCCGTGG | TCATGAGCTC |
| 19441 | CTCCGAGCTG | TACTGCGCGC | CGCCCACCGC | GGCGGCCCAC | GAGGACGACG | ACTTCCGCCG |
| 19501 | ATCCATGCGG | TACACGGACA | ACGGCTACGT | CCTGTCCAAG | ACCTACGGCG | AGATCCTGGC |
| 19561 | CAGGCTCCAC | CGCGAGCAGT | TCGGCACCAA | CGTCTTCCTG | GTGCGACCGG | GCAACGTCTA |
| 19621 | CGGGCCGGGA | GACGGCTACG | ACCCCTCCCG | GGGCCGGGTG | ATCCCCAGCA | TGCTGGCCAA |
| 19681 | GGCCGACGCC | GGCGAGGAGA | TAGAGATCTG | GGGGGACGGC | AGTCAGACCC | GGTCCTTCAT |
| 19741 | CCACGTCACC | GACCTGGTAC | GGGCCTCACT | GCGCCTGCTG | GAGACCGGCA | AGTACCCCGA |
| 19801 | GATGAACGTG | GCCGGCGCGG | AACAGGTCTC | CATCCTGGAG | CTCGCCCGGA | TGGTGATGGC |
| 19861 | CGTCCTGGGA | CGGCCCGAGC | GCATCCGCCT | CGACCCCGGC | CGCCCCGTCG | GCGCCCCGAG |
| 19921 | CAGACTTCTG | GATCTGACCA | GGATGTCGGA | AGTGATCGAC | TTCGAGCCCC | AGCCCCTGCG |
| 19981 | GACCGGGCTG | GAAGAGACCG | CTCGCTGGTT | CCGCCACCAC | ACGCGCTGAA | CCTCCTCTCA |
| 20041 | TACCCCCCTG | GAAGGTAACT | CGTGGTCACA | CACGCCCCGA | ACTCGCTGAT | CAGTGACATA |
| 20101 | ATCCGCGCCT | CCGGCGGGCA | TGACGCCGAC | CTCAAGGACC | TGGCCGCCCG | ACACGATCCG |
| 20161 | GCCGACATCG | TCCGCGTACT | CCTGGACGAG | ATCACCTCAC | GCTGCCCCGC | TCCCGTGAAC |
| 20221 | GACGTCCCCG | TCCTCGTCGA | GCTGGCCGTC | CGGGCGGGAG | ACCGCCTCTT | CCCCACCTAT |
| 20281 | CTGTACGTCC | TCAAGGGCGG | CCCGGTGCGC | CTCGCGGCCA | AGGACGAGGC | GTTCGTCGCC |
| 20341 | ATGCGCGTCG | AGTACGAGCT | GGGCGAACTG | GCCCGCGAGC | TGTTCGGACC | GGTGCGGGAG |
| 20401 | AACGTCACCG | GCGTCCGCGG | AACGACTCTC | TTCCCCTACG | TCGGGGACAC | GGCGTCGGAA |
| 20461 | GGCGAGGAGG | ACTCGGGTGC | CGAGCACATC | GGCACGCACT | TCCTGGCCGC | GCAGCAGGGC |
| 20521 | TCCCAGACCG | TGCTCGCCGG | CTGCCATTCC | CACAAGCCGG | ACCTCAGCGA | GCTCTCCTCG |
| 20581 | CGCTACCTCA | CCCCGAAGTG | GGGCTCGCTG | CACTGGTTCA | CCCCCCACTA | CGACCGCCAC |
| 20641 | TTCAGGAGCT | ACCGGGACCA | GCCCGTACGC | GTTCTGGAGA | TCGGCATCGG | CGGCTACAAG |
| 20701 | CACCCCGAGT | GGGGCGGCGG | CTCCCTGCGC | ATGTGGAAGC | ACTTCTTCCA | TCGCGGCGAG |
| 20761 | ATCTACGGCC | TGGACATCGT | CGACAAGTCG | CACTTCGACG | CGCCGCGCAT | CACGACCCTG |
| 20821 | CGCGGCGACC | AGAGCGACCC | CGACCACCTG | CGGTCGATCG | CCGAGAAGTA | CGGACCGTTC |
| 20881 | GACATCGTCA | TCGACGACGG | AAGCCACATC | AACGACCACA | TCCGGACCTC | GTTCCAGGCA |
| 20941 | CTGTTCCCGC | ATGTGCGGCC | CGGCGGCCTC | TACGTGATCG | AGGACCTGTG | GACCGCCTAC |
| 21001 | TGGTCCGGCT | TCGGCGGCGA | CGAGGACCCG | AAGCGGTACA | GCGGGACGAG | CCTGGGCCTG |
| 21061 | CTCAAGTCCC | TCGTCGACTC | GATCCAGCAC | GAGGAACTGC | CGGAGGCCGG | CGACCACCGT |
| 21121 | CCCAGTTACG | CGGACCAGCA | CGTGGTCGGC | ATGCACCTCT | ACCACAACCT | GGCGTTCATC |
| 21181 | GAGAAGGGCA | CCAACGCCGA | GGGCGGCATC | CCCCCGTGGA | TCCCACGCGA | CTTCGAGACC |
| 21241 | CTCGTCGCCG | TCTCCTCCGG | GGGCCACGCA | TGAGGAGCCG | TCGGCACCAG | CCACCCGAAC |
| 21301 | ACACCCGACC | GGACCGCAGG | AGGCCCGCAT | GCGCGTGACC | CTGCTGAGCG | TCGGATCCCG |
| 21361 | AGGCGACGTC | CAGCCGTTCG | TCGCCCTCGG | CATCGGCCTC | AAGGCCCGCG | GCCACGACGT |
| 21421 | CACCCTGGCC | GCCCCCGCCA | CGCTGCGGCC | ACTGGTCGAG | CGCGCGGGAC | TGACGTACAG |
| 21481 | GCTGTCCCCC | GGGGATCCCG | ACGGATTCTT | CACCATGCCC | GAGGTCGTCG | AAGCGCTGCG |
| 21541 | GCGCGGCCCC | TCGTTCAAGA | ACATGCTCGC | GGGGATGCCC | GAGGCGCCCG | AGAGCTACAC |
| 21601 | ACAGCAGGTC | GTCGACGCGA | TCCACGACGC | CGCCGAGGGC | GCCGACCTCA | TAGTGAACGC |
| 21661 | GCCCCTCACC | CTGGCCGCCG | CGTACGGGCA | CCCGCCCGCC | CCGTGGGCCT | CGGTGTCCTG |
| 21721 | GTGGCCCAAC | AGCATGACCT | CGGCCTTCCC | GGCCGTCGAA | TCCGGGCAGC | GCCACCTCGG |
| 21781 | ACCGCTGACG | TCCCTGTACA | ACCGCTACAC | CCATCGCAGG | GCGGCACGCG | ACGAGTGGGA |
| 21841 | GTGGCGACGC | CCCGAGATCG | ACGGCTACCG | CCGACGCCTC | GGCCTCCGGC | CCTTCGGCGA |
| 21901 | CGAGTCCCCG | TTCCTCCGAC | TGGGGCACGA | CCGCCCGTAC | CTGTTCCCCT | TCAGCCCCAG |
| 21961 | CGTGCTGCCC | AAGCCGCGGG | ACTGGCCGCG | CCAGAGCCAC | GTCACCGGCT | ACTGGTTCTG |
| 22021 | GGACCAGCAC | GGGGAGCCGC | CCGCCGAGCT | GGAGTCGTTC | CTGGAGGACG | GGGAGCCCCC |
| 22081 | GGTGGCGCTC | ACCTTCGGCA | GCACCTGGTC | ACTCCACCGG | CAGGAGGAGG | CCCTCCAGGC |
| 22141 | CGCCCTCGAC | GCCGTCCGTG | GCGTCGGACG | CCGACTGGTC | ATGGTCGACG | GACCGGACAG |
| 22201 | CGACCTGCCC | GACGACGTGC | TCCGCCTGCA | CCAGGTGGAC | TACGCCACCC | TCTTCCCCAG |
| 22261 | GATGGCCGCG | GTGATCCACC | ACGGCGGCGC | CGGCACGACC | GCCGAGGTCC | TCCGGGCCGG |
| 22321 | AGTGCCCCAG | GTCATCGTGC | CGGTCTTCGC | CGATCACCCC | TTCTGGGCGG | CTCGACTGTC |
| 22381 | CAGAACAGGC | GTCGCCGCCC | GGCCGGTCCC | CTTCGCCCGC | TTCAGCCGAG | AGGCACTGGC |
| 22441 | GCAGAGCGTC | CGCCAGGCGG | TCACCGATCC | CGCGATGGCG | GGCCGGGCCA | GGCGACTGGG |
| 22501 | CGAACGGGTC | TCCAAGGAAC | GGGGAGTGGA | CACCGCCTGC | GACATCCTCG | AGAAGTGGGC |
| 22561 | GGAGACGGCA | CGCGCCACGG | CCTGACACGG | CCACCGGCGG | GCGGGCCCGG | AAGCCGCACG |
| 22621 | CCCGCCGGCC | GACGGGTCCC | GGGACCGCGC | CGCTACGCCG | ACAACCGGTA | GGCGGAGAGC |
| 22681 | CGCACGGAGA | GCGTGACCCG | AGTCGGCGCC | GGCAGCCGCT | GGATCGTCTC | CAGATCCCGC |
| 22741 | TGCGCGTCAC | GGTGCCAGGC | ATTGGGCCCC | ATGCCGACCA | GGTGCGCCAG | AGCCTGGTGG |
| 22801 | TCGAGAGCCA | TGGTCCGGGT | CAACTCCTCC | GTGGCGACGG | CCGAGAAGTG | CGGAGCGAGC |
| 22861 | TGCTCCGCGA | GACGCGACTC | CTTGCCTTCG | TCCACCTGCA | GCAGGCCGAG | GGCGCCGATC |
| 22921 | ACCTCCCGCA | GGTGATCGGG | CAGAGGAGTG | ACAACCAGGA | GAACGCCACG | GGGATGGAGA |
| 22981 | ACGCGATGCA | GTTCAGGACC | GTTGCGGGGA | GCGAACGTGT | TGATCACCAT | GCCGGCTGCG |
| 23041 | GCATCCCGCA | GCGGAAGCGT | CTGCCAGGCG | TCGGTCACCG | CGGCCGCGAT | CCGCGGATGC |
| 23101 | GCCTTCGCGG | CACGCCGCAC | GGCGTACTTG | GAGATGTCCA | GCAGCAGGCC | CTGGGCATCG |
| 23161 | GGGAACGCTT | CCATGACCCC | GGCGTGATAG | TGGCCCGTCC | CCCCACCGAT | GTCGACCACA |
| 23221 | CAGCCGGGCA | CGGCCGGATC | GGCCGTCCGC | CGCGCCAGAT | CGACCAGCGC | ATCCATCACG |
| 23281 | GGGTCGTAGT | GACCCGCCGA | CAGGAATGCG | TCCCGGGCCT | CCACCATTTC | CTTGGTGTCG |
| 23341 | GCCCGCAGCT | TCGTCGCCCT | GGGAAGCAGA | TTCACATAAC | CCTGCTTCGC | GATGTCGAAG |
| 23401 | GAGTGTCCGG | CGGGGCAGAA | AAGTGCGCGG | TCGCCCTGAG | CCAGCGAAGC | ACCGCAGTGC |
| 23461 | GGGCAGGCGA | GGTAGCGCAC | GATCCTGTTG | AGCATGGGGC | ACCGTTCCTT | CGGGCGAGTC |
| 23521 | GGCAGACCGG | CCAACCCTAA | CGCAGGCGCC | CGGCCGCCCA | CCGCCCGGCG | CAGGGCCGAC |
| 23581 | GAACCACCGT | GACGTGCACC | AGCCGCGCGT | GAGAACTCCT | CATGCGCGCA | CCCTACGGAA |
| 23641 | ATCGGCAGGT | CAACCGGCGA | TTCCTGCGGG | AATTCCGAGC | GAAACGGCCT | CACTGTGTTT |
| 23701 | CCCCGCTGCA | TTTCCTCGCT | GAATTCAGCG | AATCCCGGCA | GACGACCGGC | TCTGCCGGCG |
| 23761 | TGACAGCCCC | TATCGATCGA | CCAGGAGTTT | CGATGGCCCC | GAAGAGTGGT | GCGCAGCGTT |
| 23821 | CGAGCGACAT | AGCCGTCGTC | GGCATGTCCT | GCCGCCTTCC | GGGGGCACCG | GGCATCGATG |
| 23881 | AGTTCTGGCA | TCTGCTGACC | ACCGGAGGCA | GCGCGATCGA | GCGTCGCGCC | GACGGCACCT |
| 23941 | GGCGCGGCTC | CCTGGACGGA | GCCGCCGACT | TCGACGCCGC | CTTCTTCGAC | ATGACCCCCC |
| 24001 | GCCAGGCCGC | CGCCGCCGAC | CCGCAGCAAC | GACTCATGCT | GGAACTCGGC | TGGACGGCCC |
| 24061 | TGGAGAACGC | CGGGATCGTC | CCCGGCAGCC | TCGCCGGCAC | GGACACCGGC | GTCTTCGTCG |
| 24121 | GCATCGCGGC | CGACGACTAC | GCGGCACTCC | TGCACCGGTC | CGCCACCCCC | GTCAGCGGGC |
| 24181 | ACACCGCGAC | GGGCCTGAGC | CGGGGCATGG | CCGCCAACCG | CCTCTCCTAC | CTCCTGGGCC |
| 24241 | TGCGCGGTCC | CAGCCTCGCG | GTGGACAGCG | CGCAGTCCTC | CTCGCTCGTC | GCGGTCCACC |
| 24301 | TGGCCTGCGA | GAGCCTGCGC | CGCGGCGAGT | CCGACCTCGC | GATCGTCGGC | GGCGTCAGCC |
| 24361 | TGATCCTCGC | CGAGGACAGC | ACGGCGGGCA | TGGAGCTCAT | GGGCGCGCTC | TCGCCGGACG |
| 24421 | GCCGCTGCCA | CACCTTCGAC | GCACGCGCCA | ACGGCTACGT | ACGCGGTGAG | GGCGGAGCCT |
| 24481 | GCGTCGTCCT | CAAGCCCCTG | GAGCGGGCAC | TGGCCGACGG | GGACCGCGTC | CACTGCGTCG |
| 24541 | TCCGAGGAAG | CGCGGTCAAC | AACGACGGCG | GCGGCTCCAC | CCTGACCACC | CCCCACCGCG |
| 24601 | AGGCCCAGGC | CGCCGTCCTG | CGGGCGGCGT | ACGAACGGGC | CGGGGTCGGC | CCGGACCAGG |
| 24661 | TGTCCTACGT | CGAACTGCAC | GGTACGGGGA | CGCCGGTCGG | CGACCCCGTC | GAGGCGGCGG |
| 24721 | CTCTCGGCGC | GGTCCTCGGC | ACGGCCCACG | GCCGTAACGC | CCCGCTGTCC | GTGGGATCGG |
| 24781 | TCAAGACGAA | CGTCGGCCAC | CTGGAGGCGG | CCGCGGGCCT | CGTGGGATTC | GTGAAGGCAG |
| 24841 | CCCTGTGCGT | CCGCGAGGGC | GTGGTGCCGC | CGAGCCTGAA | CCACGCGACG | CCCAACCCTG |
| 24901 | CCATCCCCAT | GGACCGGCTA | AACCTGCGCG | TACCCACGCG | ACTGGAGCCC | TGGCCGCACC |
| 24961 | CGGACGACCG | AGCGACCGGC | CGGCTGCGAC | TGGCCGGCGT | CTCGTCCTTC | GGCATGGGTG |
| 25021 | GGACGAACGC | GCATGTGGTG | GTGGAGGAGG | CGCCGCTTCC | GGAGGCCGGG | GAGCCGGTCG |
| 25081 | GGGCCGGTGT | GCCTTTGGCT | GTGGTGCCGG | TGGTGGTGTC | GGGTCGTTCT | GCGGGTGCGG |
| 25141 | TGGCTGAACT | GGCCTCCCGC | TTGAACGAGT | CGGTTCGTTC | GGATCGGTTG | GTGGATGTGG |
| 25201 | GGTTGTCGTC | GGTGGTGTCG | CGGTCGGTGT | TCGAGCATCG | GTCCGTGGTT | CTGGCGGGGG |
| 25261 | ACTCTGCCGA | GCTGAGTGCC | GGTTTGGATG | CTCTGGCCGC | TGACGGAGTG | TCTCCTGTCC |
| 25321 | TGGTGTCGGG | TGTGGCGTCG | GTCGGGGGTG | GCCGGTCGGT | CTTCGTGTTC | CCGGGTGCGG |
| 25381 | GGGTGAAGTG | GGCGGGGATG | GCGCTCGGGT | TGTGGGCGGA | GTCTGCTGTG | TTTGCGGAGT |
| 25441 | CGATGGCGCG | GTGTGAGGCG | GCGTTCGCCG | GGTTGGTGGA | GTGGCGTCTG | GCGGATGTGC |
| 25501 | TGGGTGATGG | GGCTGCGTTG | GAGCGTGAGG | ACGTGGTGCA | GCCGGCGTCG | TTCGCGGTGA |
| 25561 | TGGTGTCGTT | GGCGGCGTTG | TGGCGGTCGT | TGGGTGTGGT | GCCGGATGCG | GTGGTGGGGC |
| 25621 | ATTCGCAGGG | GGAGATCGCT | GCTGCGGTGG | TGGCGGGTGG | TCTGTCGTTG | GAGGACGGCG |
| 25681 | CGCGTGTGGT | GGTGTTGCGT | GCGCGGGTGG | CTGAGGAGGT | TTTGTCGGGT | GGGGGGATTG |
| 25741 | CTTCGGTGCG | TCTTTCGCGG | GCCGAGGTGG | AGGAGCGGTT | GGCGGGTGGG | GGTGGTGGGT |
| 25801 | TGAGTGTGGC | GGTGGTGAAT | GCGCCGTCGT | CGACGGTGGT | GGCGGGTGAG | TTGGGGGATT |
| 25861 | TGGATCGGTT | TGTTGCGGCG | TGTGAGGCGG | AGGGGGTGCG | GGCGCGTCGG | CTGGAGTTCG |
| 25921 | GGTATGCGTC | GCATTCGAGG | TTCGTGGAGC | CGGTGCGTGA | GCGGTTGTTG | GAGGGGTTGG |
| 25981 | CCGATGTCCG | TCCGGTGAGG | GGGCGGATTC | CGTTCTATTC | GACGGTGGAG | GCTGCGGAGT |
| 26041 | TCGATACGGC | TGGTCTGGAT | GCGGAGTACT | GGTTCGGGAA | TCTGCGTCGG | CCGGTTCGCT |
| 26101 | TCCAGGAGAC | GGTCGAGCGG | CTGTTGGCGG | ATGGTTTCCG | GGTGTTCGTG | GAGTGCGGCG |
| 26161 | CGCATCCGGT | GCTGACCGGG | GCGGTGCAGG | AGACCGCGGA | GACTGCGGGC | CGGGAGATCT |
| 26221 | GCTCCGTCGG | ATCCCTGCGT | CGTGACGAGG | GTGGACTGCG | TCGCTTCCTG | ACCTCTGCGG |
| 26281 | CGGAGGCGTT | CGTCCAGGGG | GTGGAGGTGT | CCTGGCCGGT | GCTGTTCGAC | GGCACCGGCG |
| 26341 | CCCGGACGGT | CGACCTGCCC | ACCTACCCCT | TCCAACGCCG | GCACCACTGG | GCACCCGACG |
| 26401 | GCTCCGCGTC | GGCGGCGCCC | ACACGGGACA | TCCGACCGGA | CGAGACCGCC | GCGGTTCCAG |
| 26461 | CGGACACGAT | GGACCTCGCG | GGACAACTTC | GCGCGGACGT | GGCGTCGTTG | CCCACCACCG |
| 26521 | AGCAGATCGC | GCGGTTGCTC | GACCAGGTAC | GCGACGGTGT | CGCCACGGTC | CTCGGACTGG |
| 26581 | ACGCCCGGGA | CGAGGTGCGC | GCGGAGGCGA | CGTTCAAGGA | ACTGGGCGTC | GAATCGCTGA |
| 26641 | CCGGCGTCGA | GCTGAAGAAC | CACCTCCGTG | CCCGGACCGG | ACTGCACGTC | CCCACCTCGC |
| 26701 | TGATCTACGA | CTGCCCGACT | CCCCTCGCCG | CCGCTCACTA | CCTCCGCGAC | GAGCTCTTGG |
| 26761 | GCCGACCCGC | GGAGCAGGCC | GTCGTCCCCG | CCGGCATCCC | GGTCGACGAA | CCGATCGCGA |
| 26821 | TCGTGGGGAT | GGGGTGCCGT | CTGCCGGGTG | GGGTGTCGTC | GCCGGAGGGG | TTGTGGGATC |
| 26881 | TGGTGGCGTC | GGGGGTGGAT | GCGGTTTCTC | CGTTCCCCAC | GGATCGGGGT | TGGGATGTGG |
| 26941 | GGGGTCTGTT | CGATCCGGAG | CCGGGGGTGC | CGGGGCGTTC | GTATGTGCGT | GAGGGCGGGT |
| 27001 | TCCTTCATGA | GGCGGGGGAG | TTCGACGCGG | GGTTCTTCGG | TATCTCTCCG | CGTGAGGCGT |
| 27061 | TGGCGATGGA | TCCGCAGCAG | CGGTTGTTGC | TGGAGACGTC | GTGGGAGGCG | TTGGAGCGGG |
| 27121 | CGGGCATCGA | TCCGCACACG | CTTCGCGGTT | CACGGACCGG | CGTCTACGCC | GGAGTGATGG |
| 27181 | CCCAGGAATA | CGGCCCCCGA | CTCCACGAAG | GCGCAGACGG | ATACGAGGGC | TATCTGCTGA |
| 27241 | CCGGATCCTC | CAGCAGTGTC | GCCTCGGGTC | GTATCTCGTA | CGTGCTGGGT | CTGGAAGGGC |
| 27301 | CGGCGGTGAC | GGTGGACACC | GCGTGCTCGT | CGTCGCTGGT | CGCGCTGCAC | CTGGCCGTGC |
| 27361 | GGGCGCTGCG | CAGCGGTGAG | TGCGACCTCG | CCCTCGCCGG | CGGCGCGACC | GTCATGGCCG |
| 27421 | AACCCGGCAT | GTTCGTGGAG | TTCTCACGGC | AGCGCGGGCT | GTCTGCCCAC | GGACGGTGCA |
| 27481 | AGGCGTACTC | GGACTCGGCC | GATGGCACGG | GCTGGGCCGA | GGGGGCGGGT | GTGCTGCTCG |
| 27541 | TCGAGCGGTT | GTCGGATGCG | GTACGTCATG | GGCGTCGGGT | GCTGGCGGTC | GTGCGTGGTT |
| 27601 | CCGCGGTCAA | CCAGGACGGT | GCGAGCAACG | GACTGACGGC | GCCGAACGGG | CGGTCCCAGT |
| 27661 | CGCGTTTGAT | CCGTCAGGCG | TTGGCGGATG | CGCGGTTGGG | TGTGGCTGAT | GTGGATGTGG |
| 27721 | TGGAGGGCCA | CGGCACGGGG | ACGCGTCTGG | GTGATCCGAT | CGAGGCGCAG | GCGTTGTTGG |
| 27781 | CGACGTATGG | GCAGCGGGAT | GCGGGTCGGC | CTCTGCGGCT | TGGTTCGCTG | AAGTCGAACG |
| 27841 | TGGGGCATAC | GCAGGCGGCT | GCCGGTGTGG | CGGGCGTGAT | CAAGATGGTC | ATGGCGATGC |
| 27901 | GGCACGGTGT | CCTGCCGAAG | ACGCTGCACG | TGGATGAGCC | GACGGCGGAG | GTGGACTGGT |
| 27961 | CGGCCGGCGC | GGTGTCCCTG | CTGAGGGAGC | AGGAGGCGTG | GCCGCGTGGC | GAGCGTGTGC |
| 28021 | GGCGGGCCGG | AGTCTCCTCG | TTCGGCGTGA | GTGGGACGAA | CGCGCATGTG | GTGGTCGAGG |
| 28081 | AGGCGCCGGT | TCCGGAGGAC | GGGGAGGCGA | TCGAGGGCGG | TGCGCCTTTG | GCTGTGGTGC |
| 28141 | CGGTGGTGGT | GTCGGGTCGT | TCTGCGGGTG | CGGTGGCGGA | GCTGGCGGGC | CGGGTCAGCG |
| 28201 | AGGTCGCTGG | GTCTGGTCGG | TTGGTGGATG | TGGGGTTGTC | GTCGGTGGTG | TCGCGGTCGG |
| 28261 | TGTTCGAGCA | CCGGTCCGTG | GTGCTGGCGG | GGGACTCTGC | CGAGCTGAAT | GCCGGTCTGG |
| 28321 | ATGCTCTGGC | CGCTGACGGA | GTGTCTCCTG | TCCTGGTGTC | GGGTGTGGCG | TCGGGTGAGG |
| 28381 | GTGGCCGGTC | GGTGTTCGTG | TTCCCTGGTC | AGGGGACGCA | GTGGGCGGGG | ATGGCGCTCG |
| 28441 | GGTTGTGGGC | GGAGTCGGCG | GTGTTCGCGG | AGTCGATGGC | GCGGTGTGAG | GCGGCGTTCG |
| 28501 | CCGGGTTGGT | GGAGTGGCGT | CTGGCGGATG | TGCTGGGTGA | CGGGTCTGCG | TTGGAGCGGG |
| 28561 | TCGATGTGGT | GCAGCCGGCG | TCGTTCGCGG | TGATGGTGTC | GCTGGCGGAG | TTGTGGCGGT |
| 28621 | CGTTGGGTGT | GGTGCCGGAT | GCGGTGGTGG | GGCATTCGCA | GGGGGAGATC | GCTGCTGCGG |
| 28681 | TGGTGGCGGG | TGGTCTCTCG | CTGGAGGATG | GCGCGCGTGT | GGTGGTGTTG | CGTGCGCGGT |
| 28741 | TGATAGGCCG | TGAGCTGGCC | GGGCGCGGTG | GGATGGCGTC | GGTGGCGCTG | CCCCTCGCGG |
| 28801 | TGGTGGAGGA | GCGTCTGGCG | GGGTGGGCGG | GGCGTCTGGG | TGTGGCGGTG | GTCAACGGAC |
| 28861 | CCTCCGCCAC | GGTCGTCGCG | GGTGATGTGG | ATGCGGTGGG | GGAGTTTGTG | ACCGCGTGCG |
| 28921 | AGGTGGAGGG | GGTTCGGGCG | CGTGTTCTGC | CGGTGGACTA | CGCCTCGCAC | TCGGCGCACG |
| 28981 | TGGAGGACCT | GAAAGCCGAG | CTTGAACAGA | TTCTGGCCGG | CATCGGCCCG | GTGACCGGTG |
| 29041 | GCATCCCGTT | CTATTCGACG | TCCGAAGCCG | CGCAGATCGA | CACGGCTGGT | CTGGACGCGG |
| 29101 | GGTACTGGTT | CGGGAATCTG | CGTCGGCCGG | TGCGGTTCCA | GGAGACGGTC | GAGCGGTTGC |
| 29161 | TGGCGGATGG | TTTCCGGGTG | TTCGTGGAGT | GTGGCGCGCA | TCCGGTGCTG | ACGGGGGCGG |
| 29221 | TGCAGGAGAC | CGCGGAATCC | ACCGGTCGCC | AGGTGTGTGC | GGTCGGATCC | CTGCGTCGTG |
| 29281 | ACGAGGGAGG | TCTGCGCCGC | TTCCTGACCT | CTGCGGCGGA | GGCGTTCGTC | CAGGGGGTCG |
| 29341 | GGGTGTCCTG | GCCGGCACTG | TTCGACGGCA | CCGGCGCCCG | CACGGTCGAC | CTGCCCACCT |
| 29401 | ATCCCTTCCA | GCGTCGGCGT | TACTGGCTGG | AGTCACGTCC | TCCTGCGGCG | GTTGTTCCGT |
| 29461 | CGGGGGTCCA | GGACGGATTG | TCGTATGAGG | TGGTGTGGAA | GAGCCTGCCG | GTACGGGAGT |
| 29521 | CGTCGCGTCT | TGACGGCCGG | TGGCTGCTCG | TCGTGCCCGA | AACCCTGGAC | GCCGACGGCA |
| 29581 | CGCGGATCGC | CCACGACCTC | CAGCACGCCC | TCACCACCCA | CGGCGCCACG | GTCTCCCGTG |
| 29641 | TGTCGGTCGA | CGTGACGACG | ATCGACCGCG | CCGACCTGTC | GGCGCGGCTC | ACCACGAGCG |
| 29701 | CGGCCGAAGA | CCAGGAACCG | CTTGGGCGAG | TGGTGTCCCT | CCTGGGGTGG | GCCGAGGGAG |
| 29761 | TACGGGCCCA | TGGCCCGAAC | GTACCGACTT | CCGTCGCCGC | CTCCCTGGCA | CTCGTGCAGG |
| 29821 | CTGTCGGCGA | TGCCGGGTTC | GGTGTTCCGG | TGTGGGCGGT | GACGCGGGGT | GCGGTGTCCG |
| 29881 | TGGTGCCTGG | TGAGGTGCCG | GAGACGGCGG | GTGCGCAACT | GTGGGCGCTC | GGCCGGGTCG |
| 29941 | CCGGTCTCGA | ACTTCCCGAC | CGTTGGGGTG | GTCTGATCGA | TCTTCCGGCG | GATGCCGATG |
| 30001 | CGCGTACGGC | GGGGCTTGCG | GTGCGGGCCC | TGGCCGCCGG | GATCGCCGAT | GGTGAGGACC |
| 30061 | AGGTGGCGGT | GCGCCCCTCG | GGTGCCTACG | GCCGGCGTGT | AGTTCAGGCA | GCCCACCGGG |
| 30121 | AGCCGTCGGG | AGCGAAGACG | GAGTGGCGAC | CGCGTGGCAC | CGTGCTCGTC | ACCGGGGGAA |
| 30181 | TGGGCGCCAT | CGGCACTCGG | GTGGCCCGCT | GGCTGGCCCG | GAACGGAGCC | GAACACCTGG |
| 30241 | TGCTGACCGG | CCGCCGCGGT | GCCGGGACCC | CCGGCGCGGA | CGAGCTGGCG | GGAGAGCTGA |
| 30301 | GGGCGTCCGG | AGTCCAGGTC | ACGCTCGCCG | CCTGCGACGT | GTCCGATCGT | GCCGCGCTGG |
| 30361 | CCGCGCTGCT | CGACGCGCAT | CCGCCGACCG | CCGTCTTCCA | CACGGCCGGT | GTACTGAACG |
| 30421 | ACGGAACGGT | CGACACGCTC | ACTCCCGCTC | ACCTGGACGG | GGTCCTGAGC | CCCAAGGCGA |
| 30481 | CGGCCGCCGT | TCACCTGCAC | GAGCTCACCG | CCCACCTGGA | CCTGGACGCC | TTCGTCCTCT |
| 30541 | TCGCCTCCGT | CACCGGCGTA | TGGGGTAACG | GCGGCCAGGC | CGGGTACGCC | ATGGCCAACG |
| 30601 | CGGCTCTGGA | CGCGCTCGCC | GAGCAGCGCC | GTGCCGGCGG | ACTTGCGGCG | ACCTCCATCA |
| 30661 | GTTGGGGCCT | CTGGGGTGGC | GGCGGCATGG | CCGAGGGTGA | CGGCGAGGTG | AGCCTCAACC |
| 30721 | GTCGTGGAAT | CCGCGCTCTT | GAGCCCGCCA | CCGGCATCGA | GGCGCTGCAG | CGGACGCTCG |
| 30781 | ACCAGGGCGC | CACCTGCCGC | ACCGTCGTCG | ACGTGGACTG | GGGTCAGTTC | GCTCCTCGTA |
| 30841 | CGGCGGCGCT | GCGGCGCGGG | CGGCTCTTCG | CGGATCTGCC | CGAGGTGCGG | CGTGTCCTGG |
| 30901 | AGTCCGAGGG | GGTTGCACGG | GAGGACGCCG | GAACCGTCGA | GCCCGGCGCC | GTGCTCGCCG |
| 30961 | AGCGCCTCGC | ATCGCGCTCC | GAGGCCGAAC | AGCGACGCAT | GCTCGTCGAG | TTGGTACGAG |
| 31021 | CCGAAGCGGC | TGCCGTCCTG | CGTCACGACA | CGACGGACCT | CCTGGCGCCG | CGCAGGTCGT |
| 31081 | TCAAGGACGC | CGGGTTCGAC | TCCTTGACCG | CGCTGGAGCT | CCGTAACCGG | CTGAACACCG |
| 31141 | CCACCGGTGT | CGTCCTTCCC | GTCACCGTCG | TCTTCGACCA | CCCGAACCCC | GGTGCACTGG |
| 31201 | CGGACTTCCT | GTACGGCGAA | GCACTGGGCC | TGTCCGCGGC | CAGGTCTTCC | GCGAGCGATA |
| 31261 | CGGCCGACAC | GACCCGCCCG | GCCGCCGCCC | CCGAAGAGCC | GATCGCGATC | GTCGGAATGG |
| 31321 | CCTGCCGCTA | CCCGGGCGAG | GCCCGTTCCC | CCGAGGPACT | GTGGAAGTTG | CTCATCGACG |
| 31381 | AACGGGACGT | CATCGGCCCC | ATGCCCACGG | ATCGGGGTTG | GGATGTGGGG | GGTCTGTTCG |
| 31441 | ATCCGGAGCC | GGGGGTGCCG | GGGCGTTCGT | ATGTGCGTGA | GGGCGGGTTC | CTTCATGAGG |
| 31501 | CGGGGGAGTT | CGACGCGGGG | TTCTTCGGTA | TTTCTCCGCG | TGAGGCGTTG | GCGATGGATC |
| 31561 | CGCAGCAGCG | GTTGTTGCTG | GAGACGTCGT | GGGAGGCGTT | GGAGCGGGCG | GGCATCGATC |
| 31621 | CGCACACGCT | CCGCGGCTCA | CAGACCGGCG | TCTACGCGGG | GATGTTCCAC | CAGGAGTACG |
| 31681 | CGACCCGGCT | GCACGAGGCA | CCCGTGGAGT | TCGAAGGCCA | CTTGCTGACG | GGGACGTCCG |
| 31741 | GGAGTGTGGC | TTCGGGTCGT | ATCTCGTATG | TGCTGGGTCT | GGAGGGGCCG | GCGGTGACGG |
| 31801 | TGGACACGGC | GTGTTCGTCG | TCGTTGGTGG | CGCTGCATCT | GGCGGTGCGG | GCGTTGCGGA |
| 31861 | GTGGTGAGTG | TGACCTTGCT | CTTGCGGGTG | GGGTGACGGT | GATGGCGGAG | CCGGGTGTGT |
| 31921 | TCGTGGAGTT | CTCGCGGCAG | CGGGGGTTGG | CTGCGGACGG | GCGGTGCAAG | GCGTTCGCGG |
| 31981 | CTGCTGCCGA | TGGCACGGGC | TGGGCCGAGG | GTGTGGGCGT | GCTGGCGGTG | GAGCGGTTGT |
| 32041 | CGGATGCGGT | GCGTCATGGG | CGTCGGGTGC | TGGCGGTGGT | GCGTGGCTCG | GCGGTGAATC |
| 32101 | AGGACGGTGC | GAGCAATGGG | TTGACGGCTC | CGAACGGTCC | GTCGCAGCAG | CGGGTGATTC |
| 32161 | GTCAGGCGTT | GGCGGATGCG | CGGTTGGGTG | TGGCTGATGT | GGATGTGGTG | GAGGGGCATG |
| 32221 | GGACGGGGAC | GCGTCTGGGT | GATCCGATCG | AGGCGCAGGC | GTTGTTGGCG | ACGTATGGGC |
| 32281 | AGCGGGATGC | GGGTCGGCCT | CTGCGGCTTG | GTTCGCTGAA | GTCGAATGTG | GGGCATACGC |
| 32341 | AGGCGGCTGC | CGGTGTGGCG | GGCGTGATCA | AGATGGTCAT | GGCGATGCGG | CACGGGGTCC |
| 32401 | TGCCGAAGAC | GCTGCACGTC | GATGAGGTCT | CTCCGCACGT | CGACTGGTCG | GCGGGCGCGG |
| 32461 | TGTCCCTGCT | GACGGAGCAG | GAGCCGTGGC | CCCGTGGTGA | GCGCGTACGG | CGGGCTGGTG |
| 32521 | TCTCCGCGTT | CGGTGTGAGT | GGGACGAACG | CGCATGTGGT | GGTGGAGGAG | GCACCTGCTT |
| 32581 | CGGAGGCGCC | GGTCGCGGTG | GAGCCGGTGG | AGCCGGGGGC | TGTGGGGCTT | CTTCCGGTGG |
| 32641 | TGCCCGTGGT | GGTGTCGGGT | CGTTCTGCGG | GTGCGGTGGC | TGAACTGGCC | TCCCGCTTGA |
| 32701 | ACGAGTCGGT | TCGTTCGGAT | CGGTTGGTGG | ATGTGGGGTT | GTCGTCGGTG | GTGTCGCGGT |
| 32761 | CGGTGTTCGA | GCATCGGTCC | GTGGTTCTGG | CGGGGGACTC | TGCCGAGCTG | AGTGCCGGTT |
| 32821 | TGGATGCTCT | GGCCGCTGAC | GGAGTGTCTC | CTGTCCTGGT | GTCGGGTGTG | GCGTCGGTCG |
| 32881 | GGGGTGGCCG | GTCGGTGTTC | GTGTTCCCGG | GTGCGGGGGT | GAAGTGGGCG | GGGATGGCGC |
| 32941 | TCGGGTTGTG | GGCGGAGTCT | GCTGTGTTTG | CGGAGTCGAT | GGCGCGGTGT | GAGGCGGCGT |
| 33001 | TCGCCGGGTT | GGTGGAGTGG | CGTCTGGCGG | ATGTGCTGGG | TGATGGGGCT | GCGTTGGAGC |
| 33061 | GTGAGGACGT | GGTGCAGCCG | GCGTCGTTCG | CGGTGATGGT | GTCGTTGGCG | GCGTTGTGGC |
| 33121 | GGTCGTTGGG | TGTGGTGCCG | GATGCGGTGG | TGGGGCATTC | GCAGGGGGAG | ATCGCTGCTG |
| 33181 | CGGTGGTGGC | GGGTGGTCTG | TCGTTGGAGG | ACGGGGCGCG | TGTGGTGGTG | TTGCGGGCGC |
| 33241 | GGGTGGCTGA | GGAGGTTTTG | TCGGGTGGGG | GGATTGCTTC | GGTGCGTCTT | TCGCGGGCCG |
| 33301 | AGGTGGAGGA | GCGGTTGGCG | GGTGGGGGTG | GTGGGTTGAG | TGTGGCGGTG | GTGAATGCGC |
| 33361 | CGTCGTCGAC | GGTGGTGGCG | GGTGAGTTGG | GGGAGTTGGA | TCGGTTTGTT | GCGGCGTGTG |
| 33421 | AGGCGGAGGG | GGTGCGGGCG | CGTCGGCTGG | AGTTCGGGTA | TGCGTCGCAT | TCGAGGTTCG |
| 33481 | TGGAGCCGGT | GCGTGAGCGG | TTGTTGGAGG | GGTTGGCCGA | TGTCCGTCCG | GTGAGGGGGC |
| 33541 | GGATTCCGTT | CTATTCGACG | GTGGAGGCTG | GGGAGTTCGA | TACGGCTGGT | CTGGATGCGG |
| 33601 | AGTACTGGTT | CGGGAATCTG | CGTCGGCCGG | TTCGGTTCCA | GGAGACGGTC | GAGCGGTTGT |
| 33661 | TGGCGGATGG | TTTCCGGGTG | TTCGTGGAGT | GTGGTGCGCA | TCCGGTGCTG | ACCGGGGCGG |
| 33721 | TGCAGGAGAC | CGCGGAGACT | GCGGGCCGGG | AGGTGTGTGC | GGTTGGTTCG | CTGCGTCGTG |
| 33781 | ACGAGGGAGG | TCTCCGTCGC | TTCCTGACCT | CTGCGGCGGA | GGCGTTCGTC | CAGGGGGTGG |
| 33841 | AGGTGTCCTG | GCCGGTGCTG | TTCGACGGCA | CCGGCGCCCG | CACGGTCGAC | CTGCCCACCT |
| 33901 | ACCCCTTCCA | ACGCCGCCAC | TACTGGGCAC | AGTCCTCGCC | CGCCGGCGCC | GGCAGCTCTG |
| 33961 | CTGCGGCCCG | CTTCGGTATG | ACCTGGGAGG | AGCATCCGCT | CCTCGGCGGC | GCGCTGCCGC |
| 34021 | TCGCGGACTC | CGGCGAGCTG | CTGCTCGTCG | GGAGGATCTC | CCCGGCCTCC | CACTCCTGGA |
| 34081 | TCGCCGACCA | CACCGTGGCC | GGGACCGCCC | TGCTGCCCGG | GACGGCCTTC | GTCGACATGG |
| 34141 | CACTGCACGC | CGCCGCGGTC | GCGGGCTGTG | CGGGTGTGGA | GGAGCTGAGC | ATCGAGGCCC |
| 34201 | CGCTGCCGGT | GCACGGCGGG | ATCCGGCTCC | AGGTGGTGAT | CGACGAGCCC | GACTCCTCCG |
| 34261 | CGCGGCGCCG | CGTGAGCGTG | TTCGCGAGGC | CGGAAGAGGA | AGACGGGGAC | GCCGGCCGCT |
| 34321 | GGACCCGACA | CGCCACCGGC | GTGCTGACCC | CCGACGTCGC | CCCCGAGCCG | GGCCGGCCGC |
| 34381 | AGTGGTGCCG | GCAGGCCTGG | CCGCCGAGCG | GCTCCGTCCG | GGTGGAGGCG | TCGGAGCTCT |
| 34441 | ACGACCGGTT | CTCCGCGCTG | GGATACGACT | ACGGCGAGGT | CTTCGCCGGG | GTCGAGGCCG |
| 34501 | TCTGGCTGCG | CGAGGGCGAG | GCCTTCGCCG | AGGTCCGCCT | GCCCACGGGC | GCGGCGCCCG |
| 34561 | ACGCCGAGCG | GTTCGGGGTG | CACCCCGGCC | TCCTCGACGC | GGCTCTGCAC | CCCTGGCTGC |
| 34621 | TGGGCGACTT | CCTGTCGCGG | CCCGACGGCG | GATCCGTACT | GCTGCCGTTC | GCGTGGCGCG |
| 34681 | GCATCACGCT | CCACACGGCC | GGTGCCGACG | CGCTGCGGGT | CCGTCTCGGA | CCGGCCGGAG |
| 34741 | AAGGCGCTCT | GTCGCTCGAA | GCCGTCGACC | TCTCCGGTGC | CCCGGTGCTG | TCGATGGACG |
| 34801 | CACTGGTGCT | GCGTCCGCTC | GCCCAGGACC | GCCTGGCGGA | ACTGGTCGGC | GGCACGACCT |
| 34861 | CCACCCCGCT | GTACCGCGTG | GACTGGCAGC | GGAGCCCGAT | CGCGAGGACG | GCGCCGTCGG |
| 34921 | CCACGGGGCT | CTTCGGCTCC | CTCCCGTCCG | GTGCCGTCCG | CCGCTGGGCG | GTCGTCGGGC |
| 34981 | AGGGCGGTGT | CGCCGCACGG | TACGCGACGG | CGGAACCCGG | CACGGGGTGC | GTCGGGGTCT |
| 35041 | TCCCCGACCT | GGACGCACTG | CGTACGACGC | TGGACTCCGG | AGCGGACGGC | CCCGACATCG |
| 35101 | TCCTCGCCGA | CTTCGGGGCC | CGGCCAGGCG | ACGCCGCGCC | GCACGGGACG | GATCCGGCCG |
| 35161 | ACGGCGCACG | CGACACGGTC | CGGCGGGGGC | TCGCCCTCAT | ACAGGGCTGG | CTGTCCGACG |
| 35221 | AGCGCTTCGC | CGCCGCGCGT | CTCGCCGTGC | TCACCGAGCA | CGCCGTCGCC | ACCGAGGCGG |
| 35281 | ACACCCGCCG | GACGGACCTC | GCGGGCTCGG | CACTGTGGGG | GCTGATGCGT | TCGGCGCAGA |
| 35341 | CCGAGCACCC | CGACCGCTTC | GTCCTCGTCG | ACCACGACGG | GCAGGACGCC | TCGTACCGGA |
| 35401 | CGCTGCCCAC | CGCGCTCGAC | AGCGAAATCC | CGCAACTGGC | GCTCCGAGCC | GGGGAGACGC |
| 35461 | TGGCCCCCGA | ACTGGCGGTC | CTGCCGAGTC | CGGCCGACGG | GGGGCCCGCG | ACAAGCGCGG |
| 35521 | CGTTCGATCC | CGAAGGCACG | GTACTCGTCA | CCGGAGCCAC | CGGCACCCTC | GGCAGCCTGC |
| 35581 | TGGCCCGGCA | CCTGGTCACG | GCACACGGCG | TACGGCATCT | GCTGCTGCTC | AGCCGCAGCG |
| 35641 | GACGCGAAGC | CGCGGGGGCC | GCCGAACTGG | AGCGTGAACT | CCGTCAACGG | GGAGCCGAGT |
| 35701 | TCCAGCTCCT | CTCCTGTGAC | GCGACGGACC | GGGCAGCGCT | GAAGGAGGCC | CTCGCCACCG |
| 35761 | TCCCCGCCGC | CCACCCGCTG | ACCGCGGTGA | TCCACACGGC | CGGCGTCCTC | GACGACGGCG |
| 35821 | TCGTCGAGGC | GCTGACCCCC | GAACGGCTGG | ACCGCGTGCT | GCGCCCCAAG | GCGGACGCCG |
| 35881 | CGCTGAACCT | GCACGACCTG | ACCGAGGGAA | TGCCGCTGAA | GGCGTTCGTC | CTGTACTCCG |
| 35941 | GGGCGGTCGG | ACTGCTGGGT | GGAGCGGGCC | AGGCCAACTA | CGCGGCAGCC | AACGCGTTCC |
| 36001 | TGGACGGCCT | GGCCCAACAC | CGGCACGCGC | AAGGGCTGCC | CGCGGTGTCC | CTGGCATGGG |
| 36061 | GACTCTGGAG | CGCCACCAGC | ACGTTCACCG | ACCATCTCGG | CGAGGTGGAC | CTGCGGCGCA |
| 36121 | TGGAGCGGTC | CGGCATCACG | CCGCTCACGG | ACGAGCAGGG | CCTTGACCTG | TTCGACCGGG |
| 36181 | CCCTCGGCGC | CGCGGTGGAC | GCGCCGCAGC | TCTGCGTGAT | GGGGCTGGAC | ACGGCGGCGC |
| 36241 | TGCGCCGGCA | GGCGGCCGAG | CACGGGCCGA | CTTCGATGCC | TCCTCTGCTG | CGTACGCTGG |
| 36301 | CGGCGCCTCC | CGTACGGCGC | GGCGCGGGGC | GCTCCGGCCG | GGGCGGACGG | GCGGCGTCCG |
| 36361 | CCACGGACGC | GCCGAGCCGG | GCGCAGGCCC | TGCGCGAGCG | ACTGACGGGC | CTGGACGCGG |
| 36421 | CGGCACGGCG | GGACGAACTC | CTGGTCCTGT | CGCAGGCGCA | GTTGGCCGAT | GTGCTGGGCT |
| 36481 | TCGCCGACAA | GACCGCGGTG | GACCCCGTTC | GTTCCTTCCG | CGAGATCGGT | CTGGACTCGC |
| 36541 | TGACCGCCGT | CGAGCTGCGC | AACCGGCTCG | GTGTCGTCAC | CGGACTGCGG | CTGCCGCCGG |
| 36601 | CGCTGGTCTT | CGACCACCCC | AACCTCGACG | CGCTCGCGGC | CCACCTGGCG | GAGCTCCTCG |
| 36661 | CGGCTGAGGG | CCGGGACGAC | GCGGGCGCCG | CGGCGCTCTC | GGGAATCGAC | GCCCTGGACC |
| 36721 | GGGCGGTCCG | GGAGATGGCG | GCCGACGACA | CGCGCCGTGA | CGCCGTCCGC | CGACGCCTCG |
| 36781 | CGGAACTGCT | GGCGGTGGTC | GGCGACGCCC | CGCGGGACGG | CGGCCGCGCC | CCACGGGCGG |
| 36841 | CCGCCGACGC | GGGAGGCCGC | GACGCTCAAG | CGGACCCCGA | CCTGCTGGGC | CGGCTGGACT |
| 36901 | CCGCCTCCGA | CGACGATCTG | TTCGCGTTCA | TCGAAGACCA | GCTGTGAGCG | GGACGCCGCG |
| 36961 | CGCGTTCCCC | ACCCGTCCCT | AAGCGCCGCA | TCAGGCGCAC | TCGCACCGAC | ACGAGCACGC |
| 37021 | AGGCCAGGAG | GGTCCGGTCG | ATGACGGCCA | ACGATGACAA | GATCCGCGAC | TACCTGAAGC |
| 37081 | GGGTCGTCGC | CGAGCTGCAC | AGCACGCGGC | AACGGCTCAA | CGCCTTGGAG | CACGACGCCC |
| 37141 | GCGAGCCCAT | CGCCATCGTG | GGGATGAGCT | GCCGGCTGCC | CGGCGGGGTG | ACCACCCCCG |
| 37201 | AGAGCCTGTG | GAGGCTGGTC | GACTCCGGCA | CCGACGCCGC | CTCGCCGTTC | CCCGACGACC |
| 37261 | GGGGCTGGGA | CCTGGACGCG | CTCCACCATC | CGGAGTCGGG | AGCCGTCCAC | TCCCGCGAGG |
| 37321 | GCGGATTCCT | CCACGACAGC | GCGGACTTCG | ACGCGGAGTT | CTTCGGCATC | AGCCCGCGAG |
| 37381 | AGGCCCTGGC | CATGGACCCG | CAGCAGCGGC | TGCTGCTGGA | GACCGCCTGG | GAGGTGTTCG |
| 37441 | AGCGCGCCGG | CATCGACCCG | GTCTCCGCCC | GCGGCTCCCG | CACGGGGGTG | TACGCGGGCG |
| 37501 | TCATGTACCA | CGACTACGGC | GCCCGGCTGA | ACGAGATCCC | GCCGGGCCTC | GAGGGCTACC |
| 37561 | TGGTCAACGG | CAGCGCGGGC | AGCATCGCCT | CGGGCCGGGT | GGCCTACACC | CTCGGTCTGG |
| 37621 | AGGGCCCCGC | CGTCACCGTC | GACACGGCCT | GCTCCTCGTC | ACTGGTCGCC | GTGCACCTGG |
| 37681 | CGGCACAGGC | ACTGCGGCGG | CGGGAGTGCG | ACATGGCGCT | CGCGGGCGGC | GCGACCGTCC |
| 37741 | TGTCCACCCC | CGACCTGTTC | ATCGACTTCG | CGCGACTCGG | CGGCCTGGCC | TCCGACGGGC |
| 37801 | GCTGCAAGGC | GTTCTCCGAC | GCCGCCGACG | GCACCAGCTT | CGCCGAGGGC | GCCGGCCTGC |
| 37861 | TGCTCCTCAT | GCGGCTGTCG | GACGCGGTGG | CCGAGGGCCA | CACCGTCCTG | GCGGTCGTCC |
| 37921 | GAGGCTCCGC | CGTCAACCAG | GACGGGGCGA | GCAACGGCCT | GACGGCCCCC | AACGGCCTCG |
| 37981 | CCCAGCAACG | CGTGATCCGC | GAGGCGCTCG | CCGACGCGGA | CCTGGACCCC | GACCAGATCG |
| 38041 | ACGCGGTGGA | GGCGCACGGC | ACCGGAACCA | GGCTCGGCGA | CCCCATCGAG | GCGCAGGCCC |
| 38101 | TGCTGCACAC | GTACGGCACG | AGCCGCAGCC | CCGAACGACC | CCTGTGGCTC | GGTTCGCTGA |
| 38161 | AGTCCAACAT | CGGCCACACC | CAGGCCGCCG | CCGGAGTGGC | CGGAGTCATC | AAGACGGTGC |
| 38221 | TGGCGATGCG | CCACGGACGG | CTGCCCCGCA | CACTGCACGT | CACCCGCCCC | TCCAGCCGGG |
| 38281 | TGGAATGGTC | GGCGGGCGCG | GTGGAACTGC | TCACGCGGGC | ACAGGACTGG | CCCGGCCAGG |
| 38341 | GGAACGCCCC | GCGCCGCGCC | GGAGTGTCGT | CCTTCGGTGC | CAGCGGCACC | AACGCACACC |
| 38401 | TGATCCTGGA | AGGCGTCCCG | GACGGCGACA | TCACGGTCGC | GGAGACCCGA | CCGGCCACCG |
| 38461 | GCGGCGGCGC | CTGGCCGCTC | GCGGGCCGGA | CCGAAGCGGC | CCTGCGCGCA | CAGGCCCGGC |
| 38521 | GGCTCCACGA | CCACCTCGCC | GCCCGCCCCC | ACGTCTCACC | CGCCGCGGTC | GGGCGGACGC |
| 38581 | TGGTCCGCTC | CCGCACGGCG | TTCGAGCACC | GGGCCGTCGT | CCTCGGACAG | GACACCGCCG |
| 38641 | ACCTCCTGAG | CGGCCTCGCG | GAGCTGGCGT | CCGGCGGCGC | TCACGGACCC | GGCGTGATCA |
| 38701 | CCGGCCGGGC | CGCGCGCGGC | CGCCGTACCG | CACTGCTCTT | CACCGGACAG | GGCAGCCAGC |
| 38761 | GGCCGGGTGC | CGGACGGCAT | CTCTACGAGC | GGTACGAGGT | GTTCGCCCGC | GCCCTGGACG |
| 38821 | AGACGGCCGC | GGCACTCGAC | CGGCACCTCG | ACCGCCCGCT | GCGCGACGTG | ATGTTCGCGG |
| 38881 | AGCCGGGCGG | CGCGACGGCC | GGACTCCTCG | ACCGCACCGA | GTACACCCAG | CCCGCACTGT |
| 38941 | TCGCCCTGGA | AGTCGCCCTC | TTCCGACTGG | TGACCGCCGG | GGGCCTGCGC | CCCGACGCAC |
| 39001 | TCCTCGGGCA | CTCCGTCGGC | GAACTGGCCG | CCGCCCACGT | CGCCGGAGTG | TTCACCCTGC |
| 39061 | CCGACGCCGC | CCGCCTGGTG | ACGGCGCGAG | GCCGACTGAT | GGGCGAGCTG | CCGGCCGGTG |
| 39121 | GCGCCATGAT | GGCGATCCAG | GCGAGCGGCC | CGGAGATCGA | GGAGACGATC | ACGGCGCTCG |
| 39181 | CGGCCCACCG | GTCGGCGCGC | GTCGCCGTCG | CCGCACTCAA | CGGTCCCGAC | GCCACCGTGA |
| 39241 | TCTCGGGCGA | CGAGGACGTG | GTCGCCGAAC | TCGCCACGCT | GTGGCGGGAG | CGGGGCCGCC |
| 39301 | GCACCAGGGC | GCTGCCCGTC | AGCCACGCCT | TCCACTCGCC | GCACATGGAC | GCCGCACTGG |
| 39361 | AACCGTTCGC | CCGGATCGCG | CGCGACGTCT | CCTACGCCGA | ACCGCGCATC | CCGGTGGTCT |
| 39421 | CCAACCTGAC | CGGCGGCATC | GCGTCGGCCA | CGACGCTGTG | CGCCCCCGAG | TACTGGGTGC |
| 39481 | GCCACGCGCG | CGAGGCCGTG | CGCTTCAGCG | ACGGCTTCCG | CGCCCTGCGC | GACCAGGGGA |
| 39541 | TCGACACCTT | CATCGAGCTC | GGACCGGACG | GCGTGCTGTC | CGCCCTGGGC | CGCGACTGCC |
| 39601 | TCCGCGAGGA | GGAAGGAGAC | GCCCCACGCC | AGGACGGCTC | GGCGGACCCC | GACACGACCG |
| 39661 | GCTCCCGCGC | CGACGGGGGG | CGGCGACCCG | TGCTGACGGT | GCCGCTGCTG | CGCCGGGACC |
| 39721 | GCGACGAGAC | GACCACCTGC | CTCGGGGCCC | TGGCCACCGT | CCACACCCAC | GGCGTCCCCG |
| 39781 | TCGACCTCGC | GGCCGTGCAC | GGCGCCCCCG | AGGGGCCCGC | CGTCGAGCTC | CCCACCTACG |
| 39841 | CCTTCCAACG | CACGCGCTAC | TGGCTGGACG | CCCCGGCCCC | CGCCGCCGGC | CCCACCGCCA |
| 39901 | CCGGTCTGGA | GGCGACGGAC | CAGCCCCTGC | TCCCGGCCGT | CATCGACCTG | CCCGACGGGG |
| 39961 | AAGGCACCGT | ACGGACCGGA | CTGCTCTCCC | TGCGCACCCA | TCCGTGGATC | GCCGACCACC |
| 40021 | GCGTCCGCGA | CCACGCCGTG | GTGCCCGGAG | CGGCCCTGCT | GGACGTCGCC | GCCTGGGCGG |
| 40081 | GCACCGAAGC | GGGCTGCCCC | CGGGTCGCCG | AACTGACCTT | CGCCACGCCC | CTCGTCCTGC |
| 40141 | CCGAGAACGG | AGAAGGAGTC | CGACTGCGCG | TGACGGTCAG | CGGCCCCGAC | GCGGAAGGCA |
| 40201 | TCCGTTCGCT | ACGCATCGAC | TCCCGGCCCG | CCGACACGGT | CCGTACCGCC | GATGCCCCCT |
| 40261 | CCGACTGGAC | CCGCCACGCG | TCCGGCACCC | TCGTCCCCGC | ACCCGAGGAG | GCCGGCGACG |
| 40321 | GCACCGGCGT | GCCGACCGAA | CTGCTCGGCG | CCTGGCCCCC | GGCCGACGCG | ACCCCGGTCG |
| 40381 | CCCTCGACGC | GGACGCCGTC | GCGGCCGAGT | ATCAGCGCCT | CGCGGCCGGC | GGCGTGACGT |
| 40441 | ACGGCCCCGC | GTTCCGGGCA | CTGCGCGCCG | TCTGGCGCCG | CGGCGCAGAG | GTCTTCGCCG |
| 40501 | AGGTCCGGCT | TCCCGGCCAG | GCGGCCGCCG | ACGCCTCGCG | GTACGGCATG | CACCCGGCCC |
| 40561 | TGCTGGACGC | CCTGACGCAC | GCCACCGGGT | TCGGCGAGCG | GTCCACCGAG | GCCCGCGGCC |
| 40621 | TGGTCCCGTT | CGCCTGGAGT | GACGTCCGGA | TCCACGTCCG | CGGCGCCGAC | TCCCTGCGCG |
| 40681 | TACGCATCGC | GCCGGCCGGC | CCCGACGCCG | TGACCGTCGC | CGCGGTCGAC | CCGACGGGCC |
| 40741 | GCCCGGTCCT | CGCCGCCCGC | TCGCTCACGC | TGCGCCCCCT | GGCGGAGAGC | CGGTTCCAGG |
| 40801 | ACCCGGAGGC | GGACAGCACG | CCGCTGTACC | GACTGGAGTG | GACACCGGCC | CCCGGTTCCG |
| 40861 | TGACCGGGCA | CGCCGGTCCA | AGGCAGGCGG | CAGCGGAGTG | GGGCGTCCTC | GGCGACCCGG |
| 40921 | TCCAGGCCCT | CCTCGACGCC | GTGCGCGACG | GGGCGGAGGC | ACCCGTGCGA | ACCCATGACG |
| 40981 | ACCTGCTCGC | GCTCGCGGCC | TCCGACACGG | CCCCGCCCGA | CCATGTGCTG | GCGCTGCTGG |
| 41041 | GCCACGACGG | GGACGCTCTC | GCCACGGGCG | CCCACGACCT | GGCCGCACGC | GCCCTGGCCC |
| 41101 | TGGTACAGGG | CTGGCTGACC | CACGCCCGCT | TCGCCGACGC | GCGACTGGTC | GTGCTGACGC |
| 41161 | AGGGGGCGGT | GACGGCCGGC | ACGAGCCCCG | TCCACCCGGC | CGCGGCCGCC | GCCTGGGGGC |
| 41221 | TGCTGCGCAG | CGCACAGTCC | GAGCACCCGG | GCCGCTTCGT | CCTCGTCGAC | GCGGACCCCG |
| 41281 | CCGACCCGGC | CGCCTCGTAC | CGTTCCCTGC | CACGGGCCGT | CGCCTCCGGG | GCGTCCCAAC |
| 41341 | TCGCCCTGCG | CGGCGCCGAG | ATCCTCGTCC | CCCGGCTCGC | CCGGGGAACG | GACCGACAGG |
| 41401 | CCACCGTGCC | CGGACACCCC | GGCGACGTCA | CCGCACCGGA | GACGACCGCT | GCCCCCGAGC |
| 41461 | CGGCCCCGTC | CGGCACCCCC | TCCGGCCCCT | GGCCCGdGGA | CGGCACCGTG | CTCGTCACGG |
| 41521 | GCGGAACCGG | GACCCTGGGC | AAGGCCGTGG | CCCGGCACCT | CGTGACCAAG | CACGGTGTCC |
| 41581 | GGCACCTGAT | CCTGGCCGGT | CGGCGAGGCG | CGGACACCCC | CGGGGCGGCC | GACCTGGCCA |
| 41641 | CCGAACTGAC | CGGCCTGGGC | GCCACCGTGA | ACATCGTCCG | CTGCGATGCC | GCCGACCGCT |
| 41701 | CGGCGCTCGA | AGGCGTCCTG | GCCGCCGTCC | CCGCCGCGCA | CCCGCTCACC | GCCGTCGTGC |
| 41761 | ACACCGCCGG | AGTGCTCGAC | GACGGCATCG | TCACGGCGCA | GACGCCCCGA | CGCCTCTCGG |
| 41821 | CGGTCCTGCG | CGCCAAGGCG | GACGCGGTCA | GCCACCTGCA | CGAACTGACC | CGCGACCTGG |
| 41881 | ACCTGTCCGC | CTTCGTCCTC | TTCTCATCGG | CCGCCGGAAC | CCTCGGCAGC | CCCGGCCAGT |
| 41941 | CCGGCTACGC | GGCCGCCAAC | AGCTTCCTCG | ACGCGTTCGC | CGCCTGGCGG | CGAGCGCAGG |
| 42001 | GCCTCCCCGC | CGTGTCCCTG | GCATGGGGAC | TGTGGGGTGA | CGGCGGTGAC | GGTCGTGACG |
| 42061 | GCGGTGGCTC | GGCGGCCGAC | GGCATGGGAG | CGAGCCTGGC | CGCCGCCGAC | CTGGCACGGC |
| 42121 | TGCGCCGTTC | CGGCATCCTC | CCGCTCGACC | CGGCCGAAGC | GCTGCGCCTG | TTCGACGAGG |
| 42181 | CCTGCGACCC | CGCCAGGACC | GAGGCCGTAC | TGCTGCCGAT | CCGCCTCGAC | CTGACCGGCC |
| 42241 | TGCGCGCCCG | TTCCGCCCGC | GGCGCCGTAC | ACGCGAGCGT | GGTGCCCGAA | GTGCTGCACA |
| 42301 | CCTTGGTGCC | CCCGCCCGCC | GGTGCCGGAT | CCCCGGCCGG | TGCCGACGCG | TCGGATCCCG |
| 42361 | CGGCGGGCCA | GGCGCCCCCG | GCCCCGGCGT | CCGACACCCT | GGCCGAACGG | CTCGCCGGGA |
| 42421 | AGCCCCGAGG | CGAACGGCTC | ACCGCCCTCA | CCCAACTGGT | ACGCACCGAG | ATCGCCTCGG |
| 42481 | TACTCGGGCA | CCCCGACTCC | GGCCGCGTCC | AGCTCCAGTC | CTCCTTCAAG | GAGTCCGGCT |
| 42541 | TCGACTCGCT | CACCGCCGTC | GAACTCCGCA | ACCGGCTCAC | CGCGGCCACC | GGAACGAAGC |
| 42601 | TTCCCGCCAC | CCTCGTCTTC | GACCATCCGA | CACCCGCGGC | ACTCGTCGAC | CACCTGGAAC |
| 42661 | AGGAACTGCC | GAAGGCAGCA | CAGGAGATCC | CGGCGGACCT | CCCGGCCGTT | CTCGACGCAC |
| 42721 | TCGACCGGAT | CCGGGACGGA | CTCGCCACCG | CCGCCACCGA | CGACAGCAGC | CGCGACCACA |
| 42781 | TCGCGGAGCG | TCTCCAGGCG | TTGCTCGGCA | CGCTCACCTC | GGCTGCGGGC | GTCAGCCGCC |
| 42841 | CGACCGGCAG | CCCGGGCGAG | CACGACCGGC | AGGGCCCCGA | TGAGCTGTCG | CTCGGCCAAC |
| 42901 | GACTCGCCGC | CAGCAGCGAC | GACGAACTCT | TCGACCTCTT | CGACAGCGAC | TTCCGATCGA |
| 42961 | CGTAGCCCAG | GGCCACCCTT | CCGCCTCCGC | CGCCCGCCCC | ACACCCCTGG | AGAACAGCAC |
| 43021 | GATGTCTTCC | ACATCGCCCG | CCACCAACGA | AGAGAAGCTC | CGCGACTACC | TCCGCCGCGC |
| 43081 | CATGACCGAC | CTGCACGAGG | CCCGCGAGCA | GATCCGCCGG | ACCGAGTCGG | CCAGGCACGA |
| 43141 | GCCGATCGCG | ATCGTGGGGA | TGGGGTGCCG | TCTGCCGGGT | GGGGTGTCGT | CGCCGGAGGG |
| 43201 | GTTGTGGGAT | CTGGTGGCGT | CGGGGGTGGA | TGCGGTTTCT | CCGTTCCCCA | CGGATCGGGG |
| 43261 | TTGGGATGTG | GGGGGTCTGT | TCGATCCGGA | GCCGGGGGTG | CCGGGGCGTT | CGTATGTGCG |
| 43321 | TGAGGGCGGG | TTCCTTCATG | AGGCGGGGGA | GTTCGACGCG | GGGTTCTTCG | GTATCTCTCC |
| 43381 | GCGTGAGGCG | TTGGCGATGG | ATCCGCAGCA | GCGGTTGCTG | TTGGAGACGT | CGTGGGAGGC |
| 43441 | GTTGGAGCGG | GCGGGCATCG | ATCCGCACAC | GCTCCGCGGC | TCACGGACCG | GCGTCTACGC |
| 43501 | CGGAGTGATG | TACCACGACT | ACGGCAGCAC | CGCGACCGTC | TCCGTCGCCT | CCGACGACGA |
| 43561 | GACCGCCGGA | TTCCTCGGCA | CGGGGACGTC | CGGGAGTGTG | GCTTCGGGTC | GTATCTCGTA |
| 43621 | TGTGCTGGGG | CTGGAGGGGC | CGGCGGTGAC | GGTGGACACG | GCGTGTTCGT | CGTCGTTGGT |
| 43681 | GGCGCTGCAT | CTGGCGGTGC | GGGCGCTGCG | GAGTGGTGAG | TGTGACCTTG | CTCTTGCGGG |
| 43741 | TGGGGTGACG | GTGATGGCGG | AGCCGGGTGT | GTTCGTGGAG | TTCTCGCGGC | AGCGGGGGTT |
| 43801 | GGCTGCGGAC | GGGCGGTGCA | AGGCGTTCGC | GGCTGCTGCC | GATGGCACGG | GCTGGGCCGA |
| 43861 | GGGTGTGGGC | GTGCTGGCGG | TGGAGCGGTT | GTCGGATGCG | GTGCGTCACG | GGCGCCGGGT |
| 43921 | CCTGGCGGTC | GTGCGTGGTT | CCGCGGTCAA | CCAGGACGGT | GCGAGCAATG | GGTTGACGGC |
| 43981 | TCCGAACGGT | CCGTCGCAGC | AGCGGGTGAT | TCGTCAGGCG | TTGGCGGATG | CGCGGTTGGG |
| 44041 | TGTGGCTGAT | GTGGATGTGG | TGGAGGGGCA | TGGGACGGGG | ACGCGTCTGG | GTGATCCGAT |
| 44101 | CGAGGCGCAG | GCGTTGTTGG | CGACGTATGG | GCAGCGGGAT | GCGGGTCGGG | CTTTGCGGCT |
| 44161 | TGGTTCGCTG | AAGTCGAACG | TGGGGCATAC | GCAGGCGGCT | GCCGGTGTGG | CGGGCGTGAT |
| 44221 | CAAGATGGTC | ATGGCGATGC | GGCACGGTGT | CCTGCCGAAG | ACGCTGCACG | TGGATGAGCC |
| 44281 | GACGGCGGAG | GTGGACTGGT | CGGCGGGCGC | GGTGTCCCTG | CTGAGGGAGC | AGGAGGCGTG |
| 44341 | GCCTGAGGTG | GGGCGTCTGC | GTAGGGCTGC | GGTGTCTTCG | TTCGGTGTGA | GTGGGACGAA |
| 44401 | CGCGCATGTG | GTGGTGGAGG | AGGCACCTGC | TTCGGAGGCG | CCGGTCGCGG | GGGAGCCGGT |
| 44461 | GGAGCCGGTG | GAGCCGGGGG | CTGTGGGGCT | TCTTCCGGTG | GTGCCGGTGG | TGGTGTCGGG |
| 44521 | TCGTTCTGCG | GGTGCGGTGG | CGGAGCTGGC | CTCCCGCTTG | AACGAGTCGG | TTCGTTCGGA |
| 44581 | TCGGTTGGTG | GATGTGGGGT | TGTCGTCGGT | GGTGTCGCGG | TCGGTGTTCG | AGCACCGGTC |
| 44641 | CGTGGTTCTG | GCGGAGGACT | CTGCCGAGCT | GCATACCGGT | CTGGTTGCTG | TCGGGACTGG |
| 44701 | GGTGCCGTCG | CCTGGCGTGG | TGTCGGGTGT | GGCGTCGGTC | GAGGGTGGCC | GGTCGGTGTT |
| 44761 | CGTGTTCCCT | GGTCAGGGGA | CGCAGTGGGC | GGGGATGGCG | CTCGGGTTGT | GGGCGGAGTC |
| 44821 | GGCGGTGTTC | GCGGAGTCGA | TGGCGCGGTG | TGAGGCGGCG | TTCGCCGGGT | TGGTGGACTG |
| 44881 | GCGTCTGGCG | GATGTGCTGG | GTGACAGGTC | TGCGTTGGAG | CGGGTCGATG | TGGTGCAGCC |
| 44941 | GGCGTCGTTC | GCGGTGATGG | TGTCGCTGGC | CGAGCTGTGG | CGGTCGCTGG | GTGTGGTGCC |
| 45001 | CGATGCGGTG | GTGGGGCATT | CGCAGGGGGA | GATCGCTGCT | GCGGTGGTGG | CGGGTGGTCT |
| 45061 | CTCGCTGGAG | GACGGCGCGC | GTGTGGTGGT | GTTGCGTGCG | CGGTTGATAG | GCCGTGAGCT |
| 45121 | GGCCGGGCAC | GGTGGGATGG | CGTCGGTGGC | GCTGCCGGTC | GCGGTGGTGG | AGGAGCGTCT |
| 45181 | GGCGGCGTGG | GCGGGGCGTC | TGGGTGTGGC | GGTGGTCAAC | GCACCCTCCG | CCACGGTCGT |
| 45241 | CGCGGGTGAT | GTGGACGCGG | TGGCGGAGTT | TGTGACCGCG | TGCGAGGTGG | AGGGGGTTCG |
| 45301 | GGCGCGTGTT | CTGCCGGTGG | ACTACGCCTC | GCACTCGGCG | CACGTGGAGG | AGCTGAGGGC |
| 45361 | CGAGCTTGAA | CAGATTCTGG | CCGGCATCGA | CCCGGTGGCC | GGTGAGACCC | CCCTGTACTC |
| 45421 | CACGGTGGAG | GCGGGTGTCG | TGGATACGGC | GTCGATGGAT | GCGGGGTACT | GGTTCAGGAA |
| 45481 | TCTGCGTCGG | CCGGTTCGTT | TCCAGGAGAC | GGTCGAGCGG | TTGCTGGCGG | ATGGTTTCCG |
| 45541 | GGTGTTCGTG | GAGTGCGGCG | CGCATCCGGT | GCTGACGGGG | GCGGTGCAGG | AGACCGCGGA |
| 45601 | ATCCACCGGT | CGCCAGGTGT | GTGCGGTCGG | ATCCCTGCGT | CGTGACGAGG | GTGGTCTGCG |
| 45661 | ACGCTTCCTG | ACCTCTGCGG | CGGAGGCGTT | CGTCCAGGGG | GTGGAGGTGT | CCTGGCCGGT |
| 45721 | GCTGTTCGAT | GGCACCGGCG | CCCGCACGGT | CGACCTGCCC | ACCTACCCCT | TCCAGCGTCG |
| 45781 | GCGTTACTGG | CTGGAGTCAC | GTCCTCCTGC | GGCGGTTGTT | CCGTCGGGGG | TCCAGGACGG |
| 45841 | ATTGTCGTAT | GAGGTGGTGT | GGAAGAGCCT | GCCGGTACGG | GAGTCGTCGC | GTCTTGACGG |
| 45901 | CCGGTGGCTG | CTCGTCGTGC | CCGAAACCCT | GGACGCCGAC | GGCACGCGGA | TCGCCCACGA |
| 45961 | CCTCCAGCAC | GCCCTCACCA | CCCACGGCGC | CACCGTGCAC | ACTCTTGCTC | TTGACCCCAG |
| 46021 | CGCGGCGCAC | TTCGACGGTC | TCTTTGACGG | GATACTCCAG | GAAGAAACAG | ATGTCACGGG |
| 46081 | CATCTTCTCT | CTCCTCGGAC | TGGCATCGGG | CCCGCACCCG | GATCACGGCG | AGGTGGAGCT |
| 46141 | CGCGGGAGCC | GCGTCGCTGA | CGTTGATGCG | CCAAGCCCAG | CGAGACGGCT | TCCGTGCTCC |
| 46201 | GGTGTGGGCG | GTGACGCGGG | GTGCGGTGTC | CGTGGTGCCT | GGTGAGGTGC | CGGAGACCGC |
| 46261 | GGGTGCGCAA | CTGTGGGCGC | TCGGCCGGGT | CGCCGGTCTC | GAACTCCCCG | ACCGTTGGGG |
| 46321 | TGGTCTGATC | GATCTCCCGG | CGGATGCCGA | TGCGCGTACG | GCGGGGCTTG | CGGTGCGGGC |
| 46381 | CCTGGCCGCC | GGGATCGCCG | ATGGTGAGGA | CCAGCTGGCG | GTGCGCCCCT | CAGGTGCCTA |
| 46441 | CGGCCGGCGC | CTCGTACGAG | CCACCGCGCG | CCGGGGACGG | AAGGACTGGC | GCCCGCAGGG |
| 46501 | TACGGTGCTG | CTCGCCGGGC | ACCTCGACGC | CGTCGGTGAA | CCACTGGCCC | GATGGCTGCT |
| 46561 | CACCGGCGGC | GCGGACCACG | TCGTCCTTGC | GGATCCCGCC | CTGACCGAAC | TCCCGGCCAC |
| 46621 | CCTCGCGGAT | CTGGCCCAGA | CCGTGACGAC | CGCTGCGGCA | CCCGACCTTG | CCGACCGTGC |
| 46681 | AGTCCTCGCC | GCCCTGGTCA | CCGAGTACGT | ACCCGCCACC | GTGGTCGTCG | TTCCGCCCGC |
| 46741 | GGCGGAGCTC | GCTCCGCTGG | CGAGTATCAG | CCCGGCCGAC | CTCGCGGCGG | CCGTCACCGC |
| 46801 | CAAGTCCGCG | ACCGCGGCGC | ACTTCGACGC | GCTGCTCGAC | GGACCCCACG | CACCGGAGCT |
| 46861 | GGTGCTGATC | TCCTCGGTCG | CGGGGATCTG | GGGTGGTGTC | CGGCAGGGTG | CGTACGCCGT |
| 46921 | CGGTGCCGCT | CACCTCGATG | CCCTGGCCGC | CCGCCGCAGG | GCCCGCGGTC | TGTCGGCGGC |
| 46981 | CTCCGTCGCG | TGGACGCCCT | GGGCGGGTTC | CGTCACCGCG | GACGGCTCCG | CCGCCGAGTC |
| 47041 | GCTGCGGCAG | TACGGCATCG | CTCCGCTGGA | GCCGCAGGCG | GCGCTCGCGG | AGCTGGACCG |
| 47101 | GGCGCTGAAC | CAGCAGCTGC | ACGGCGGCGG | GGGCGACGCG | GCGGTGGCCG | ACATCGACTG |
| 47161 | GGAGCGGTTC | CTCGCGTCGT | TCACCTCCGT | ACGTCCCAGC | GTTCTCTTCG | ACGAGCTGCC |
| 47221 | CGAGGTACGC | CGTCTCCGCG | AGGCGGAGGC | GGCGGCCATG | GCGGACCAGG | CCGCCGCCCG |
| 47281 | GACGGGAGCG | CCCGGCGGAA | CGGAGCTGGC | GCGCTCTCTG | CGGGCCAAGT | CCCTGAACGC |
| 47341 | CCAGCGAACT | GCGCTCCTGG | AATTGGTCAC | TGCCCACGTG | GCGGCCGTGC | TGGGAGAGAG |
| 47401 | CGTTCCCGAG | GCGATCGACC | GGAGCCGGGC | GTTCAAGGAC | ATCGGCTTCA | CCTCCATGAC |
| 47461 | CGCGATGGAA | CTGCGCAACC | GGCTCAAGGA | GGCCACCGGG | CTCGCCCTTC | CTGCCTCCCT |
| 47521 | CGTCTTCGAC | CACCCCCACC | CCGGCGCACT | CGCCGACCAC | CTGCGCGAGG | AACTCCTGGG |
| 47581 | CGAGGACGGT | GCGGCGGGCG | CCGACTCCGC | GGCGGAGGAA | CCGAGCGCTA | CCTCTCCGAC |
| 47641 | GGTCCAGGAC | GAGCCGATCG | CCATCATCGG | CATGGCCTGC | CGCCTCCCTG | GTGACGTCGG |
| 47701 | AACACCCGAC | GAACTCTGGG | AGCTGCTGGA | AACCGGCCGC | GACGCGATGT | CGGACCTGCC |
| 47761 | CGTCAACCGC | GGGTGGGACG | TGGCGGGGCT | CTACGACCCG | GATCCAGACG | CGGCGGGGCG |
| 47821 | TTCCTACGTC | CGGGAGGGCG | GGTTCCTCCA | CGACGCGGGG | GAGTTCGACG | CGGAGTTCTT |
| 47881 | CGGCATCTCG | CCGCGTGAGG | CGCTGGCGAT | GGACCCGCAG | CAGCGCATCG | TCCTCGAACT |
| 47941 | CGCCTGGGAA | TCGTTCGAAC | GTGCGGGCCT | GGACCCGGCC | GGCCGCCGCG | GCAGCCGTAC |
| 48001 | CGGCGTGTTC | ATGGGAACCA | ACGGCCAGCA | CTACATGCCG | CTGCTGCAGA | ACGGCAACGA |
| 48061 | CAGCTTCGAC | GGCTACCTCG | GCACCGGGAA | CTCGGCCAGT | GTCATGTCGG | GCCGCATCTC |
| 48121 | GTACACCCTC | GGCTTGGAGG | GGCCGGCGCT | GACGGTGGAC | ACGGCGTGTT | CGTCGTCGCT |
| 48181 | GGTCGCGCTG | CATCTGGCGG | TGCGGGCGCT | GCGCAACGGT | GAGTGCGACC | TCGCCCTCGC |
| 48241 | CGGCGGCGCG | ACCGTGATGT | CGACGCCGGA | AGTCCTGGTG | GAGTTCTCCC | GGCAGCGTGC |
| 48301 | AGTCTCCGCA | GACGGTCGCT | GCAAGGCGTT | CTCCGCCTCG | GCCGACGGCT | TCGGACCGGC |
| 48361 | CGAGGGTGCG | GGCGTGTTGC | TCGTCGAGCG | GTTGTCGGAT | GCGGTGCGTC | ATGGGCGTCG |
| 48421 | GGTGTTGGCG | GTCGTGCGTG | GTTCGGCGGT | GAATCAGGAC | GGTGCGAGTA | ATGGGTTGAC |
| 48481 | GGCTCCGAAC | GGTCCGTCGC | AGCAGCGGGT | GATTCGTCAG | GCGTTGGCGG | ATGCGCGGTT |
| 48541 | GGGTGTGGCT | GATGTGGATG | TGGTGGAGGG | GCATGGGACG | GGGACGCGTC | TGGGTGATCC |
| 48601 | GATCGAGGCG | CAGGCGTTGT | TGGCGACGTA | TGGGCAGCGG | GATGCGGGTC | GGCCGTTGCG |
| 48661 | GCTTGGTTCG | TTGAAGTCGA | ACGTGGGGCA | TACGCAGGCG | GCTGCCGGTG | TGGCGGGCGT |
| 48721 | GATCAAGATG | GTCATGGCGA | TGCGGCACGG | TGTCCTGCCG | AAGACGCTGC | ACGTCGATGA |
| 48781 | GGTCTCTCCG | CACGTCGACT | GGTCGGCGGG | TGCGGTGTCC | CTGCTGACGG | AGCAGGAGCC |
| 48841 | GTGGCCGGAG | GTGGGGCGCC | CTCGCAGGGC | TGCGGTCTCT | TCGTTCGGGC | TCAGCGGGAC |
| 48901 | GAATGCGCAT | GTGGTGGTCG | AGGAGGCGCC | GGTCGGGGAG | GCGGGGCAGG | CCGCCGGGGA |
| 48961 | TGCTCGGTTG | GCTGTGGTGC | CGGTGGTGGT | GTCGGGCCGG | TCTGCGGGTG | CGGTTGCTGA |
| 49021 | ACTGGCCTCC | CGCTTGAACG | AGTCGATTCG | TTCGGATCGG | TTGGTGGATG | TGGGGTTGTC |
| 49081 | GTCGGTGGTG | TCGCGGTCGG | TGTTCGAGCA | CCGGTCCGTG | CTACTGGCGG | GGGACTCTGG |
| 49141 | CGAGCTGCAT | ACCGGTCTGG | TTGCTGTCGG | GACTGGTGTG | CCGTCGCCTG | GTGTGGTGTC |
| 49201 | GGGTGTGGCG | TCGGTCGGGG | GTGGCCGGTC | GGTGTTCGTG | TTCCCTGGTC | AGGGGACGCA |
| 49261 | GTGGGCGGGG | ATGGCGCTCG | GGTTGTGGGC | GGAGTCGTCG | GTGTTCGCGG | ACTCGATGGC |
| 49321 | GCGGTGTGAG | GCGGCGTTCG | AGGGGTTGGT | GGACTGGAGT | CTGGCGGATG | TGCTGGGTGA |
| 49381 | CGGGTCCGCG | TTGGAGCGGG | TCGATGTGGT | GCAGCCGGCG | TCGTTCGCGG | TGATGGTGTC |
| 49441 | GCTTGCTGAG | CTGTGGCGGT | CGTTGGGTGT | GGTGCCGGAT | GCGGTGGTGG | GGCATTCGCA |
| 49501 | GGGGGAGATC | GCTGCTGCGG | TGGTGGCGGG | TGGTCTGTCG | TTGGAAGACG | GGGCGCGTGT |
| 49561 | GGTGGTGTTG | CGTGCGCGGT | TGATCGGCCG | TGAGCTGGCC | GGGCGCGGTG | GGATGGCGTC |
| 49621 | GGTGGCGCTG | CCGGTCGCGG | TGGTGGAGGA | GCGTCTGGCG | GGGTGGGCGG | GGCGTCTGGG |
| 49681 | TGTGGCGGTG | GTCAACGGAC | CGTCCGCCAC | GGTCGTCGCG | GGTGATGTGG | ATGCGGTGGC |
| 49741 | GGAGTTTGTG | ACCGCGTGCG | AGGTGGAGGG | GGTTCGGGCG | CGTGTTCTGC | CGGTGGACTA |
| 49801 | CGCCTCGCAC | TCGGCGCACG | TGGAGGACCT | GAAGGCCGAG | CTTGAAGAGG | TGCTGGCCGG |
| 49861 | CATCGGCCCG | GTGACCGGTG | GGATCCCGTT | CTATTCGACG | TCCGAAGCCG | CGCAGATCGA |
| 49921 | CACGGCTGGT | CTGGACGCGG | GGTACTGGTT | CGGGAATCTG | CGTCGGCCGG | TGCGGTTCCA |
| 49981 | GGAGACGGTC | GAGCGGTTGT | TGGCGGATGG | TTTCCGGGTG | TTCGTGGAGT | GTGGTGCGCA |
| 50041 | TCCGGTGCTG | ACGGGGGCGG | TGCAGGAGAC | CGCGGAATCC | ACCGGTCGCC | AGGTGTGTGC |
| 50101 | GGTCGGATCC | CTGCGTCGTG | ACGAGGGAGG | TCTGCGCCGC | TTCCTCACCT | CTGCGGCGGA |
| 50161 | GGCGTTCGTC | CAGGGGGTCG | GGGTGTTCTG | GCCGGCACTG | TTCGACGGCA | CCGGCGCCCG |
| 50221 | TATCGTCGAC | CTGCCCACCT | ACCCCTTCCA | ACGACGGCAC | TACTGGTACA | ACGACCCTGC |
| 50281 | CCGCCGCACG | GGCGATGCCA | CCTCCTTCGG | TATGGCGCAG | GCCGGTCATC | CCTTGCTCGA |
| 50341 | TGCCGGTACG | GAGCTCCCTG | AATCAGGCGA | GCACCTCTAC | ACCGCTCGGC | TCGCCGCCGA |
| 50401 | CTCGCATCCT | TGGCTGCTGG | AACACACCCT | GCTGGGTGCG | CCGTTGCTGC | CCGGTGCGGC |
| 50461 | GTTCGTCGAC | CTCGTCCTGT | GGGCCGGCGG | GGAGGTCGGA | TGCGACCTGA | TCGAAGAGCT |
| 50521 | GACGCTGACC | TCGCCGCTGC | TGTTGTCCGA | CAGCGCTGCC | CTTCAACTGC | GGCTGGTCGT |
| 50581 | GGGCACGGCG | GACGCCGAGG | GACGTCGTAC | GATCACCGTC | CACTCGCGGC | CGGACGGAGA |
| 50641 | CCCGCGTACG | ACGCGCACAC | CTGCGGCATC | GTCCGAGACC | AGCCCGGACG | CGGAGTCGGA |
| 50701 | CACGGAGATC | CGTAGGGACA | CGTCCGCCTG | GACGAAGCAC | GCTCAGGCGA | CGGTCGCCCC |
| 50761 | CGCCCCTGAC | GTCCCCCCTT | CCGGGGTGGA | CGCGGAAGGG | GACGCCGTTC | GCCCCGCAGT |
| 50821 | GGAATGGAGC | GTGGCGGCGA | CGGAGTCGGA | TGCCTTCCAG | GCCGAGGACT | TCTACGCGTC |
| 50881 | CTTCGCCGCA | CACGGCTATG | GCTACGGCCC | GCTGTTCCAG | GGCGTACGGT | CAGGCCGTCA |
| 50941 | GGACGGGACC | GACGTCTACG | CCGAAGTCGC | CCTGGATCAC | GACCGCTTGC | CGTCTGCCGA |
| 51001 | GCAGTTCGGC | CTGCACCCCG | CGCTGCTCGA | CGCGGCGTTC | CAGACGATGC | GTCTGGGATC |
| 51061 | GTTCTTCCCC | GACGACGGAC | AGGCACGTGT | GCCGTACACC | TTCCGGGGGA | TTCGTCTCTA |
| 51121 | CGCCCCGGGA | GCCGCGCGCC | TGCGGGTCCG | TGTCTCGGCG | GTCGGGGCCG | ATGCCGTACG |
| 51181 | CGTGGAGTGC | GCCGATGAGC | GGGGGCGGCT | CGTCTGTGAG | ATCGACGCCC | TCGTCGTCAG |
| 51241 | CACGGTCTCC | CCGGACCAGT | TGCGGCCGGC | CGGACAGGAC | GCGACCCAGG | ACATGCTGCA |
| 51301 | CCGGATCGAG | TGGCCCGTCC | TCTCCCCGCC | GACCGGCAGC | GCCACCTCCC | CTGCTCCGCC |
| 51361 | CCGCTGGATC | GTGGTCGGGG | GCGAGGACGA | GGGCCTCGGG | CTCGGGGGCC | TTCGACTCGA |
| 51421 | CGGTCCGAGG | CTTGACGGTC | CCGGGCTTGC | GGAAGCGCTG | TCCGAAGCCG | GTATGGGGAC |
| 51481 | CGAGCGTCAC | CGGAACCTGG | CCGACGCGCT | GTCGGCCGTA | CGGACGCCGG | TGGACACGGC |
| 51541 | AGGCTCCGCT | GCCGCCGCCG | GCACGACCTC | CCTCATAGCC | GTCCCCGTAC | CGCAGTCGCC |
| 51601 | CACCATGGAC | GCCGGTGCCG | TGCGCCACGC | CGTCCACCGA | GCCCTGGAGC | TGGTGCAGGG |
| 51661 | CTGGGTGGCG | GCGGACGAGG | CGGCGGAAGA | GGGCGGGAGC | GACGGTGCCG | CGGCCGACCG |
| 51721 | GCGGCTGGTG | CTGGTCACGA | GCGGAGCGGT | GTCCACGGGT | GACGCCGACC | CGCTGCGCGA |
| 51781 | CCCGGTGGCC | GCGGCCGTCT | GGGGTCTGAT | CAAGTCCGCC | CAGTCGGAGC | AGCCCGGCCG |
| 51841 | CATCGTCCTC | GTTGACCTCG | ACGAGGGAGC | CGTGGACGGG | GCGGCCTTGG | CAGCCGCGAT |
| 51901 | CTCGACCGGC | GAACCACAAC | TCGCCCTGCG | CGACGGCGAT | GTGCACGTGC | CCAGGCTGGC |
| 51961 | ACCCCTGTCC | GTGCGGGACT | CGCAGACGCT | GCTGCCGCCC | GCCGGTACGC | GCGCCTGGCA |
| 52021 | TCTGGTCGGC | GCCGGCACCG | GAACCCTTTC | GGACCTCGCG | CTCGTACCGG | CGCAGACCGA |
| 52081 | CACCGTCGCG | CTCGCACCTG | GGCAGGTGCG | GATCGCGGTG | CGAGCCGCCG | GACTCAACTT |
| 52141 | CCGGGACACG | CTCATCGCGC | TCGGTATGTA | TCCGGGCGAG | GGCGTGATGG | GCGCCGAGGG |
| 52201 | CGCCGGAGTG | ATCACCGAGG | TCGGCCCGGA | CGTGGTGAGC | CTCGCCGTCG | GGGACCGCGT |
| 52261 | CCTGGGCATG | TGGACCGACG | GGTTCGGGCC | GTACGTCGTG | GCCGACCACC | GCATGGTGGC |
| 52321 | CCCGATGCCG | CGCGACTGGT | CCTACGCGGA | GGCCGCTTCG | GTACCCGCCG | TCTTCCTCAG |
| 52381 | TGCCTACTAC | GGACTCAGGC | ACCTGGCCGG | TCTGCGCGCC | GGCCAGTCGG | TGCTGGTGCA |
| 52441 | CGCGGCGGCG | GGCGGTGTGG | GCATGGCTGC | CGTCCAACTC | GCCCGGCACT | TCGGGGCCGA |
| 52501 | GGTCTTCGGC | ACGGCCGGCA | CGGCCAAATG | GGACGCACTG | CGGGCACAGG | GCCTGGACGA |
| 52561 | CCGGCACATC | GCCGGTTCAC | GGACGCTGGA | CTTCGCGGAC | CGGTTTCTCG | ACGCGACCGA |
| 52621 | GGGGCGCGGC | GTGGACGTCG | TCCTGAACTC | GCTGGCGGGC | GACTTCGTCG | ACGCCTCCCT |
| 52681 | GCGACTGCTG | CCGCGCGGAG | GGCGGTTCGT | GGAACTGGGC | AACCCGGACG | TACGCGACGC |
| 52741 | CGCGCAGGTC | GCCGCCGACC | GGCCGGGAAC | CGTCTACCGG | GCCTTCGAGC | TGATGGAGGC |
| 52801 | CGGGCCGGAG | CTGATCGGCC | GCATGCTGAA | CGAACTGCTG | GAACTGTTCG | AGTCCGGGGC |
| 52861 | GCTGCGCCTG | CTGCCCGTCA | CCCCGTACGA | CATCCGGCGG | GCACCCGACG | CCTTCCGCAC |
| 52921 | GCTCAGCCAG | GCCGGTCACG | TCGGCAAACT | GGTCCTGACG | ATGCCACCGG | CCTTCGAACC |
| 52981 | CCACGGCACG | GTCCTGATCA | CCGGCGGCAC | CGGGAACCTG | GGCGGAACAC | TCGCCCGCCA |
| 53041 | TCTCGTGACC | GAACACGGAG | TGCGCCACCT | GCTCCTGGCC | GGACGTAGGG | GGCCCGAGGC |
| 53101 | CGAAGGCGCC | GCCGAACTCG | TACGGGAACT | GCACGACCTG | GGCGCCTCCG | TCACGGTCGC |
| 53161 | CGCCTGTGAC | GTGGCCGACC | GAGCGGCGCT | CCGGAAACTC | CTCGGCGGCA | TACCGCCGGA |
| 53221 | GCGCCCGCTC | ACCGGAGTCG | TCCACGCGGC | GGGCGTTCTC | GACGACGGCG | TGGTCACGTC |
| 53281 | ATTGACCCCG | GACAGGGTCG | ACGGCGTCCT | GCGGCCCAAG | GTGGATGCCG | CTCTCAACCT |
| 53341 | CCACGAAGCG | GCCCTCGATC | CCGAACTCGG | TCTCGACATC | ACCGCGTTCG | TCCTGTTCTC |
| 53401 | GTCCGTCGCT | GCCCTGCTCG | GCGGCTCGGG | TCAGGGAAGC | TACGCCGCCG | CCAACGGCTT |
| 53461 | CCTCGACGGA | CTCGCCCAGT | ACCGGCGTGG | CCGCTCGCTG | CCCGCGCTCT | CCCTCGGCTG |
| 53521 | GGGCCTGGCC | GGGAGCGGCC | GGATGACATC | CCACCTGGAC | AGCCGGGCCC | TGCTCCGGCG |
| 53581 | CATGGCCAGG | GGCGGTGTCC | TGCCGCTGTC | CCCGGCGGAG | AGCATGGCAC | TGTTCGACGC |
| 53641 | GGCCCAGGGC | TTCGACGAGG | CGCTCCAGGT | GCCGGCGCGC | TTCCACACCG | CCGCACTGGG |
| 53701 | CGCCGACGGC | AACGTCCCGC | CGCTCTTCAA | CGGACTGATC | CGGGGCGGGA | CGGCGCATGC |
| 53761 | CGAGGCCCGG | CGCAGGACGG | TCGGCGCGTC | GCCCGCGGGT | GGTCCTGCCG | GAGGCGAGCC |
| 53821 | GGTGAACCTC | GCCGACCGGC | TGTCCGGACT | GACGGAGGAC | GAACAGCGGG | CGCTGCTCCT |
| 53881 | CGACACGGTG | CGCACGCACG | CGGCTCTCGT | CCTGGGCCAC | ACGGGCACGG | ACGGCATCCA |
| 53941 | GGCCGACCGG | GCGTTCAAGG | ACCTGGGATT | CGACTCGCTG | ACGGCCGTCG | AGATGCGCAA |
| 54001 | CCGGCTCACC | GCCGCCACGG | GGCTGCACCT | CGCGGCGACG | CTGGTCTTCG | ACCACCCCGC |
| 54061 | TCCGGCGGAC | CTCGCCGAGC | ACCTCCGCTC | TCGACTCGTC | CCCGAGGGGA | CGGACGTACC |
| 54121 | GCCGCTCCTC | GCGGAACTCG | GTCGGCTCGA | AACGGCGTTC | AAGAAGCTGA | CCACCGCGGA |
| 54181 | CCTCGCCTCG | GTCGTGCCCG | ACGACATCGC | CCGCGACGAG | ATCGCCGTAC | GTCTCGCCGC |
| 54241 | CCTCGGTTCC | CTGTGGAACG | GGCTCCATGG | CAACGGCCTC | AGCGGAGACG | CGGCGCAGAA |
| 54301 | GCACGGCGAC | TCGATCGTCG | AGGACATCGA | CTCCGCCGAC | GACGACGAGA | TCTTCGCCTT |
| 54361 | CCTCGACGAG | AGCTTCGGCG | ACTCCTGACC | GCAGGCACCT | CCGTACGGAC | CGACGACTCT |
| 54421 | GCAGACGGGT | GATGAGAGAT | GGCGACCGAA | CACGAGCAGA | AGCTCCGCGA | CTACCTCAAG |
| 54481 | CGGGCCACCA | CCGAACTCCA | CAAGGCCACG | GAACGGCTGA | AGGAGGTCGA | ACAACGCGCT |
| 54541 | CACGAGCCGG | TTGCGATCGT | GGGGATGGGA | TGCCGGTTCC | CGGGCGGGGC | GTCCTCGCCT |
| 54601 | GAGGAGTTGT | GGGACCTGGT | GGCGGCGGAG | ACGGACGCGG | TCTCCCCCTT | CCCGGTGGAC |
| 54661 | CGGGGGTGGG | ACGTGACGGG | GCTGTACGAC | CCGGATCCGG | ACGCGGCAGG | GCGTGCCTAC |
| 54721 | GTCCGCGAGG | GCGGGTTCCT | CCACGACGCG | GGGGAGTTCG | ACGCGGGGTT | CTTCGGAATC |
| 54781 | TCTCCGCGTG | AGGCGTTGGC | GATGGATCCG | CAGCAGCGGT | TGCTGCTGGA | GACGTCGTGG |
| 54841 | GAGGCGTTGG | AGCGGGCGGG | CATCGATCCG | CACACGCTGC | GCGGCACGCG | GACCGGGGTC |
| 54901 | TACATGGGTG | CCTGGAACGG | CGGATACGCC | GAGGGGATTC | CCCAACCCAC | GGCGGAACTG |
| 54961 | GAGGCCCAGC | TCCTCACCGG | CGGCGTGGTG | AGCTTCACCT | CGGGCCGTGT | GTCCTACCTC |
| 55021 | CTGGGTCTGG | AGGGGCCGGC | GGTGACGGTG | GACACGGCGT | GTTCGTCGTC | GCTGGTCGCG |
| 55081 | CTGCACCTGG | CGGTGCGGGC | GCTGCGCAGT | GGTGAGTGCG | ACCTCGCCCT | CGCCGGCGGC |
| 55141 | GCGACGGTGA | TGTCGACGCC | CGACGTGTTC | GTGCGCTTCT | CCCGGCAGCG | AGGAGTGGCC |
| 55201 | GCGGACGGTC | GCTGCAAGGC | GTTCTCCGCG | TCGGCCGACG | GATTCGGACC | GGCTGAGGGT |
| 55261 | GTGGGCGTGC | TGGCGGTGGA | GCGGTTGTCG | GATGCGGTGC | GTCATGGGCG | TCGGGTGCTG |
| 55321 | GCGGTCGTGC | GTGGTTCCGC | GGTCAACCAG | GACGGTGCGA | GCAACGGACT | GACGGCGCCG |
| 55381 | AGCGGACGAG | CTCAGGCCCT | TCTGATTCGT | CGAGCGTTGG | CGGATGCGCG | GTTGGGTGTG |
| 55441 | GCTGATGTGG | ATGTGGTGGA | GGGGCATGGG | ACGGGGACGC | GTCTGGGTGA | TCCGATCGAG |
| 55501 | GCGCAGGCGT | TGTTGGCGAC | GTATGGGCAG | CGGGATGCGG | GTCGGCCGTT | GCGGCTTGGT |
| 55561 | TCGTTGAAGT | CGAATGTGGG | GCATACGCAG | GCGGCTGCCG | GTGTGGCGGG | CGTGATCAAG |
| 55621 | ATGGTCATGG | CGATGCGGCA | CGGTGTCCTG | CCGAAGACGC | TGCACGTGGA | TGAGCCGACG |
| 55681 | GCGGAGGTGG | ACTGGTCGGc | CGGCGCGGTG | TCTTTGCTGA | GGGAGCAGGA | GGCGTGGCCT |
| 55741 | GAGGTGGGGC | GTCTGCGTAG | GGCTGCGGTG | TCTTCGTTCG | GTGTGAGTGG | GACGAACGCG |
| 55801 | CATGTGGTGG | TGGAGGAGGC | GCCGGTTCCG | GAGGACGGGG | AGGCGGTCGG | GGGCGGTGTG |
| 55861 | CCTTTGGCTG | TGGTGCCGGT | GGTGGTGTCG | GGTCGTTCTG | CGGGTGCGGT | GGCGGAGCTG |
| 55921 | GCGGGCCGGG | TCAGCGAGGT | GGCTGCGTCT | GGTCGGTTGG | TGGATGTGGG | GTTGTCGTCG |
| 55981 | GTGGTGTCGC | GGTCGGTGTT | CGAGCACCGG | TCCGTGGTAC | TGGCGGGGGA | CTCTGCCGAG |
| 56041 | CTGAATGCCG | GTTTGGATGC | TGTGGCCGGT | GGTGTGCCGT | CGCCTGGTGT | GGTGTCGGGT |
| 56101 | GTGGCGTCGG | GTGAGGGTGG | CCGGTCGGTG | TTCGTGTTCC | CTGGTCAGGG | GACGCAGTGG |
| 56161 | GCGGGGATGG | CGCTCGGGTT | GTGGGCGGAG | TCGTCGGTGT | TCGCGGAGTC | GATGGCGCGG |
| 56221 | TGTGAGGCGG | CGTTCGTCGG | CTTGGTGGAC | TGGCGCTTGT | CGCAGGTTTT | GAGCGATGGG |
| 56281 | TCGGCGCTGG | AGCGGGTGGA | GGTGGTGCAG | CCGGCGTCGT | TCGCGGTGAT | GGTGTCGCTT |
| 56341 | GCTGAGCTGT | GGCGGTCGTT | GGGTGTGGTG | CCGGATGCGG | TGGTGGGGCA | TTCGCAGGGG |
| 56401 | GAGATCGCTG | CTGCGGTGGT | GGCGGGTGGT | TTGTCGCTGG | AGGACGGGGC | GCGTGTGGTG |
| 56461 | GTGTTGCGTG | CGCGGTTGAT | CGGTCGTGAG | CTGGCCGGGC | GCGGTGGGAT | GGCGTCGGTG |
| 56521 | GCGCTGCCGG | TCGCGGTGGT | GGAGGAGCGT | CTGGCGGGGT | GGGCGGGGCG | TCTGGGTGTG |
| 56581 | GCGGTGGTCA | ACGGACCGTC | CGCCACGGTC | GTCGCGGGTG | ATGTGGATGC | GGTGGCGGAG |
| 56641 | TTCGTGGCCG | CGTGCGAGGT | GGAGGGGGTT | CGGGCGCGTG | TTCTGCCGGT | GGACTACGCC |
| 56701 | TCGCACTCGG | CGCACGTGGA | GGACCTGAAA | GCCGAGCTTG | AACAGATTCT | GGCCGGCATC |
| 56761 | GGCCCGGTGA | CCGGTGGGAT | CCCGTTCTAT | TCGACGTCCG | AAGCCGCGCA | GATCGACACG |
| 56821 | GCTGGTCTGG | ACGCGGGGTA | CTGGTTCGGG | AATCTGCGTC | GGCCGGTGCG | GTTCCAGGAG |
| 56881 | ACGGTCGAGC | GGTTGTTGGC | GGATGGTTTC | CGGGTGTTCG | TGGAGTGTGG | CGCGCATCCG |
| 56941 | GTGCTGACGG | GGGCGGTGCA | GGAGACCGCG | GAATCCACCG | GTCGCCAGGT | GTGTGCGGTC |
| 57001 | GGATCCCTGC | GTCGTGACGA | GGGAGGTCTG | CGCCGCTTCC | TCACCTCGGC | CGCGGAGGCA |
| 57061 | TTCGTCCAAG | GCGTGGAGGT | GTCCTGGCCG | GCACTGTTCG | AAGGCACCGG | CGCCCGCACG |
| 57121 | GTCGACCTGC | CCACCTACCC | CTTCCAACGT | CGGCGCTACT | GGCTGGAGTC | GCGCCCTCCC |
| 57181 | GCGGCGCCGA | TCGAGACTGC | CGCAGCCTCT | GGCATCGAGA | GCTGGCGCTA | CCGCGTGGCG |
| 57241 | TGGAAGAGCC | TGTCGCTGTC | GGAGTCGTCG | CGTCTTGACG | GCCGGTGGCT | GCTCGTCGTG |
| 57301 | CCCGAAACCC | TGGACGCCGA | CGGCACGCGG | ATCGCCCACG | ACATCCAGCA | CGCCCTCACC |
| 57361 | ACCCACGGCG | CCACGGTCTC | CCGTCTGACG | GTCGACGTGA | CGACGACCGA | CCGCGCCGAC |
| 57421 | CTGTCGGCGC | GGCTCACCAC | CACCGCGGCC | GAAGACCAGG | GGCCTCTCCG | GGGCGTCCTC |
| 57481 | TCCCTCCTGT | CCACCGATGA | ACGGCAGCAC | CCGGATCATC | CCGGTGTCGA | CCGTGCCACG |
| 57541 | GCGGGCACGA | TGCTGCTCGC | CCAGGCGTGC | GGGGATCTGG | TCGTGGCCCG | GGGCGTGGAG |
| 57601 | CCGAGGCTGT | GGGTCGTGAC | CCGCGGGGCG | GTCGCGGTGT | CCCCCGCCGA | GCGTCCGTCG |
| 57661 | TCAGCCGGCG | CCCAGGTCTG | GGGCCTGGGG | CGCTGCGCGG | CGCTCGAACT | TCCCACTCGG |
| 57721 | TGGGGTGGGA | TGGTCGACCT | TCCCCCGGCG | GCCCGGGATG | CTGGAAGGCA | CGTACGGCGG |
| 57781 | CTCGTGCGTC | TGCTGTCGGA | GACCTGTGCG | GAGGACCAGG | TGGCGCTGCG | TGCGTCGGGT |
| 57841 | GCGTACGGCC | GCAGGCTGCT | GCCCGCGTCC | AGCCCCTCCG | TATCCGTCCC | CCGGACCGCG |
| 57901 | AAGAGCGGCT | ACCAGCCGCG | CGGCACGGTG | CTGGTGACCG | GCGGAACCGG | TGCCCTCGGT |
| 57961 | GGCCACTTGG | CACGGTGGCT | GGCCCGCAAC | GGCGCCGAGC | ACATCGTTCT | GGCCGGGCGT |
| 58021 | CGGGGCGAGG | GTGCTCCAGG | AGCCGCGGAA | CTGTCGGCGG | AGCTCAAGGA | GCTGGGTGCG |
| 58081 | GAGGTCACCG | TCGCGGCCTG | CGACGTGGCG | GACCGGAACG | CGTTGCGTGA | CATGCTGGAA |
| 58141 | TCCCTGCCGG | CCGACCGGCC | GCTGTCGGGG | GTGTTCCACG | CTGCCGGTGT | CCCGCACTCG |
| 58201 | GCGCCGCTGG | CCGAGACGGA | TGTGGCGGGG | CTCGCCGCCG | TGCTCCCGGG | GAAGGTCGTC |
| 58261 | GGGGCACGGC | ACCTGCACGA | ACTCACCAGG | GAGAAGGAAC | TGGACGCGTT | CGTGCTGTAC |
| 58321 | GCGTCGGGCG | CCGGGGTGTG | GGGGAGCGGC | GGGCAGAGCG | CGTACGGAGC | CGCCAACGCC |
| 58381 | GCACTGGACG | CGCTGGCCGA | ACAGCGCCGG | GCTGAGGGAC | TGCCCGCCAC | TTCGGTCTCC |
| 58441 | TGGGGCCTGT | GGGACGGCGG | AGGCATGGCC | GGCGAGCGAG | GCGAGGAGTT | CCTCACCGCC |
| 58501 | CTCGGCCTGC | GGGCCATGGA | GCCCGAGTCG | GCTGTCGCCG | CCCTGGAGGA | GGCCCTGGAT |
| 58561 | CGTGGGGACA | CCTGCGTGAG | CGTGGTCGAC | GTCGACTGGT | CCCGGTTCGC | CGAGTCGTTC |
| 58621 | ACCGCCTTCC | GGCCCAGCCC | GCTGATCGGG | GAGCTCCCCG | GGGTACGTGC | CGTGCCCGAC |
| 58681 | GGATCGGCGG | GCGGACCGTC | GGACGACCTC | GCGGACGCTG | CGCGGCACGG | CGGGGCAGCC |
| 58741 | GACCGGGGTG | TGCCTGCAGG | GCTCGCCCGG | GCGACGGGCG | ACGACCGGCA | GGACATCCTG |
| 58801 | CTCGATCTCG | TACGCCGCCA | TGCCGCCGCC | GTCCTCGGTC | ACCCGGGACC | GCAGCACATC |
| 58861 | GAGCCCGACG | CCGGTTTCCG | GACCCTGGGG | TTCAGTTCGG | TCACCGCGGT | GGAACTGGCC |
| 58921 | AACAAGCTCG | GTGCGGCCGT | GGGAACGAAG | ATCCCCGCCA | CCTTCGCGTT | CGACCACCCC |
| 58981 | AACGCCCGTG | CCGCGGCGTC | CCGCCTCGAC | GTCCTGTTGG | CGGCGTCGAG | CGATGAGACC |
| 59041 | GCGCAGGAGG | CGGAGATCCG | GCAGGCACTG | CGGACTGTGC | CGCTGGCCCG | GCTGCGGGCT |
| 59101 | GCGGGGCTCC | TCGACGGCCT | GCTCGAACTC | GCCGGGCTGG | AAGCGGAGCC | CGGCCTGCCG |
| 59161 | GGCGACGTAC | CGGATCGCGG | TGCGGCCACG | CCGGACGAGG | AGTCCGCCCT | GGCGGAAGTC |
| 59221 | GACGGCCTGG | ACGCCGAAGC | ACTGGTCGAC | CTCGTCCTCA | ACCAGTCCGA | CTCCTGACCG |
| 59281 | CCGGCGGCGG | CGCCGCGGCC | CGCCGTGCCG | TCGCCGCCCT | CGGCCGTACG | AAGAACCCCA |
| 59341 | CAGACCTGAC | CGGGTCACGG | CCCGGTGCTC | AGCAAGGAGA | CCACTCATGG | CTCTGTCCCA |
| 59401 | AGAGAAGGTA | CTGGAGGCAC | TGCGCACCTC | CGTCAAGGAC | GCCGAACGGC | TGCGCAAGCG |
| 59461 | CAACCGCGAA | CTCCTCGCGG | CCCGCCACGA | GCCCATCGCC | GTCGTCGGCA | TGGCCTGCCG |
| 59521 | CTATCCCGGC | GGGGTCCGTT | CGCCCGAGGA | CCTCTGGGAA | CTCGTCGTGT | CGGGCACGGA |
| 59581 | CGCGGTGGGT | CCCTTTCCCG | AGGACCGTGG | CTGGGACGTG | GAGCGGATCT | ACGACCAGGA |
| 59641 | CCCGTCCGTC | CCGGGCACCA | CGTACTGCCG | CGAGGGCGGA | TTCCTTTACG | ATGCGGGGGA |
| 59701 | CTTCGACGCG | GCTTTCTTCG | GGATAGGGCC | GCGCGAGGCC | ACCGTGATGG | ACCCCCAGCA |
| 59761 | GCGCCAGCTG | CTGGAGGCGT | CCTGGGAAGC | CCTGGAGCAG | GCCGGGCTGG | ACCCCCGGGC |
| 59821 | GCTCCGCGGC | AGCCAGGGGG | GTGTGTTCGT | CGGCGCGGCG | AACCAGGGCT | ACGTACCGGG |
| 59881 | TGACGCGGCG | GCGTCGGGAC | GTCTGCCGGA | GGGTTCCGAC | GGCTATCTGC | TCACCGGCAA |
| 59941 | CGCCGACGCC | GTCCTGTCGG | GCCGGATCAG | CTACTTCCTG | GGCCTGGAAG | GCCCGTCCAT |
| 60001 | GACCGTCGAG | ACGGCCTGCT | CCTCCTCCCT | GGTGGCACTG | CACCTGGCGG | TGCAGGCGCT |
| 60061 | GCGCCGTGAG | GAGTGCGAGT | TCGCCCTGGC | CGGAGGGGTC | GCCGTGCTCG | CCAACCCGGC |
| 60121 | CGCCTACGTG | GAGTTCGCCC | GGCAGCGGGG | ACTCGCCCCG | GACGGGCGCT | GCAAGGCGTT |
| 60181 | CGACGACGCG | GCGGACGGTA | CGGGCTGGGC | CGAGGGCGTC | GGCGTCCTGG | TGGTGGAGCG |
| 60241 | GCTGTCGGAC | GCGGTACGCA | AGGGGCACCG | GGTCCTCGCC | GTCGTGCGGG | GCACGGCGGT |
| 60301 | GAACCAGGAC | GGTGCCAGCA | GCGGTCTGTC | CGTGCCCAAC | GGGCCCTCCC | AGCAGCGGGT |
| 60361 | CATCCGCCGA | GCGCTGGCCG | ACGCCCGGCT | GGAGGCCGGC | CAGATCGACG | CGGTGGAGGC |
| 60421 | CCACGGCACC | GGCACTCGGC | TGGGGGACCC | CATCGAGGCG | CAAGCCCTGC | TGGACACGTA |
| 60481 | CGGAGAGGAG | CGGAGCCCCG | AACGCCCTCT | GTGGGTCGGG | TCGTTGAAGT | CGAACTTCGG |
| 60541 | TCACGCACAG | GCGGCAGCCG | GAGTCGGCGG | CGTCATCAAG | ACGGTGATGG | CGCTCCGGCA |
| 60601 | CGGCCTGCTT | CCCCGCACGC | TCCATGTGAC | CAGCCCGACG | CGGCACGTCG | ACTGGGGCGA |
| 60661 | CGGACAGGTG | CGGCTGCTGA | CCGAGCCGGT | CGACTGGCCG | CGGACCGGCG | CCCCCCGGCG |
| 60721 | GGCCGCGGTC | TCGGCGTTCG | GCGTGAGCGG | CACCAACGGG | CACATCATCC | TCGAGGAGGC |
| 60781 | GCCGCCGCCC | ACCCGGCCCG | AAGCGGTCCG | GCAGGCCGGG | GAGCGGCGGC | CGGTCCTGGT |
| 60841 | CCCGTGGACG | CTGTCCGGCC | GTACGAGGCC | GGCGCTGTGC | CGGCAGGCCG | CGCGCCTGGC |
| 60901 | GGCGCACCTC | GAACAGCACC | CGGACCTCGA | CCCGCTGGAC | GTCGGGTTCT | CGCTCGCCAC |
| 60961 | GACGCGCACC | CACTTCGAGC | ACCGGGCCGT | GCTGCTCGCG | GACGCCGCCA | CCGAGGGCGG |
| 61021 | CTCCCGTGCC | GACGCGCTCG | GGGCGTTGCG | GGCGATCGCG | GAGGACCGCG | ACCCGGGCGG |
| 61081 | GGCGGTACGG | GACACCGCGC | GGGGCGAAGG | GCGTATCGCC | TTCCTGTTCT | GCGGGCAGGG |
| 61141 | CAGCCAGCGG | CCCGGCATGG | CGGAGCAGCT | GTACGCGCAG | TACCCGGCGT | TCGCGCGGGA |
| 61201 | ACTGGACACG | ATCGCGACGC | ATCTGGACGC | CCATCTGGAC | CGTCCGTTGG | CGACGGTGAT |
| 61261 | GTTCGCGCCG | GCCGGTACGG | CGGAGGCCGC | GCTGCTCGAC | GGCACGCAGT | ACGCCCAGGC |
| 61321 | GGCCCTGTTC | GCCGTAGAGG | TCGCGTTGTT | CCGGCTCTTC | GAGGGCTGGG | GGCTGCGCCC |
| 61381 | CGACGTACTG | CTGGGCCATT | CCGTGGGCGA | GCTGGCCGCC | GCCCACGTGG | CCGGGGTGTT |
| 61441 | CGGGCCGGCG | GACGCCTGCT | CGCTGGTCGC | CGCACGCGGC | CGGCTCATGC | AGGAGCTGCC |
| 61501 | GGCCGGCGGC | GCGATGCTCT | CGGTCCGTGC | CGCCGAGCAC | GAGGTGCGGG | AGCTGATCGC |
| 61561 | CGGGCAGGAG | GACCGCATCG | CGGTGGCGGC | CGTCAACGGG | CCCCGCTCCG | TGGTCGTATC |
| 61621 | CGGGGACGAG | GACGCGGTCT | CGGCGCTCGC | CGAGGAGCTG | ACCGAATACG | GCGTGCGCAC |
| 61681 | CAAGCGCCTC | AACGTCAGCC | ACGCCTTCCA | CTCCCCACGT | CTGGACTCCA | TGCTGGAGAC |
| 61741 | GTTCCGCCGG | GTCGCGGAGA | CGGTGGAGTA | CCGCGAGCCG | ACGCTCGACG | TGATCAGCGG |
| 61801 | CCTGACCGGC | CGCCCGGCCG | ACGCCGGGGA | ACTCGCCACC | GCCGACTACT | GGGTCCGGCA |
| 61861 | GGCGAGGGAG | ACCGTCCGGT | TCCACGACGG | GGTGCGCGCC | GCGCACGCGC | GCGGCGTCAG |
| 61921 | CACCTTCGTG | GAGCTGGGGC | CGGACGGCGT | GCTGTGCGGC | CTGGCCCTGG | AGACCCTGGC |
| 61981 | GGAGGAGACC | GACGGGGAAG | CGGCCGCCGA | GACGCCCGGC | CGGGCGCGGG | CGGCGCTGGT |
| 62041 | GCCCGTGATG | CGTCGGGAGC | GGCCGGAGGG | CAGTACCCTC | CTGACGGCGC | TCGCCACGGC |
| 62101 | CCACGCGCGC | GGGGCGGAGG | TGGACTGGTC | CCGGTTCTAC | GCCGACACCG | GCGCCCGCCA |
| 62161 | CACCACACTG | CCCACCTATG | CCTTCCAGCG | CCAGCGGTTC | TGGCTGGAGA | CGGCGGCCCC |
| 62221 | CGCCGCGCCC | GCGGCGGGCC | AGGGGGCCGG | ACCGGCCGAC | CCGCAGGACA | GCACCGGTCC |
| 62281 | GGCCGCCCGG | CCCACGCTGA | CGGAACAGGA | CCTCCTCCTG | CTCGTGCGGA | CGGAAGCGGC |
| 62341 | GGCCGCACTC | GGCCACGCCG | AACTGGAGGA | CGTACCGGCC | GACAGCCTCT | TCGGCGACAT |
| 62401 | CGGCTTCGAC | TCGCTCGCGG | CCATCGAACT | GGGCGCCGCC | CTGACCGGCG | CCACCGGGCT |
| 62461 | GGAAGTGCCG | TCGTCCCTCG | TCCTCGACCA | CCCCACGCCC | AGGGAGCTGG | CCGCGCACCT |
| 62521 | GGCAGCCGCC | CGGACGGCCG | CCGACAGCGA | CGACACGTCC | CCCGAAGGCC | CGGACACGGC |
| 62581 | CGGTGAGAGC | AGCCTGTCGG | CGATGTACCG | GCGGGCCGTG | CGGCTCGGCC | GGGCCGAGCC |
| 62641 | GTTCATCGGC | ACACTCGCCG | AACTCGCCGC | CTTCCGGCCC | GTCTTCCCCG | CCGATCACAC |
| 62701 | CCTCGCGGAC | GGCGAGACCG | TCGGACAGGC | GGCCGCCGCC | TGGCAGCCGG | CTCCGGTGCG |
| 62761 | CCTGGCCACC | ACGGACGGTG | AGGGACCGGA | GCTGATCTGC | TGCGCGGGTA | CGGCGGTGGC |
| 62821 | GTCGGGACCG | GAGGAGTTCA | CCGCGCTGGC | CGCGGCCCTG | GGCGACCGGC | TGACCGTGTC |
| 62881 | GGCACTGCGC | CAGCCCGGCT | TCCGCGCGAA | CGAGTTGCTG | CCCGGCTCCC | TGGACGGGCT |
| 62941 | GCTCGACGCG | CAGGCGGACG | CGGTGCTGCG | GCACACGGGT | GACAGGCCCT | ACGCCCTCCT |
| 63001 | CGGCCACTCG | GCGGGCGGGG | CGCTGGCGCA | CGCGCTGGCC | TGCCGACTGG | AGGAGCTCGG |
| 63061 | CGCGGGTCCC | GCGGCGCTGG | TCCTGGCCGA | CGTCTATCTG | CCCAGCTCGC | CGGGGGCGAT |
| 63121 | GGGGGTGTGG | CGCAACGAGA | TGCTCGACTG | GGTCATGCGG | CGTTCCGTGG | TGTCCATCGA |
| 63181 | CGATGCCCGG | TTGACGGCCA | TGGGCGCCTA | CAACCAGATG | CTCCTGGAGT | GGACACCGCG |
| 63241 | GCCCACGAAG | GCGCCGGTCC | TGTTCCTGCG | CGCGACGGAG | CCGGTGAGGC | CGTGGTCCGG |
| 63301 | AGAACCGGAG | AGCTGGCGGG | CGCACTGGGA | CGGCGGCGAC | CACACCGCCG | TCGACGTGCC |
| 63361 | CGGCACCCAC | CTGACGCTGA | TGACCGAGCA | CGCCCGCCAC | CTCGCGGCGA | CCCTCCACAC |
| 63421 | CTGGCTCGGC | ACCCTGTGAA | CCACGCCCGG | GGCGGCTTCG | CCGCGCGTAG | GACTGCCGCC |
| 63481 | TCCCCCGACT | TCCGTACACC | GCGACACCTT | GGAGGACTCC | CGTGACAACG | CAGTGGACCA |
| 63541 | CCCCGTCCGT | GCTCGGCCGC | AGACTGCAAC | GCACCTACGT | GGGGCACTGG | TTCGCAGGAA |
| 63601 | CGCAGGGAGA | CCCCTACGCG | CTGATCCTGC | GCGCCCAGCG | GGACGACACC | ACCCCCTACG |
| 63661 | AGGAGGACGT | CCGCGCACGC | GGACCGGTGT | TCCACAGCGA | GGTGCTCGAC | ACCTGGGTGA |
| 63721 | TCACGGACGG | CGCTCTCGCC | CGGTCCGTCC | TGACCGACGC | CCGCTTCGGC | GGGCTGACGC |
| 63781 | GCGCGGGAGG | ACGGTATCGC | GCGGAGCTTC | TCCCTCCGGC | GGGCCCCGAG | GTCGGTCCGG |
| 63841 | CCCGCGCAGG | GGTACGCGGC | GGCGTGCGGG | CCGACGCCGA | TCCGGCGGTG | TCGGCGCAGG |
| 63901 | ACGAGGTGGT | GGTGGAGGCC | CTCGCCGAGC | AGCTCTCACG | CACCCTCCTG | GGCGGACTCG |
| 63961 | GCGACGACTT | CGACCTCGTC | GCCGCCTTTG | CGCGACGCCT | GCCGGCACAG | GTCCTGGCGG |
| 64021 | AATTCCTCGG | GCTGCCCGCA | GCCGCGCGCA | GCCGGTTCGA | GGAACTGCTG | GCCGGCTGCG |
| 64081 | CCCACAGCCT | CGACAGCCGG | CTCTGTCCGC | AGACGCTCGA | CATCACACGG | ACCGGCCTCG |
| 64141 | GAGCGGCGGC | CGAGCTCCGG | GAACTGCTCG | CGCGCCACCT | CGGCGGGAGC | GGACCACGCT |
| 64201 | CCGCTCAAGC | GGCAGTCTCC | CTGGCAGTCG | AGGTGGCCGC | ACCCGCCGGC | GCGCTCATCT |
| 64261 | GCAACGCGGT | CGAGGCGCTG | AGCAGCTCTC | CCGGGCAGTG | GAACGCCCTC | CGCCAGAACC |
| 64321 | CGGAGAAGGC | CGACGCCGTC | GTGGCGGAGA | CCTGGTGGCG | ACGACCGCCG | GTGCGGGTGG |
| 64381 | AGAGCCGGAT | CGCCCAGGAG | GACGTCGACG | TGGCCGGAGT | GCCCGTCCCC | GCGGACGGGC |
| 64441 | ACGTGGCGAT | CCTCGTCGCC | GCCGCCCAGC | GCGACCCGGC | GATCACCCCG | GCCCCGACGA |
| 64501 | AGGACGACAC | CGGCACCCCC | GGACAGGGCG | ACTGCGGCGT | GCCCCTGGGG | CTCGTCGGCG |
| 64561 | ACGCGCACGC | CACCTCCGCC | GCCCGGACGG | TCCGCGCCCT | CTGCCGCGGT | GCGCTGCGAG |
| 64621 | CGCTCGCGCA | GGAGGCACCG | GGCCTGCGGC | CGAACGGGAC | CCCGGTGCGC | CTCAGGCGGG |
| 64681 | CACCCGTCAC | GCTCGGCCAC | GCCCGCTTCC | CCGTCGCCCG | GACGGGCCGG | GGGACACCGA |
| 64741 | CCGACGCGGG | CGCGGCATGA | GCACCCGCGA | CGACCACCGA | CTGCCGAACG | GGGAGACGAG |
| 64801 | CCGATGCGCG | TCCTGATGAC | GTCGATCGCC | CACAACACGC | ACTACTACCA | CCTGGTGCCG |
| 64861 | CTCGCCTGGG | CCCTGAAGGC | CGCGGGCCAC | GAGGTGCGCG | TCGCCGGCCA | GCCCCGCGTC |
| 64921 | ACGGACATCA | TCACCGGGTC | CGGACTGACC | GCCGTGCCGG | TCGGTGACGA | CGAGGACATG |
| 64981 | ATGGAGCTGT | TCGCCGAGAT | CGGCGGAGAC | ATCACCCCCT | ATCAGGAGGG | ACTGGACTTC |
| 65041 | GCCGAGGAGC | GGCCCGAGGC | ACGGTCCTGG | GAACATCTGC | TCGGACAGCA | GACCGTTCTG |
| 65101 | ACCTCGCTGT | GCTTCGCACC | GCTCAACGGC | GACTCGACGA | TGGACGACAT | CGTCGCGCTG |
| 65161 | GCCCGCTCCT | GGCAGCCGGA | CCTGGTGATC | TGGGAACCCT | TCACCTTCGC | CGGAGCGGTC |
| 65221 | GCCGCCCACG | CCGTGGGCGC | GGCGCACGCC | CGCGTCCTGT | GGGGTCCCGA | TGTCATCGGC |
| 65281 | CGGGCCCGGG | AACGGTTCGT | GGAGGCCAAG | GCACAGCAGG | CTCCCGAACA | CCGGGAGGAC |
| 65341 | CCGATGGCCG | AGTGGCTCGG | CTGGACCCTG | GAGAGGCTGG | GCCTCCCGGC | CGCCGGAGAC |
| 65401 | GGGATGGAGG | AGTTGCTGAA | CGGCCAGTGG | GTCATCGACC | CGGGCCCGGA | GAGCGTCCGG |
| 65461 | CTCGACCTTC | GCGAGCCGAT | CCTGCCCATG | CGTTTCGTTC | CCTACAACGG | ACCTGCCGTC |
| 65521 | GTCCCCGGAT | GGCTGTCCGA | GAAGCCGAAG | CGACCGCGCG | TCTGCCTCAC | CCAGGGAGTG |
| 65581 | TCGGGACGCG | AGACCCACGG | CAAGGACGCC | GTCCGCTTCC | AGGACCTGCT | CGCGGCGCTC |
| 65641 | GGCGACCTCG | ACATCGAGAT | CGTCGCCACC | CTGGACAGCA | CCCAGCGGGA | GAACCTGACG |
| 65701 | GAGGTCCCCG | ACAACGTCCG | GATCGTCGAC | TTCGTCTCGA | TGGACGTGCT | GCTGCCGAGT |
| 65761 | TGCGCCATGA | TCATCTACCA | CGGTGGCGCC | GGCACCTCGG | CGACGGCCCT | CCTGCACGGC |
| 65821 | GTTCCGCAGG | TCGTCATCGG | AGCGCACTGG | GACGTGCCGG | TCAGGGCACG | GCAGCTCGAC |
| 65881 | GACCTGGGCG | CCGGCATCTT | CATCCGGCCC | GAGGACCTCG | ACGCCGCCAC | ACTGCGCGCG |
| 65941 | GCGGTTCAGC | GCGTGCTCAC | CGAGCCCTCC | CTCCAGCGGG | CCGCGGACCG | GCTGCGGGCC |
| 66001 | GAGATGCGCT | CCAACCCCAC | GCCGGCCGAG | ACCGTCACGG | TGCTGGAGCG | GCTCTCCCGG |
| 66061 | AGCCACCGAC | AGCCCCGCTG | ACCACACGCG | GTACACGGTG | CGGGCCCACG | TGCCGGGGGC |
| 66121 | TCCACCGTCG | CCGGCGGTCG | TCGGATCGCC | GTCCGGCCAT | GTCCCGGCAC | CCAAGGACGG |
| 66181 | AGCAGAGCAG | AACATGGAAT | TCGAAGGTCA | GGTCGCGCTC | GTCACCGGGG | CCGGCAGGGG |
| 66241 | GATCGGCCGT | GCGACGGTCG | TCCGCCTCGC | GGAGGCCGGA | TGTGACATCG | CCCTCCACTA |
| 66301 | CAACCAAGCG | AAAGCGCAGG | CCGAGGAAGT | CGCCGAGCGC | ATCGCCGCAC | TGGGCCGCAC |
| 66361 | GGTCGAACTG | TTCCCGGGCG | ACCTCTCCCG | CCCCGAGACC | GGGCGACAGC | TCGTGGCCGC |
| 66421 | GGTGCAGCAG | AAGTTCGACC | GGATCGACAT | CCTGGTGAAC | AGCGCGGGCA | TCACACGGGA |
| 66481 | CAAACTCCTG | CTGTCCATGG | AGGCGGACGA | CATCCACCAG | GTCATCGCCA | CCAACCTCGT |
| 66541 | CGGCCCGATG | TTCCTCACCC | AGGCGGTCGC | GCTCACCATG | CTGCGTCAAC | GCTCCGGGCG |
| 66601 | CATCGTCAAC | ATCTCCTCCG | CCGCCGCGAG | CAGGCCCGGA | AAGGGCCAGT | CCAACTACGC |
| 66661 | CGCGTCCAAG | GCCGGTCTGG | AGGCCTTCAC | CAGGGCCATG | GCGGTGGAAC | TCGGATCCCG |
| 66721 | CGGAATTCTC | GTCAACGCGG | TCGCTCCCGG | CATCGTCAAG | ACCGGCCTGA | CCGAGGCTCT |
| 66781 | CCGCGAGGGG | GCGGAGCCCG | AACTCCTGGC | CCGGCAGGTG | ATCGGTTCCT | TCGCCGAACC |
| 66841 | CGAGGCGGTG | GCGGAGGCGG | TGGCCTACTT | GGCGAGCCCG | CGCAACACGC | ACACGACGGG |
| 66901 | CACGGTCCTC | ACCGTCGACG | GCGGGCTCAA | GATGGTGTGA | GGCCCACCGG | GCATCGGACA |
| 66961 | GCCGGTGGAT | CCGCCGGAGG | CGGAGACCCG | CGCACCGCGG | GCGTCGACCC | GCGCACCGCG |
| 67021 | GGCGCGCGTG | GGGGCGCCCG | CGGTGCGCGA | AGGCCGCCTC | TGGCTCGCGT | CACCGGAAGA |
| 67081 | AGCCCGATTC | CCCAGCGGGC | GACCGTAGCC | GGAGCGTTGG | ACCGCCCTGC | TCCGCACGTC |
| 67141 | GAGCCCCACC | GTGGTAGCGG | CGGACATGCC | CAGGGGGCGC | ACGCCCGGAT | CCTGCCGCAC |
| 67201 | GACCAGGCGC | ACCAGGGTTT | CGGCGAACAC | GGCGGCCGAC | GCCGCGGGGT | TGCCGCCGCG |
| 67261 | GCACGGCCGA | CGCGCGCTGG | TCGCACGGGG | TGAGCCCGCG | GGAGCGGAGG | GCGCCGGTTC |
| 67321 | GCTCAGGACG | CCGGGATCTC | CGCCGGGACG | ACGGACTCGG | GGGCGCCGTC | GGGGCCGGGC |
| 67381 | CTCGTGCCAC | CGTCGGCGTC | GAGCTGCGGC | GGATCGGAGA | GCTGCTCGCG | CACGTACCCC |
| 67441 | CACACCACGG | CGACGAGGGC | GGCGACGGGC | ACGGCGAGGA | GGCTGCCGAC | GATGCCCGCC |
| 67501 | AGACTGCCGC | CCAGGGTGAC | GGCGAGCAGG | ACGACGGCGG | CGTGCAGTCC | GAGTCCGCGG |
| 67561 | CTCTGGATCA | TGGGCTGGAA | GACGTTCCCC | TCCAGCTGCT | GCACCACCAC | GATGATCGCG |
| 67621 | AGCACGATCA | GGGCGTCGGT | CAGGCCGTTG | GAGACCAGGG | CGATGAGCAC | GGCGACGAAT |
| 67681 | CCTGCTAACA | GCGCGCCGAT | GATCGGCACG | AAGGCGCTGA | CGAAGGTGAG | TACGGCGAGC |
| 67741 | GGCAGGACGA | GCGGGACGCC | GAGGATCCAC | AGGCCGATGC | CGATGAGGAC | GGCGTCGAGG |
| 67801 | AGGCCCACCG | CGGCCTGGGA | GCGGACGAAG | GCGCCGAGGG | TGTCCCAGCC | GCGTTCGGCG |
| 67861 | ATCGTGGTGG | CGTCGGTCGC | GAGGCGGCCG | GGGAGCTGAC | GGGCGAGCCA | GGGCAGGAAG |
| 67921 | CGCGGCCCGT | CCTTGAGGAA | GAAGAACATC | AGGAAGAGCG | CGAGGACGGC | CGTGACGACC |
| 67981 | CCGTTCACCA | CCGTGCCGAC | GCCGGTGGCG | AGGGTGGTCA | GCAGGGATCC | GACGCTGTTC |
| 68041 | TGGAGACGGT | CGGTCGCGGT | GTCCAGGGCG | CCGGTGATCT | GGTCGTCGCC | GATGTTCAGG |
| 68101 | GGCGGACCCG | CGGTCCACTC | GCGGAGACGC | TGGATGCCCT | CGACGACACC | GTCGGCCAGT |
| 68161 | TCGCCGGACT | GCGAGGCGAC | GGGCACGGCG | ATGAGCGCGA | CGGTGCCGGC | GGTGACGGCG |
| 68221 | AGGAAGAGGA | CGGTGACCAC | CGAGGCGGCG | AGCGCCGGCG | GCCAGCCGAG | ACGGCGCAAC |
| 68281 | AAGCGGGCGA | AGGGCCAGGT | CAGCGTGGTG | ATCAGCAGAC | CGATGACGAG | TGGCCACACG |
| 68341 | ATCGACCACA | TCCGGCCGAG | CAGCCAGATC | ACCGCCGCCG | TGCCCAAGAG | CACCAGCAGA |
| 68401 | AGCTCCGTCG | ATATGCGGGC | CGAGGCCCGT | AGCGCGGCAC | GTGTTCTCGC | AGGACTGAGA |
| 68461 | GAGGCAGACA | TGGCGATCAC | CCTAGAGCGG | CCCGGGCGGC | CCGCTGCCCC | CGTGCCCCGA |
| 68521 | TCCTTCGCGC | CGGGGTGACG | CGCATCGGGG | GTTCCGGCAC | TGCCTGAGCG | CCTGCACGGA |
| 68581 | CGGGGCGGGT | TACGCCGAGG | GGAAGCAGCC | GGCTCCGGAT | CGACAGGAGT | GCGGGTGACG |
| 68641 | GTCGTACACC | GGCGCTACGT | CGCCCACTCC | GCGGCGCACA | AGGGCGGTCG | CCGTCTGGCC |
| 68701 | CCTCCCCGCG | TCCGACGACC | GCCCACCCCT | CGTCAGGGAG | CGGGGTCCGG | GGCGAGGTGG |
| 68761 | ATACGGGCCG | CTACCGGGAG | GTGGTCGCTG | GCCGTCGCGG | GAAGGGTCCA | TGCCGAGGCG |
| 68821 | GCCCGGACGC | CCCCGAGAAG | GATCTGGTCG | ATCCGGACGA | CCGGCAGGCG | CGCCGGCCAG |
| 68881 | GTGAAGCCGA | AGCCCGCGCC | CGCCGCGGCC | TGTGCGGAGA | CGAGACGGTC | GGTGAGGGGG |
| 68941 | CGCAGTGCGC | GGTCGTCCGT | GGAGCCGTTG | AGGTCGCCCA | GGAGGACGAC | GCGCCGGACG |
| 69001 | GGCTCGGCCC | GCACCTCCGC | CGCCAGCAGC | CCCAGCGCCT | CGTCGCGCGC | CCCGGCGGTG |
| 69061 | AATCCGCCCG | GGCCCACGCG | GACGGACGGC | AGATGGGCGA | CATAGACGGC | CAGCGGCCCG |
| 69121 | CCGGGCGCGT | CCACCGTGGC | CCGCATCGCC | CGCGTCCACG | GCATGATCGG | CACAGCCCGC |
| 69181 | GCGTCGCTCA | GCGGATGCAC | GCTCCACAAG | CCCACCGTGC | CCTCGTAGAA | GTGGTACGGG |
| 69241 | TACGACTCCG | CAAGGGCCCG | CTCGTAGGCG | GGCGCCGTGG | CCGGGCTCAG | CTCCTCCAAC |
| 69301 | GCCAGTACGT | CCGCCCCGGC | GGCGAGCAGG | CTCCGCACCG | TTCCGGCGGG | GTCGGGGTTG |
| 69361 | GCCTGCTCGA | CGTTGTGACT | GACCAGCGTG | AGGTCCCCAC | CGGGCGTCGT | CTTGTCGGTC |
| 69421 | AGCGCTCCGC | CGAAGGCCGT | CAGCCAGGCC | ACGGCCGGCA | CCACCAGGGC | CACCACCGCC |
| 69481 | GCCACCCGCG | CGCGCCACAG | CAGGGCCGCG | GTCACCAGCA | CGGGCACGGC | CAGCGCGCTC |
| 69541 | CAGGGCAGCA | AGGTCTCTGC | CAGACTGCTC | AGCCGTCCGG | GCAGTCCGGG | GAGCCGCCCA |
| 69601 | TGGCCCGCGA | CGCTCACCGC | GAGGAAGACG | GAACAGCCCG | TGACCGCCGC | CCCGCGGCGT |
| 69661 | CGCCACCACC | GCCGGCGCGG | ACCGGCGTCC | CCCACGCCGA | CCGCGGCTCC | GGGAGCGCGG |
| 69722 | CCCCTGCCGT | CCGCCGTGCC | GTCCGCAGCC | AGGGAGCAGA | CGGTCTGTTC | CGCCCTGCCG |
| 69781 | TCCGCCGCAG | TGGAGGGGGC | GCTCTCCGCT | GCCCCGCTGT | CCGGCGCCGC | CCGGGAGGGG |
| 69841 | ACAGTCTGTG | CCGCCCTGCC | GTCCGGCGCC | GCGAGGAAGA | CGGAACAGCC | CGTGACCGCC |
| 69901 | GCCCCGCGGC | GTCGCCACCA | CCGCCGGCGC | GGACCGGCGT | CCCCCACGCC | GACCGCGGCT |
| 69961 | CCGGGAGCGC | GGCCCCTGCC | GTCCGCCGTG | CCGTCCGCAG | CCAGGGAGCA | GACGGTCTGT |
| 70021 | TCCGCCCTGC | CGTCCGCCGC | AGTGGAGGGG | GCGCTCTCCG | CTGCCCCGCT | GTCCGGCGCC |
| 70081 | GCGGGGGAGG | GGACAGTCTG | TGCCGCCCTG | CCGTCCGGCG | CCGCGGGGGA | GGGGACGGGC |
| 70141 | TCCGCCGTCC | CGCCGTCCGG | CACCGCGGCC | GGATCCCGGC | GTGTCGTCGC | CGTCATCGTT |
| 70201 | CCCCGCCCTG | GGTTCCGGCG | GCGGCCAGCC | GCTCGCGGAC | GGCGGTGAGC | AGGCCACGGG |
| 70261 | CCGCCTCGAC | CGCGGCCCGG | AGCCCCTCGG | CGGGCGTCGT | GTTCGCCCGC | TGGTGCAGGA |
| 70321 | CGGCGCCCAC | CAGCAGCCGG | GGCGCGCCGC | CCACGCGCAC | CTCGTAGGCC | CACAGGAGGT |
| 70381 | TCCCGCCCGC | CGGGGTGCTG | GAGCCCGTCT | TCACACCGAG | GACGCCCGGG | GTGTCCAGCA |
| 70441 | GGGGATTGGT | GTTGGTGATC | GTCCCGAGGC | CCGGGACGGT | GGTCTCGCGG | GTGGCGACGA |
| 70501 | CCGCCCGGAA | GACAGGGTCC | TCCATCGCGG | CCCGCGTCAG | CCGCACCTGG | TCGGCCGCGG |
| 70561 | TGCTTGTGGT | CGTCGGCTCG | ATGCCGCTCG | CCCCCGTGTA | GACCGTGTCC | TTCATCCCGA |
| 70621 | GCCGTACGGC | GGCGCGCCGC | ATCTTCGTCA | CGAAGGCCGC | CTGGCTGCCG | GAGTCCCAGC |
| 70681 | GGGCCAGCAG | ACGGGCGACG | TTGTTGCCGG | AGGGGATGAG | GAGCAGTTCC | AGGAGCCGGC |
| 70741 | GCTGGGAGTG | GCGTTCGCCG | GACCGTACCG | GGACGGTCGA | CTCGCCGCCG | ACCCCCGCCT |
| 70801 | CGTGCGCGGC | CGTCCGGTCC | ACTTCGATCA | GGGGGCCGTC | CTCGTCGGGC | CTCAGCGGAT |
| 70861 | GCTCTTCGAG | GATGACGTAG | GCGGTCATCA | CCTTCGTCAG | GCTCGCGATC | GGTACGGGCC |
| 70921 | GCCGCTCGCC | CCGCTCGCCC | AGCGAGCCGG | TTCCTTCGAG | CTCGACGGCG | CTCTGGCCGT |
| 70981 | CCTGCGGCCA | GGGGAGCGGC | CCGATGTCGG | AGACCGGTAG | TCGCTCGCCT | CCCGCCGCCG |
| 71041 | GAGGCGCGCC | GGAGGGCGAG | GCGACGGTGA | TGCCCAGGGC | CGTCAGCAGG | GCCACGGACA |
| 71101 | GGGCACCGCC | GACGAGGCGG | TGACGGGGGG | TGCGGGGCAG | GGACACGGGC | CGCCTCCAGG |
| 71161 | GGCTGCGGTA | CGGGATCGGT | ACGGCAGCAA | GACTCCGGGG | GCTACGTCTC | CGCCTCACGG |
| 71221 | TCGGGAGAGC | GCGGCCCGCG | CTCGGGAACC | CATCGGTCGT | GTATCGGCGG | GGCGGCGGCG |
| 71281 | ACCGGCCGCC | GGGCGACGCG | GAGGGAGCGC | CTGAGGGGCG | GGGCGTACCG | ACAGGCGACC |
| 71341 | GTCTGGGGTG | GGGAGGCCCG | CGGGCTGTCC | CGGGGACCGG | TTCACGCCTC | GGACGTCTGC |
| 71401 | CCGTCCTCGG | GCAGGCTCAG | GGTCGCGACC | GCTCCTCCGT | CCCGGGCGTT | GGCGAAGGCC |
| 71461 | AGGGTCGCGC | CGATCACCCT | CGCCTGGCCG | GACGCGATCG | TCAGGCCAAG | GCCGTGCCCG |
| 71521 | TGCCCCCGTT | CGGCCGAGCC | CGTGCGGAAC | CGCTGGGGGC | CGTGGGACAG | CAGGTCGGCG |
| 71581 | GGGAAGCCGG | GGCCGTGGTC | CCGGACGGTC | ACGGTCCGGC | CGGCGACCGT | GACCTCCACC |
| 71641 | CGACCCGCGC | CGTGCCGGTG | GGCGTTGACG | ACGAGGTTGG | AGACGATGCG | GTCCAGGCGC |
| 71701 | CGGGGGTCCG | ACTCGACCAC | TGCCGCCCCC | TGTCGCGTCA | CCTGCGCCGC | GAGGCCCGTC |
| 71761 | CGCGCCACCG | AGTCCCGGAC | GAGGGCGCCC | AGGTCGACCG | GTCCCTGCTG | GGCCGTCTCG |
| 71821 | GCGCCCGCGT | CGAGCCGGGA | GACCTCCAGG | AGGTCCTCCA | CCAGGTCGCG | CAGCACCCGT |
| 71881 | ACCCGGCTCT | GGACCATGTC | CGTCACCTCG | CCCTCGGGCA | GCAGTTCCGC | CGAGGTGACC |
| 71941 | AGGCCCATCA | GCGGGGTGCG | CAGCTCGTGT | GCGACGTCGG | CGGTGAAGCG | CTGCTCGGTG |
| 72001 | TCGATCCGCT | GCTGGAGGCT | GTCGGCCATC | GAGTCGACCA | CGGCCGAGAT | CTCGGCGACC |
| 72061 | TCGTCGCCTC | CGCGGACCGT | TCCCGTCCGG | GCGTCGAGGT | CGCCCGCCGT | GATGCGCCGG |
| 72121 | GCCGTGCGGG | CGACCCGGCG | CAGCCGCCGG | GCGGGGAGTT | CCGTCGCCAG | GGCCGTGGCG |
| 72181 | GGGACGACGA | CGCCCAGGGT | GAGCAGCGAG | TACTTCCACA | TGTGACGGTC | CAGGGCCTGC |
| 72241 | CGGGTCAGCA | GGTCGGCGGT | CATGTCGACC | TCGACCGCGT | ACAGCTTCCC | GCCCTCCCGG |
| 72301 | CGGGCCGCCC | GGAAGACGGG | GGCCGGGGGC | CCGTCCTCGT | AGAGGGTGGC | CTCGCCGCCG |
| 72361 | TGCTCGATCT | GCCTCAGCAG | GGCCTCGGGC | AGTTCCTCGG | GGGACACCCG | GGGCCCTTCC |
| 72421 | TCACCTGCTG | CGTCGGCGTC | CTCCAAGGCC | GTTGCCAGCG | CCACGTGGGC | CCTGCCCGCG |
| 72481 | CCCTCGTGCA | GGGAGCGCCG | CAGCACCGAG | TCGTGCACCA | GCACTCCGAC | GGTCAGCGCC |
| 72541 | ATCGAGGCAC | AGGCGAGCGC | CACCAGGAGG | ACGATCTTCC | AGCGCAGCGA | GCGGGAGCGC |
| 72601 | GGTACCAGGC | CCGTGACGGC | TCCCCGGGGG | GCCCGGCTCA | CCGCTTCCAC | TTGTAGCCGA |
| 72661 | AGCCCCGGAC | CGTCTCGACG | CGCTCGGCGC | CGATCTTCTT | GCGCAGCCGC | TGCACGCACA |
| 72721 | GGTCGACGAC | CCGGGTGTCC | CCGTCCCAGC | CGTAGTCCCA | CACCTCGCGC | AGGAGGGTCT |
| 72781 | GACGGTCCAG | CACGATCCCG | GGGTGCGCGG | CGAACTGGAG | CAGCAGCCGC | AGCTCGGTCG |
| 72841 | GGGCGAGCGC | GATCCGCTCG | CCGCCCCGGC | GGACCTCCAG | GGCCGCGGGG | TCGAGGGAGA |
| 72901 | GGTCGCCGAA | GAGCAGCGGG | CCGGCCGGCG | TCGCGGGGTC | GGCGGGTCCG | GGGGCGGGGG |
| 72961 | ACACGAAGGC | GGCCCGGCGC | AGCAGCGAAC | GGATGCGCGC | CACCAGGACG | GCGGTGTCGA |
| 73021 | CGGGCTTCAC | CACGTAGTCG | TCGGCCCCGG | CCTCCAGGCC | GGACACCACG | TCGAGGGCGT |
| 73081 | CACCGCGCGC | CGACATCATC | AGGATCGGGT | CCGTGGCCGT | CTCCCGGATG | CGCCGGCACA |
| 73141 | GTCCGATGCC | GTCCAGGCCC | GGCAGCATCA | CGTCGAGCAG | CACCAGGTCG | TGCCGTCCCT |
| 73201 | CCCGGAAGAG | TTCGAGCCCG | GTCAGTCCGT | CGGCCGCGAC | GCGCACGGGG | TAGCCGTAGC |
| 73261 | GCTCCAGGGA | CATCGCGACG | GACCGCCGGA | TCACCTCGTC | GTCCTCGACC | AGCAGGACGG |
| 73321 | TCACGGTCGC | AGGCGCGGGC | GCCGACACGG | AATCAGACAT | GTCCATCTCT | CGGGCACGGG |
| 73381 | GCGGGGCGGG | CCGACGCGCG | GCCCGGCCCT | CCATCATGCC | TCACTCGCGC | GCCTCCCCCG |
| 73441 | GCGCCGCCGG | AGAGCCGTCG | GCACGCCTCT | GAGCTGGTCT | GATACCTGAC | TGATACATCA |
| 73501 | TGGCGCGGTG | ACCGACACAC | GCCCGAGCGG | GGACGGACAC | GGAGGCGGCG | CGCCGGACAC |
| 73561 | AAGATGCCGC | GGGAGCCGAT | CCGGCGCCCA | CCGAGGCCGT | GTGAACGACG | CCGTCCGGAA |
| 73621 | CCGCACGCCC | GCGGGGGCTC | GTGGCGGGTG | TCAGTGGTGT | GCGGCAGGCG | CGGGCAGCGT |
| 73681 | CTCCGGAGCG | GGGTCGGTCC | TGCGGCCGAG | GTGGTTGAAG | GCGAGGTTGA | GGAGGACGGC |
| 73741 | GACCACACAT | CCGGTGCTGA | TGCCTGAGTC | GAGGACGATG | CGGGCGCCTT | CGGGGAAGGC |
| 73801 | GTGGTAGAAG | TCCGGGGCGG | CGATGGGGAT | GATGCCGGCG | CCCAGGGAGA | TGGCGACGAT |
| 73861 | GAGGACGTTG | TCGCCGCGTT | CGAGGGCGGC | TCCGGCGAGG | GTCTGGATGC | CGCTGGCGGC |
| 73921 | GACGGTGCCG | AAGAGGGCGA | TGCCCACTCC | GCCGAGGACG | GGCTGGGGGA | TGAGCGCGAC |
| 73981 | GACGGAGGCG | AGCAGCGGGC | AGAGGCCGAG | CAGGAGGAGG | ATGCCGCCGG | CCGCGGCTAC |
| 74041 | GACGAAGCGG | CTGCGGACCT | TGGTGATCGC | GACGAGGCCG | ACGTTCTGGG | CGAAGGCGCT |
| 74101 | GGCGGCGAAG | CCGTTGAAGA | GCGGGCTGAG | GGCGGTGCCG | AGGCCGTCGG | CGCGGAGCGC |
| 74161 | GGCGGCCAGG | GTCTTCTCGT | CGGCCGGCCG | CTCGACGATC | TCACCGAGGG | CGAGGACGTC |
| 74221 | GGCGGTGGAC | TCGGTCATCG | ACACGAGCAT | CACGATGCAC | ATGGAGATGA | TCGCCGCGGC |
| 74281 | CGCGAACTGC | GGAGCGCCGA | AGTGGAACGG | GGTGGGGAGT | CCGATGACGT | CGGCGTCGCC |
| 74341 | TACGGCGCTG | AAGTCGGCGA | CGCCCAGCGG | CAGCGAGAGG | AGCGTGCCGG | CGACGAGGCC |
| 74401 | CAGCAGGATG | GAGATCTGCT | TGAGGAAGCC | CGTCAGGACG | CGGCGCAGGA | CCACGGTGAT |
| 74461 | CAGCAGGGTG | GCGGTGGCGA | GGCCGATGTA | CGTGAGGCTG | CCGTAGTCCG | GTGCCTGCGC |
| 74521 | GTTGCCGCCC | TGGGCCCAGT | TGAACGCGAC | GGGCAGCAGG | GAGACACCGA | TGAGGGTGAT |
| 74581 | GACCGTGCCC | GTGACCACGG | GCGGGAAGAA | GCGGATCAGT | TTGCAGAAGA | AGGGGGCGAG |
| 74641 | GAGGAATCCG | AAGACGCCGG | CGACGATCAC | GGCGCCGTAG | ATGACGGGCA | GCGCGTCGTC |
| 74701 | GGGGCCTTCG | GCCTTCGCTA | TCGCGAGCAT | CGGTGCGACG | CCGGCGAAGG | AGACGCCGTT |
| 74761 | GACGAACGGG | AGCCGTGCTC | CGACCTTCCA | GAAGCCGAGC | GTCTGCAGGA | GCGTGGCGAT |
| 74821 | GCCCGAGGTG | AACAGGCTGG | CGCTCATGAG | GAACGCGATG | TCCGCGGTGG | ACAGGCCGAC |
| 74881 | GCCGATGCCG | ACGACCAGCG | GCGGCGCGAC | GACGCCGGCG | TACATGGCGG | CGACGTGCTG |
| 74941 | CAGACCGGCG | CTGAAGAGCT | TCAGCGGGGG | CAGCATCTGG | TCGACCGGAT | GGGTGTCGCC |
| 75001 | CGGTCTTGCG | GGGGTGTCGG | GACGAGCGTC | GGTTCCGTCC | TCGGTCTTCG | TCGGTTCCGG |
| 75061 | GGCGGGGCGG | GATTCCGGCA | TGGCGTGTCT | CCTGGCCGCG | GCGGTCTCGT | GGGAGCGCGG |
| 75121 | GCAGCCCTGT | GGGAGAGCCG | GCGTCGCTGA | CGCACGGCTC | TGCGGATGGG | GGTGGTGACG |
| 75181 | GGGTGGGGAG | GATGCGGCAC | CGGGCGTGGG | ACGAGAGGCC | GTCGTGCCTG | GGTGCCGCGG |
| 75241 | CCGGAGAGCG | GGGAGTGGGG | CGCCGGAGAA | CGGGCGGGAG | GGATTCGTGT | CCCGGGGTTC |
| 75301 | CCGCGGTCCT | CCGGCCGGCC | GGCGCGCAGC | TGTCCGTGAT | GCGCGTCGTG | CCACTCCGTC |
| 75361 | CGGCGAAGTC | GGAACAGTTC | CTCACGGGGC | GGCGTGGGTG | TCAAGAGGCG | GGACGGGCCG |
| 75421 | GGTTGAGGAC | CTCCCGGACG | TGGTGCCAGA | GGTGCAGGTC | GATGTGTTCG | AGGAAGTCCG |
| 75481 | CCGCCCGGGC | CTCCGCGCGG | GCCGCCTGAC | GGGGCGGGCC | GCCGGCCGCG | CCCTCCCATT |
| 75541 | GCTGGTAGGT | GAGGTGGAGG | CGTTCGAGGG | CGTGGCCGAG | TACGGCGGGG | TCGAGCGGCA |
| 75601 | CGATGCGGGA | AAGGGCCCCC | ACGTAGGGGG | GCCACCAGGT | GGTGGCGGCC | TCTCTGAGCA |
| 75661 | GGACGCGGAG | CCGGCCGGTC | CTGCCGCACG | CGGCGACGAA | GCAGGCCGGT | GGCGGGCAGA |
| 75721 | GGACTCTCAG | GAGGGCGTCC | GTCTGGGCGG | GGGTCCCCTG | CCAGCGGTTC | CGGCGTTCGG |
| 75781 | CGTCCTCCCA | CTGGCCGCGG | GTGAGGCCGA | GCTCGGTCGC | GGTCTCGTTG | ACGGTGAGTC |
| 75841 | CGGCGACGGT | ACGGCACTGG | GCGAGGGTGC | CGGGCTCTTC | GACGAGATCG | ACCGGAGAGC |
| 75901 | ACCAGAGGGC | CGCCGCGAGG | GCCTTCACCT | GGGTGTTGTC | GGGGGTGGCG | GTGCCGGCTT |
| 75961 | CCCACGCCTC | GACGAGGCCG | GGGTGCGCGG | GGTGTCCGCA | GTAGGCGGTG | ACGGCCCAGG |
| 76021 | CGACCTGGCC | GAGCGTCAGG | CCGAGCTGGG | CGCGGACGGC | CGCGGCACGG | GTGGGATCGA |
| 76081 | AGCGGGGCAG | GGCATACGGA | TCGTCTGCGG | GTGGCATAGC | GGGACACCGT | AGGGACCCCG |
| 76141 | CGGTCCACGG | CCAAGGGCCG | GAACGGTCTC | GATGTCGGCT | CCGCCGCCCG | GCGTTCGGCT |
| 76201 | TCTTCGTCGG | ATCTCCTGAG | CGGGCAGACC | GTCGATGACC | TGCTCTTTCA | TGGGAGGAGG |
| 76261 | CGGGCAAGCG | GAGAGAAGAG | GAGGCTGAGG | CGACTGTCGG | TTCCTCCAGA | ATCCGCGAGG |
| 76321 | AACTGGCGCC | TTCTCTTGGG | GGAGTTGACG | AGGTTCAGGC | CGGGCCATAA | AGTCCCGTCC |
| 76381 | CGTCCACAAC | GGAAAACTTC | TTCCGTCATG | CGAAACCTGT | GGTGACAGCC | GCCCACCCCC |
| 76441 | GCGGCTCGCC | CCCATCGGCC | ATGCTCCCGG | TTCGGGCCGT | CCCCCGGCAC | CGGTCACTCT |
| 76501 | GCCCACACGC | CCCCGGCAGT | CCGCTGCCCG | CGGGCGGTGT | CTCAAGGACC | TGCCTTGCTG |
| 76561 | CCCCGCGCCA | CTGACCACGC | ATCGCCGACC | CCACCCCATC | CGGTCGACGA | GATCCTTCCC |
| 76621 | GCCCGCCGCA | TGCTGCCCGC | CGCCCTCCAA | CACGTGGCGA | GCATGTACCC | CGGCCTGACC |
| 76681 | GCACCACCGC | TGATCATCAG | CAGCGCCCTG | GGGCTGACCC | CGGCCCAGCT | CTCCGCCCTC |
| 76741 | CTGGCCGCCG | CGCTGCTGAT | CGCCGGGCTC | GGCACGATCG | CCCAGACCCT | CGGCGTCTAC |
| 76801 | GGCGTCGGCG | CCGGGTTGCC | CCTCGTCAAC | GGCGTCTCGT | TCGCCGTCGT | GTCACCGGCG |
| 76861 | CTCGCCACCG | CCGCCACCCA | GGGGCGCGAC | GGCGCCCTCC | CGGCGATCTT | CGGGGCCACC |
| 76921 | CTCGTGGCGG | GACTCCTCTG | CCTGCTCCTC | GCTCCCGTCT | TCTGCCGACT | GGTCAGGTTC |
| 76981 | TTCCCACCGG | TCGTCAGCGG | CTGCGTCATC | ACCCTGGTCG | GCATCTCCCT | CCTGCCGGTC |
| 77041 | GCCGGCACCT | GGGCCCGGGG | CGGAGACGCC | GAAGCCGCCG | GCTTCGGCTC | CCCCGCCGAC |
| 77101 | CTGGCCCTGG | CGGCGACGAC | CCTCGTCATC | ACCCTGACCG | TGCACCGCAT | GCTCTCGGGC |
| 77161 | CGCTTCCTCG | GGCGGGTCGC | CATCCTCATC | GGCATGCTCG | CGGGCACCCT | GATCGCGATC |
| 77221 | CCGCTGGGCA | AGGTCGACCT | CGACCCCCTC | GCCCAGGCGC | CCCTCTTCGC | CCTGCCCACG |
| 77281 | CCCTTCGGCT | TCGGCACCCC | GCAGTTCGTC | CCCACCGTGA | TCGCCACCGC | CGCGGTCGTG |
| 77341 | ATGATCGTGT | CCATGATGGA | GTCCACCGCC | GCGCTGCTGG | CGCTGGGCGC | GGTCGCCGAA |
| 77401 | CGGCCGGTCC | GGGACCGGAC | CATCGCCGGA | AGCCTCCGCG | CCCTCGGCCT | CGCCACGGTC |
| 77461 | CTCGGCGGCG | TCCTCGGCTC | GTTCACCAGC | ACGTCGTACG | CGCAGAACGT | CGGCCTGGTC |
| 77521 | TCCCTCAGCC | GGATCCGCAG | CCGCTATGTG | GTCACGCTCT | GCGGCGCCGT | CCTCGTCCTG |
| 77581 | ATGGGCTTCG | TGCCCGTCCT | GGGCTCGTTC | GTCGCCCTCG | TCCCTCTGCC | CGTCCTCGGC |
| 77641 | GGTGCGGGGG | TGGTCTTCTT | CGGCTCCGTC | GCCGTCACGG | GCATCCGTAC | GCTGGCCAAG |
| 77701 | GCCGCCCTCG | GCACCGGACA | CAACGCTGTG | ATCGTCTCCG | TCACCCTCGC | CTTCGGTCTC |
| 77761 | TTCCCCGTCC | TGGACCCGGA | CTTCTACGCC | CGTCTTCCCG | CCCCGGTGGC | GACCGTGCTC |
| 77821 | GGCTCGGGGA | TCACCGCCGG | CTGCCTGGTC | GCGGTCCTCC | TCAACTACCT | CCTGAACCAC |
| 77881 | CTGGGCCGCG | GCACCGAGGC | CGACCCCGAC | GCGATCTCCG | CGGAACAGGT | CACCGCCCTC |
| 77941 | GACACCGCGG | ACACCGTCCT | CGGGCCGAAG | CGTTCCTCCG | ACTGGACGCC | CTTCCAGCCC |
| 78001 | TCCGGCAGCC | CCTCCGGCAC | CCCTGACCAC | GGCCGTCACA | CCAGGGGCAC | GGCACGGCCC |
| 78061 | GCTCCCGCCT | GGCCCTACGT | GACCGGCCCC | GTGGACCCCA | CCGACACCGG | GCGGCACCAC |
| 78121 | CGGCCGCACG | AGGTTCCGGC | GCCGCCCCAC | CGACCAGACG | AGGTGCCCCC | GCCGCTCCAC |
| 78181 | CCCTCTGCCG | CTCACGAAGG | CGAACCCCCG | CCCGCCGTCA | CCGAGAACGC | GGTCTTTCCC |
| 78241 | GGACCGCTCC | ACCCGCTCCA | CCCGCTCCAC | CCCCGGCCCA | CCGGTCGTCC | CGACCGTCCC |
| 78301 | CGGCAACGGC | ACAGTGCGGA | GGCCGACCCC | TGGCAGCATC | CGCAGACCCC | CTCCGCATCC |
| 78361 | GGCGACAGCC | AGTAGAGACG | ACCTCCCCCG | ACCTCTTCGC | AGAGCCCGGC | ATCGCACAGG |
| 78421 | CCCGGCGGAG | AGGTGGCGCC | GACCCACCCG | ACCACCGCCG | GAAGCGCCCC | CGGGGACCCG |
| 78481 | TGTCCCACGG | ATTCCCCGGC | GACAAGACGA | GGTAGCCCCG | ATGACCACCG | TTTCCGCCGC |
| 78541 | CCGCCACCGT | GCGGGCGGCT | CCCCGCGCGG | CGGCACGTCC | CGCCCGGGCC | CCGACGAGAG |
| 78601 | AATCGCCCAG | GTCGTGGCCG | AGGCCCTCGG | ATCCGCCCGG | ACGGTCCTCG | ACCCGGATGC |
| 78661 | GCTGCCCGGC | CTCGGCACCA | CGCGACTCCC | GTTCGGCGAC | GGGAGGTTCG | ACGCGGCGAT |
| 78721 | GATGCTCTGC | AACGCCCCCG | GCGTCCCCGA | CGCGCTCTCG | CGGCTCGGGG | AACTGCGCCG |
| 78781 | CGTGACACGG | GGCCCCGTCG | TGGTCCTCGC | GACCGACCCC | TCGCGCGTCC | GCTCGTTCTG |
| 78841 | GCTGGACCGG | TACGCCCCCG | AGGTCCTGGC | CGTCGAAGCG | CGGCGTCATC | CGCCGATCGC |
| 78901 | CGATCTGACC | GCCGTCCTCG | GGGGTTCCGC | CGAGGTGCGG | AGCGTCCCCG | TTCCCCTCGA |
| 78961 | CTGCACCGAC | ACCTTCGACG | AGGCGTACTA | CGGAAGACCC | GAGAAGCTCC | TGGACCCGTC |
| 79021 | GGCCCGCCAG | GCGGGGTCGG | CCTGGAGCTT | CGTGGACGAC | CGGGTCCGCG | AGGAGTTCGA |
| 79081 | CACGACCCTG | CGCCGCGAAC | TCCGGTCGGG | GGAGTGGGAC | GAGCGCTTCG | GCCACCTCCG |
| 79141 | CCGCCGGCCC | GTCTACGAGG | GATCACTGGT | GATCGTCCGT | GCCGTCCCCT | GACGTCCTCC |
| 79201 | CGGGGACGCG | CCACCCCCGG | GGTCGCGTCA | GCGGTCTCCG | GCCAGGTGCC | CCGAGAGCTC |
| 79261 | GCGCATCAGC | GTCTCGTGCT | GCTGGAGCAG | GTAGAAGTGG | CCGCCGGGCA | GGACCCGTAC |
| 79321 | CCGGAAGCCC | TCGGGTGCGA | CATCCGCCCA | GGCGTCCATG | TCCCCTACCG | CCACGTTCGG |
| 79381 | ATCCGTGTCG | CCGATCCAGG | CGTGCACCGG | ACAGCCGACG | GCGGTGGGGA | CGCGGGGGCC |
| 79441 | GTAGGTGCTC | ACCACGGTGA | AGTCCGCCCG | GACCGCGGGC | AGCACGAGCT | GTCGGATGTC |
| 79501 | CGGGTCGTCA | AGCAGGGCGG | TATCGGTCCC | GCCGAGCCCG | CGCAGTACGG | CGACCAGCTC |
| 79561 | GTCGTCCCCC | TTCCGGTGCA | GGTCGAGCGG | GGTCAGCCGG | TGCGGGGCCT | TGCGGCTGGA |
| 79621 | GACGTGGAGG | GCGGCGGGCG | TGACGCGGTG | CCGCTCCTCC | AGGCGCAGCG | CCACCTCGTA |
| 79681 | GGCCAGGGAC | GCGCCCATGC | TGTGGCCGAA | GAGCGTCAGG | GGCACGTCCG | CGAGCGGCAG |
| 79741 | CAGTGCCGCC | GTGACCCGGT | CGGCGAGCAC | GTCCATCCGG | TCGACGAACG | GCTCGTTGAA |
| 79801 | CCGCTCCTGG | CGTCCGGGGT | ACTGCGCCAC | CAGGACCTCG | GTGTCGCCGC | CGAAGGCGCT |
| 79861 | GCCCCAGGCG | TGGAAGAAGC | TCGCGGAACC | GCCCGCGTGC | GGCAGGACCG | CCAGCCGCCG |
| 79921 | CCGGGGCGCG | GGAGTGCTGG | AGTACCTGCG | GAACCACGTC | GTGCTGTCCG | TGCCGGTCGT |
| 79981 | CATGTGTGCG | TACACCCCGT | CCTCGGGTTC | TTGGGGTGCC | AGTGTCCCCG | CAGGGCCCGG |
| 80041 | TGTCCGGACG | CGGTGGGGGT | CCGGTGGCGA | GCCGCTTACG | TGTCCCCGCG | CTTCCGGGAC |
| 80101 | CGGCGGCCGC | ACACGTGTCG | GCCCCCACGA | ACACCAGGGT | GCGTGGGGGC | CGATGCGTGT |
| 80161 | TTCGAGTCCT | GGTCTGACGA | TTTCAGGCCG | AAAGATATGT | CGGACTTTAC | AGCTGCGATC |
| 80221 | GAAGCCGATC | GATAATGCCG | TGGACGGGTA | ACGTCGGAAT | CACTCGGTGC | TCTTGAGCGC |
| 80281 | ACCACTCACG | TTGACGACCT | CGTGGCACTC | CCGCGCCTGC | TGTCCCGTCG | CGGGCGTACC |
| 80341 | GGGCTTCGTG | CAGGTCACGT | CGATGGTCAC | GGTGTCCTTC | GCGCCGATCC | TGATGGGAGT |
| 80401 | CACCCAGTGG | TAGTCCTGGT | TGCGGAACGT | CTCCAGCGCG | ATCGTGGTGA | TCTTGCGGTC |
| 80461 | CCCGAAGGTG | ATCGTCATCA | CCCCTTCGTC | ACCCTGGAAG | TTCGCGACAA | CGATGTCCGT |
| 80521 | GATCCCGAAC | ACGGTCTTCT | CGGGGACCGT | GTACGTCCCG | GTCCTGGACT | GTCCCGCTCC |
| 80581 | CGACCTCAGG | TCGATGGTGG | CCGAGCTCTG | CCGTCCCCCG | CCGGTCGCGG | TGCCGTCGCC |
| 80641 | CGTGCCGGCC | GAACCGTCGT | CCCCGCCGCC | CGCTCCACCG | CCCGATCCCG | GCTTCTGGCT |
| 80701 | GCTGCCGCCG | GACGGCCGGC | CCGGGCCGGG | GGAGGAGCCG | TCCGAGCCGC | TCTCCGTCGG |
| 80761 | GACCGGGCGG | GGCTGCACCG | CCTGGGTGGC | CGCCTCCTCG | GCGGCGCTGC | GCACGGCCGG |
| 80821 | CCGGACCAGC | GTGAACCAGG | CGATCAGCAG | CGCGATCAGC | GCCGCGAGCA | GCAGGAGCAG |
| 80881 | CCACTTGGGG | AAGACCGGTA | TCTGGACGAA | CTCCGCGTCC | AGCGTCGGCG | CCGTGTGGGG |
| 80941 | CTCCTCGGCG | CGGTCCTCGG | TCTGCTCCGG | CTCGCCGGTC | TCGCGGGCGT | CCACGGTGAA |
| 81001 | CGGCCAGACC | ACGGGGTCGC | CGAACCACAC | CGGGCTCGCG | GTGCGGACCC | GCAGCCGGAG |
| 81061 | TTCCTTCGAC | TCGCCCGGCT | CCAGCGCCGG | CTCCGCCGGC | GTGAAGGCGA | ACCGGAGCTC |
| 81121 | CTCGCCCGCC | TGCCCGGGCG | TGAACCCCAC | CCGTACCGGG | GTGTTGCCCT | GGTTGCGGAC |
| 81181 | GGCCAGCAGA | TAGCGGCCCC | GGAGCCAGCC | GCGCCGGCGG | CGCGGCGAGA | GGTCGGTCCG |
| 81241 | CAGCTCGTGG | AACGCGCCGA | CGCGCACCAC | GGTCTCCAGG | ACCTTGACCG | ACTCGGGCTG |
| 81301 | CTCGTTCGGG | AGGATCCGTA | CACCGAGGGG | CAGCTCGCCG | GCCCGTGTCT | CCGGCGAGCG |
| 81361 | CGGCGGTGCC | AGACGGAGCG | TCACCGTCTC | GGACGTGCCG | GGATAGAGGG | AGAGCCGCTC |
| 81421 | GGGCTCGACG | GTGGTCCATT | CGGCACCGTC | ACCGACGACC | TTCAGGTCGT | ACGCCTCGAC |
| 81481 | GATGTCACTG | TCGTTGCGGA | CGGTCAGGGT | GGTGGTGGCG | ATGTCGCCCG | GCGTCACGGA |
| 81541 | CACGGCCGGG | ATGTCGAGGC | CGGGCGCACC | GGGACCGGAG | GAGGCTGCGG | AAGGCGTCAC |
| 81601 | CCGCCCCACC | GTAGGAGACC | TGACAGATCC | GTACGAGGCA | CGCGAGGGCA | ATGTCCGGGC |
| 81661 | AGCTCGGCTG | CCCGGCAAGC | ACAAGTCAAC | TCTCCGGTAA | CAATGGATTT | CTAGTCTGGA |
| 81721 | GAGCCGCCTT | CGGCACACCA | CCGGCCCGTG | GTCGGCTCGT | GTCGTGTCCG | CCTTCCCCCC |
| 81781 | ACCGACCCAG | GAAAACAGGT | ATCCGATGTT | CCGCACCGAG | GAGAAGAGGC | CGGTCGCGAC |
| 81841 | CGGCACTACG | GCGCATGACG | CCGTCCGGGG | CCACCCGGAC | GCCCATGCCG | CCGGCTTCGG |
| 81901 | CCGCCCGCGC | CGCGTCACCG | TGGCGGTCTA | CGCCGCCGAC | CCCGTGCTGC | GGGTCGGCGT |
| 81961 | CGTCCAACAG | CTCCGCCAGC | GCCCCGAGAC | CGAGCTCGTC | GACGACGCGG | ACGCGGAGAA |
| 82021 | CGCGCAGGTC | TCCCTGGTCG | TCGTCGACGC | CCTCGACGAC | GACGTGACCG | CCCTGCTGAC |
| 82081 | CCGGCTGAGC | TACAACGGCG | CCACCCGCGC | GGGACTCGTG | ATCGGCACCC | TCGGCGTCGG |
| 82141 | GGCGCTCCAA | CGCGTCGTCG | AGTGCGGGGT | GTCGGCGGTG | CTGCGCCGCG | CCGAGGCCGA |
| 82201 | CCAGGACCAG | CTCGTCCAGC | TGGTCCTGGC | GGTGGCCAAC | GGCGAGGGCG | TGCTCCCGGG |
| 82261 | CGACCTGCTC | GGCGAGTTAC | TGGGACACGT | CGGCAGCCTG | CGCCGCGCGG | CCCTCGACCC |
| 82321 | CGGCGCCCTG | CCCCTCTCCA | CCCTCACCAG | CAGGGAGGCG | GAGATGCTGC | GCCTGGTCTC |
| 82381 | GGAGGGCCTG | GACACCGCGG | CGATCGCCCG | CAAGACCTCG | TACTCCGAGC | GGACCGTGAA |
| 82441 | GAACGTCCTG | CACGAGATCA | CCACCCGCCT | CCAACTGCGC | AACCGCGCCC | ACGCCGTGGG |
| 82501 | CTACGCGCTC | CGCAACGGGC | TGATATGACC | GTCCCGTCCG | GACCGCGGCC | CGGCGGCCGG |
| 82561 | CGCGACAGCC | GGAGGGAAGG | CGGCGCTGCC | CCAAAGTGCA | TCCCGCCCTT | CCCCCGGGTG |
| 82621 | CGGCCCCCGG | GCCCTCCCGC | CGCGTGCGCC | GCCGCCGCAC | GATGACGGGG | GGCACCTCCC |
| 82681 | GGTGCCGCAC | CGGACGGAGA | AGGGCACCGT | GATGAAGACC | GCTGGCCCCG | GTGGACGGCA |
| 82741 | CCGCCGGGGG | AGACTCGCCT | CGGCGCTCCT | GCTGCTCGTC | CCCCTGCTGG | GCGCGACGGG |
| 82801 | CGTGGCCGGG | CCGGACGACC | CCCGGACCGC | GGCGGCCGCG | GCGGACGCCG | CCGAGACCAC |
| 82861 | CCGCATCGCC | TACGCGGGCA | CCGGCCACCG | CAGCCTCGGC | GAACCGGCCT | CCACCGACTC |
| 82921 | CAGCACCCCG | CTGTTCGGAG | CGGGACCCAC | CCACTACGAC | ACCGACCCGT | CCGCCCTCGG |
| 82981 | CGACCGGCTG | GTCTTCGCGA | GCCGCCGCGA | CGAGAAGCAC | CCCCAGATCT | ATCTGCGGGG |
| 83041 | CGCCGACGGC | GGAGTCCTGC | GGCTCACCAG | CGGCCTGGAC | GCGGCCCGTC | CCCGGCTCAC |
| 83101 | CCCGGACGGC | GGGTCGGTGC | TCTTCGACGC | CGCCGACCCG | GCCGGCGGCT | CCCAGCGCGA |
| 83161 | CCTGTGGCTG | GTGCGCACCG | ACGGCACCGG | GCTGACCCGG | CTGACGGACA | CGCCCGCCAG |
| 83221 | CGAGGAGGAC | CCGGCGGTCT | CCCCCGACGG | CGCCCGGATC | GCCTACTCCA | GCGACGCCGA |
| 83281 | CCCCCTGGCC | GGGCGGCAGA | TCTACGTCCG | CGCCCTCACG | GGCGGCATCC | CCACCCGGCT |
| 83341 | CACCGACCCG | GCCCGCGGCA | CGGCCTCCGA | GCCCGCCTGG | AACCCCGTCG | ACGACGACGT |
| 83401 | CAACCGCGCG | TGGATCGCGT | ACACGTCGAC | CACGACCGAG | GACGGGCGGA | CCAGGCAGCG |
| 83461 | GCTGCGGATC | ACCGACGGCA | CCACCGACGA | GACCCTGTTC | ACCGGCGCGT | ACGCGAACTG |
| 83521 | GCAGGGCCAC | GGGGCGGCAT | GGCTGCCCGA | CGGGGACGGG | ATCGTGTTCC | TCAGCCCCGA |
| 83581 | GACCACCTGC | ACCTGCAGGA | CCCCCTACGA | CCACGTCTTC | CGGTCGGTCG | TGCACGCCGA |
| 83641 | CCGGGAACCC | TCCCTGGTGC | TCGACGAGGA | CCGCGACGTC | CTCTCGCCCA | CCTGGATCGG |
| 83701 | CACCGCCGAG | GGCGGCCACG | CGATCGTCGA | GCGCAGCTCG | GCGGCGACCG | CGCACACGGC |
| 83761 | GACCCTCCAG | GACATCCGCG | CGGACGGTTC | CGACCCGCGC | GACCTGCAGC | GGAAGATCCT |
| 83821 | GCGCGAGGAC | CCCCAGGCCG | ACACCAACAC | CGACCCCGCC | AAGGATCCGC | TCTTCCAGCC |
| 83881 | CGCGCCCCCG | TTCGACCCGT | GGACCGAACG | GCAGAACTAC | ACCCCCGACG | GGCGCCGCCT |
| 83941 | CGTCCTCACC | CGCTTCGAGG | GCCCCGACGA | CGCGCGGATC | GAGCGGATCT | GGACGGCCGA |
| 84001 | CGCCGACGGT | ACGAACGAGG | CGCCGATGCC | CCTCGACGGG | CGCGGCGCGC | GGGACTGGGA |
| 84061 | CACCGACCCG | ACGTTCTCCC | CGGACGGCAC | CCGCCTGGCC | TTCACCCGCA | CCTCGCCCGG |
| 84121 | CGGGGTCGGC | GAGGCCGCGG | GAGACAGCCG | CATCCTCCTC | GCCGAGGTCG | CCACCGGCCG |
| 84181 | GATCACCGGA | GAGATCGTGC | CCCCGGCCGG | TGAACTCCGC | GGCGGGGACG | CCCAGCCGAC |
| 84241 | CTGGTCCTCC | GACGGCACCA | CCCTGGCCTT | CACCCGCGCC | CGGCAGATCG | CCGGGGGCGG |
| 84301 | CGGCAGCAAG | CACGTGTGGA | CCGCGTCCAC | GGCTGACCTG | ACCCGGCAGC | GCGACCTGAG |
| 84361 | CGCGACGCAC | TGCCCGCGCG | ACTGCGACGT | CATCGACGAC | AGCCCCGCCT | TCTCGCCCGA |
| 84421 | CGGACGCTCC | CTCGCCTTCA | ACCGCAAGAA | CGGCGGCGGG | CGGATCGACG | AGCGCAACGG |
| 84481 | ACTGCTCCTG | ACCACCCTGT | CCGGCGACGC | CTGCCAGGTC | CTGCTGCCCA | CCGCCGCCCG |
| 84541 | CGGCCAGGAC | GGCGCGTGCG | AGCGGGAACT | GCCGGACACC | ACGCTCACCG | GTCCGCACCA |
| 84601 | GCCGCGCGAC | GCCGCCTGGA | CCGCCGACGG | CAAGAGGCTG | GTCTTCAGCT | CCCGGGCCGC |
| 84661 | GGCCGCGGTC | AACAGCCCGG | AGAAGCTGAA | CGTCCTGGAC | GTCGGCTCCG | GTGACATCAC |
| 84721 | CCCGCTCACC | GCCGAGCTCG | CCGGACGCCA | GAAGGAACCC | ACCGTCCAGC | AGTCCGTGGA |
| 84781 | CCTCGCCGTC | GAGGCACCCG | CCACGACGCC | CGACGTCACC | GTCGGCGCGT | CCGGCACGGT |
| 84841 | CACCGTCCAC | GTGGTCAACC | ACGGTCCCGC | CGCCTCGCCC | GGCACCCGGC | TCACCGTCGT |
| 84901 | CCCGCCGTCC | GGTGTGCGGA | TCACCGGGAT | CGAGTGGCCC | GGCGGCACCT | GCGACGCCGC |
| 84961 | CTCCCTCCAG | TGCGACCTGG | GCGTCGTCGA | GGCCGGAGCC | CAGGTCCCCG | TGGACGTCAC |
| 85021 | GCTCACCGGC | GTCACCGCCG | GCGACGCACC | CGTCGACTGG | TCGGTCACCG | GCGCCGTCCT |
| 85081 | CGACCCCCGG | CCCGGCGACA | ACGACGGCCG | GAGCGTGATC | CCCGTACGCG | AGGCACCCCC |
| 85141 | GACGCCGACC | CCCACGCCGA | CGCCGACCCC | CACGCCCACC | CCGACCCCGA | CTCCGACGCC |
| 85201 | GACCCCCACC | CGGACCCCGA | CGCCCACCCC | GACTCCGACC | CGGCCCCCGC | AGCCCCCCGC |
| 85261 | GCCGAAGGCC | GGACCCGGGG | TGCGGATCAC | CGTCCAGCCC | GAGCCCGGCT | ACGTCGGCGG |
| 85321 | ACGCGTCGTC | GTCACGTACA | GCGTCCGCAA | CGGCCGCAAC | GCGCTCGCCA | CCGGACTCCG |
| 85381 | GCTCAGGATC | GGACTGCCCG | CCGGGGTGCC | CCACGGCGGA | CTTCCGGCGG | GCTGCGACCG |
| 85441 | GAACGGCGCG | TGCGCGCTGC | CCGACCTCAC | CCCGGGCACG | ACCGCCGTCC | TGCGGGTCGT |
| 85501 | CCTCAGCCCG | AAGAAGGCGA | TGACCGCCCG | CGTCACGGCC | GTGCTCGACA | CCACCGGCAC |
| 85561 | GGACGCCGAC | CGCAGCGACA | ACACCGCCCG | GGAGCGGCTG | CGCGTCCTCC | AGCCGCGCAT |
| 85621 | CGTCGCCGTG | CCCGACATCG | GCAAGCCCGG | ATTCGTCACC | TCCGTCCGAG | GCGTGGACTT |
| 85681 | CCCGCCCGGC | GTCCCGGTGC | GCTTCAGCTG | GAACCCCGGG | ATCACCGCCG | CCGCCTCGCC |
| 85741 | GACCTTCCCG | GAGGCCGACG | GCACGTTCAT | CGGACAGCTC | CTCATCCTCG | CCAAGGACCA |
| 85801 | GACCGGGCCG | CGCACCATCA | CGGCCTCGGG | CCCCGGATTC | TCCCCGGTGA | AGACCGACTT |
| 85861 | CCTGGTCGTC | AGCGGCACCG | TCCAGCCGCC | GGACGGGGTG | ACTCGCCGGT | GATCC |

### Example 2

### Construction of a “Clean” Host Strain, S. fradiae K159-1

This example describes the preparation of the clean host, Streptomyces fradiae K159-1, a strain in which the tylGI, tylGII, tylGIII, tylGIV, and tylGV genes have been deleted. Plasmid pKOS159-5 was first constructed as follows. Two fragments flanking the tylG genes were PCR amplified from S. fradiae genomic DNA using the following primers:

tylGI left flank:

forward 5′-TTTGCATGCGATGTTGACGATCTCCTCGTC [SEQ ID NO:_];

reverse 5′-GGAAGCTTCATATGTTCTCTCCGGAATGTG [SEQ ID NO:_];

tylGVI right flank:

forward 5′-TTAAGCTTTCTAGAGAGGAGAGGCCGTGAAC [SEQ ID NO:_];

reverse 5′- AAAGAATTCGAACTCGAGCACGGACTCGTTG [SEQ ID NO:_].

The Sph I, Hind III and EcoR I restriction sites are underlined. The two fragments were then cloned into pSET152 using the underlined restriction sites and corresponding sites in pSET152 [see Bierman, M., Logan, R., O'Brien, K., Seno, E. T., Nagaraja, R. & Schoner, B. E. (1992). Plasmid cloning vectors for the conjugal transfer of DNA from Escherichia coli to Streptomyces spp. Gene 116:43-49]. The resulting plasmid was named pKOS159-5. This plasmid no longer contains the int-φC31 gene and attP locus from pSET152 and therefore serves as a suicide vector for delivery by homologous recombination.

Spores of S. fradiae 99 (Russia) were prepared by harvesting from strain grown on 1-2 AS-1 plates [see Wilson, V. T. W. and Cundliffe, E. (1998). Characterization and targeted disruption of a glycosyltransferase gene in the tylosin producer, Streptomyces fradiea. Gene 214: 95-100], filtering the spores through sterile cotton, and resuspending in 1 ml of 20% glycerol [see Hopwood, D. A., et al. (1985). Genetic Manipulation of Streptomyces: A Laboratory Manual. The John Innes Foundation, Norwich, UK]. Spore suspensions were stored at −20° C. A 20 μl aliquot of the spore suspension was added to 5 mL of 2×YT and incubated at 30° C. with shaking. After two days, the cultures were diluted 1:50 and incubated at 30° C. with shaking for 3 h. After that 1 mL of the cultures were collected by centrifugation (recipient cells). Donor cells were prepared by transforming E. coli S17-1 with pKOS159-5 and selecting for apramycin resistance only. Several colonies were picked off the primary transformation plate and used to inoculate 5 ml of LB with chloramphenicol (10 μg/ml) kanamycin (100 μg/ml) and apramycin (60 μg/ml). The cells were grown at 37° C. for 4 h (OD₆₀₀ of 0.4-0.6), collected by centrifugation, washed in 5 mL LB, centrifuged, and resuspended in 100 μl of LB. Conjugal transfer between the donor and recipient cells was performed by resuspending the recipient cells in the 100 μl donor suspension and spreading the cells on AS-1 plates. After incubated at 37° C. for 16-20 h the plates were then overlayed with 3 mL of soft nutrient agar containing 1 mg nalidixic acid and 1.5 mg apramycin. Exconjugants were observed after 48 h of further incubation at 30° C.

Apramycin resistant colonies were analyzed by PCR to confirm single crossovers at both flanking regions. One colony was selected for carrying out a double crossover as follows. The strain was grown on AS-1 plates non-selectively until well-sporulated. Spores were harvested, dilutions were plated on AS-1 plates, and single colonies were screened for loss of apramycin resistance. A single apramycin sensitive colony was isolated which did not produce tylosin. The double crossover was confirmed by PCR. This strain was named S. fradiae K159-1.

Streptomyces fradiae K159-1 was deposited under the terms of the Budapest Treaty with the American Type Culture Collection, 10801 University Blvd., Manassas, Va., 20110-2209, on Mar. 12, 2003, with accession number PTA-5060.

### Example 3

### Construction of S. fradiae K159-1/244-17a, A “Clean” Host Expressing Methoxymalonyl Biosynthetic Enzymes

Streptomyces fradiae K159-1/244-17a is derived from strain K159-1 (Example 2) by addition of the fkbGHIJK genes from Streptomyces hygroscopicus var. ascomyceticus ATCC 14891, which encode proteins catalyzing the biosynthesis of methoxymalonyl-ACP.

The putative methoxymalonyl-ACP biosynthetic genes from S. hygroscopicus ATCC 14891 (fkbGHIJK) are arranged with the 3′ end of fkbG (encoding an O-methyl transferase) overlapping by 6 codons the 3′ end of fkbH (encoding an unknown function), which is the last gene of a convergent operon that begins with fkbB (one of the PKS genes) and ends with the genes fkbK, J, I and H. To facilitate expression of these genes in S. fradiae, an operon was constructed beginning with fkbK and ending with fkbG, all in the same direction. This was done using PCR to clone fkbG with flanking restriction sites to allow its 5′ end, with its existing Shine-Dalgarno sequence, to be fused to the 3′ end of fkbH. This operon was then placed behind the tylG promoter in a pSAM2-based vector, which was introduced in S. fradiae clean host K159-1 by conjugation. Exconjugants were selected and named K159-1/244-17a.

Streptomyces fradiae K159-1/244-17a was deposited under the terms of the Budapest Treaty with the American Type Culture Collection, 10801 University Blvd., Manassas, Va., 20110-2209, on Mar. 12, 2003, with accession number PTA-5053.

### Example 4

### Construction of an Operon Containing All Five Chalcomycin PKS Genes and Expression in S. fradiae

A construct comprising the genes encoding chmGI-Vwas constructed as follows: The 3′ end of ChmGV was obtained by PCR with pKOS146-185.10 as the template and the following primers (Chalco-1A: GACACGGCCGGTGAGAGCAGC [SEQ ID NO:_] and Chalco-1B: CTTCTAGATGTCGCGGTGTACGG [SEQ ID NO:_]. The 942 bp PCR product was digested with NcoI and XbaI, the 309 bp fragment was gel isolated, and the fragment ligated into the same sites of Litmus29 to give pKOS342-33. That plasmid was cut with NcoI and XhoI and ligated to a 2.4 kb NcoI-XhoI fragment from pKOS146-185.10 to give pKOS342-35. That plasmid was digested with BglII and XhoI and ligated with a 6.4 kb BglII-XhoI fragment (including chmGIV and the 5′ region of chmGV) from pKOS146-185.10 to create pKOS342-36 (containing chmGIV and GV).

A 5.4 kb HindIII/ PstI fragment containing the 5′ half of chmGIII was isolated from pKOS146-185.1 and a 6.3 kb PstI/BglII fragment containing the 3′ half of chmGIII was isolated from pKOS146-185.10. These two pieces were ligated into Litmus28 cut with HindIIl and BglII to obtain pKOS342-38. Plasmid pKOS342-36 was cut with BglII and SpeI, the 9 kb fragment was gel isolated and the fragment ligated to the BglII and SpeI sites of pKOS342-38 to obtain pKOS342-39.

Plasmid pKOS232-172 (described in Example 5), containing chmGI and GII was cut with NdeI and HindIII and the 19 kb fragment was isolated. Plasmid pKOS342-39 was digested with HindIII and SpeI and the 20 kb fragment was isolated. These two fragments were then ligated into the vector portion of an expression cosmid, pKOS244-20 (gel isolated 8 kb NdeI-SpeI fragment). The resulting plasmid (pKOS342-45) was recovered using in vitro λ phage packaging (Stratagene) and infection of E. coli DH5α. The correct clone was identified by restriction enzyme analysis and the plasmid was moved into E. coli DH5α/pUB307 and conjugated into S. fradiae.

Expression of PKS genes in S. fradiae (in this and the following examples) were under the control of the tylosin PKS promoter (tylGIp, see Rodriguez et al., “Rapid engineering of polyketide overproduction by gene transfer to industrially optimized strains” J Ind Microbiol Biotechnol).

Apramycin resistant colonies were obtained, shown to secrete bioactive compounds, and grown vegetatively in 5 mL TSB medium (+30 μg/ml apramycin) at 30° C. for 48 h. Two mL of seed culture was inoculated into 40 ml Russia (R) medium (15 g/L whole wheat flour, 10 g/L corn gluten hydrolyzate (Sigma), 25 g/L beet molasses, 2.5 g/L brewer's yeast, 1 g/L (NH4)2HPO4, 1 g/L NaCl, 2 g/L CaCO3, and 34 g/L soybean oil) in 250 ml baffled shake flasks. After a 7 days growth at 30° C., the culture broth was analyzed for 16-membered macrolide production by HPLC (Metachem Metasil Basic column, 4.6×150 mm, 5 μm particle) using linear gradient from 15 to 100% organic phase (56% methanol, 5 mM ammonium acetate) at 1 ml/mm over 7 min. The HPLC used simultaneous detection by electrospray mass spectrometry (Turbo Ionspray source) and UV absorption at 282 nm. LC-MS analysis of the broth showed that several chalconolide derivatives were produced. The most abundant compounds were purified and shown to have the structures below. The 3-keto also forms the enol tautomer.

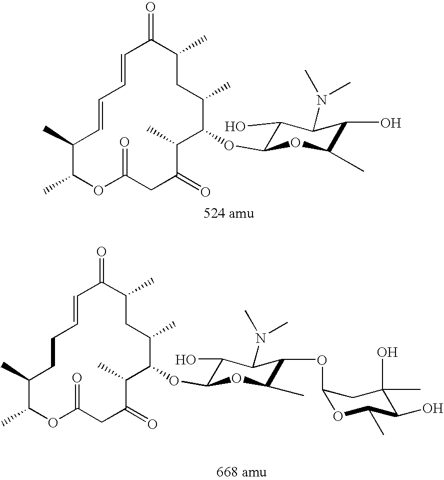

### Example 5

### Construction of Streptomyces fradiae K232-192 Expressing a Hybrid Chalcomycin-Spiramycin PKS

Streptomyces fradiae K232-192 is derived from strain K159-1/244-17a (Example 3) by addition of hybrid chalcomycin-spiramycin PKS genes, which encode proteins catalyzing the biosynthesis of 14-methylplatenolide. The chalcomycin genes were obtained from cosmid pKOS146-185.1, which was deposited under the terms of the Budapest Treaty with the American Type Culture Collection, 10801 University Blvd., Manassas, Va., 20110-2209, on Feb. 19, 2003, with accession number PTA-4961.

The first two genes of the chalcomycin PKS were isolated from the cosmid pKOS146-185.1 as EcoRI/XhoI and XhoI/BspHI fragments, and a coding sequence for a spiramycin PKS C-terminal linker attached to 3′ end. The EcoRI site is near the 5′ end of chmG1. The EcoRI/XhoI fragment was cloned into a modified Litmus28 with a synthetic linker inserted in order to create an appropriate translation start sequence. The altered region of the Litmus28 polylinker between the AflII and EcoRI sites in this plasmid (pKOS232-165) is given below. The plasmid with the chmG fragment was pKOS232-168A.

| AflII PacI SD NdeI EcoRI | |
|---|---|
| CTTAAGGGTTAATTAAGGAGGACACATATGTCCGGAGAATTC | [SEQ ID NO. _] |

M S G E F

The XhoI/BspHI fragment was ligated between the XhoI and NcoI sites of Litmus28 to give pKOS232-156. The two fragments were then joined to give pKOS232-172.

Starting with overlapping cosmid pKC1306 (described in U.S. Pat. No. 5,945,320 to Eli Lilly Company), a cassette containing the last three ORFs of the spiramycin PKS was constructed as follows. An AvrII site was introduced at the 3′ end of srmG5 by PCR from a natural MluI site to the 3′ end. The PCR product was cut with MluI and AvrII, gel isolated and ligated into a Litmus-based vector (pKOS232-75B) between the same sites to give pKOS231-118A. The 7 kb BamHI/MluI fragment from cosmid pKC1306 was subcloned in Litmus38 (New England Biolabs) to give pKOS231-113A. The 3.8 kb BamHI/MluI fragment of pKOS231-118A was gel isolated and ligated with the 7 kb BamHI/MluI fragment of pKOS231-113A to give pKOS231-120. The 7 kb BsrGI/BamHI fragment from pKC1306 was subcloned in Litmus38 to give pKOS231-113B. The 6.2 kb PstI/BamHI fragment from pKOS231-113B was cloned into Litmus28 to give pKOS231-122. The 7.5 kb BamHI/AvrII fragment was isolated from pKOS231-120 and ligated with pKOS231-122, which was cut with BamHI and AvrII and dephosphorylated, to give pKOS231-124. The 3.1 kb BamHI/SpeI fragment from pKOS231-118B (which contained a PCR fragment that created a 5′ end for srmG3) and the 7.5 kb BamHI/AvrII fragment from pKOS231-120 were isolated and ligated to give pKOS231-130. The 14 kb BamHI fragment was isolated from pKC1306 and subcloned in Litmus28 to give pKOS231-111B. The 14 kb BamHI fragment was isolated from pKOS231-111B and ligated to pKOS231-130 cut with BamHI and dephosphorylated, to give pKOS231-132.

To attach a coding sequence for a spiramycin PKS C-terminal linker to the 3′ end of chmGII, a HindIII site was introduced at the 3′ end of srmG2 using PCR with PKOS231-112B as template. The engineered HindIII site was positioned with respect to the reading frame to match that of the natural HindIII site in the chalcomycin chmGII gene. The resulting PCR product was cut with HindIII and BamHI (a natural site) and ligated into the same sites of pKOS231-114A to give pKOS232-178. This was then joined to pKOS231-132 at the BsrGI site to give pKOS232-182. The chmG1,2 cassette was isolated from pKOS232-172 as an 18 kb NdeI/HindIII fragment and the srmG3,4,5 cassette was isolated from pKOS232-182 as a 20 kb HindIII/AvrII fragment. These fragments plus a pSET152-based vector having the tylG promoter (the vector portion gel isolated from pKOS244-20) were joined in a three-piece ligation and recombinants were recovered by in vitro lambda phage packaging and infection of E. coli. Correct constructs were identified by restriction analysis (pKOS232-184A) and transferred into E. coli DH5α/pUB307.

The resulting E. coli DH5α/pUB307 cells were conjugated with S. fradiae K159-1/244-17a (Example 3) to produce Streptomyces fradiae K232-192. Streptomyces fradiae K232-192 was deposited under the terms of the Budapest Treaty with the American Type Culture Collection, 10801 University Blvd., Manassas, Va., 20110-2209, on Mar. 12, 2003, with accession number PTA-5052. Conjugation was performed as described in Practical Streptomyces Genetics (Kieser et al., 2000) except that plates were left overnight at 37° C. before overlaying with the selective agent (apramycin and naladixic acid). Apramycin resistant exconjugants were streaked for single colonies and a set of clones were patched onto R5 plates and inoculated into tryptic soy broth (40 ml in 250 ml shake flasks). Both the solid and liquid media contained apramycin (to select for pKOS232-184A) and kanamycin (to select for pKOS244-17A). Liquid and solid cultures were grown at 30° C. Agar plugs taken from most patches on R5 showed bioactivity when placed on an M. luteus test lawn. The agar was extracted with ethyl acetate and found to contain a compound of 730 amu. TSB seed cultures at 2-3 days were used to inoculate fermentation media and these cultures were grown for 7-10 days at 28° C. Upon extraction with ethyl acetate the 730 amu compound (730-I) was isolated and its structure verified by NMR as shown below. In addition, LC-MS analysis of the filtered culture broth showed abundant production of a 586 amu and a 730 amu (730-II) compound, and a 714 amu compound and a smaller amount of a 904 amu compound (most likely representing 14-methyl-platenolide with all three sugars attached). Thus, the chalcomycin-spiramycin hybrid PKS synthesized the predicted 14-methyl platenolide.

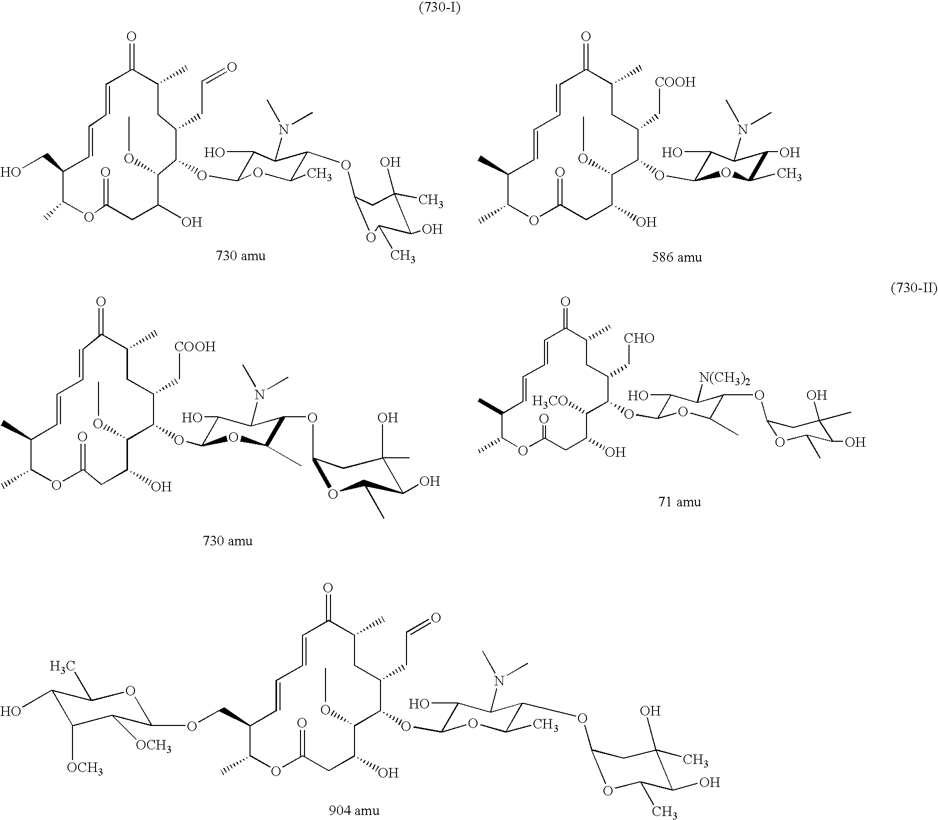

### Example 6

### Construction of Streptomyces fradiae K344-51 Expressing a Hybrid Chalcomycin-Spiramycin PKS and the chmH Gene

The chmH gene was cloned from cosmid pKOS146-185.1, which was deposited under the terms of the Budapest Treaty with the American Type Culture Collection, 10801 University Blvd., Manassas, Va., 20110-2209, on Feb. 19, 2003, with accession number PTA-4961.

A 6.3 Kb EcoRI fragment containing the chmH gene and a small downstream ferredoxin gene was cloned from cosmid pKOS146-185.1 into Litmus28 to give pKOS344-10B. This was then cut with SacI and religated to give pKOS344-016 having a 2.1 Kb insert. An NdeI site was introduced at the start of translation and an internal NdeI site was simultaneously replaced with a PstI site (without changing the amino acid sequence) in a three-piece ligation using two PCR products ligated between the EcoRI and BamHI sites of pKOS344-016 to give pKOS344-022B. The unique FseI site in pKOS344-022B was changed to an XbaI site with a synthetic linker and the chmH gene plus ferredoxin gene were excised with NdeI and XbaI and ligated into the expression vector, pKOS342-108D, between the NdeI and AvrII sites to give pKOS344-037B. This vector was transferred into DH5α/pUB307, and conjugated into K232-192 (Example 5). Exconjugants were selected with thiostrepton and streaked for single colonies to yield S. fradiae K344-51.

The vector for integration of chmH, pKOS342-108D, uses the int and att functions of Streptomyces phage φBT1. All S. fradiae strains were plated on AS1 agar for sporulation, R5 agar for solid media production, or grown in liquid TSB for vegetative growth and Russia medium for production. All appropriate antibiotics for selection of integrated markers were added to the media, except for the production stage.

Expression of the chmH gene (with its downstream ferredoxin) dramatically increased hydroxylation of the 14-methyl. A 906 amu compound was detected. This is the structure expected when all post-PKS tailoring of the tylosin pathway occurred, along with an additional reduction adding two hydrogen atoms. The methanol adduct characteristic of the aldehyde is not seen for the 906 amu compound, and reduction of the aldehyde most likely accounts for the addition of the two hydrogen atoms.

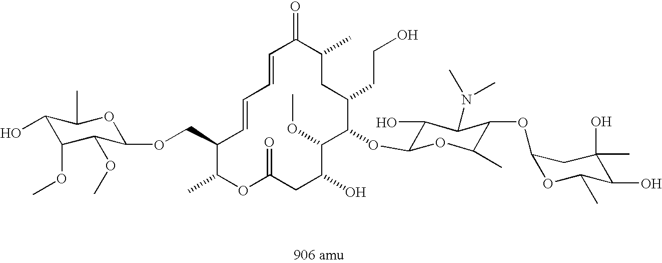

In addition, there appear to be a significant amount of 730 amu (730-I) compound that has the aldehyde and is hydroxylated on the 14-methyl. This is deduced by the presence of the methanol adduct (762 amu) and the fact that the 730 amu compound now elutes later from the C18 column compared with the 730 amu carboxylic acid seen prior to expression of chmH.

Without intending to be bound by a specific mechanism, FIG. 2 shows proposed pathways for post-PKS modification of the chalcomycin-spiramycin hybrid PKS macrolide product in the absence or presence of ChmH. When ChmH is present, the post-PKS reaction sequence from the Chm/Srm hybrid essentially follows that for tylosin and gives the 904 amu structure, which is converted by reduction of the aldehyde to a 906 amu compound. This reduction of the aldehyde has been described for tylosin (to give relomycin). Knockout of genes for allose biosynthesis or its transfer (tylJ), would give the demycinosyl compound of 730 amu (with the 14-hydroxymethyl and the aldehyde).

### Example 7

### Expression of a chmGI-GII Operon with the tvlG2 C-terminal Linker in S. fradiae K105-2

a) Construction of the S. fradiae tvlD Knockout Strain K168-173

The tylD knockout plasmid was constructed from two PCR products encompassing 1.8 kb regions upstream and downstream of the tyID gene using PCR primers that introduced new restriction sites. The upstream PCR product was cut with EcoRI and PstI and the downstream product was cut with PstI and SphI. These were then ligated together between the EcoRI and SphI sites of pUC19 and the sequence was verified. The resulting plasmid, pKOS168-106, has about 80% of the tylD gene deleted between the artificial PstI sites. This plasmid was introduced into S. fradiae by conjugation from E. coli DH5α/pUB307 and apramycin resistant exconjugants were obtained. Three were found by PCR to be the result of homologous recombination at the expected tylD locus and these were grown in the absence of selection and screened for the second crossover. Apramycin sensitive clones were isolated and some were found that produced demycinosyltylosin (DMT) by LC-MS analysis of the fermentation broths. The strain was designated S. fradiae K168-173.

b) Construction of S. fradiae tylD K105-2

The S. fradiae DMT (demycinosyltylosin) producer (K168-173) described above was used to introduce a KS-1 null mutation in the tylosin PKS. The plasmids pKOS168-190 and pKOS268-145 were digested with EcoRI and EcoRV and the 6.2 kb and 2.6 kb fragments, respectively, were gel isolated and ligated together to give pKOS264-65. A mutation was introduced into pKOS264-65 using PCR to change the active site cysteine of the tylosin KS1 to alanine, with the simultaneous introduction of an NheI site, to give pKOS325-8. Finally, pKOS325-8 and pKOS241-52 were digested with PvuII and XbaI and ligated together to give pKOS264-76. Plasmid pKOS264-76 was conjugated into the DMT producer strain S. fradiae K168-173 (Example 5) from E. coli DH5α/pUB307 and exconjugants were selected for apramycin resistance. Clones that underwent the correct first crossover event were identified by Southern blot analysis and one of these was propagated without selection to allow a second crossover. DNA from clones that had become apramycin sensitive was digested with XmaI/NheI and analyzed by Southern blot. Three clones had the pattern consistent with that expected for the desired second crossover to leave the KS1 -null mutation in the chromosome. This strain was designated S. fradiae K105-2, and was shown to produce no tylosin-related structure, but could convert O-mycaminosyl-tylonolide (OMT) into demycinosyl-tylosin (DMT).

c) Construction of a chmGI-GII Operon with the tylG2 C-terminal Linker

The tylG2 C-terminal linker region was isolated by PCR from a pSET-based vector encoding the including the entire tylosin PKS (pKOS168-190). The primers used were TylLink-A: 5′-TGAAGCTTCCCGCCACGCTGGTG-3′[SEQ ID NO:_] and TylLink-B: 5′-CGTCTAGACAGGGTGTGAAACCG-3′) [SEQ ID NO:_]. This created a HindIII site at the sane position of the encoded sequence corresponding to the natural HindIII site in the linker region of chmGII. The amplimer was cut with HindIII and XbaI and ligated into Litmus29 to give pKOS342-78. The tylosin linker region of pKOS342-78 was excised using HindIII and XbaI, and then ligated into HindIII and XbaI-digested pKOS232-172 (Example 5) to create pKOS342-82. This hybrid piece was then cut out with NdeI and XbaI and ligated to a pSET-based vector (pKOS232-189) cut with NdeI and SpeI to create the pSET152-based expression vector, pKOS342-84.

d) Expression of a chmGI-GII Operon with the tvlG2 C-terminal Linker in S. fradiae K105-2

The expression vector pKOS342-84 was transferred to E. coli DH5α/pUB307 and conjugated into S. fradiae K105-2 (this example, above). Apramycin resistant colonies were isolated and fermented in production medium. The broth was analyzed by LC-MS and found to contain the compound shown below. The chm/tyl hybrids differ from the chm/srm hybrids only by having a 4-methyl in place of a 4-methoxy, apparently making the chm/tyl good substrates for the TylH hydroxylase.

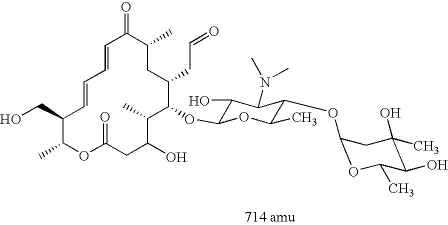

All publications and patent documents cited herein are incorporated herein by reference as if each such publication or document was specifically and individually indicated to be incorporated herein by reference.

Although the present invention has been described in detail with reference to specific embodiments, those of skill in the art will recognize that modifications and improvements are within the scope and spirit of the invention, as set forth in the claims which follow. Citation of publications and patent documents is not intended as an admission that any such document is pertinent prior art, nor does it constitute any admission as to the contents or date of the same. The invention having now been described by way of written description, those of skill in the art will recognize that the invention can be practiced in a variety of embodiments and that the foregoing description are for purposes of illustration and not limitation.

What is claimed is:

1. A recombinant DNA molecule that encodes a polypeptide, module or domain derived from a chalcomycin polyketide synthase (PKS) gene cluster.

2. The recombinant DNA molecule of claim 1 that comprises a sequence encoding a chalcomycin polyketide synthase module selected from the group consisting of modules 0 to 7.

3. The recombinant DNA molecule of claim 2 that comprises a sequence encoding a chalcomycin polyketide synthase polypeptide selected from the group consisting of ChmGI, ChmGII, ChmGIII, ChmGIV, and ChmV.

4. The recombinant DNA molecule of claim 1 that comprises a coding sequence for a chalcomycin modifying enzyme.

5. The recombinant DNA molecule of claim 4 that comprises a coding sequence for a chalcomycin P450 hydrolase enzyme selected from the group consisting of ChmHI, ChmPI, and ChmPII.

6. A vector that comprises a DNA molecule of claim 1.

7. The vector of claim 6 that is an expression vector.

8. A recombinant host cell comprising the vector of claim 6

9. A recombinant host cell comprising a DNA molecule of claim 1 integrated into the cell chromosomal DNA.

10. A chimeric PKS that comprises at least one domain of a chalcomycin PKS.

11. A cell comprising the chimeric PKS of claim 10

12. A modified functional chalcomycin PKS that differs from the S. bikiniensis chalcomycin PKS by the inactivation of at least one domain of the chalcomycin PKS and/or addition of at least one domain of a non-chalcomycin PKS.

13. The modified functional chalcomycin PKS of claim 12, wherein the domain of the chalcomycin PKS or the non-chalcomycin PKS is selected from the group consisting of a loading domain, a thioesterase domain, an AT domain, a KS domain, an ACP domain, a KR domain, a DH domain, and an ER domain.

14. A cell comprising the PKS of claim 12

15. A method to prepare an chalcomycin derivative which method comprises providing extender units to the cell of claim 14.

16. A recombinant expression system capable of producing a chalcomycin synthase domain in a host cell, said system comprising an encoding sequence for a chalcomycin polyketide synthase domain, and said encoding sequence being operably linked to control sequences effective in said cell to produce RNA that is translated into said domain.

17. A host cell modified to contain a recombinant expression system of claim 16.

18. An isolated polypeptide encoded by a recombinant polynucleotide of claim 1.

19. A recombinant host cell comprising a S. bikiniensis chalcomycin PKS polypeptide selected from the group consisting of ChmGI, ChmGII, ChmGIII, ChmGIV, and ChmV.

20. The host cell of claim 19 that is S. fradiae.

21. A recombinant S. bikiniensis cell in which a chmGI, chmGII, chmGIII, chmGIV, or chmV is disrupted so as to reduce or eliminate production of chalcomycin.

22. A recombinant DNA molecule encoding a first protein comprising one or more modules of a chalcomycin PKS and a second protein comprising one or more modules of a tylosin PKS or spiramycin PKS.

23. The DNA molecule of claim 22 wherein the hybrid polyketide synthase comprises one or more polypeptides of a chalcomycin PKS and one or more polypeptides of a tylosin PKS or spiramycin PKS.

24. A recombinant host cell comprising a hybrid polyketide synthase comprising one or more modules of a chalcomycin PKS and one or more modules of a tylosin PKS or spiramycin PKS.

25. A recombinant DNA molecule, comprising a sequence of at least about 200, optionally at least about 500, basepairs with a sequence identical or substantially identical to a protein encoding region of SEQ ID NO:1.

26. The DNA molecule of claim 25 that encodes a polypeptide, module or domain derived from a chalcomycin polyketide synthase (PKS) gene cluster.

27. A method of producing a polyketide, which method comprises growing the recombinant host cell of claim 17 or 24 under conditions whereby a polyketide synthesized by a PKS comprising a protein encoded by said recombinant DNA molecule is produced in the cell.

28. The method of claim 27 further comprising recovering the synthesized polyketide.

29. The method of claim 28 further comprising chemically modifying said polyketide.

30. The method of claim 28 further comprising formulating said polyketide for administration to a mammal.
